(19) United States
(12) Patent Application Publication
(10) Pub. No.: US 2012/0065195 A1
(43) Pub. Date: Mar. 15, 2012

(54) COMPOUNDS FOR TREATING NEURODEGENERATIVE DISEASES

(76) Inventors: Christopher T. Clark, Boulder, CO (US); Adam Cook, Boulder, CO (US); Indrani W. Gunawardana, Boulder, CO (US); Kevin W. Hunt, Boulder, CO (US); Nicholas C. Kallan, Boulder, CO (US); Michael Siu, South San Francisco, CA (US); Allen A. Thomas, Boulder, CO (US); Matthew Volgraf, South San Francisco, CA (US)

(21) Appl. No.: 13/077,477

(22) Filed: Mar. 31, 2011

Related U.S. Application Data
(60) Provisional application No. 61/319,341, filed on Mar. 31, 2010.
(60) Provisional application No. 61/439,723, filed on Feb. 4, 2011.

(51) Int. Cl. A61K 31/547; A61K 31/527; A61K 31/4439; A61K 31/423; A61K 31/4184; C07D 279/08; C07D 413/10; C07D 401/14; C07D 263/52; C07D 235/02; C07D 417/10; A61P 25/28; C07D 403/10; C07D 401/10; C07D 491/107; A61K 31/506

(52) U.S. Cl. 514/227.2; 514/256; 514/272; 514/278; 514/377; 514/389; 544/6; 544/230; 544/231; 546/15; 548/216; 548/301.1

(57) ABSTRACT

The invention provides novel spirotetrahydronaphthalene compounds of Formula α that inhibit β-secretase cleavage of APP and are useful as therapeutic agents for treating neurodegenerative diseases.

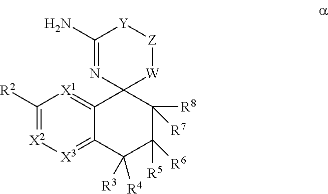

### BACKGROUND OF THE INVENTION

1. Field of the Invention

The present invention relates to organic compounds useful for inhibition of β-secretase enzymatic activity and the therapy and/or prophylaxis of neurodegenerative diseases associated therewith. More particularly, certain spirotetrahyrdonaphthalene compounds useful in the treatment and prevention of neurodegenerative diseases, such as Alzheimer's disease, are provided herein.

2. Description of the State of the Art

Alzheimer's disease (AD) is a neurological disorder thought to be primarily caused by amyloid plaques, an accumulation of abnormal protein deposits in the brain. It is believed that an increase in the production and accumulation of amyloid beta peptides (also referred to as Aβ or A-beta) in plaques leads to nerve cell death, which contributes to the development and progression of AD. Loss of nerve cells due to amyloid plaques in strategic brain areas, in turn, causes reduction in the neurotransmitters and impairment of memory. The proteins principally responsible for the plaque build up include amyloid precursor protein (APP) and presenilin I and II (PSI and PSII). Mutations in each of these three proteins have been observed to enhance proteolytic processing of APP via an intracellular pathway that produces Aβ peptides ranging from 39 to 43 amino acids. The Aβ 1-42 fragment has a particularly high propensity of forming aggregates due to two very hydrophobic amino acid residues at its C-terminus. Thus, Aβ 1-42 fragment is believed to be mainly responsible for the initiation of neuritic amyloid plaque formation in AD and is therefore actively being pursued as a therapeutic target. Anti-AP antibodies have been shown to reverse the histologic and cognitive impairments in mice which overexpress Aβ and are currently being tested in human clinical trials. Effective treatment requires anti-Aβ antibodies to cross the blood-brain barrier (BBB), however, antibodies typically cross the BBB very poorly and accumulate in the brain in low concentration.

Different forms of APP range in size from 695-770 amino acids, localize to the cell surface, and have a single C-terminal transmembrane domain. Aβ is derived from a region of APP adjacent to and containing a portion of the transmembrane domain. Normally, processing of APP by α-secretase cleaves the midregion of the Aβ sequence adjacent to the membrane and releases a soluble, extracellular domain fragment of APP from the cell surface referred to as APP-α. APP-α is not thought to contribute to AD. On the other hand, pathological processing of APP by the proteases β-secretase (also referred to as “β-site of APP cleaving enzyme” (BACE-1), memapsin-2 and Aspartyl Protease 2 (Asp2)) followed by γ-secretase cleavage, at sites which are located N-terminal and C-terminal to the α-secretase cleavage site, respectively, produces a very different result than processing at the α site, i.e. the release of amyloidogenic Aβ peptides, in particular, Aβ 1-42. Processing at the β- and γ-secretase sites can occur in both the endoplasmic reticulum and in the endosomal/lysosomal pathway after reinternalization of cell surface APP. Dysregulation of intracellular pathways for proteolytic processing may be central to the pathophysiology of AD. In the case of amyloid plaque formation, mutations in APP, PS1 or PS2 consistently alter the proteolytic processing of APP so as to enhance Aβ 1-42 formation.

The initial processing of APP by β-secretase results in a soluble N-APP which has recently been implicated in neuronal cell death through a pathway independent of amyloid plaque formation. N-APP is involved in normal pruning of neurons in early development in which relatively unused neurons and their nerve-fiber connections (axons) wither and degenerate. Recently, however, it has been shown that N-APP binds to and activates the apoptotic death receptor 6 (DR6) in vitro which is expressed on axons in response to trophic factor (e.g., nerve growth factor) withdrawal resulting in axonal degeneration. The aging process can lead to a reduction in the levels of growth factors in certain areas of the brain and/or the ability to sense growth factors. This in turn would lead to the release of N-APP fragment by cleavage of APP on neuronal surfaces, activating nearby DR6 receptors to initiate the axonal shrinkage and neuronal degeneration of Alzheimer's.

See also, Rauk, Arvi. “The chemistry of Alzheimer's disease.” Chem. Soc. Rev. 38 (2009): p. 2698-2715; Vassar, Robert, Dora M. Kovacs, Riqiang Yan and Philip C. Wong. “The •-Secretase Enzyme BACE in Health and Alzheimer's disease: Regulation, Cell Biology, Function, and Therapeutic Potential. J. Neurosci. 29(41) (2009): 12787-12794; and Silvestri, Romano. “Boom in the Development of Non-Peptidic β-Secretase (BACE1) Inhibitors for the Treatment of Alzheimer's Disease.” Medicinal Research Reviews. Vol. 29, No. 2 (2009): p. 295-338.

Since β-secretase cleavage of APP is essential for both amyloid plaque formation and DR6-mediated apoptosis, it is a key target in the search for therapeutic agents for treating AD.

### SUMMARY OF THE INVENTION

In one aspect of the present invention there is provided novel compounds having the general Formula α:

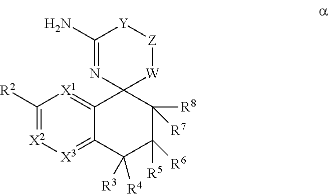

and stereoisomers, diastereomers, enantiomers, tautomers and pharmaceutically acceptable salts thereof, wherein W, X¹, X², X³, Y, Z, R², R³, R⁴, R⁵, R⁶, R⁷ and R⁸ are as defined herein.

In another aspect of the present invention there is provided novel compounds having the general Formula I:

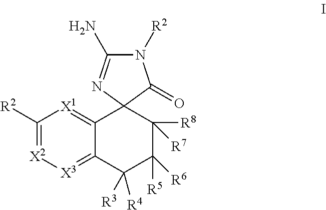

and stereoisomers, diastereomers, enantiomers, tautomers and pharmaceutically acceptable salts thereof, wherein X¹, X², X³, R¹, R², R³, R⁴, R⁵, R⁶, R⁷ and R⁸ are as defined herein.

In another aspect of the invention, there are provided pharmaceutical compositions comprising compounds of Formula α, α′, α″, I, I′, I″, II, II′, II″, III, III′, III″, IV, IV′, IV″, V, V′, V″, VI, VI′, VI″ or VII and a carrier, diluent or excipient.

In another aspect of the invention, there is provided a method of inhibiting cleavage of APP by β-secretase in a mammal comprising administering to said mammal an effective amount of a compound of Formula α, α′, α″, I, I′, I″, II, II′, II″, III, III′, III″, IV, IV′, IV″, V, V′, V″, VI, VI′, VI″ or VII.

In another aspect of the invention, there is provided a method for treating a disease or condition mediated by the cleavage of APP by β-secretase in a mammal, comprising administering to said mammal an effective amount of a compound of Formula α, α′, α″, I, I′, I″, II, II′, II″, III, III′, III″, IV, IV′, IV″, V, V′, V″, VI, VI′, VI″ or VII.

In another aspect of the invention, there is provided a use of a compound of Formula α, α′, α″, I, I′, I″, II, II′, II″, III, III′, III″, IV, IV′, IV″, V, V′, V″, VI, VI′, VI″ or VII in the manufacture of a medicament for the treatment of neurodegenerative diseases, such as Alzheimer's disease.

In another aspect of the invention, there is provided a use of a compound of Formula α, α′, α″, I, I′, I″, II, II′, II″, III, III′, III″, IV, IV′, IV″, V, V′, V″, VI, VI′, VI″ or VII in the treatment of neurodegenerative diseases, such as Alzheimer's disease.

Another aspect provides intermediates for preparing compounds of Formula α, α′, α″, I, I′, I″, II, II′, II″, III, III′, III″, IV, IV′, IV″, V, V′, V″, VI, VI′, VI″ or VII. Certain compounds of Formula α, α′, α″, I, I′, I″, II, II′, II″, III, III′, III″, IV, IV′, IV″, V, V′, V″, VI, VI′, VI″ or VII may be used as intermediates for other compounds of Formula α, α′, α″, I, I′, I″, II, II′, II″, III, III′, III″, IV, IV′, IV″, V, V′, V″, VI, VI′, VI″ or VII.

Another aspect includes processes for preparing, methods of separation, and methods of purification of the compounds described herein.

### DETAILED DESCRIPTION OF THE INVENTION

### Definitions

The term “acyl” means a carbonyl containing substituent represented by the formula —C(O)—R in which R is hydrogen, alkyl, a carbocycle, a heterocycle, carbocycle-substituted alkyl or heterocycle-substituted alkyl, wherein the alkyl, alkoxy, carbocycle and heterocycle are as defined herein. Acyl groups include alkanoyl (e.g., acetyl), aroyl (e.g., benzoyl), and heteroaroyl.

The term “alkoxycarbonyl” means the group —C(═O)OR in which R is alkyl. A particular alkoxycarbonyl group is C₁-C₆ alkoxycarbonyl, wherein the R group is C₁-C₆ alkyl.

The term “alkyl” means a branched or unbranched, saturated or unsaturated (i.e., alkenyl, alkynyl) aliphatic hydrocarbon group, having up to 12 carbon atoms unless otherwise specified. When used as part of another term, for example “alkylamino”, the alkyl portion may be a saturated hydrocarbon chain, however also includes unsaturated hydrocarbon carbon chains such as “alkenylamino” and “alkynylamino. Examples of particular alkyl groups are methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The terms “lower alkyl” “C₁-C₄ alkyl” and “alkyl of 1 to 4 carbon atoms” are synonymous and used interchangeably to mean methyl, ethyl, 1-propyl, isopropyl, cyclopropyl, 1-butyl, sec-butyl or t-butyl. In other examples, the alkyl group is C₁-C₂, C₁-C₃, C₁-C₄, C₁-C₅ or C₁-C₆. Unless specified otherwise, substituted alkyl groups contain one, two, three or four substituents which may be the same or different. Alkyl substituents are, unless otherwise specified, halogen, amino, hydroxyl, protected hydroxyl, mercapto, carboxy, alkoxy, nitro, cyano, amidino, guanidino, urea, oxo, sulfonyl, sulfinyl, aminosulfonyl, alkylsulfonylamino, arylsulfonylamino, aminocarbonyl, acylamino, alkoxy, acyl, acyloxy, an optionally substituted carbocycle and an optionally substituted heterocycle. Examples of the above substituted alkyl groups include, but are not limited to; cyanomethyl, nitromethyl, hydroxymethyl, trityloxymethyl, propionyloxymethyl, aminomethyl, carboxymethyl, carboxyethyl, carboxypropyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-amino(iso-propyl), 2-carbamoyloxyethyl and the like. The alkyl group may also be substituted with a carbocycle group. Examples include cyclopropylmethyl, cyclobutylmethyl, cyclopentylmethyl, and cyclohexylmethyl groups, as well as the corresponding -ethyl, -propyl, -butyl, -pentyl, -hexyl groups, etc. Substituted alkyls include substituted methyl, e.g., a methyl group substituted by the same substituents as the “substituted Cₙ-Cₘ alkyl” group. Examples of the substituted methyl group include groups such as hydroxymethyl, protected hydroxymethyl (e.g., tetrahydropyranyloxymethyl), acetoxymethyl, carbamoyloxymethyl, trifluoromethyl, chloromethyl, carboxymethyl, bromomethyl and iodomethyl.

The terms “alkenyl” and “alkynyl” also include linear or branched-chain radicals of carbon atoms.

The term “amidine” means the group —C(NH)—NHR in which R is hydrogen, alkyl, a carbocycle, a heterocycle, carbocycle-substituted alkyl or heterocycle-substituted alkyl wherein the alkyl, alkoxy, carbocycle and heterocycle are as defined herein. A particular amidine is the group —NH—C(NH)—NH₂.

The term “amino” means primary (i.e., —NH₂), secondary (i.e., —NRH) and tertiary (i.e., —NRR) amines in which R is hydrogen, alkyl, a carbocycle, a heterocycle, carbocycle-substituted alkyl or heterocycle-substituted alkyl wherein the alkyl, alkoxy, carbocycle and heterocycle are as defined herein. Particular secondary and tertiary amines are alkylamine, dialkylamine, arylamine, diarylamine, aralkylamine and diaralkylamine wherein the alkyl is as herein defined and optionally substituted. Particular secondary and tertiary amines are methylamine, ethylamine, propylamine, isopropylamine, phenylamine, benzylamine dimethylamine, diethylamine, dipropylamine and diisopropylamine.

The term “amino-protecting group” as used herein refers to a derivative of the groups commonly employed to block or protect an amino group while reactions are carried out on other functional groups on the compound. Examples of such protecting groups include carbamates, amides, alkyl and aryl groups, imines, as well as many N-heteroatom derivatives which can be removed to regenerate the desired amine group. Particular amino protecting groups are acetyl, trifluoroacetyl, t-butyloxycarbonyl (“Boc”), benzyloxycarbonyl (“CBz”) and 9-fluorenylmethyleneoxycarbonyl (“Fmoc”). Further examples of these groups, and other protecting groups, are found in T. W. Greene, et al. Greene's Protective Groups in Organic Synthesis. New York: Wiley Interscience, 2006.

The term “aryl” when used alone or as part of another term means a carbocyclic aromatic group whether or not fused having the number of carbon atoms designated or if no number is designated, up to 14 carbon atoms. Particular aryl groups are phenyl, naphthyl, biphenyl, phenanthrenyl, naphthacenyl, and the like (see e.g., Dean, J. A. Lange's Handbook of Chemistry. 15th ed. New York: McGraw-Hill Professional, 1998). A particular aryl is phenyl. Substituted phenyl or substituted aryl means a phenyl group or aryl group substituted with one, two, three, four or five substituents, for example 1-2, 1-3 or 1-4 substituents chosen, unless otherwise specified, from halogen (F, Cl, Br, I), hydroxy, protected hydroxy, cyano, nitro, alkyl (for example C₁-C₆ alkyl), alkoxy (for example C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, alkylsulfonylaminoalkyl, arylsulfonylamino, arylsulfonylaminoalkyl, heterocyclylsulfonylamino, heterocyclylsulfonylaminoalkyl, heterocyclyl, aryl, or other groups specified. One or more methyne (CH) and/or methylene (CH₂) groups in these substituents may in turn be substituted with a similar group as those denoted above. Examples of the term “substituted phenyl” includes but is not limited to a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(isopropyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such as 4-carboxyphenyl; a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; a mono- or di(N-(methylsulfonylamino))phenyl such as 3-(N-methylsulfonylamino))phenyl; disubstituted phenyl groups such as 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl and 2-hydroxy-4-chlorophenyl; trisubstituted phenyl groups such as 3-methoxy-4-benzyloxy-6-methylsulfonylamino and 3-methoxy-4-benzyloxy-6-phenylsulfonylamino; tetrasubstituted phenyl groups such as 3-methoxy-4-benzyloxy-5-methyl-6-phenyl sulfonylamino. Particular substituted phenyl groups include the 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. Fused aryl rings may also be substituted with any, for example 1, 2 or 3, of the substituents specified herein in the same manner as substituted alkyl groups.

The terms “carbocyclyl”, “carbocyclic”, “carbocycle” and “carbocyclo” alone and when used as a moiety in a complex group such as a carbocycloalkyl group, refer to a mono-, bi-, or tricyclic aliphatic ring having 3 to 14 carbon atoms, for example 3 to 7 carbon atoms or 3 to 6 carbon atoms, which may be saturated or unsaturated, aromatic or non-aromatic. Particular saturated carbocyclic groups are cyclopropyl, cyclobutyl, cyclopentyl and cyclohexyl groups. A particular saturated carbocycle is cyclopropyl. Another particular saturated carbocycle is cyclohexyl. Particular unsaturated carbocycles are aromatic, e.g., aryl groups as previously defined, for example phenyl. The terms “substituted carbocyclyl”, “carbocycle” and “carbocyclo” mean these groups substituted by the same substituents as the “substituted alkyl” group.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, alkyl such as t-butyl or t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. The species of carboxy-protecting group employed is not critical so long as the derivatized carboxylic acid is stable to the condition of subsequent reaction(s) on other positions of the molecule and can be removed at the appropriate point without disrupting the remainder of the molecule. In particular, it is important not to subject a carboxy-protected molecule to strong nucleophilic bases, such as lithium hydroxide or NaOH, or reductive conditions employing highly activated metal hydrides such as LiAlH₄. Such harsh removal conditions are also to be avoided when removing amino-protecting groups and hydroxy-protecting groups, discussed below. Particular carboxylic acid protecting groups are the alkyl (e.g., methyl, ethyl, t-butyl), allyl, benzyl and p-nitrobenzyl groups. The term “protected carboxy” refers to a carboxy group substituted with one of the above carboxy-protecting groups. Further examples are found in Greene's Protective Groups in Organic Synthesis, supra.

The terms “comprise” and “comprising” when used herein are non-limiting in scope, i.e., are intended to specify the presence of the stated features, integers, components, or steps, but do not preclude the presence or addition such features, integers, components, steps, or groups thereof.

The term “guanidine” means the group —NH—C(NH)—NHR in which R is hydrogen, alkyl, a carbocycle, a heterocycle, carbocycle-substituted alkyl or heterocycle-substituted alkyl, wherein the alkyl, alkoxy, carbocycle and heterocycle are as defined herein. A particular guanidine is the group —NH—C(NH)—NH₂.

The term “hydroxy-protecting group” as used herein refers to a derivative of the hydroxy group commonly employed to block or protect the hydroxy group while reactions are carried out on other functional groups on the compound. Examples of such protecting groups include tetrahydropyranyloxy, benzoyl, acetoxy, carbamoyloxy, benzyl, and silylethers (e.g., TBS, TBDPS) groups. Further examples are found in Greene's Protective Groups in Organic Synthesis, supra. The term “protected hydroxy” refers to a hydroxy group substituted with one of the above hydroxy-protecting groups.

The term “heterocyclic group”, “heterocyclic”, “heterocycle”, “heterocyclyl”, or “heterocyclo” alone and when used as a moiety in a complex group such as a heterocycloalkyl group, are used interchangeably and refer to any mono-, bi-, or tricyclic, saturated or unsaturated, aromatic (heteroaryl) or non-aromatic ring having the number of atoms designated, generally from 5 to about 14 ring atoms, where the ring atoms are carbon and at least one heteroatom (nitrogen, sulfur or oxygen), for example 1 to 4 heteroatoms. The sulfur heteroatoms may optionally be oxidized (e.g., SO, SO₂), and any nitrogen heteroatom may optionally be quaternized. Typically, a 5-membered ring has 0 to 2 double bonds and 6- or 7-membered ring has 0 to 3 double bonds. In a particular embodiment, heterocyclic groups are four to seven membered cyclic groups containing one, two or three heteroatoms selected from the group consisting of nitrogen, oxygen and sulfur. Particular non-aromatic heterocycles are morpholinyl (morpholino), pyrrolidinyl, oxiranyl, oxetanyl, tetrahydrofuranyl, 2,3-dihydrofuranyl, 2H-pyranyl, tetrahydropyranyl, thiiranyl, thietanyl, tetrahydrothietanyl, aziridinyl, azetidinyl, 1-methyl-2-pyrrolyl, piperazinyl and piperidinyl. A “heterocycloalkyl” group is a heterocycle group as defined above covalently bonded to an alkyl group as defined above. Particular 5-membered heterocycles containing a sulfur or oxygen atom and one to three nitrogen atoms are thiazolyl, in particular thiazol-2-yl and thiazol-2-yl N-oxide, thiadiazolyl, in particular 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Particular 5-membered ring heterocycles containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Particular benzo-fused 5-membered heterocycles are benzoxazol-2-yl, benzthiazol-2-yl and benzimidazol-2-yl. Particular 6-membered heterocycles contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are a particular group. Substituents for “optionally substituted heterocycles”, and further examples of the 5- and 6-membered ring systems discussed above can be found in W. Druckheimer et al., U.S. Pat. No. 4,278,793. In a particular embodiment, such optionally substituted heterocycle groups are substituted with hydroxyl, alkyl, alkoxy, acyl, halogen, mercapto, oxo, carboxyl, acyl, halo-substituted alkyl, amino, cyano, nitro, amidino and guanidino.

The term “heteroaryl” alone and when used as a moiety in a complex group such as a heteroaralkyl group, refers to any mono-, bi-, or tricyclic aromatic ring system having the number of atoms designated where at least one ring is a 5-, 6- or 7-membered ring containing from one to four heteroatoms selected from the group nitrogen, oxygen, and sulfur, and in a particular embodiment at least one heteroatom is nitrogen (see Lange's Handbook of Chemistry, supra). In a particular embodiment, the heteroaryl is a 5-membered aromatic ring containing one, two or three heteroatoms selected from nitrogen, oxygen and sulfur. Included in the definition are any bicyclic groups where any of the above heteroaryl rings are fused to a benzene ring. Particular heteroaryls incorporate a nitrogen or oxygen heteroatom. In a particular embodiment, the heteroaryl is a 5-membered aromatic ring containing one, two or three heteroatoms selected from nitrogen, oxygen and sulfur. In a particular embodiment, the heteroaryl group is a 6-membered aromatic ring containing one, two or three heteroatoms selected from nitrogen, oxygen and sulfur. The following are examples of the heteroaryl groups (substituted and unsubstituted): thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example benzoxazolyl, benzofuryl, benzothiazolyl, benzothiadiazolyl, benzotriazolyl, benzoimidazolyl and indolyl. In a particular embodiment the heteroaryl group may be: 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl. An alternative group of “heteroaryl” includes; 4-(carboxymethyl)-5-methyl-1,3-thiazol-2-yl, 4-(carboxymethyl)-5-methyl-1,3-thiazol-2-yl sodium salt, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl sodium salt, 2,5-dihydro-5-oxo-6-hydroxy-2-methyl-as-triazin-3-yl, tetrazolo[1,5-b]pyridazin-6-yl, or 8-aminotetrazolo[1,5-b]pyridazin-6-yl. Heteroaryl groups are optionally substituted as described for heterocycles.

The term “inhibitor” means a compound which reduces or prevents the enzymatic cleavage of APP by β-secretase. Alternatively, “inhibitor” means a compound which prevents or slows the formation of beta-amyloid plaques in mammalian brain. Alternatively, “inhibitor” means a compound that prevents or slows the progression of a disease or condition associated with β-secretase enzymatic activity, e.g., cleavage of APP. Alternatively, “inhibitor” means a compound which prevents Alzheimer's disease. Alternatively, “inhibitor” means a compound which slows the progression of Alzheimer's disease or its symptoms.

The term “optionally substituted” unless otherwise specified means that a group may be unsubstituted or substituted by one or more (e.g. 0, 1, 2, 3 or 4) of the substituents listed for that group in which said substituents may be the same or different. In a particular embodiment, an optionally substituted group has 1 substituent. In another embodiment an optionally substituted group has 2 substituents. In another embodiment an optionally substituted group has 3 substituents. In another embodiment, an optionally substituted group has 1 to 3 substituents.

The term “pharmaceutically acceptable” indicates that the substance or composition is compatible chemically and/or toxicologically, with the other ingredients comprising a formulation, and/or the mammal being treated therewith.

The term “pharmaceutically acceptable salts” include both acid and base addition salts.

The term “pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid and the like.

The term “pharmaceutically acceptable base addition salts” include those derived from inorganic bases such as sodium, potassium, lithium, ammonium, calcium, magnesium, iron, zinc, copper, manganese, aluminum salts and the like. Particularly base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly organic non-toxic bases are isopropylamine, diethylamine, ethanolamine, trimethamine, dicyclohexylamine, choline, and caffeine.

The term “sulfanyl” means —S—R group in which R is alkyl, a carbocycle, a heterocycle, carbocycle-substituted alkyl or heterocycle-substituted alkyl, wherein the alkyl, alkoxy, carbocycle and heterocycle are as defined herein. Particular sulfanyl groups are alkylsulfanyl (i.e., —SO₂-alkyl), for example methylsulfanyl; arylsulfanyl, for example phenylsulfanyl; aralkylsulfanyl, for example benzylsulfanyl.

The term “sulfinyl” means —SO—R group in which R is hydrogen, alkyl, a carbocycle, a heterocycle, carbocycle-substituted alkyl or heterocycle-substituted alkyl, wherein the alkyl, alkoxy, carbocycle and heterocycle are as defined herein. Particular sulfonyl groups are alkylsulfinyl (i.e., —SO-alkyl), for example methylsulfinyl; arylsulfinyl, for example phenylsulfinyl; aralkylsulfinyl, for example benzylsulfinyl.

The term “sulfonyl” means a —SO₂—R group in which R is hydrogen, alkyl, a carbocycle, a heterocycle, carbocycle-substituted alkyl or heterocycle-substituted alkyl wherein the alkyl, alkoxy, carbocycle and heterocycle are as defined herein. Particular sulfonyl groups are alkylsulfonyl (i.e., —SO₂-alkyl), for example methylsulfonyl; arylsulfonyl, for example phenylsulfonyl; aralkylsulfonyl, for example benzylsulfonyl.

The terms “treat” or “treatment” refer to therapeutic, prophylactic, palliative or preventative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable.

“Treatment” can also mean prolonging survival as compared to expected survival if not receiving treatment. Those in need of treatment include those already with the condition or disorder, as well as those prone to have the condition or disorder or those in which the condition or disorder is to be prevented.

The phrases “therapeutically effective amount” or “effective amount” mean an amount of a compound described herein that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art. The “effective amount” of the compound to be administered will be governed by such considerations, and is the minimum amount necessary to inhibit cleavage of APP by β-secretase, for example by 10% or greater in situ. In a particular embodiment an “effective amount” of the compound inhibits cleavage of APP by β-secretase by 25% or greater in situ. In a particular embodiment the effective amount inhibits cleavage of APP by β-secretase by 50% or greater in situ. In a particular embodiment the effective amount inhibits cleavage of APP by β-secretase by 70% or greater in situ. In a particular embodiment the effective amount inhibits cleavage of APP by β-secretase by 80% or greater in situ. In a particular embodiment the effective amount inhibits cleavage of APP by β-secretase by 90% or greater in situ. Such amount may be below the amount that is toxic to normal cells, or the mammal as a whole. Alternatively, an “effective amount” is the amount of compound necessary to reduce A-beta levels in plasma or cerebrospinal fluid of a mammal, for example, by 10% or greater. In a particular embodiment, an “effective amount” is the amount of compound necessary to reduce A-beta levels in plasma or cerebrospinal fluid of a mammal by 25% or greater. In a particular embodiment, an “effective amount” is the amount of compound necessary to reduce A-beta levels in plasma or cerebrospinal fluid of a mammal by 50% or greater. In a particular embodiment, an “effective amount” is the amount of compound necessary to reduce A-beta levels in plasma or cerebrospinal fluid of a mammal by 75% or greater. Alternatively, an “effective amount” of the compound may be the amount of compound necessary to slow the progression of AD or symptoms thereof.

Abbreviations are sometimes used in conjunction with elemental abbreviations and chemical structures, for example, methanol (“MeOH”), ethanol (“EtOH”) or ethyl acetate (“EtOAc”). Additional abbreviations used throughout the application may include, for example, benzyl (“Bn”), phenyl (“Ph”) and acetate (“Ac”).

Spirotetrahydronaphthalene Compounds

Provided herein are compounds, and pharmaceutical formulations thereof, that are potentially useful in the treatment of diseases, conditions and/or disorders modulated by BACE-1.

One embodiment provides compounds of Formula a:

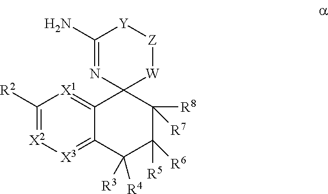

and stereoisomers, diastereomers, enantiomers, tautomers and pharmaceutically acceptable salts thereof, wherein:
W is a bond or CR¹⁰R¹¹;
Y is O, S or NR¹;
Z is CR¹²R¹³ or C(═O), provided when Z is C(═O) then Y is NR¹;
X¹, X² and X³ are independently selected from CR⁹ and N, wherein only one of X¹, X² or X³ may be N;
R¹ is selected from hydrogen, alkyl, aralkyl, heteroaryl or heteroaralkyl;
R² is selected from hydroxy, halogen, amino, cyano, nitro, alkyl, alkoxy, acyl, acyloxy, alkoxycarbonyl, sulfonyl, sulfinyl, sulfanyl, aryloxy, a carbocycle or a heterocycle wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl, optionally substituted carbocycle or heterocycle;
R³ and R⁴ are independently selected from hydrogen, halogen and alkyl, or
R³ and R⁴ together form an oxo group;
R⁵ and R⁶ are independently hydrogen, hydroxy, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, an optionally substituted carbocycle and an optionally substituted heterocycle, or
R⁵ and R⁶ together form a 3 to 6 member carbocycle or heterocycle optionally substituted with hydroxy, halogen, amino, cyano, nitro, alkyl, alkoxy, acyl and haloalkyl;
R⁷ and R⁸ are independently hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, a carbocycle and an optionally substituted heterocycle, or
R⁷ and R⁸ together form a 3 to 6 member carbocycle or heterocycle optionally substituted with hydroxy, halogen, amino, cyano, nitro, alkyl, alkoxy, acyl and haloalkyl;
R⁹ is independently is hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl and haloalkyl,
or R⁵ and R⁷ together form a 3 to 4 member carbocycle or heterocycle optionally substituted with hydroxy, halogen, amino, cyano, nitro, alkyl, alkoxy, acyl and haloalkyl;
each R⁹ is independently selected from hydrogen, halogen or methyl;
R¹⁰ and R¹¹ are independently selected from hydrogen and alkyl, or
R¹⁰ and R¹¹ together with the atom to which they are attached form a 3 to 6 membered carbocycle or heterocycle; and
R¹² and R¹³ are independently selected from hydrogen, alkyl and a carbocycle.

In certain embodiments:
W is a bond or CR¹⁰R¹¹;
Y is O, S or NR¹;
Z is CR¹²R¹³ or C(═O), provided when Z is C(═O) then Y is NR¹;
X¹, X² and X³ are independently selected from CR⁹ and N, wherein only one of X¹, X² or X³ may be N;
R¹ is selected from hydrogen, alkyl, aralkyl, heteroaryl or heteroaralkyl;
R² is selected from hydroxy, halogen, amino, cyano, nitro, alkyl, alkoxy, acyl, acyloxy, alkoxycarbonyl, sulfonyl, sulfinyl, sulfanyl, aryloxy, a carbocycle or a heterocycle wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl and optionally substituted carbocycle;
R³ and R⁴ are independently selected from hydrogen, halogen and alkyl;
R⁵ and R⁶ are independently hydrogen, hydroxy, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, an optionally substituted carbocycle and an optionally substituted heterocycle, or
R⁵ and R⁶ together form a 3 to 6 member carbocycle or heterocycle optionally substituted with hydroxy, halogen, amino, cyano, nitro, alkyl, alkoxy, acyl and haloalkyl;
R⁷ and R⁸ are independently hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, a carbocycle and an optionally substituted heterocycle, or
R⁷ and R⁸ together form a 3 to 6 member carbocycle or heterocycle optionally substituted with hydroxy, halogen, amino, cyano, nitro, alkyl, alkoxy, acyl and haloalkyl;
R⁹ is independently is hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl and haloalkyl;
each R⁹ is independently selected from hydrogen, halogen or methyl;
R¹⁰ and R¹¹ are independently selected from hydrogen and alkyl, or
R¹⁰ and R¹¹ together with the atom to which they are attached form a 3 to 6 membered carbocycle or heterocycle; and
R¹² and R¹³ are independently selected from hydrogen, alkyl and a carbocycle.

In certain embodiments:
W is a bond or CR¹⁰R¹¹;
Y is O, S or NR¹;
Z is CR¹²R¹³ or C(═O), provided when Z is C(═O) then Y is NR¹;
X¹, X² and X³ are independently selected from CR⁹ and N, wherein only one of X¹, X² or X³ may be N;
R¹ is selected from hydrogen, benzyl or C₁-C₃ alkyl optionally substituted with R^{a};
R² is halogen, CN, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated heterocyclyl optionally substituted with R^{d}, a 3 to 6 membered saturated or unsaturated carbocyclyl optionally substituted with R^{d}, a 9 to 10 membered bicyclic heteroaryl optionally substituted with R^{c}, a 9 to 10 membered bicyclic heterocyclyl optionally substituted with R^{c}, phenylamino, phenoxy optionally substituted with R^{e}, or —NHC(═O)R^{o};
R³ and R⁴ are independently selected from hydrogen, halogen and C₁-C₆ alkyl, or
R³ and R⁴ together form an oxo group;
R⁵ and R⁶ are independently selected from hydrogen, a 3 to 6 membered saturated or unsaturated carbocyclyl, or C₁-C₆ alkyl optionally substituted with R^{f}, or
R⁵ and R⁶ together with the atom to which they are attached form a 3 to 6 membered carbocyclyl or heterocyclyl;
R⁷ and R⁸ are independently selected from hydrogen, halogen or C₁-C₆ alkyl optionally substituted with R^{f}, or
R⁷ and R⁸ together with the atom to which they are attached form a 3 to 6 membered carbocyclyl or heterocyclyl, or
R⁵ and R⁷ together with the atoms to which they are attached form a 3 to 4 membered carbocyclyl or heterocyclyl, wherein only one of the pairs of R⁵ and R⁶, R⁷ and R⁸ or R⁵ and R⁷ may together form a ring;
each R⁹ is independently selected from hydrogen, halogen or methyl;
R¹⁰ and R¹¹ are independently selected from hydrogen and C₁-C₃ alkyl, or
R¹⁰ and R¹¹ together with the atom to which they are attached form a 3 to 6 membered carbocycle or heterocycle;
R¹² and R¹³ are independently selected from hydrogen, C₁-C₆ alkyl and C₃-C₆ carbocyclyl;
each R^{a} is independently selected from OH, OCH₃, halogen, a 5 to 6 membered heteroaryl, and a 3-6 membered heterocyclyl optionally substituted with C₁-C₃ alkyl optionally substituted with oxo;
each R^{b} is independently selected from halogen, CN, OH, OCH₃, cyclopropyl and phenyl optionally substituted with halogen, OH or OCH₃;
each R^{c} is independently selected from halogen, CN, a 3 to 6 membered carbocyclyl, a 5 to 6 membered heteroaryl, a 3 to 6 membered heterocyclyl, phenyl, OR^{g}, SR^{h}, NRⁱR^{j}, C₁-C₈ alkyl optionally substituted with R^{k}, C₁-C₈ alkynyl optionally substituted with R^{k};
each R^{d} is independently selected from halogen, oxo, C₁-C₆ alkyl, and C₁-C₆ alkoxycarbonyl;
each R^{e} is independently selected from halogen and benzyl;
each R^{f} is independently selected from halogen, oxo, OH, NR^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with halogen, C₁-C₆ alkyl optionally substituted with halogen, —O(C₁-C₆ alkyl) optionally substituted with halogen, phenyl or a 5 to 6 membered heteroaryl;
each R^{g} is independently selected from hydrogen and C₁-C₆ alkyl optionally substituted with halogen or phenyl;
each R^{h} is C₁-C₆ alkyl;
each Rⁱ and R^{j} are independently selected from hydrogen and C₁-C₆ alkyl;
each R^{k} is independently selected from halogen, OH, OCH₃, phenyl and a 3 to 6 membered carbocyclyl;
each R^{m} and Rⁿ are independently selected from hydrogen and C₁-C₆ alkyl; and
R^{o} is C₁-C₆ alkyl, C₃-C₆ cycloalkyl, a 4 to 6 membered heterocyclyl, phenyl or a 5 to 6 membered heteroaryl, wherein the alkyl, cycloalkyl, heterocyclyl, phenyl and heteroaryl are optionally substituted with halogen, C₁-C₃ alkyl, and C₁-C₃ alkoxyl.

In certain embodiments:
W is a bond or CR¹⁰R¹¹;
Y is O, S or NR¹;
Z is CR¹²R¹³ or C(═O), provided when Z is C(═O) then Y is NR¹;
X¹, X² and X³ are independently selected from CR⁹ and N, wherein only one of X¹, X² or X³ may be N;
R¹ is selected from hydrogen, benzyl or C₁-C₃ alkyl optionally substituted with R^{a};
R² is halogen, CN, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated heterocyclyl optionally substituted with R^{d}, a 3 to 6 membered saturated or unsaturated carbocyclyl optionally substituted with R^{d}, a 9 to 10 membered bicyclic heteroaryl optionally substituted with R^{c}, a 9 to 10 membered bicyclic heterocyclyl optionally substituted with R^{c}, phenylamino, phenoxy optionally substituted with R^{e}, or —NHC(═O)R^{o};
R³ and R⁴ are independently selected from hydrogen, halogen and C₁-C₆ alkyl;
R⁵ and R⁶ are independently selected from hydrogen, a 3 to 6 membered saturated or unsaturated carbocyclyl, or C₁-C₆ alkyl optionally substituted with R^{f}, or
R⁵ and R⁶ together with the atom to which they are attached form a 3 to 6 membered carbocyclyl or heterocyclyl;
R⁷ and R⁸ are independently selected from hydrogen, halogen or C₁-C₆ alkyl optionally substituted with R^{f}, or
R⁷ and R⁸ together with the atom to which they are attached form a 3 to 6 membered carbocyclyl or heterocyclyl, wherein only one of the pairs of R⁵ and R⁶ or R⁷ and R⁸ may together form a ring;
each R⁹ is independently selected from hydrogen, halogen or methyl;
R¹⁰ and R¹¹ are independently selected from hydrogen and C₁-C₃ alkyl, or
R¹⁰ and R¹¹ together with the atom to which they are attached form a 3 to 6 membered carbocycle or heterocycle;
R¹² and R¹³ are independently selected from hydrogen, C₁-C₆ alkyl and C₃-C₆ carbocyclyl;
each R^{a} is independently selected from OH, OCH₃, halogen, a 5 to 6 membered heteroaryl, and a 3-6 membered heterocyclyl optionally substituted with C₁-C₃ alkyl optionally substituted with oxo;
each R^{b} is independently selected from halogen, CN, OH, OCH₃, cyclopropyl and phenyl optionally substituted with halogen, OH or OCH₃;
each R^{c} is independently selected from halogen, CN, a 3 to 6 membered carbocyclyl, OR^{g}, SR^{h}, NRⁱR^{j}, C₁-C₈ alkyl optionally substituted with R^{k}, C₁-C₈ alkynyl optionally substituted with R^{k};
each R^{d} is independently selected from halogen, oxo, C₁-C₆ alkyl, and C₁-C₆ alkoxycarbonyl;
each R^{e} is independently selected from halogen and benzyl;
each R^{f} is independently selected from halogen, oxo, OH, NR^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with C₁-C₆ alkyl optionally substituted with halogen and —O(C₁-C₆ alkyl) optionally substituted with halogen;
each R^{g} is independently selected from hydrogen and C₁-C₆ alkyl optionally substituted with halogen or phenyl;
each R^{h} is C₁-C₆ alkyl;
each Rⁱ and R^{j} are independently selected from hydrogen and C₁-C₆ alkyl;
each R^{k} is independently selected from halogen, OH, OCH₃, phenyl and a 3 to 6 membered carbocyclyl;
each R^{m} and Rⁿ are independently selected from hydrogen and C₁-C₆ alkyl; and
R^{o} is C₁-C₆ alkyl, C₃-C₆ cycloalkyl, a 4 to 6 membered heterocyclyl, phenyl or a 5 to 6 membered heteroaryl, wherein the alkyl, cycloalkyl, heterocyclyl, phenyl and heteroaryl are optionally substituted with halogen, C₁-C₃ alkyl, and C₁-C₃ alkoxyl.

In certain embodiments:
W is a bond or CR¹⁰R¹¹;
Y is O, S or NR¹;
Z is CR¹²R¹³ or C(═O), provided when Z is C(═O) then Y is NR¹;
X¹ and X² are selected from CR⁹ and N, and X³ is CR⁹, wherein only one of X¹ or X² may be N;
R¹ is C₁-C₃ alkyl optionally substituted with R^{a};
R² is halogen, C₁-C₈ alkyl optionally substituted with R^{h}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated carbocyclyl optionally substituted with R^{d}, a 9 to 10 membered bicyclic heterocyclyl, or —NHC(═O)R^{o};
R³ and R⁴ are independently selected from hydrogen and C₁-C₆ alkyl, or
R³ and R⁴ together form an oxo group;
R⁵ and R⁶ are independently selected from hydrogen or C₁-C₆ alkyl optionally substituted with R^{f}, or
R⁵ and R⁶ together with the atom to which they are attached form a 3 to 6 membered carbocyclyl or heterocyclyl;
R⁷ and R⁸ are independently selected from hydrogen, halogen or C₁-C₆ alkyl optionally substituted with R^{f}, or
R⁷ and R⁸ together with the atom to which they are attached form a 3 to 6 membered heterocyclyl, or
R⁵ and R⁷ together with the atoms to which they are attached form a 3 to 4 membered carbocyclyl or heterocyclyl, wherein only one of the pairs of R⁵ and R⁶, R⁷ and R⁸ or R⁵ and
R⁷ may together form a ring;
each R⁹ is hydrogen;
R¹⁰ and R¹¹ are hydrogen;
R¹² and R¹³ are independently selected from hydrogen and C₁-C₆ alkyl;
each R^{a} is halogen;
each R^{b} is independently selected from CN and cyclopropyl;
each R^{c} is independently selected from halogen, CN, OR^{g}, SR^{h}, C₁-C₈ alkyl and a 5 to 6 membered heteroaryl;
each R^{f} is independently selected from halogen, OH, phenyl, a 5 to 6 membered heteroaryl and a 4 to 6 membered heterocyclyl, wherein the phenyl, heteroaryl and heterocyclyl are optionally substituted with halogen, C₁-C₆ alkyl optionally substituted with halogen, or a 5 to 6 membered heteroaryl;
R^{g} is C₁-C₆ alkyl optionally substituted with halogen;
R^{h} is C₁-C₆ alkyl; and
R^{o} is phenyl or a 5 to 6 membered heteroaryl, wherein the phenyl and heteroaryl are optionally substituted with halogen, C₁-C₃ alkyl, and C₁-C₃ alkoxyl.

In certain embodiments:
W is a bond or CR¹⁰R¹¹;
Y is O, S or NR¹;
Z is CH₂ or C(═O), provided when Z is C(═O) then Y is NR¹;
X¹ and X² are selected from CR⁹ and N, and X³ is CR⁹;
R¹ is C₁-C₃ alkyl;
R² is halogen, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated carbocyclyl optionally substituted with R^{d}, a 9 to 10 membered bicyclic heterocyclyl, or —NHC(═O)R^{o};
R³ and R⁴ are independently selected from hydrogen and C₁-C₆ alkyl;
R⁵ and R⁶ are independently selected from hydrogen or C₁-C₆ alkyl optionally substituted with R^{f}, or
R⁵ and R⁶ together with the atom to which they are attached form a 3 to 6 membered carbocyclyl or heterocyclyl;
R⁷ and R⁸ are independently selected from hydrogen or C₁-C₆ alkyl optionally substituted with R^{f}, or
R⁷ and R⁸ together with the atom to which they are attached form a 3 to 6 membered heterocyclyl;
each R⁹ is hydrogen;
R¹⁰ and R¹¹ are hydrogen;
each R^{b} is independently selected from CN and cyclopropyl;
each R^{c} is independently selected from halogen, CN, OR^{g}, SR^{h}, and C₁-C₈ alkyl;
each R^{f} is independently selected from OH, a 5 to 6 membered heteroaryl and a 4 to 6 membered heterocyclyl optionally substituted with C₁-C₆ alkyl optionally substituted with halogen;
R^{g} is C₁-C₆ alkyl optionally substituted with halogen;
R^{h} is C₁-C₆ alkyl; and
R^{o} is phenyl or a 5 to 6 membered heteroaryl, wherein the phenyl and heteroaryl are optionally substituted with halogen, C₁-C₃ alkyl, and C₁-C₃ alkoxyl.

In certain embodiments, W is a bond or CR¹⁰R¹¹. In certain embodiments, W is a bond. In certain embodiments, W is CR¹⁰R¹¹. In certain embodiments, R¹⁰ and R¹¹ are independently selected from hydrogen and C₁-C₃ alkyl, or R¹⁰ and R¹¹ together with the atom to which they are attached form a 3 to 6 membered carbocycle or heterocycle. In certain embodiments, R¹⁰ and R¹¹ are hydrogen. In certain embodiments, R¹⁰ and R¹¹ together with the atom to which they are attached form a 3 to 6 membered carbocycle or heterocycle. In certain embodiments, R¹⁰ and R¹¹ together with the atom to which they are attached form a C₃-C₆ carbocycle. In certain embodiments, R¹⁰ and R¹¹ together with the atom to which they are attached form a 3 to 6 membered heterocycle, wherein the heterocycle contains one or two heteroatoms selected from nitrogen, oxygen and sulfur.

In certain embodiments, Y is O, S or NR¹. In certain embodiments, Y is O. In certain embodiments, Y is S. In certain embodiments, Y is NR¹.

In certain embodiments, Z is CR¹²R¹³ or C(═O), provided when Z is C(═O) then Y is NR¹. In certain embodiments, Z is C(═O) and then Y is NR¹. In certain embodiments, Z is CR¹²R¹³. In certain embodiments, R¹² and R¹³ are independently selected from hydrogen and C₁-C₆ alkyl. In certain embodiments, R¹² and R¹³ are independently selected from hydrogen and methyl. In certain embodiments, R¹² and R¹³ are hydrogen. In certain embodiments, R¹² is methyl and R¹³ is hydrogen. In certain embodiments, Z is CH₂. in certain embodiments, Z is CH(CH₃).

In certain embodiments, Z is CR¹²R¹³ or C(═O), provided when Z is C(═O) then Y is NR¹. In certain embodiments, R¹² and R¹³ are independently selected from hydrogen, C₁-C₆ alkyl and C₃-C₆ carbocyclyl. In certain embodiments, R¹² and R¹³ are hydrogen. In certain embodiments, Z is CH₂ or C(═O), provided when Z is C(═O) then Y is NR¹. In certain embodiments, Z is CH₂. In certain embodiments, Z is C(═O) and Y is NR¹.

One embodiment provides compounds of Formula I:

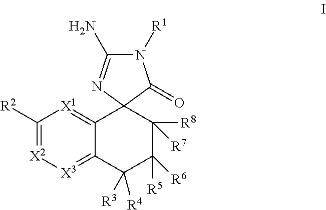

and stereoisomers, diastereomers, enantiomers, tautomers and pharmaceutically acceptable salts thereof, wherein X¹, X², X³, R¹, R², R³, R⁴, R⁵, R⁶, R⁷ and R⁸ are as defined herein.

One embodiment provides compounds of Formula II:

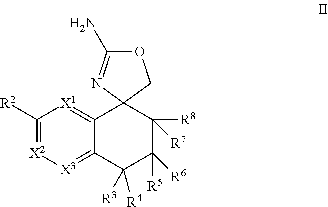

and stereoisomers, diastereomers, enantiomers, tautomers and pharmaceutically acceptable salts thereof, wherein X¹, X², X³, R², R³, R⁴, R⁵, R⁶, R⁷ and R⁸ are as defined herein.

One embodiment provides compounds of Formula III:

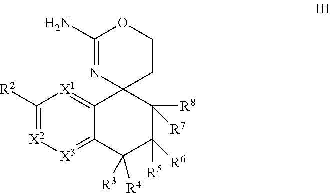

and stereoisomers, diastereomers, enantiomers, tautomers and pharmaceutically acceptable salts thereof, wherein X¹, X², X³, R², R³, R⁴, R⁵, R⁶, R⁷ and R⁸ are as defined herein.

One embodiment provides compounds of Formula IV:

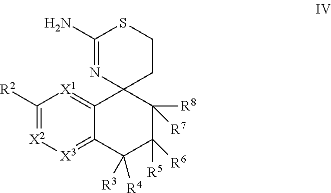

and stereoisomers, diastereomers, enantiomers, tautomers and pharmaceutically acceptable salts thereof, wherein X¹, X², X³, R², R³, R⁴, R⁵, R⁶, R⁷ and R⁸ are as defined herein.

One embodiment provides compounds of Formula V:

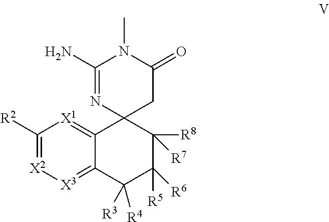

and stereoisomers, diastereomers, enantiomers, tautomers and pharmaceutically acceptable salts thereof, wherein X¹, X², X³, R², R³, R⁴, R⁵, R⁶, R⁷ and R⁸ are as defined herein.

One embodiment provides compounds of Formula VI:

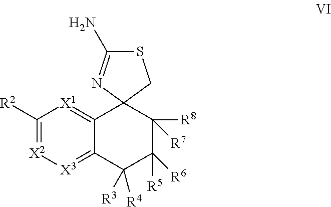

and stereoisomers, diastereomers, enantiomers, tautomers and pharmaceutically acceptable salts thereof, wherein X¹, X², X³, R², R³, R⁴, R⁵, R⁶, R⁷ and R⁸ are as defined herein.

In certain embodiments, W is a bond. When W is a bond, the compounds of Formula a have the structure of Formula VII:

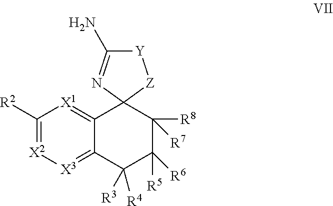

wherein X¹, X², X³, Y, Z, R², R³, R⁴, R⁵, R⁶, R⁷ and R⁸ are as defined herein.

One embodiment provides compounds of Formula I:

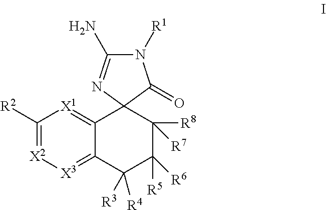

and stereoisomers, diastereomers, enantiomers, tautomers and pharmaceutically acceptable salts thereof, wherein:
X¹, X² and X³ are independently selected from CR⁹ and N, wherein only one of X¹, X² or X³ may be N;
R¹ is selected from hydrogen, alkyl, aralkyl, heteroaryl or heteroaralkyl;
R² is selected from hydroxy, halogen, amino, cyano, nitro, alkyl, alkoxy, acyl, acyloxy, alkoxycarbonyl, sulfonyl, sulfinyl, sulfanyl, aryloxy, a carbocycle or a heterocycle wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl and optionally substituted carbocycle;
R³ and R⁴ are independently selected from hydrogen, halogen and alkyl;
R⁵ and R⁶ are independently hydrogen, hydroxy, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, an optionally substituted carbocycle and an optionally substituted heterocycle, or
R⁵ and R⁶ together form a 3 to 6 member carbocycle or heterocycle optionally substituted with hydroxy, halogen, amino, cyano, nitro, alkyl, alkoxy, acyl and haloalkyl;
R⁷ and R⁸ are independently hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, a carbocycle and an optionally substituted heterocycle, or
R⁷ and R⁸ together form a 3 to 6 member carbocycle or heterocycle optionally substituted with hydroxy, halogen, amino, cyano, nitro, alkyl, alkoxy, acyl and haloalkyl;
R⁹ is independently is hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl and haloalkyl.

In certain embodiments of Formula I:
X¹, X² and X³ are independently selected from CR⁹ and N, wherein only one of X¹, X² or X³ may be N;
R¹ is selected from hydrogen, benzyl or C₁-C₃ alkyl optionally substituted with R^{a};
R² is halogen, CN, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated heterocyclyl optionally substituted with R^{d}, a 3 to 6 membered saturated or unsaturated carbocyclyl optionally substituted with R^{d}, a 9 to 10 membered bicyclic heteroaryl optionally substituted with R^{e}, a 9 to 10 membered bicyclic heterocyclyl optionally substituted with R^{e}, phenylamino, or phenoxy optionally substituted with R^{e};
R³ and R⁴ are independently selected from hydrogen, halogen and C₁-C₆ alkyl;
R⁵ and R⁶ are independently selected from hydrogen, a 3 to 6 membered saturated or unsaturated carbocyclyl, or C₁-C₆ alkyl optionally substituted with R^{f}, or
R⁵ and R⁶ together with the atom to which they are attached form a 3 to 6 membered carbocyclyl or heterocyclyl;
R⁷ and R⁸ are independently selected from hydrogen, halogen or C₁-C₆ alkyl optionally substituted with R^{f}, or
R⁷ and R⁸ together with the atom to which they are attached form a 3 to 6 membered carbocyclyl or heterocyclyl, wherein only one of the pairs of R⁵ and R⁶ or R⁷ and R⁸ may together form a ring;
each R⁹ is independently selected from hydrogen, halogen or methyl;
each R^{a} is independently selected from OH, OCH₃, halogen, a 5 to 6 membered heteroaryl, and a 3-6 membered heterocyclyl optionally substituted with C₁-C₃ alkyl optionally substituted with oxo;
each R^{b} is independently selected from halogen, CN, OH, OCH₃, cyclopropyl and phenyl optionally substituted with halogen, OH or OCH₃;
each R^{c} is independently selected from halogen, CN, a 3 to 6 membered carbocyclyl, OR^{g}, SR^{h}, NRⁱR^{j}, C₁-C₈ alkyl optionally substituted with R^{k}, C₁-C₈ alkynyl optionally substituted with R^{k};
each R^{d} is independently selected from halogen, oxo, C₁-C₆ alkyl, and C₁-C₆ alkoxycarbonyl;
each R^{e} is independently selected from halogen and benzyl;
each R^{f} is independently selected from halogen, oxo, NR^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with C₁-C₆ alkyl optionally substituted with halogen and —O(C₁-C₆ alkyl) optionally substituted with halogen;
each R^{g} is independently selected from hydrogen and C₁-C₆ alkyl optionally substituted with halogen or phenyl;
each R^{h} is C₁-C₆ alkyl;
each Rⁱ and R^{j} are independently selected from hydrogen and C₁-C₆ alkyl;
each R^{k} is independently selected from halogen, OH, OCH₃, phenyl and a 3 to 6 membered carbocyclyl; and
each R^{m} and Rⁿ are independently selected from hydrogen and C₁-C₆ alkyl.

In certain embodiments:
X¹ and X² are selected from CR⁹ and N, and X³ is CR⁹;
R¹ is C₁-C₃ alkyl;
R² is halogen, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 9 to 10 membered bicyclic heterocyclyl;
R³ and R⁴ are independently selected from hydrogen and C₁-C₆ alkyl;
R⁵ and R⁶ are independently selected from hydrogen or C₁-C₆ alkyl, or
R⁵ and R⁶ together with the atom to which they are attached form a 3 to 6 membered carbocyclyl or heterocyclyl;
R⁷ and R⁸ are independently selected from hydrogen or C₁-C₆ alkyl optionally substituted with R^{f}, or
R⁷ and R⁸ together with the atom to which they are attached form a 3 to 6 membered heterocyclyl;
each R⁹ is hydrogen;
each R^{b} is independently selected from CN and cyclopropyl;
each R^{c} is independently selected from halogen, CN, OR^{g}, SR^{h}, and C₁-C₈ alkyl;
each R^{f} is independently selected from a 5 to 6 membered heteroaryl and a 4 to 6 membered heterocyclyl optionally substituted with C₁-C₆ alkyl optionally substituted with halogen;
R^{g} is C₁-C₆ alkyl optionally substituted with halogen; and
R^{h} is C₁-C₆ alkyl.

In certain embodiments, X¹, X² and X³ are independently selected from CR⁹ and N, wherein only one of X¹, X² or X³ may be N. In certain embodiments, X¹ is N and X² and X³ are CR⁹. In certain embodiments, X² is N and X¹ and X³ are CR⁹. In certain embodiments, X³ is N and X¹ and X² are CR⁹. In certain embodiments, X¹, X² and X³ are CR⁹.

In certain embodiments, X¹, X² and X³ are CR⁹. In certain embodiments, each R⁹ is independently selected from hydrogen, halogen and methyl. In certain embodiments, each R⁹ is hydrogen. In certain embodiments, X¹, X² and X³ are CR⁹, and each R⁹ is hydrogen.

In certain embodiments, X¹ is N and X² and X³ are CR⁹. In certain embodiments, each R⁹ is independently selected from hydrogen, halogen and methyl. In certain embodiments, each R⁹ is hydrogen. In certain embodiments, X¹ is N and X² and X³ are CR⁹, and each R⁹ is hydrogen.

In certain embodiments, X² is N and X¹ and X³ are CR⁹. In certain embodiments, each R⁹ is independently selected from hydrogen, halogen and methyl. In certain embodiments, each R⁹ is hydrogen. In certain embodiments, X² is N and X¹ and X³ are CR⁹, and each R⁹ is hydrogen.

In certain embodiments, X¹ and X² are selected from CR⁹ and N, and X³ is CR⁹. In certain embodiments, X¹ is selected from CR⁹ and N, X² and X³ are CR⁹, and each R⁹ is hydrogen. In certain embodiments, X² is selected from CR⁹ and N, X¹ and X³ are CR⁹, and each R⁹ is hydrogen.

In certain embodiments, R¹ is selected from hydrogen, benzyl or C₁-C₃ alkyl optionally substituted with R^{a}. In certain embodiments, each R^{a} is independently selected from OH, OCH₃, halogen, a 5 to 6 membered heteroaryl, and a 3-6 membered heterocyclyl optionally substituted with C₁-C₃ alkyl optionally substituted with oxo. In certain embodiments, R¹ is selected from hydrogen, benzyl, methyl, ethyl, —CH₂CH₂OH, —CH₂CH₂CH₂OH, —CH₂CH₂OCH₃, —CH₂CH₂CH₂OCH₃, —CH₂CF₃, pyridin-2-ylmethyl, pyridin-4-ylmethyl and (1-acetylpiperidin-4-yl)methyl. In certain embodiments, R¹ is selected from benzyl, methyl, ethyl, —CH₂CH₂OH, —CH₂CH₂CH₂OH, —CH₂CH₂OCH₃, —CH₂CH₂CH₂OCH₃, —CH₂CF₃, pyridin-2-ylmethyl, pyridin-4-ylmethyl and (1-acetylpiperidin-4-yl)methyl. In certain embodiment, R¹ is methyl.

In certain embodiments, R¹ is C₁-C₃ alkyl. In certain embodiment, R¹ is methyl.

In certain embodiments, X¹ and X² are selected from CR⁹ and N, X³ is CR⁹, each R⁹ is hydrogen, and R¹ is methyl.

In certain embodiments, X¹, X² and X³ are CR⁹, each R⁹ is hydrogen, and R¹ is methyl.

In certain embodiments, X¹ is N and X² and X³ are CR⁹, each R⁹ is hydrogen, and R¹ is methyl.

In certain embodiments, X² is N and X¹ and X³ are CR⁹, each R⁹ is hydrogen, and R¹ is methyl.

In certain embodiments, R² is halogen, CN, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated heterocyclyl optionally substituted with R^{d}, a 3 to 6 membered saturated or unsaturated carbocyclyl optionally substituted with R^{d}, a 9 to 10 membered bicyclic heteroaryl optionally substituted with R^{c}, a 9 to 10 membered bicyclic heterocyclyl optionally substituted with R^{c}, phenylamino, phenoxy optionally substituted with R^{c}, or —NHC(═O)R^{o}. In certain embodiments, each R^{b} is independently selected from halogen, CN, OH, OCH₃, cyclopropyl and phenyl optionally substituted with halogen, OH or OCH₃. In certain embodiments, each R^{c} is independently selected from halogen, CN, a 3 to 6 membered carbocyclyl, a 5 to 6 membered heteroaryl, a 3 to 6 membered heterocyclyl, phenyl, OR^{g}, SR^{h}, NRⁱR^{j}, C₁-C₈ alkyl optionally substituted with R^{k}, C₁-C₈ alkynyl optionally substituted with R^{k}. In certain embodiments, each R^{d} is independently selected from halogen, oxo, C₁-C₆ alkyl, and C₁-C₆ alkoxycarbonyl. In certain embodiments, each R^{e} is independently selected from halogen and benzyl. In certain embodiments, each R^{g} is independently selected from hydrogen and C₁-C₆ alkyl optionally substituted with halogen or phenyl. In certain embodiments, each R^{h} is C₁-C₆ alkyl. In certain embodiments, each Rⁱ and R^{j} are independently selected from hydrogen and C₁-C₆ alkyl. In certain embodiments, each R^{k} is independently selected from halogen, OH, OCH₃, phenyl and a 3 to 6 membered carbocyclyl. In certain embodiments, R^{o} is phenyl or a 5 to 6 membered heteroaryl, wherein the phenyl and heteroaryl are optionally substituted with halogen, C₁-C₃ alkyl, and C₁-C₃ alkoxyl.

In certain embodiments, R² is halogen, CN, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated heterocyclyl optionally substituted with R^{d}, a 3 to 6 membered saturated or unsaturated carbocyclyl optionally substituted with R^{d}, a 9 to 10 membered bicyclic heteroaryl optionally substituted with R^{c}, a 9 to 10 membered bicyclic heterocyclyl optionally substituted with R^{c}, phenylamino, phenoxy optionally substituted with R^{e}, or —NHC(═O)R^{o}. In certain embodiments, each R^{b} is independently selected from halogen, CN, OH, OCH₃, cyclopropyl and phenyl optionally substituted with halogen, OH or OCH₃. In certain embodiments, each R^{c} is independently selected from halogen, CN, a 3 to 6 membered carbocyclyl, OR^{g}, SR^{h}, NRⁱR^{j}, C₁-C₈ alkyl optionally substituted with R^{k}, C₁-C₈ alkynyl optionally substituted with R^{k}. In certain embodiments, each R^{d} is independently selected from halogen, oxo, C₁-C₆ alkyl, and C₁-C₆ alkoxycarbonyl. In certain embodiments, each R^{e} is independently selected from halogen and benzyl. In certain embodiments, each R^{g} is independently selected from hydrogen and C₁-C₆ alkyl optionally substituted with halogen or phenyl. In certain embodiments, each R^{h} is C₁-C₆ alkyl. In certain embodiments, each Rⁱ and R^{j} are independently selected from hydrogen and C₁-C₆ alkyl. In certain embodiments, each R^{k} is independently selected from halogen, OH, OCH₃, phenyl and a 3 to 6 membered carbocyclyl. In certain embodiments, R^{o} is phenyl or a 5 to 6 membered heteroaryl, wherein the phenyl and heteroaryl are optionally substituted with halogen, C₁-C₃ alkyl, and C₁-C₃ alkoxyl.

In certain embodiments, R² is halogen, CN, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated heterocyclyl optionally substituted with R^{d}, a 3 to 6 membered saturated or unsaturated carbocyclyl optionally substituted with R^{d}, a 9 to 10 membered bicyclic heteroaryl optionally substituted with R^{c}, phenylamino, or phenoxy optionally substituted with R^{e}. In certain embodiments, each R^{b} is independently selected from halogen, CN, OH, OCH₃, cyclopropyl and phenyl optionally substituted with halogen, OH or OCH₃. In certain embodiments, each R^{c} is independently selected from halogen, CN, a 3 to 6 membered carbocyclyl, OR^{g}, SR^{h}, NRⁱR^{j}, C₁-C₈ alkyl optionally substituted with R^{k}, C₁-C₈ alkynyl optionally substituted with R^{k}. In certain embodiments, each R^{d} is independently selected from halogen, oxo, C₁-C₆ alkyl, and C₁-C₆ alkoxycarbonyl. In certain embodiments, each R^{e} is independently selected from halogen and benzyl. In certain embodiments, each R^{g} is independently selected from hydrogen and C₁-C₆ alkyl optionally substituted with halogen or phenyl. In certain embodiments, each R^{h} is C₁-C₆ alkyl. In certain embodiments, each Rⁱ and R^{j} are independently selected from hydrogen and C₁-C₆ alkyl. In certain embodiments, each R^{k} is independently selected from halogen, OH, OCH₃, phenyl and a 3 to 6 membered carbocyclyl.

In certain embodiments, R² is a 5 to 6 membered heteroaryl optionally substituted with R^{c}. In certain embodiments, R² is a 5 to 6 membered heteroaryl optionally substituted with R^{c}, wherein the heteroaryl contains one, two, three or four heteroatoms selected from the group consisting of oxygen, nitrogen and sulfur. In certain embodiments, R² is a 5 to 6 membered heteroaryl optionally substituted with R^{c} wherein the heteroaryl contains one or two nitrogen heteroatoms. In certain embodiments, R² is a 5 to 6 membered heteroaryl optionally substituted with R^{c}, wherein the heteroaryl is selected from group consisting of pyridine and pyrimidine.

In certain embodiments, R² is a 9 to 10 membered bicyclic heterocyclyl optionally substituted with R^{c}. In certain embodiments, R² is a 9 to 10 membered bicyclic heterocyclyl optionally substituted with R^{c}, wherein the heterocyclyl contains one or two heteroatoms selected from oxygen, nitrogen and sulfur. In certain embodiments, R² is a 9 to 10 membered bicyclic heterocyclyl optionally substituted with R^{c}, wherein the heterocyclyl contains two oxygen heteroatoms. In certain embodiments, R² is a 9 to 10 membered bicyclic heterocyclyl optionally substituted with R^{c}, wherein the heterocyclyl is benzodioxolyl.

In certain embodiments, R² is —NHC(═O)R^{o}. In certain embodiments, R^{o} is phenyl or a 5 to 6 membered heteroaryl, wherein the phenyl and heteroaryl are optionally substituted with halogen, C₁-C₃ alkyl, and C₁-C₃ alkoxyl. In certain embodiments, R^{o} is phenyl or a 5 to 6 membered heteroaryl, wherein the phenyl and heteroaryl are optionally substituted with halogen, C₁-C₃ alkyl, and C₁-C₃ alkoxyl, and wherein the heteroaryl contains one, two, three or four heteroatoms selected from oxygen, nitrogen and sulfur. In certain embodiments, R^{o} is phenyl or a 5 to 6 membered heteroaryl, wherein the phenyl and heteroaryl are optionally substituted with halogen, C₁-C₃ alkyl, and C₁-C₃ alkoxyl, and wherein the heteroaryl contains one or two heteroatoms selected from oxygen and nitrogen. In certain embodiments, R^{o} is phenyl or a 5 to 6 membered heteroaryl, wherein the phenyl and heteroaryl are optionally substituted with halogen, C₁-C₃ alkyl, and C₁-C₃ alkoxyl, and wherein the heteroaryl is selected from the group consisting of pyridine, pyrimidine, oxazole, furan and pyrazine.

In certain embodiments, R^{c} is a 5 to 6 membered heteroaryl. In certain embodiments, R^{c} is a 5 to 6 membered heteroaryl, wherein the heteroaryl contains one, two, three or four heteroatoms selected from the group consisting of oxygen, nitrogen and sulfur. In certain embodiments, R^{c} is a 5 to 6 membered heteroaryl, wherein the heteroaryl contains one or two nitrogen heteroatoms. In certain embodiments, R^{c} is a 5 to 6 membered heteroaryl, wherein the heteroaryl is pyrazole.

In certain embodiments, R² is Br, 4-(butanenitrile), isopentyl, cyclopropylvinyl, 3,3-dimethylbut-1-enyl, cyclopropylethynyl, 6-(hex-5-ynenitrile), 3-chlorophenyl, 3-methoxyphenyl, 3-chloro-5-fluorophenyl, 3-(difluoromethoxy)phenyl, 3-cyanophenyl (3-benzonitrile), 3-fluoro-5-methoxyphenyl, 3-chloro-2-fluorophenyl, 3-(trifluoromethoxy)phenyl, 3-methylphenyl, 3-(methylthio)phenyl, 2,5-dichlorophenyl, 5-chloro-2-fluorophenyl, pyridin-3-yl, 5-chloropyridin-3-yl, 5-methoxypyridin-3-yl, 2-fluoropyridin-3-yl, 5-nicotinonitrile (5-cyanopyridin-3-yl), 5-fluoropyridin-3-yl, 5-methylpyridin-3-yl, 5-trifluoromethylpyridin-3-yl, 2-fluoro-5-methylpyridin-3-yl, 2-(5-pyridin-3-yloxy)acetonitrile (5-cyanomethoxypyridin-3-yl), 2-(1H-pyrazol-1-yl)pyridin-3-yl, 4-methoxypyridin-2-yl, 2-isonicotinonitrile (4-cyanopyridin-2-yl), 4-trifluoromethylpyridin-2-yl, 4-methylpyridin-2-yl, 4-chloropyridin-2-yl, pyrimidin-5-yl, cyclohexyl, benzo[d][1,3]dioxol-5-yl, N-5-bromopicolinamide, N-5-chloropicolinamide, N-2-methyloxazole-4-carboxamide, N-2,5-dimethylfuran-3-carboxamide, N-5-methylpyrazine-2-carboxamide, N-pyrazine-2-carboxamide, N-benzamide, N-5-methoxypyrazine-2-carboxamide, N-4-methyloxazole-5-carboxamide and N-pivalamide.

In certain embodiments, R² is Br, 4-(butanenitrile), isopentyl, cyclopropylvinyl, 3,3-dimethylbut-1-enyl, cyclopropylethynyl, 6-(hex-5-ynenitrile), 3-chlorophenyl, 3-methoxyphenyl, 3-chloro-5-fluorophenyl, 3-(difluoromethoxy)phenyl, 3-cyanophenyl (3-benzonitrile), 3-fluoro-5-methoxyphenyl, 3-chloro-2-fluorophenyl, 3-(trifluoromethoxy)phenyl, 3-methylphenyl, 3-(methylthio)phenyl, 2,5-dichlorophenyl, 5-chloro-2-fluorophenyl, 5-chloropyridin-3-yl, 5-methoxypyridin-3-yl, 2-fluoropyridin-3-yl, 5-nicotinonitrile (5-cyanopyridin-3-yl), 5-fluoropyridin-3-yl, pyrimidin-5-yl, cyclohexyl, benzo[d][1,3]dioxol-5-yl, N-5-bromopicolinamide, N-5-chloropicolinamide, N-2-methyloxazole-4-carboxamide, N-2,5-dimethylfuran-3-carboxamide, N-5-methylpyrazine-2-carboxamide, N-pyrazine-2-carboxamide, N-benzamide, N-5-methoxypyrazine-2-carboxamide, and N-4-methyloxazole-5-carboxamide.

In certain embodiments, R² is Br, 4-(butanenitrile), isopentyl, cyclopropylvinyl, 3,3-dimethylbut-1-enyl, cyclopropylethynyl, 6-(hex-5-ynenitrile), 3-chlorophenyl, 3-methoxyphenyl, 3-chloro-5-fluorophenyl, 3-(difluoromethoxy)phenyl, 3-cyanophenyl (3-benzonitrile), 3-fluoro-5-methoxyphenyl, 3-chloro-2-fluorophenyl, 3-(trifluoromethoxy)phenyl, 3-methylphenyl, 3-(methylthio)phenyl, 2,5-dichlorophenyl, 5-chloropyridin-3-yl, 5-methoxypyridin-3-yl, 2-fluoropyridin-3-yl, pyrimidin-5-yl, and benzo[d][1,3]dioxol-5-yl.

In certain embodiments, R³ and R⁴ are independently selected from hydrogen, halogen and C₁-C₆ alkyl. In certain embodiments, R³ and R⁴ are independently selected from hydrogen, F and methyl. In certain embodiments, R³ and R⁴ are hydrogen. In certain embodiments, R³ and R⁴ are methyl. In certain embodiments, R³ and R⁴ are F.

In certain embodiments, R³ and R⁴ are independently selected from hydrogen, halogen and C₁-C₆ alkyl. In certain embodiments, R³ and R⁴ are independently selected from hydrogen and C₁-C₆ alkyl. In certain embodiments, R³ and R⁴ are independently selected from hydrogen and methyl. In certain embodiments, R³ and R⁴ are hydrogen. In certain embodiments, R³ and R⁴ are methyl.

In certain embodiments, R³ and R⁴ together form an oxo group.

In certain embodiments, R⁵ and R⁶ are independently selected from hydrogen, a 3 to 6 membered saturated or unsaturated carbocyclyl, or C₁-C₆ alkyl optionally substituted with R^{f}. In certain embodiments, each R^{f} is independently selected from halogen, oxo, OH, N^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with halogen, C₁-C₆ alkyl optionally substituted with halogen, —O(C₁-C₆ alkyl) optionally substituted with halogen, phenyl or a 5 to 6 membered heteroaryl. In certain embodiments, R⁵ and R⁶ are independently selected from hydrogen or C₁-C₆ alkyl optionally substituted with R^{f}. In certain embodiments, each R^{f} is independently selected from halogen, OH, phenyl, a 5 to 6 membered heteroaryl and a 4 to 6 membered heterocyclyl, wherein the phenyl, heteroaryl and heterocyclyl are optionally substituted with halogen, C₁-C₆ alkyl optionally substituted with halogen, or a 5 to 6 membered heteroaryl. In certain embodiments, R^{f} is phenyl optionally substituted with a 5 to 6 membered heteroaryl, wherein the heteroaryl contains one, two, three or four heteroatoms selected from oxygen, nitrogen and sulfur. In certain embodiments, R^{f} is phenyl optionally substituted with a 5 to 6 membered heteroaryl, wherein the heteroaryl contains one or two heteroatoms selected from oxygen, nitrogen and sulfur. In certain embodiments, R^{f} is phenyl optionally substituted with a 5 to 6 membered heteroaryl, wherein the heteroaryl contains one or two nitrogen heteroatoms. In certain embodiments, R^{f} is phenyl optionally substituted with a 5 to 6 membered heteroaryl, wherein the heteroaryl contains two nitrogen heteroatoms. In certain embodiments, R^{f} is phenyl optionally substituted with a 5 to 6 membered heteroaryl, wherein the heteroaryl is pyrimidine. In certain embodiments, R⁵ and R⁶ are independently selected from hydrogen, methyl, CH₂OH, benzyl, 4-bromobenzyl and 4-(pyrimidin-5-yl)benzyl. In certain embodiments, R⁵ and R⁶ are hydrogen. In certain embodiments, R⁵ and R⁶ are methyl. In certain embodiments, R⁵ is CH₂OH and R⁶ is methyl. In certain embodiments, R⁵ is hydrogen, methyl, CH₂OH, benzyl, 4-bromobenzyl or 4-(pyrimidin-5-yl)benzyl, and R⁶ is hydrogen or methyl. In certain embodiments, R⁵ is CH₂OH, benzyl, 4-bromobenzyl or 4-(pyrimidin-5-yl)benzyl, and R⁶ is hydrogen or methyl. In certain embodiments, R⁵ is CH₂OH, benzyl, 4-bromobenzyl or 4-(pyrimidin-5-yl)benzyl, and R⁶ is methyl.

In certain embodiments, R⁵ and R⁶ are independently selected from hydrogen, a 3 to 6 membered saturated or unsaturated carbocyclyl, or C₁-C₆ alkyl optionally substituted with R^{f}. In certain embodiments, each R^{f} is independently selected from halogen, oxo, OH, NR^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with C₁-C₆ alkyl optionally substituted with halogen and —O(C₁-C₆ alkyl) optionally substituted with halogen. In certain embodiments, R⁵ and R⁶ are independently selected from hydrogen or C₁-C₆ alkyl optionally substituted with OH. In certain embodiments, R⁵ and R⁶ are independently selected from hydrogen, methyl or CH₂OH. In certain embodiments, R⁵ and R⁶ are hydrogen. In certain embodiments, R⁵ and R⁶ are methyl. In certain embodiments, R⁵ is CH₂OH and R⁶ is methyl.

In certain embodiments, R⁵ and R⁶ are independently selected from hydrogen, a 3 to 6 membered saturated or unsaturated carbocyclyl, or C₁-C₆ alkyl optionally substituted with R^{f}. In certain embodiments, each R^{f} is independently selected from halogen, oxo, NR^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with C₁-C₆ alkyl optionally substituted with halogen and —O(C₁-C₆ alkyl) optionally substituted with halogen. In certain embodiments, R⁵ and R⁶ are independently selected from hydrogen or C₁-C₆ alkyl. In certain embodiments, R⁵ and R⁶ are independently selected from hydrogen or methyl. In certain embodiments, R⁵ and R⁶ are hydrogen. In certain embodiments, R⁵ and R⁶ are methyl.

In certain embodiments, R⁵ and R⁶ together with the atom to which they are attached form a 3 to 6 membered carbocyclyl or heterocyclyl. In certain embodiments, R⁵ and R⁶ together with the atom to which they are attached form a 3 to 6 membered heterocyclyl, wherein the heterocyclyl contains one or two heteroatoms selected from oxygen, nitrogen and sulfur. In certain embodiments, R⁵ and R⁶ together with the atom to which they are attached form a 3 to 6 membered heterocyclyl, wherein the heterocyclyl contains one oxygen heteroatom. In certain embodiments, R⁵ and R⁶ together with the atom to which they are attached form a 3 to 6 membered heterocyclyl, wherein the heterocyclyl is tetrahydropyran. In certain embodiments, R⁵ and R⁶ together with the atom to which they are attached form cyclobutyl or tetrahydropyran-4-yl.

In certain embodiments, R⁷ and R⁸ are independently selected from hydrogen, halogen or C₁-C₆ alkyl optionally substituted with R^{f}. In certain embodiments, each R^{f} is independently selected from halogen, oxo, NR^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with C₁-C₆ alkyl optionally substituted with halogen and —O(C₁-C₆ alkyl) optionally substituted with halogen. In certain embodiments, each R^{f} is independently selected from a 5 to 6 membered heteroaryl and a 4 to 6 membered heterocyclyl, wherein the heteroaryl or heterocyclyl are optionally substituted with C₁-C₆ alkyl optionally substituted with halogen. In certain embodiments, R^{f} is a 4 to 6 membered heterocyclyl optionally substituted with C₁-C₆ alkyl optionally substituted with halogen. In certain embodiments, R^{f} is a 4 to 6 membered heterocyclyl optionally substituted with C₁-C₆ alkyl optionally substituted with halogen, wherein the heterocyclyl contains one or two heteroatoms selected from oxygen, nitrogen and sulfur. In certain embodiments, R^{f} is a 4 to 6 membered heterocyclyl optionally substituted with C₁-C₆ alkyl optionally substituted with halogen, wherein the heterocyclyl contains one nitrogen heteroatom. In certain embodiments, R^{f} is a 4 to 6 membered heterocyclyl optionally substituted with C₁-C₆ alkyl optionally substituted with halogen, wherein the heterocyclyl is piperidine. In certain embodiments, R^{f} is a 5 to 6 membered heteroaryl, wherein the heteroaryl contains one, two or three heteroatoms selected from oxygen, nitrogen and sulfur. In certain embodiments, R^{f} is a 5 to 6 membered heteroaryl, wherein the heteroaryl contains one nitrogen heteroatom. In certain embodiments, R^{f} is a 5 to 6 membered heteroaryl, wherein the heteroaryl is pyridine. In certain embodiments, R⁷ is selected from hydrogen, halogen and C₁-C₆ alkyl optionally substituted with R^{f}, and R⁸ is selected from hydrogen, halogen and C₁-C₆ alkyl. In certain embodiments, R⁷ and R⁸ are independently selected from hydrogen, F, methyl, 1-(2,2-difluoroethyl)piperidin-4-yl)methyl, and pyridin-3-ylmethyl. In certain embodiments, R⁷ is selected from hydrogen, F, methyl, 1-(2,2-difluoroethyl)piperidin-4-yl)methyl, and pyridin-3-ylmethyl, and R⁸ is selected from hydrogen, F and methyl. In certain embodiments, R⁷ and
R⁸ are hydrogen. In certain embodiments, R⁷ and R⁸ are methyl. In certain embodiments, R⁷ is F and R⁸ is hydrogen. In certain embodiments, R⁷ and R⁸ are F.

In certain embodiments, R⁷ and R⁸ are independently selected from hydrogen, halogen or C₁-C₆ alkyl optionally substituted with R^{f}. In certain embodiments, each R^{f} is independently selected from halogen, oxo, NR^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with C₁-C₆ alkyl optionally substituted with halogen and —O(C₁-C₆ alkyl) optionally substituted with halogen. In certain embodiments, R⁷ and R⁸ are independently selected from hydrogen or C₁-C₆ alkyl optionally substituted with R^{f}. In certain embodiments, each R^{f} is independently selected from a 5 to 6 membered heteroaryl and a 4 to 6 membered heterocyclyl, wherein the heteroaryl or heterocyclyl are optionally substituted with C₁-C₆ alkyl optionally substituted with halogen. In certain embodiments, R^{f} is a 4 to 6 membered heterocyclyl optionally substituted with C₁-C₆ alkyl optionally substituted with halogen. In certain embodiments, R^{f} is a 4 to 6 membered heterocyclyl optionally substituted with C₁-C₆ alkyl optionally substituted with halogen, wherein the heterocyclyl contains one or two heteroatoms selected from oxygen, nitrogen and sulfur. In certain embodiments, R^{f} is a 4 to 6 membered heterocyclyl optionally substituted with C₁-C₆ alkyl optionally substituted with halogen, wherein the heterocyclyl contains one nitrogen heteroatom. In certain embodiments, R^{f} is a 4 to 6 membered heterocyclyl optionally substituted with C₁-C₆ alkyl optionally substituted with halogen, wherein the heterocyclyl is piperidine. In certain embodiments, R^{f} is a 5 to 6 membered heteroaryl, wherein the heteroaryl contains one, two or three heteroatoms selected from oxygen, nitrogen and sulfur. In certain embodiments, R^{f} is a 5 to 6 membered heteroaryl, wherein the heteroaryl contains one nitrogen heteroatom. In certain embodiments, R^{f} is a 5 to 6 membered heteroaryl, wherein the heteroaryl is pyridine. In certain embodiments, R⁷ is selected from hydrogen or C₁-C₆ alkyl optionally substituted with R^{f}, and R⁸ is selected from hydrogen and C₁-C₆ alkyl. In certain embodiments, R⁷ and R⁸ are independently selected from hydrogen, methyl, 1-(2,2-difluoroethyl)piperidin-4-yl)methyl, and pyridin-3-ylmethyl. In certain embodiments, R⁷ is selected from hydrogen, methyl, 1-(2,2-difluoroethyl)piperidin-4-yl)methyl, and pyridin-3-ylmethyl, and R⁸ is selected from hydrogen and methyl. In certain embodiments, R⁷ and R⁸ are hydrogen. In certain embodiments, R⁷ and R⁸ are methyl.

In certain embodiments, R⁷ and R⁸ together with the atom to which they are attached form a 3 to 6 membered carbocyclyl or heterocyclyl. In certain embodiments, R⁷ and R⁸ together with the atom to which they are attached form a 3 to 6 membered heterocyclyl. In certain embodiments, R⁷ and R⁸ together with the atom to which they are attached form a 3 to 6 membered heterocyclyl, wherein the heterocyclyl contains one or two heteroatoms selected from oxygen, nitrogen and sulfur. In certain embodiments, R⁷ and R⁸ together with the atom to which they are attached form a 3 to 6 membered heterocyclyl, wherein the heterocyclyl contains one oxygen heteroatom. In certain embodiments, R⁷ and R⁸ together with the atom to which they are attached form a 3 to 6 membered heterocyclyl, wherein the heterocyclyl is tetrahydropyran. In certain embodiments, R⁷ and R⁸ together with the atom to which they are attached form tetrahydropyran-4-yl.

In certain embodiments, R⁵ and R⁷ together with the atoms to which they are attached form a 3 to 4 membered carbocyclyl or heterocyclyl. In certain embodiments, R⁵ and R⁷ together with the atoms to which they are attached form a 3 to 4 membered carbocyclyl or heterocyclyl; R⁶ is hydrogen, a 3 to 6 membered saturated or unsaturated carbocyclyl, or C₁-C₆ alkyl optionally substituted with R^{f}; and R⁸ is hydrogen, halogen or C₁-C₆ alkyl optionally substituted with R^{f}. In certain embodiments, R⁵ and R⁷ together with the atoms to which they are attached form a 3 to 4 membered carbocyclyl or heterocyclyl; R⁶ is hydrogen or C₁-C₆ alkyl optionally substituted with R^{f}; and R⁸ is hydrogen, halogen or C₁-C₆ alkyl optionally substituted with R^{f}. In certain embodiments, R⁵ and R⁷ together with the atoms to which they are attached form a 3 to 4 membered carbocyclyl or heterocyclyl; R⁶ is hydrogen or C₁-C₆ alkyl; and R⁸ is hydrogen. In certain embodiments, R⁵ and R⁷ together with the atoms to which they are attached form a cyclopropyl ring. In certain embodiments, R⁵ and R⁷ together with the atoms to which they are attached form a cyclopropyl ring; R⁶ is hydrogen or methyl; and R⁸ is hydrogen.

In certain embodiments:
(i) R⁵ and R⁶ are independently selected from hydrogen, a 3 to 6 membered saturated or unsaturated carbocyclyl, C₁-C₆ alkyl optionally substituted with R^{f},
or R⁵ and R⁶ together with the atom to which they are attached form a 3 to 6 membered carbocyclyl or heterocyclyl; and
R⁷ and R⁸ are hydrogen; or
(ii) R⁵ and R⁶ are hydrogen; and
R⁷ and R⁸ are independently selected from hydrogen, halogen or C₁-C₆ alkyl optionally substituted with R^{f}, or
R⁷ and R⁸ together with the atom to which they are attached form a 3 to 6 membered carbocyclyl or heterocyclyl, wherein only one of the pairs of R⁵ and R⁶ or R⁷ and R⁸ may together form a ring, or
(iii) R⁵ and R⁷ together with the atoms to which they are attached form a 3 to 4 membered carbocyclyl or heterocyclyl, wherein only one of the pairs of R⁵ and R⁶, R⁷ and R⁸ or R⁵ and R⁷ may together form a ring;
R⁶ is hydrogen, a 3 to 6 membered saturated or unsaturated carbocyclyl, or C₁-C₆ alkyl optionally substituted with R^{f}; and
R⁸ is hydrogen, halogen or C₁-C₆ alkyl optionally substituted with R^{f}.

In certain embodiments:
(i) R⁵ and R⁶ are independently selected from hydrogen, a 3 to 6 membered saturated or unsaturated carbocyclyl, or
C₁-C₆ alkyl optionally substituted with R^{f}, or R⁵ and R⁶ together with the atom to which they are attached form a 3 to 6 membered carbocyclyl or heterocyclyl; and
R⁷ and R⁸ are hydrogen; or
(ii) R⁵ and R⁶ are hydrogen; and
R⁷ and R⁸ are independently selected from hydrogen, halogen or C₁-C₆ alkyl optionally substituted with R^{f}, or
R⁷ and R⁸ together with the atom to which they are attached form a 3 to 6 membered carbocyclyl or heterocyclyl, wherein only one of the pairs of R⁵ and R⁶ or R⁷ and R⁸ may together form a ring.

In certain embodiments:
(i) R⁵ and R⁶ are independently selected from hydrogen or C₁-C₆ alkyl, or
R⁵ and R⁶ together with the atom to which they are attached form a 3 to 6 membered carbocyclyl or heterocyclyl; and
R⁷ and R⁸ are hydrogen; or
(ii) R⁵ and R⁶ are hydrogen; and
R⁷ and R⁸ are independently selected from hydrogen or C₁-C₆ alkyl optionally substituted with R^{f}, or
R⁷ and R⁸ together with the atom to which they are attached form a 3 to 6 membered heterocyclyl.

Compounds of the invention contain one or more asymmetric or chiral centers, e.g., a chiral carbon atom. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention. In a particular embodiment, compounds of the invention have the stereochemical orientation represented by Formula I′:

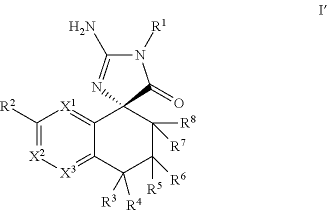

wherein X¹, X², X³, R¹, R², R³, R⁴, R⁵, R⁶, R⁷ and R⁸ are as defined herein.

In a particular embodiment, compounds of the invention have the stereochemical orientation represented by Formula I″:

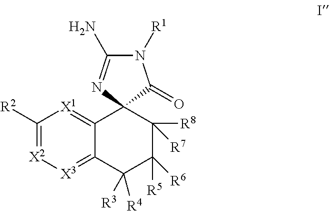

wherein X¹, X², X³, R¹, R², R³, R⁴, R⁵, R⁶, R⁷ and R⁸ are as defined herein.

In a particular embodiment, compounds of the invention have the stereochemical orientation represented by Formula II′:

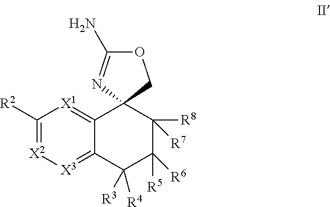

wherein X¹, X², X³, R², R³, R⁴, R⁵, R⁶, R⁷ and R⁸ are as defined herein.

In a particular embodiment, compounds of the invention have the stereochemical orientation represented by Formula II″:

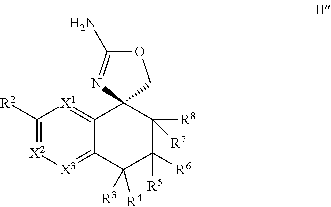

wherein X¹, X², X³, R², R³, R⁴, R⁵, R⁶, R⁷ and R⁸ are as defined herein.

In a particular embodiment, compounds of the invention have the stereochemical orientation represented by Formula III′:

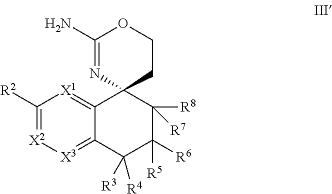

wherein X¹, X², X³, R², R³, R⁴, R⁵, R⁶, R⁷ and R⁸ are as defined herein.

In a particular embodiment, compounds of the invention have the stereochemical orientation represented by Formula III″:

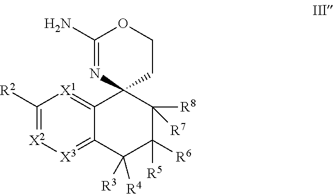

wherein X¹, X², X³, R², R³, R⁴, R⁵, R⁶, R⁷ and R⁸ are as defined herein.

In a particular embodiment, compounds of the invention have the stereochemical orientation represented by Formula IV′:

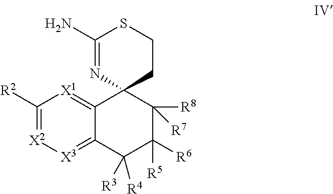

X¹, X², X³, R², R³, R⁴, R⁵, R⁶, R⁷, and R⁸ are as defined herein.

In a particular embodiment, compounds of the invention have the stereochemical orientation represented by Formula IV″:

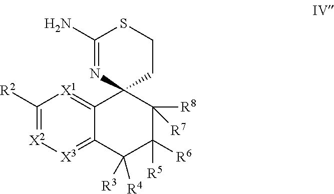

wherein X¹, X², X³, R², R³, R⁴, R⁵, R⁶, R⁷ and R⁸ are as defined herein.

In a particular embodiment, compounds of the invention have the stereochemical orientation represented by Formula V′:

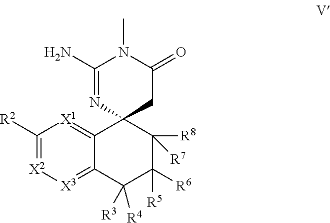

wherein X¹, X², X³, R², R³, R⁴, R⁵, R⁶, R⁷ and R⁸ are as defined herein.

In a particular embodiment, compounds of the invention have the stereochemical orientation represented by Formula V″:

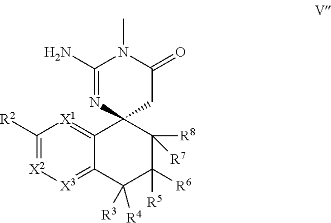

wherein X¹, X², X³, R², R³, R⁴, R⁵, R⁶, R⁷ and R⁸ are as defined herein.

In a particular embodiment, compounds of the invention have the stereochemical orientation represented by Formula VI′:

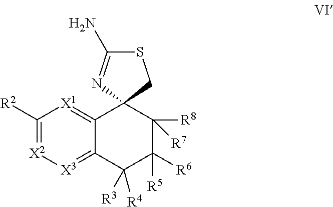

wherein X¹, X², X³, R², R³, R⁴, R⁵, R⁶, R⁷ and R⁸ are as defined herein.

In a particular embodiment, compounds of the invention have the stereochemical orientation represented by Formula VI″:

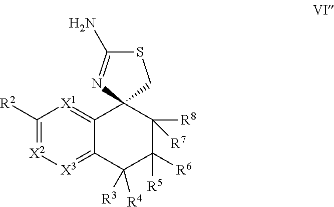

wherein X¹, X², X³, R², R³, R⁴, R⁵, R⁶, R⁷ and R⁸ are as defined herein.

In a particular embodiment, compounds of the invention have the stereochemical orientation represented by Formula α′:

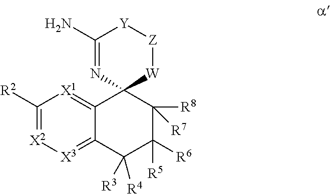

wherein W, X¹, X², X³, Y, Z, R², R³, R⁴, R⁵, R⁶, R⁷ and R⁸ are as defined herein.

In a particular embodiment, compounds of the invention have the stereochemical orientation represented by Formula α″:

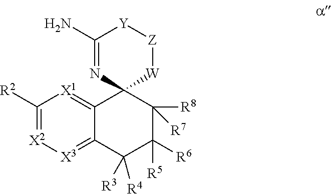

wherein W, X¹, X², X³, Y, Z, R², R³, R⁴, R⁵, R⁶, R⁷ and R⁸ are as defined herein.

The invention also encompasses prodrugs of the compounds described above. Suitable prodrugs where applicable include known amino-protecting and carboxy-protecting groups which are released, for example hydrolyzed, to yield the parent compound under physiologic conditions. A particular class of prodrugs are compounds in which a nitrogen atom in an amino, amidino, aminoalkyleneamino, iminoalkyleneamino or guanidino group is substituted with a hydroxy (OH) group, an alkylcarbonyl (—CO—R) group, an alkoxycarbonyl (—CO—OR), an acyloxyalkyl-alkoxycarbonyl (—CO—O—R—O—CO—R) group where R is a monovalent or divalent group and as defined above or a group having the formula —C(O)—O—CP1P2-haloalkyl, where P1 and P2 are the same or different and are hydrogen, lower alkyl, lower alkoxy, cyano, halo lower alkyl or aryl. In a particular embodiment, the nitrogen atom is one of the nitrogen atoms of the amidino group of the compounds of the invention. These prodrug compounds are prepared by reacting the compounds of the invention described above with an activated acyl compound to bond a nitrogen atom in the compound of the invention to the carbonyl of the activated acyl compound. Suitable activated carbonyl compounds contain a good leaving group bonded to the carbonyl carbon and include acyl halides, acyl amines, acyl pyridinium salts, acyl alkoxides, in particular acyl phenoxides such as p-nitrophenoxy acyl, dinitrophenoxy acyl, fluorophenoxy acyl, and difluorophenoxy acyl. The reactions are generally exothermic and are carried out in inert solvents at reduced temperatures such as −78° C. to about 50° C. The reactions are usually also carried out in the presence of an inorganic base such as potassium carbonate or sodium bicarbonate, or an organic base such as an amine, including pyridine, triethylamine, etc. One manner of preparing prodrugs is described in PCT publication WO 98/46576, the contents of which are incorporated herein by reference in their entirety.

Compounds of the invention may exist as stereoisomers, e.g., diastereomers and enantiomers, resonance forms, e.g., tautomers, solvates and salts, and all such stereoisomers, resonance forms, solvates and salts are within the scope of the invention herein.

It will also be appreciated that certain compounds of Formula α, α′, α″, I, I′, I″, II, II′, II″, III, III′, III″, IV, IV′, IV″, V, V′, V″, VI, VI′, VI″ or VII may be used as intermediates for further compounds of Formula α, α′, α″, I, I′, I″, II, II′, II″, III, III′, III″, IV, IV′, IV″, V, V′, V″, VI, VI′, VI″ or VII.

It will also be appreciated that certain compounds of Formula I may be used as intermediates for further compounds of Formula I.

Synthesis of Compounds

Compounds of the invention are prepared using standard organic synthetic techniques from starting materials and reagents generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Fieser, Louis F., and Mary Fieser, Reagents for Organic Synthesis. v. 1-23, New York: Wiley 1967-2006 ed. (also available via the Wiley InterScience® website), or Beilsteins Handbuch der organischen Chemie, 4, Aufl. ed. Springer-Verlag, Berlin, including supplements (also available via the Beilstein online database)). It will be appreciated that synthetic procedures employed in the preparation of compounds of the invention will depend on the particular substituents present in a compound. In preparing compounds of the invention, protection of remote functionalities (e.g., primary or secondary amines, etc.) of intermediates may be necessary but may not be illustrated in the following general Schemes. The need for such protection will vary depending on the nature of the remote functionality and the conditions of the preparation methods. The need for such protection is readily determined by one skilled in the art. For a general description of protecting groups and their use, see Greene's Protective Groups in Organic Synthesis, supra.

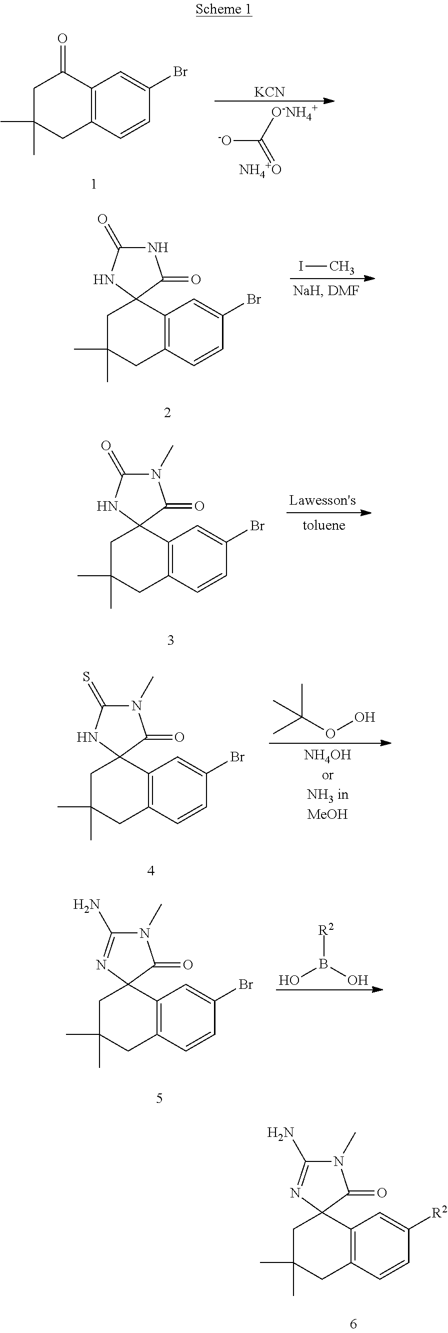

Scheme 1 shows a general scheme for the synthesis of compound 6, wherein R² is as defined herein. Compound 1 may be heated with cyanopotassium and ammonium carbonate to provide compound 2. Compound 2 may then be reacted with iodomethane (R¹ is methyl), or the desired R¹-iodide, to provide compound 3. Compound 3 may then be reacted with Lawesson's Reagent to provide compound 4, which may be converted to compound 5 by reacting with ammonium hydroxide or ammonia in methanol. A final Suzuki coupling provides compound 6.

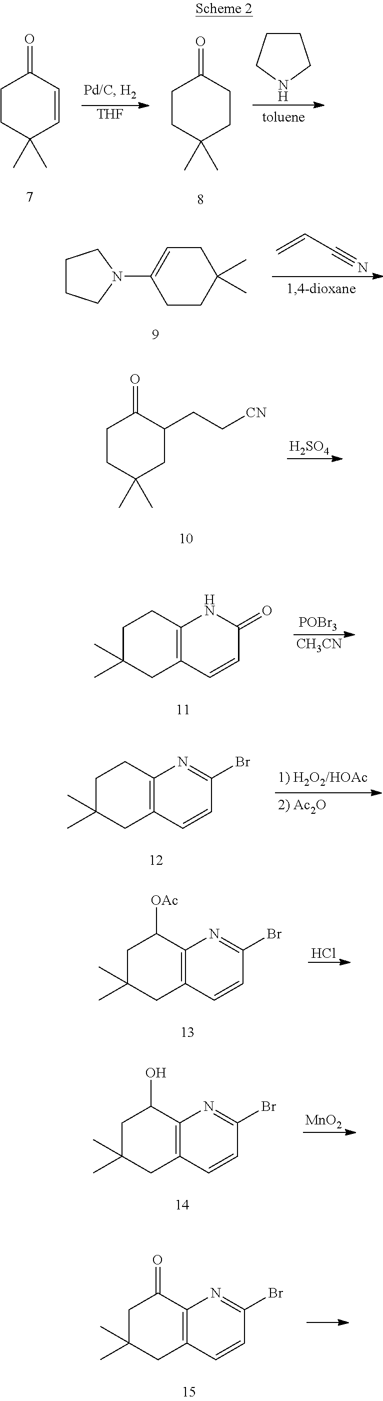

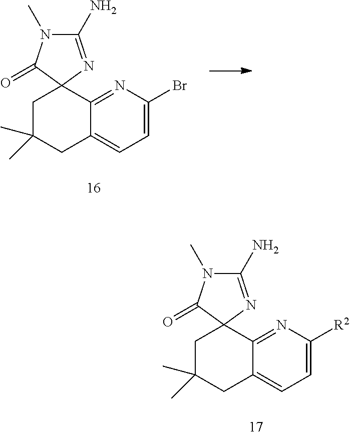

Scheme 2 shows a general scheme for the synthesis of compound 17, wherein R² is as defined herein. Compound 7 may be hydrogenated to provide compound 8, which may be further reacted with pyrrolidine to provide compound 9. Dioxane and acrylonitrile may be reacted with compound 9 to provide compound 10. Sulfuric acid may be added to compound 10 to provide compound 11, which may be treated with phosphoryl tribromide and acetonitrile (“ACN”) to provide compound 12. Compound 12 may be treated with hydrogen peroxide and acetic acid, followed by acetic anhydride to provide compound 13. Compound 13 may be treated with hydrochloric acid to provide compound 14. Compound 14 may be treated with manganese dioxide to provide compound 15. Compound 15 may be treated as compound 1 in Scheme 1, to provide compound 16, which after Suzuki coupling provides compound 17.

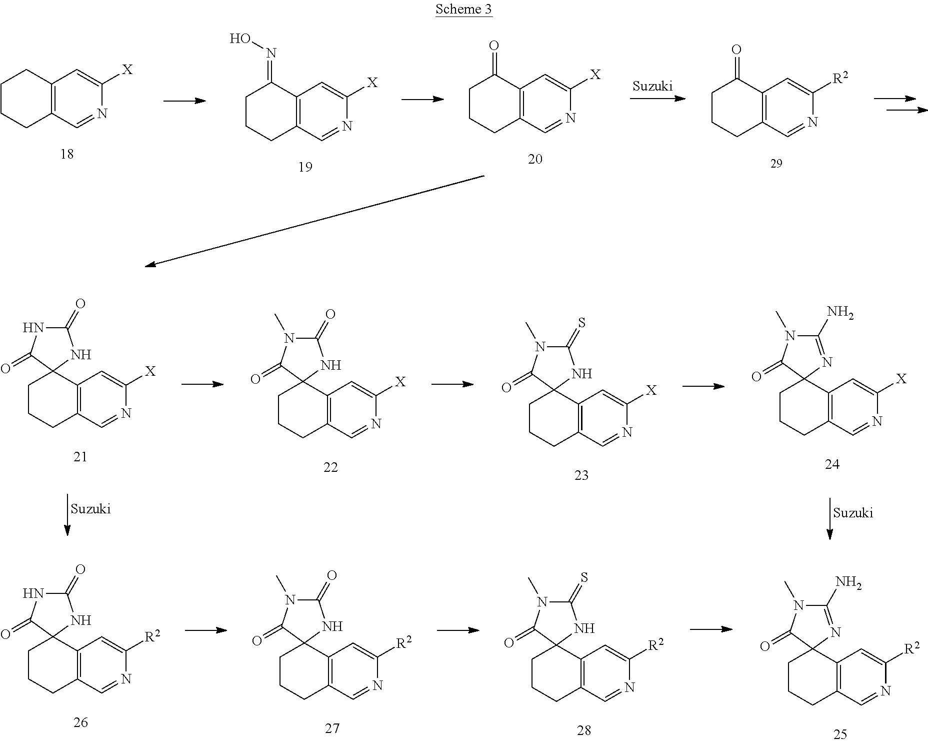

Scheme 3 shows a general scheme for the synthesis of compound 25, wherein R² is as defined herein. Oxime 19, wherein X is halogen, may be prepared as described in WO 2009/010488. Oxime 19 may then be treated with hydrochloric acid to provide compound 20. Compound 20 may be treated as compound 1 in Scheme 1, to provide intermediate compounds 21-24, which after Suzuki coupling provides compound 25. In one alternative, after preparing compound 21, the Suzuki may then be performed to provide compound 26, which is then treated as compound 1 in Scheme to provide compound 26. In another alternative the Suzuki may be performed on compound 20, which is then treated as compound 1 in Scheme to provide compound 26.

In one embodiment, a process of preparing a compound of Formula I is provided, comprising:
(a) reacting a compound of Formula A:

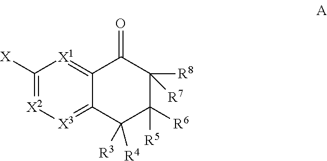

wherein X is halogen, and X¹, X², X³, R³, R⁴, R⁵, R⁶, R⁷ and R⁸ are as defined herein, with cyanopotassium and ammonium carbonate to provide a compound of Formula B:

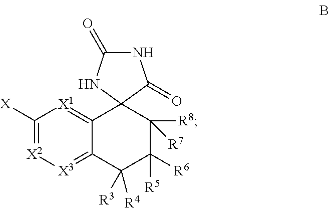

(b) reacting a compound of Formula B with I—R¹ to provide a compound of Formula C:

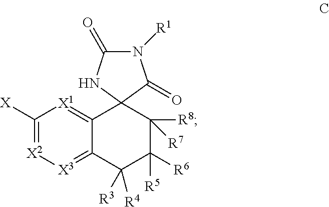

(c) reacting a compound of Formula C with Lawesson's Reagent to provide a compound of Formula D:

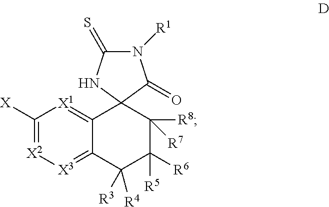

(d) reacting a compound of Formula D with ammonium hydroxide or ammonia in methanol to provide a compound of Formula E (which is a subset of Formula I, wherein R² is halogen):

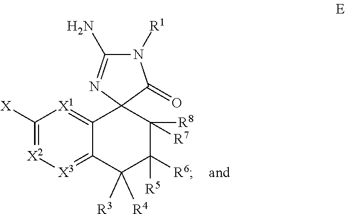

and
(e) optionally performing a Suzuki coupling before or after any of Steps (a) through (d) to convert X to R² to prepare a compound of Formula I.

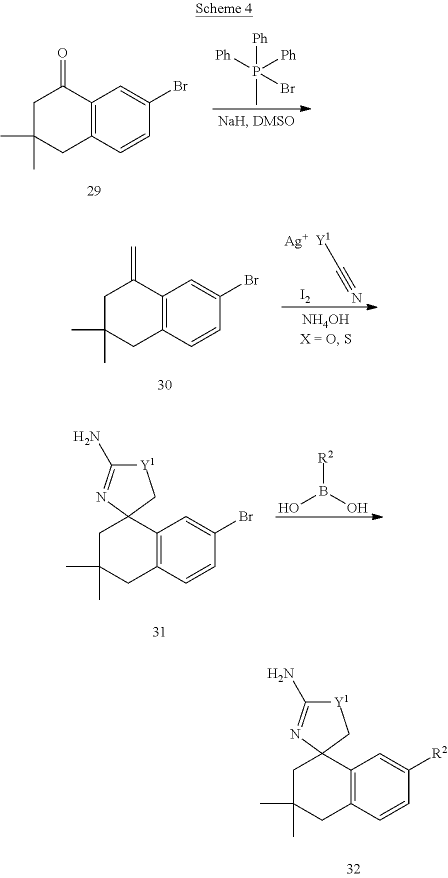

Scheme 4 shows a general scheme for the synthesis of compound 32, wherein Y¹ is O or S and R² is as defined herein. Compound 29 may be treated with typical Wittig reaction conditions to afford compound 30. Compound 30 may then be reacted with silver cyanate (Y¹ is O) and iodine using THF (“tetrahydrofuran”), acetonitrile or di-ethyl ether as a solvent. The reaction is typically filtered and treated with ammonium hydroxide in acetone, THF or another suitable solvent to afford compound 31. Silver thiocyante may be used instead of silver cyanate to provide compounds where Y¹ is S. Standard Suzuki conditions are used to afford compound 32.

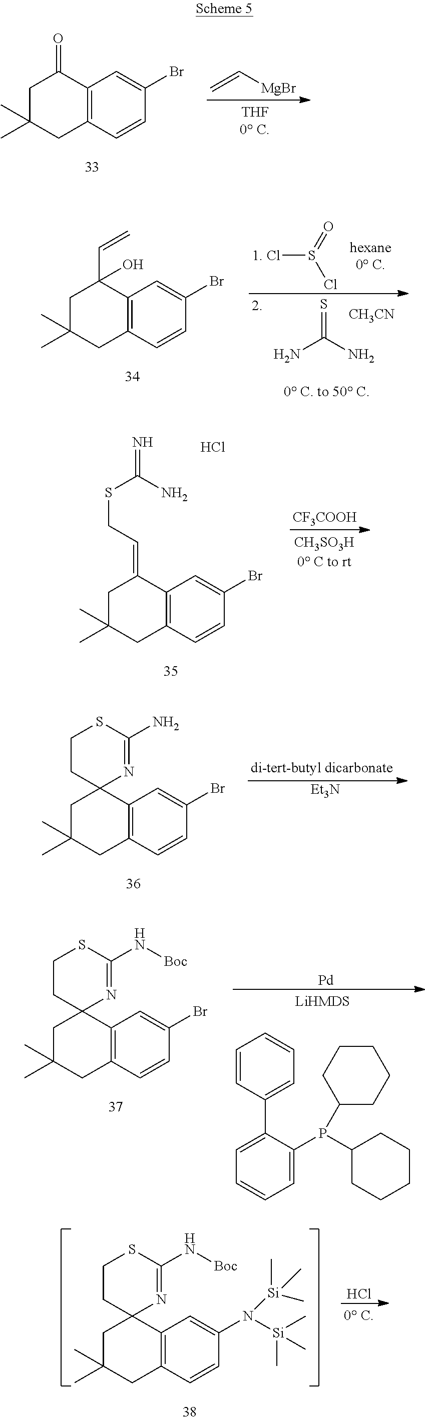

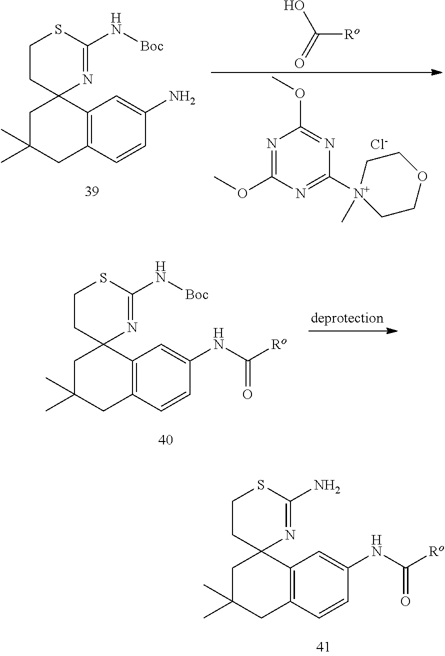

Scheme 5 shows a general scheme for the synthesis of compound 41, wherein R^{o} is as defined herein. Compound 33 may be reacted with vinylmagnesiun halide to provide compound 34, which may then be reacted with thionyl chloride and thiourea to provide compound 35. Compound 35 may then be reacted with an acid, such as TFA (“trifluoroacetic acid”), MSA (“methanesulfonic acid”) or a mixture of TFA/MSA, to provide compound 36. Compound 36 may be converted to compound 37 by reacting with di-tert-butyl dicarbonate. Compound 39 may be prepared by a coupling reaction of compound 37 with lithium bis(trimethylsilyl)amide (“LiHMDS”) in the presence of Pd source and an appropriate ligand followed by treatment of intermediate 38 with an acid, such as HCl. Compound 39 may then be reacted with an acid chloride or an acid in the presence of a coupling agent to provide compound 40. A deprotection of the Boc group provides compound 41. Other R² groups may be installed by subjecting compounds 4 or 5 to other reactions, such as a Suzuki, Buchwald, or Ullmann coupling.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and S. Wilen. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds described herein may be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., ed. Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts. Alternatively, by method (2), the substrate to be resolved is reacted with one enantiomer of a chiral compound to form a diastereomeric pair (Eliel, E., and S. Wilen. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, □-methoxy-□-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (Lough, W. J., ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. of Chromatogr. Vol. 513 (1990): pp. 375-378). Enriched or purified enantiomers can be distinguished by methods used to distinguish other chiral molecules with asymmetric carbon atoms, such as optical rotation and circular dichroism.

Indications

The compounds of the invention inhibit the cleavage of amyloid precursor protein by β-secretase which is implicated in diseases, in particular, neurodegenerative diseases such as Alzheimer's disease. In AD, processing of APP by β-secretase produces soluble N-APP which activates extrinsic apoptotic pathways by binding to death receptor 6. Furthermore, APP that is processed by β-secretase is subsequently cleaved by γ-secretase thereby producing amyloid beta peptides such as Aβ1-42 that form amyloid plaques which contribute to nerve cell death. Compounds of the invention inhibit enzymatic cleavage of APP by β-secretase.

Accordingly, in an aspect of the invention, there is provided a method of inhibiting cleavage of APP by β-secretase in a mammal comprising administering to said mammal an effective amount of a compound of Formula I.

Accordingly, in an aspect of the invention, there is provided a method of inhibiting cleavage of APP by β-secretase in a mammal comprising administering to said mammal an effective amount of a compound of Formula α, α′, α″, I, I′, I″, II, II′, II″, III, III′, III″, IV, IV′, IV″, V, V′, V″, VI, VI′, VI″ or VII.

In another aspect of the invention, there is provided a method for treating a disease or condition mediated by the cleavage of APP by β-secretase in a mammal, comprising administering to said mammal an effective amount of a compound of Formula I.

In another aspect of the invention, there is provided a method for treating a disease or condition mediated by the cleavage of APP by β-secretase in a mammal, comprising administering to said mammal an effective amount of a compound of Formula α, α′, α″, I, I′, I″, II, II′, II″, III, III′, III″, IV, IV′, IV″, V, V′, V″, VI, VI′, VI″ or VII.

In another aspect, there is provided the use of a compound of Formula I in the manufacture of a medicament for the treatment of a neurodegenerative disease. In one embodiment, the neurodegenerative disease is Alzheimer's disease.

In another aspect, there is provided the use of a compound of Formula α, α′, α″, I, I′, I″, II, II′, II″, III, III′, III″, IV, IV′, IV″, V, V′, V″, VI, VI′, VI″ or VII in the manufacture of a medicament for the treatment of a neurodegenerative disease. In one embodiment, the neurodegenerative disease is Alzheimer's disease.

In another aspect of the invention, there is provided a use of a compound of Formula I in the treatment of neurodegenerative diseases. In one embodiment, the neurodegenerative disease is Alzheimer's disease.

In another aspect of the invention, there is provided a use of a compound of Formula α, α′, α″, I, I′, I″, II, II′, II″, III, III′, III″, IV, IV′, IV″, V, V′, V″, VI, VI′, VI″ or VII in the treatment of neurodegenerative diseases. In one embodiment, the neurodegenerative disease is Alzheimer's disease.

Compounds of the invention may be administered prior to, concomitantly with, or following administration of other therapeutic compounds. Sequential administration of each agent may be close in time or remote in time. The other therapeutic agents may be anti-neurodegenerative with a mechanism of action that is the same as compounds of the invention, i.e., inhibit beta-secretase cleavage of APP, or a different mechanism of action, e.g., anti-Aβ antibodies. The compounds may be administered together in a unitary pharmaceutical composition or separately and, when administered separately this may occur simultaneously or sequentially in any order. Such sequential administration may be close in time or remote in time.

The invention also includes compositions containing the compounds of the invention and a carrier, diluent or excipient, as well as methods of using the compounds of the invention to prepare such compositions. In a particular embodiment, there is provided a pharmaceutical composition comprising a compound of Formula I and a pharmaceutically acceptable carrier, diluent or excipient.

In a particular embodiment, there is provided a pharmaceutical composition comprising a compound of Formula α, α′, α″, I, I′, I″, II, II′, II″, III, III′, III″, IV, IV′, IV″, V, V′, V″, VI, VI′, VI″ or VII and a pharmaceutically acceptable carrier, diluent or excipient.

Typically, the compounds of the invention used in the methods of the invention are formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but may range anywhere from about 3 to about 8. Formulation in an acetate buffer at pH 5 is a suitable embodiment. In an embodiment, formulations comprising compounds of the invention are sterile. The compounds ordinarily will be stored as a solid composition, although lyophilized formulations or aqueous solutions are acceptable.

Compositions comprising compounds of the invention will be formulated, dosed, and administered in a fashion consistent with good medical practice. Factors for consideration in this context include the particular disorder being treated, the particular mammal being treated, the clinical condition of the individual patient, the cause of the disorder, the site of administration, the method of administration, the scheduling of administration, and other factors known to medical practitioners.

The compounds may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

Generally, the initial pharmaceutically effective amount of the compound of the invention administered parenterally per dose will be in the range of about 0.01-100 mg/kg/day, for example about 0.1 to 20 mg/kg of patient body weight per day, with the typical initial range of compound used being 0.3 to 15 mg/kg/day. Oral unit dosage forms, such as tablets and capsules, may contain from about 25 to about 1000 mg of the compound of the invention.

The compound of the invention may be administered by any suitable means, including oral, sublingual, buccal, topical, transdermal, parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (“PVP”) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g., a salt such sodium chloride, if desired. The solution is typically filtered, e.g., using a 0.2 micron filter, to remove impurities and contaminants.

Another formulation may be prepared by mixing a compound described herein and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound described herein or pharmaceutical composition thereof) or aid in the manufacturing of the pharmaceutical product (i.e., medicament).

### EXAMPLES

The invention will be more fully understood by reference to the following examples. They should not, however, be construed as limiting the scope of the invention. For example, the synthesis of non-exemplified compounds may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds described herein. The identity and purity of compounds were checked by LCMS and ¹H NMR analysis.

Column chromatography was done on a Biotage system (Manufacturer: Dyax Corporation) having a silica gel column or on a silica SepPak cartridge (Waters) (unless otherwise stated). ¹H NMR spectra were recorded on a Varian instrument operating at 400 MHz. ¹H-NMR spectra were obtained as CDCl₃, CD₃OD, D₂O, (CD₃)₂SO, (CD₃)₂CO, C₆D₆, CD₃CN solutions (reported in ppm), using tetramethylsilane (0.00 ppm) or residual solvent (CDCl₃: 7.26 ppm; CD₃OD: 3.31 ppm; D₂O: 4.79 ppm; (CD₃)₂SO: 2.50 ppm; (CD₃)₂CO: 2.05 ppm; C₆D₆: 7.16 ppm; CD₃CN: 1.94 ppm) as the reference standard. When peak multiplicities are reported, the following abbreviations are used: s (singlet), d (doublet), t (triplet), q (quartet), m (multiplet), br (broadened), dd (doublet of doublets), dt (doublet of triplets). Coupling constants, when given, are reported in Hertz (Hz).

In the Examples described below, unless otherwise indicated all temperatures are set forth in degrees Celsius. Reagents were purchased from commercial suppliers such as Sigma-Aldrich, Alfa Aesar, or TCI, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

### Biological Example

### Cellular BACE1 Inhibition Assay

The BACE inhibition properties of the compounds of the invention may be determined by the following in vitro cellular Amyloidβ 1-40 production assay.

Inhibition of Amyloidβ1-40 production was determined by incubating cells with compound for 48 hours and quantifying the level of Amyloidβ 1-40 using an HTRF immunoassay.

Materials and Methods: HEK-293 cells stably transfected with a DNA construct containing the coding sequence for the wild type APP695 sequence were grown in DMEM supplemented with 10% fetal bovine serum, penicillin/streptomycin and 150 μg/mL G418. Cells were plated in 96-well plates at 35,000 cells/well and allowed to attach for 8-12 hours. Media was changed to DMEM supplemented with 10% fetal bovine serum, penicillin/streptomycin 15 minutes prior to compound addition. Diluted compounds were then added at a final concentration of 0.5% DMSO. After 48 hours, 4 μL of media from each well was added to a corresponding well of a 384 well plate (Perkin Elmer Cat#6008280) containing the HTRF reagents. HTRF reagents were obtained from the CisBio Amyloidβ 1-40 peptide assay kit (Cat#62B40PEC) and were prepared as follows anti-peptide β (1-40)-Cryptate and anti-peptide β (1-40)-XL655 were stored in 2 plate aliquots at −80° C. Diluent and Reconstitution buffer were stored at 4° C. Aliquots of the two antibodies were diluted 1:100 with Reconstitution buffer, and this mixture was diluted 1:2 with Diluent. 12 μL of the reagent mixture was added to the required wells of the 384 well assay plate. The assay plate was incubated at 4° C. for 17 hours and then analyzed for fluorescence at 665 and 620 nm.

The following compounds were tested in the above assay:

| Example # | IC₅₀ (nM) |
|---|---|
| Example 6 | 16.8 |
| Example 7 | 7 |
| Example 11 | 11 |
| Example 13 | 16.6 |
| Example 14 | 30.8 |
| Example 15 | 76.7 |
| Example 17 | 81.9 |
| Example 18 | 37.4 |
| Example 20 | 1429.1 |
| Example 21 | 226.4 |
| Example 22 | 263.8 |
| Example 23 | 28.5 |
| Example 24 | 188.8 |
| Example 25 | 103.5 |
| Example 26 | 19.7 |
| Example 27 | 10.1 |
| Example 28 | 92.5 |
| Example 32 | 48.6 |
| Example 35 | 192.2 |
| Example 37 | 240.7 |
| Example 42 | 120.8 |
| Example 45 | 69.6 |
| Example 56 | 360.9 |
| Example 57 | 363.4 |
| Example 62 | 306 |
| Example 74 | 257.5 |
| Example 78 | 48.5 |
| Example 83 | 145.9 |
| Example 87 | 251.3 |
| Example 88 | 46.3 |
| Example 90 | 16.8 |
| Example 95 | 275.6 |
| Example 96 | 1320.1 |
| Example 109 | 481.9 |
| Example 131 | 5.1 |
| Example 134 | 7.9 |
| Example 135 | 8.1 |
| Example 137 | 6.1 |
| Example 139 | 14.8 |
| Example 144 | 3.3 |
| Example 147 | 1.3 |
| Example 155 | 27.1 |
| Example 156 | 17.2 |
| Example 157 | 39.6 |
| Example 158 | 6.7 |
| Example 159 | 26.1 |
| Example 160 | 87.3 |

### Example 1

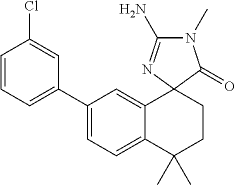

### 2-amino-7′-(3-chlorophenyl)-1,4′,4′-trimethyl-3′,4′-dihydro-2′H-spiro[imidazole-4,1′-naphthalen]-5(1H)-one

Step A: 7-Bromo-4,4-dimethyl-3,4-dihydronaphthalen-1(2H)-one (1.5 g, 5.9 mmol), ammonium carbonate (4.0 g, 41 mmol), KCN (0.77 g, 12 mmol) and NaHSO₃ (0.62 g, 5.9 mmol) were combined and diluted with ethanol (6 mL). The reaction (stainless bomb) was sealed, heated to 130° C. and stirred for 12 hours. The reaction was cooled and poured into ice water. The pH was adjusted to about 6, and the material was stirred for an additional hour. The material was filtered, rinsed with water and dried under vacuum to yield T-bromo-4′,4′-dimethyl-3′,4′-dihydro-2′H-spiro[imidazolidine-4,1′-naphthalene]-2,5-dione (1.7 g, 5.3 mmol, 89% yield).

Step B: T-Bromo-4′,4′-dimethyl-3′,4′-dihydro-2′H-spiro[imidazolidine-4,1′-naphthalene]-2,5-dione (1.7 g, 5.3 mmol) was diluted with DMF (“dimethylformamide”) (15 mL), followed by the addition of K₂CO₃ (0.654 g, 4.73 mmol) and MeI (0.295 mL, 4.73 mmol; d 2.275). After stirring for 12 hours, the reaction was diluted with ethyl acetate and washed with water and brine. The organic was dried over MgSO₄, filtered and concentrated. The material was purified on silica gel eluting with 10-40% ethyl acetate/hexanes to yield 7′-bromo-1,4′,4′-trimethyl-3′,4′-dihydro-2′H-spiro[imidazolidine-4,1′-naphthalene]-2,5-dione (1.58 g, 4.69 mmol, 89.1% yield).

Step C: 7′-Bromo-1,4′,4′-trimethyl-3′,4′-dihydro-2′H-spiro[imidazolidine-4,1′-naphthalene]-2,5-dione (1.58 g, 4.69 mmol) was diluted with toluene (20 mL), followed by the addition of Lawesson's Reagent (1.42 g, 3.51 mmol). The reaction was stirred at reflux for 12 hours. The reaction was allowed to cool diluted with ethyl acetate and washed with saturated bicarbonate, water and brine. The organics were dried over MgSO₄, filtered and concentrated. The residue was purified on silica gel eluting with 20% ethyl acetate/hexanes to afford 7′-bromo-1,4′,4′-trimethyl-2-thioxo-3′,4′-dihydro-2′H-spiro[imidazolidine-4,1′-naphthalen]-5-one (1.2 g, 3.40 mmol, 72.5% yield).

Step D: 7′-Bromo-1,4′,4′-trimethyl-2-thioxo-3′,4′-dihydro-2′H-spiro[imidazolidine-4,1′-naphthalen]-5-one (1.7 g, 4.8 mmol) was diluted with methanol (50 mL) followed by the addition of tert-butyl hydroperoxide (10 mL, 72 mmol) and NH₄OH (21 mL, 178 mmol). The reaction was heated to 40° C., stirred for 2 hours and then for 12 hours at ambient temperature. The reaction was concentrated down (remove methanol) and diluted with DCM (“dichloromethane”) and water. The layers were separated and the organic was dried over MgSO₄, filtered and concentrated. The material was purified on silica gel eluting with 1-10% methanol/DCM (1% NH₄OH) to yield 2-amino-7′-bromo-1,4′,4′-trimethyl-3′,4′-dihydro-2′H-spiro[imidazole-4,1′-naphthalen]-5(1H)-one (1.2 g, 3.6 mmol, 74% yield).

Step E: 2-Amino-7′-bromo-1,4′,4′-trimethyl-3′,4′-dihydro-2′H-spiro[imidazole-4,1′-naphthalen]-5(1H)-one (50 mg, 0.15 mmol), 3-chlorophenylboronic acid (35 mg, 0.22 mmol) and Pd(PPh₃)₄ (8.6 mg, 0.0074 mmol) were combined in a vial and diluted with dioxane (1 mL). Sodium carbonate (223 μL, 0.45 mmol) was added, and the vial was sealed, heated to 95° C. and stirred overnight. The reaction was allowed to cool and loaded onto silica gel running a gradient of 1-10% MeOH/DCM with 1% NH₄OH to yield 2-amino-7′-(3-chlorophenyl)-1,4′,4′-trimethyl-3′,4′-dihydro-2′H-spiro[imidazole-4,1′-naphthalen]-5(1H)-one (30 mg, 0.082 mmol, 55% yield). MS (APCI-pos)=368.2 (M+1).

### Example 2

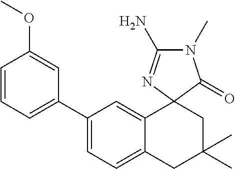

### 2-amino-7′-(3-methoxyphenyl)-1,3′,3′-trimethyl-3′,4′-dihydro-2′H-spiro[imidazole-4,1′-naphthalen]-5(1H)-one

Step A: 1-Bromo-4-(bromomethyl)benzene (10.0 g, 40.0 mmol) in diethyl ether (60 mL) was added dropwise to a suspension of magnesium (0.972 g, 40.0 mmol) in diethyl ether (20 ml) as to maintain a gentle reflux. Following the addition, it was refluxed by heating for 1 hour. It was then cooled to 0° C., and copper (I) chloride (0.0914 g, 0.923 mmol) was added with vigorous stirring. Diethyl 2-(propan-2-ylidene)malonate (6.01 mL, 30.8 mmol) was added dropwise, and the reaction was allowed to warm to ambient temperature and stir overnight. It was quenched by pouring onto 1M sulfuric acid, and it was extracted twice with diethyl ether. The combined organics were dried over anhydrous magnesium sulfate, filtered, and concentrated. It was purified by silica gel chromatography (2-30% EtOAc/hexanes linear gradient) to yield diethyl 2-(1-(4-bromophenyl)-2-methylpropan-2-yl)malonate (6.2 g, 16.70 mmol, 54.26% yield).

Step B: Potassium hydroxide (4.2 g, 75 mmol) was added to a suspension of diethyl 2-(1-(4-bromophenyl)-2-methylpropan-2-yl)malonate (6.2 g, 17 mmol) in 2:1 ethanol/water (50 mL), and it was heated to 70° C. for 24 hours. It was diluted with water and washed twice with dichloromethane. The aqueous layer was acidified with concentrated hydrochloric acid (7 mL), and it was extracted twice with dichloromethane, dried over anhydrous sodium sulfate, filtered, and concentrated to yield 2-(1-(4-bromophenyl)-2-methylpropan-2-yl)malonic acid (4.9 g, 16 mmol, 93% yield).

Step C: Neat 2-(1-(4-bromophenyl)-2-methylpropan-2-yl)malonic acid (4.9 g, 16 mmol) was heated to 200° C. for 1 hour. It was cooled to ambient temperature to yield 4-(4-bromophenyl)-3,3-dimethylbutanoic acid (3.8 g, 14 mmol, 90% yield).

Step D: A suspension of 4-(4-bromophenyl)-3,3-dimethylbutanoic acid (3.8 g, 14.0 mmol) was heated in polyphosphoric acid (38 g, 446 mmol) for 1 hour at 10.0° C. with periodic swirling. It was poured onto water and extracted twice with dichloromethane. The combined extracts were dried over anhydrous sodium sulfate, filtered, and concentrated. It was purified by silica gel chromatography (2-30% EtOAc/hexanes linear gradient) to yield 7-bromo-3,3-dimethyl-3,4-dihydronaphthalen-1(2H)-one (3.1 g, 12.2 mmol, 87.4% yield).

Step E: Sodium bisulfite (˜100 mg), cyanopotassium (2.32 g, 35.6 mmol), and ammonium carbonate (7.97 g, 83.0 mmol) were added to a suspension of 7-bromo-3,3-dimethyl-3,4-dihydronaphthalen-1(2H)-one (3.00 g, 11.9 mmol) in ethanol (12 mL). It was sealed in a stainless steel bomb and heated to 150° C. for 3 days. It was cooled to 0° C., opened, and poured onto water (500 mL) with stirring. It was allowed to stir for 30 minutes, during which material precipitated out of solution. It was then acidified with concentrated HCl until a pH of about 1 was reached, and more material came out of solution. The material was collected via filtration and washed with water and dichloromethane to yield 7′-bromo-3′,3′-dimethyl-3′,4′-dihydro-2′H-spiro[imidazolidine-4,1′-naphthalene]-2,5-dione (1.5 g, 4.64 mmol, 39.2% yield) as a solid.

Step F: Potassium carbonate (4.49 g, 32.5 mmol) and iodomethane (2.03 mL, 32.5 mmol) were added to a solution of 7′-bromo-3′,3′-dimethyl-3′,4′-dihydro-2′H-spiro[imidazolidine-4,1′-naphthalene]-2,5-dione (10.0 g, 30.9 mmol) in DMF (90 mL), and the reaction was allowed to stir overnight. It was diluted with ethyl acetate and washed with water and saturated sodium chloride. It was dried over anhydrous sodium sulfate, filtered, and concentrated. It was purified by recrystallization from EtOAc/hexanes to yield 7′-bromo-1,3′,3′-trimethyl-3′,4′-dihydro-2′H-spiro[imidazolidine-4,1′-naphthalene]-2,5-dione (8.5 g, 25.21 mmol, 81.46% yield) as a crystalline solid.

Step G: Lawesson's Reagent (5.6 g, 14 mmol) was added to a solution of 7′-bromo-1,3′,3′-trimethyl-3′,4′-dihydro-2′H-spiro[imidazolidine-4,1′-naphthalene]-2,5-dione (8.5 g, 25 mmol) in hot 1,2-dichloroethane (125 mL), and the reaction was heated to reflux overnight. It was cooled to room temperature, concentrated to ⅓ its original volume, loaded onto a Biotage SP1 system, and purified by silica gel chromatography eluting with a linear gradient of 2-50% ethyl acetate/hexanes. The impure fractions were recrystallized from EtOAc/hexanes and combined with the pure fractions to yield 7′-bromo-1,3′,3′-trimethyl-2-thioxo-3′,4′-dihydro-2′H-spiro[imidazolidine-4,1′-naphthalen]-5-one (4.6 g, 13 mmol, 52% yield).

Step H: Ammonia (47 mL, 326 mmol) in MeOH and 2-hydroperoxy-2-methylpropane (9.3 mL, 65 mmol) were added to a solution of 7′-bromo-1,3′,3′-trimethyl-2-thioxo-3′,4′-dihydro-2′H-spiro[imidazolidine-4,1′-naphthalen]-5-one (4.6 g, 13 mmol) in dichloromethane (50 mL), and the reaction was allowed to stir for 6 days at ambient temperature. At this point, 7M ammonia (25 mL) in MeOH and 2-hydroperoxy-2-methylpropane (5 mL) were added, and the reaction was allowed to stir for another 5 days. It was quenched by the addition of 25% sodium sulfite (100 mL), and it was allowed to stir for 90 minutes. It was diluted with water and extracted three times with dichloromethane. It was dried over anhydrous sodium sulfate, filtered, and concentrated. It was loaded onto a Biotage SP1 system and purified with a 2-15% methanol/dichloromethane linear gradient to yield 2-amino-7′-bromo-1,3′,3′-trimethyl-3′,4′-dihydro-2′H-spiro[imidazole-4,1′-naphthalen]-5(1H)-one (4.4 g, 13 mmol, 100% yield).

Step I: 3-Methoxyphenylboronic acid (0.033 g, 0.22 mmol), 20% aqueous sodium carbonate (0.23 g, 0.43 mmol), and tetrakis(triphenylphosphine)palladium(0) (0.0096 g, 0.0083 mmol) were added to a solution of 2-amino-7′-bromo-1,3′,3′-trimethyl-3′,4′-dihydro-2′H-spiro[imidazole-4,1′-naphthalen]-5(1H)-one (0.056 g, 0.17 mmol) in toluene (1 mL). It was degassed with argon, sealed, and heated to 110° C. overnight. It was loaded directly onto a Biotage SP1 system and eluted with linear gradient of 4-15% methanol/dichloromethane to yield 2-amino-7′-(3-methoxyphenyl)-1,3′,3′-trimethyl-3′,4′-dihydro-2′H-spiro[imidazole-4,1′-naphthalen]-5(1H)-one (0.037 g, 0.10 mmol, 61% yield). ¹H NMR (400 MHz, CDCl₃) δ 7.42 (d, J=8 Hz, 1H), 7.32 (dd, J=8.9 Hz, 1H), 7.16 (d, J=8 Hz, 1H), 7.09 (s, 1H), 7.05 (d, J=8 Hz, 1H), 7.00 (s, 1H), 6.87 (d, J=9 Hz, 1H), 3.84 (s, 3H), 3.21 (s, 3H), 2.81 (d, J=17 Hz, 1H), 2.59 (d, J=16 Hz, 1H), 2.31 (d, J=13 Hz, 1H), 1.82 (d, J=14 Hz, 1H), 1.16 (s, 3H), 1.02 (s, 3H); m/z (APCI-pos) M+1=364.2.

### Example 3

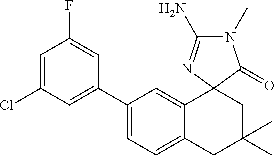

### 2-amino-7′-(3-chloro-5-fluorophenyl)-1,3′,3′-trimethyl-3′,4′-dihydro-2′H-spiro[imidazole-4,1′-naphthalen]-5(1H)-one

2-Amino-7′-(3-chloro-5-fluorophenyl)-1,3′,3′-trimethyl-3′,4′-dihydro-2′H-spiro[imidazole-4,1′-naphthalen]-5(1H)-one (58% yield) was prepared according to Example 2, Step I, substituting 3-fluoro-5-chlorophenylboronic acid in place of 3-methoxyphenylboronic acid. ¹H NMR (400 MHz, CDCl₃) δ 7.4 (m, 1H), 7.2 (m, 2H), 7.1 (m, 3H), 3.22 (s, 3H), 2.83 (d, J=16 Hz, 1H), 2.61 (d, J=16 Hz, 1H), 2.30 (d, J=14 Hz, 1H), 1.82 (d, J=14 Hz, 1H), 1.17 (s, 3H), 1.02 (s, 3H); m/z (APCI-pos) M+1=386.2.

### Example 4

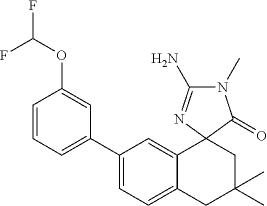

### 2-amino-7′-(3-(difluoromethoxy)phenyl)-1,3′,3′-trimethyl-3′,4′-dihydro-2′H-spiro[imidazole-4,1′-naphthalen]-5(1H)-one

2-Amino-7′-(3-(difluoromethoxy)phenyl)-1,3′,3′-trimethyl-3′,4′-dihydro-2′H-spiro[imidazole-4,1′-naphthalen]-5(1H)-one (51% yield) was prepared according to Example 2, Step I, substituting 3-(difluoromethoxy)phenylboronic acid in place of 3-methoxyphenylboronic acid. ¹H NMR (400 MHz, CDCl₃) δ 7.4 (m, 2H), 7.3 (m, 1H), 7.2 (m, 2H), 7.1 (m, 2H), 6.54 (t, J=73 Hz, 1H), 3.2 (s, 1H), 2.82 (d, J=16 Hz, 1H), 2.60 (d, J=16 Hz, 1H), 2.31 (d, J=14 Hz, 1H), 1.82 (d, J=14 Hz, 1H), 1.17 (s, 3H), 1.02 (s, 3H); m/z (APCI-pos) M+1=400.2.

### Example 5

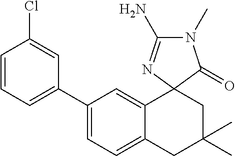

### 2-amino-7′-(3-chlorophenyl)-1,3′,3′-trimethyl-3′,4′-dihydro-2′H-spiro[imidazole-4,11-naphthalen]-5 (1H)-one

2-Amino-7′-(3-chlorophenyl)-1,3′,3′-trimethyl-3′,4′-dihydro-2′H-spiro[imidazole-4,1′-naphthalen]-5(1H)-one (72% yield) was prepared according to Example 2, Step I, substituting 3-chlorophenylboronic acid in place of 3-methoxyphenylboronic acid. ¹H NMR (400 MHz, CDCl₃) δ 7.5-7.0 (m, 7H), 3.2 (s, 3H), 2.82 (d, J=16 Hz, 1H), 2.60 (d, J=15 Hz, 1H), 2.31 (d, J=14 Hz, 1H), 1.82 (d, J=14 Hz, 1H), 1.17 (s, 3H), 1.02 (s, 3H); m/z (APCI-pos) M+1=368.1.

### Example 6

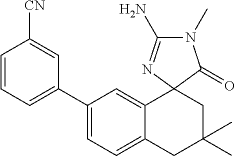

### 3-(2-amino-1,3′,3′-trimethyl-5-oxo-1,3′,4′,5-tetrahydro-2′H-spiro[imidazole-4,1′-naphthalene]-7′-yl)benzonitrile

3-(2-Amino-1,3′,3′-trimethyl-5-oxo-1,3′,4′,5-tetrahydro-2′H-spiro[imidazole-4,1′-naphthalene]-7′-yl)benzonitrile (64% yield) was prepared according to Example 2, Step I, substituting 3-cyanophenylboronic acid in place of 3-methoxyphenylboronic acid. ¹H NMR (400 MHz, CDCl₃) δ 7.74 (m, 1H), 7.69 (m, 1H), 7.50 (m, 1H), 7.38 (m, 1H), 7.20 (m, 1H), 7.06 (m, 1H) 4.0 (br s, 2H), 3.21 (s, 3H), 2.83 (d, J=16 Hz, 1H), 2.62 (d, J=16 Hz, 1H), 2.29 (d, J=14 Hz, 1H), 1.82 (d, J=14 Hz, 1H), 1.17 (s, 3H), 1.02 (s, 3H); m/z (APCI-pos) M+1=359.2.

### Example 7

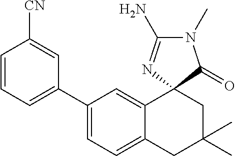

### (R)-3-(2-amino-1,3′,3′-trimethyl-5-oxo-1,3′,4′,5-tetrahydro-2′H-spiro[imidazole-4,1′-naphthalene]-7′-yl)benzonitrile

The SFC separation of 3-(2-amino-1,3′,3′-trimethyl-5-oxo-1,3′,4′,5-tetrahydro-2′H-spiro[imidazole-4,1′-naphthalene]-7′-yl)benzonitrile was carried out on a Chiralcel OD-H (3×15 cm) 07-8754 column eluting with 40% methanol (0.1% DEA)/CO₂ at 100 bar at a flow rate of 80 mL/min (injection volume 2 mL, 53 mg/mL methanol). The peaks isolated were analyzed on Chiralcel OD-H (25×0.46 cm) column eluting with 40% methanol (DEA)/CO₂, at 100 bar (flow rate 3 mL/min, 220 nm). From this separation, (R)-3-(2-amino-1,3′,3′-trimethyl-5-oxo-1,3′,4′,5-tetrahydro-2′H-spiro[imidazole-4,1′-naphthalene]-7′-yl)benzonitrile (peak-1, 617 mg, chemical purity >99%, ee >99%) was isolated.

### Example 8

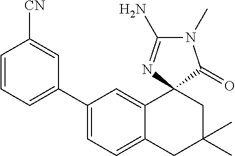

### (S)-3-(2-amino-1,3′,3′-trimethyl-5-oxo-1,3′,4′,5-tetrahydro-2′H-spiro[imidazole-4,1′-naphthalene]-7′-yl)benzonitrile

The SFC separation of 3-(2-amino-1,3′,3′-trimethyl-5-oxo-1,3′,4′,5-tetrahydro-2′H-spiro[imidazole-4,1′-naphthalene]-7′-yl)benzonitrile was carried out on a Chiralcel OD-H (3×15 cm) 07-8754 column eluting with 40% methanol (0.1% DEA (“diethylamine”))/CO₂ at 100 bar at a flow rate of 80 mL/min (injection volume 2 mL, 53 mg/mL methanol). The peaks isolated were analyzed on Chiralcel OD-H (25×0.46 cm) column eluting with 40% methanol (DEA)/CO₂, at 100 bar (flow rate 3 mL/min, 220 nm). From this separation, (S)-3-(2-amino-1,3′,3′-trimethyl-5-oxo-1,3′,4′,5-tetrahydro-2′H-spiro[imidazole-4,1′-naphthalene]-7′-yl)benzonitrile (peak-2, 681 mg, chemical purity >99%, ee >99%) was isolated.

### Example 9

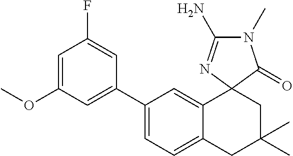

### 2-amino-7′-(3-fluoro-5-methoxyphenyl)-1,3′,3′-trimethyl-3′,4′-dihydro-2′H-spiro[imidazole-4,1′-naphthalen]-5(1H)-one

2-Amino-7′-(3-fluoro-5-methoxyphenyl)-1,3′,3′-trimethyl-3′,4′-dihydro-2′H-spiro[imidazole-4,1′-naphthalen]-5(1H)-one (69% yield) was prepared according to Example 2, Step I, substituting 3-fluoro-5-methoxyphenylboronic acid in place of 3-methoxyphenylboronic acid and dioxane in place of toluene. m/z (APCI-pos) M+1=382.2.

### Example 10

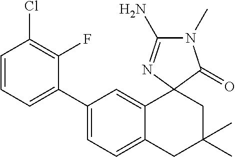

### 2-amino-7′-(3-chloro-2-fluorophenyl)-1,3′,3′-trimethyl-3′,4′-dihydro-2′H-spiro[imidazole-4,1′-naphthalen]-5(1H)-one

2-Amino-7′-(3-chloro-2-fluorophenyl)-1,3′,3′-trimethyl-3′,4′-dihydro-2′H-spiro[imidazole-4,1′-naphthalen]-5(1H)-one (69% yield) was prepared according to Example 2, Step I, substituting 2-fluoro-3-chlorophenylboronic acid in place of 3-methoxyphenylboronic acid and dioxane in place of toluene. m/z (APCI-pos) M+1=386.1.

### Example 11

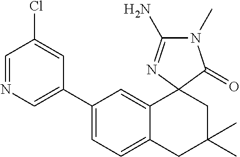

### 2-amino-7′-(5-chloropyridin-3-yl)-1,3′,3′-trimethyl-3′,4′-dihydro-2′H-spiro[imidazole-4,1′-naphthalen]-5(1H)-one

2-Amino-7′-(5-chloropyridin-3-yl)-1,3′,3′-trimethyl-3′,4′-dihydro-2′H-spiro[imidazole-4,1′-naphthalen]-5(1H)-one (41% yield) was prepared according to Example 2, Step I, substituting 5-chloropyridin-3-ylboronic acid in place of 3-methoxyphenylboronic acid. ¹H NMR (400 MHz, CDCl₃) δ 8.59 (s, 1H), 8.51 (s, 1H), 7.75 (s, 1H), 7.38, d, J=8 Hz, 1H), 7.22 (d, J=8 Hz, 1H), 7.07 (s, 1H), 3.20 (s, 3H), 2.84 (d, J=16 Hz, 1H), 2.62 (d, J=16 Hz, 1H), 2.29 (d, J=14 Hz, 1H), 1.81 (d, J=14 Hz, 1H), 1.17 (s, 3H), 1.02 (s, 3H); m/z (APCI-pos) M+1=369.2.

### Example 12

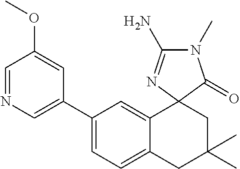

### 2 2-amino-7′-(5-methoxypyridin-3-yl)-1,3′,3′-trimethyl-3′,4′-dihydro-2′H-spiro[imidazole-4,1′-naphthalen]-5 (1H)-one

2-Amino-7′-(5-methoxypyridin-3-yl)-1,3′,3′-trimethyl-3′,4′-dihydro-2′H-spiro[imidazole-4,1′-naphthalen]-5(1H)-one (65% yield) was prepared according to Example 2, Step I, substituting 5-methoxypyridin-3-ylboronic acid in place of 3-methoxyphenylboronic acid and dioxane in place of toluene. ¹H NMR (400 MHz, CDCl₃) δ 8.33 (m, 1H), 8.24 (m, 1H), 7.40 (m, 1H), 7.25 (m, 1H), 7.21 (d, J=8 Hz, 1H), 7.09 (m, 1H), 3.89 (s, 3H), 3.23 (s, 3H), 2.83 (d, J=16 Hz, 1H), 2.61 (d, J=16 Hz, 1H), 2.29 (d, J=14 Hz, 1H), 1.83 (d, J=14 Hz, 1H), 1.17 (s, 3H), 1.03 (s, 3H); m/z (APCI-pos) M+1=365.2.

### Example 13

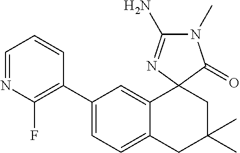

### 2-amino-7′-(2-fluoropyridin-3-yl)-1,3′,3′-trimethyl-3′,4′-dihydro-2′H-spiro[imidazole-4,1′-naphthalen]-5(1H)-one

2-Amino-7′-(2-fluoropyridin-3-yl)-1,3′,3′-trimethyl-3′,4′-dihydro-2′H-spiro[imidazole-4,1′-naphthalen]-5(1H)-one (71% yield) was prepared according to Example 2, Step I, substituting 2-fluoropyridin-3-ylboronic acid in place of 3-methoxyphenylboronic acid and dioxane in place of toluene. ¹H NMR (400 MHz, CDCl₃) δ 8.16 (m, 1H), 7.78 (m, 1H), 7.42 (m, 1H), 7.2 (m, 2H), 7.10 (s, 1H), 3.20 (s, 1H), 2.83 (d, J=16 Hz, 1H), 2.61 (d, J=16 Hz, 1H), 2.31 (d, J=14 Hz, 1H), 1.83 (d, J=14 Hz, 1H), 1.17 (s, 3H), 1.03 (s, 3H); m/z (APCI-pos) M+1=353.2.

### Example 14

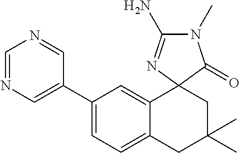

### 2-amino-1,3′,3′-trimethyl-7′-(pyrimidin-5-yl)-3′,4′-dihydro-2′H-spiro [imidazole-4,1′-naphthalen]-5(1H)-one

### 2-Amino-1,3′,3′-trimethyl-7′-(pyrimidin-5-yl)-3′,4′-dihydro-2′H-spiro[imidazole-4,1′-naphthalen]-5(1H)-one (76% yield) was prepared according to Example 2, Step I, substituting pyrimidin-5-ylboronic acid in place of 3-methoxyphenylboronic acid and dioxane in place of toluene. ¹H NMR (400 MHz, CDCl₃) δ 9.16 (s, 1H), 8.85 (s, 2H), 7.42 (m, 1H), 7.26 (m, 1H), 7.10 (m, 1H), 3.22 (s, 3H), 2.85 (d, J=16 Hz, 1H), 2.64 (d, J=16 Hz, 1H), 2.29 (d, J=14 Hz, 1H), 1.84 (d, J=14 Hz, 1H), 1.18 (s, 3H), 1.03 (s, 3H); m/z (APCI-pos) M+1=336.2.

### Example 15

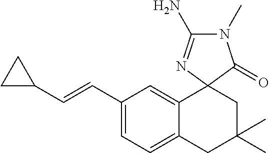

### (E)-2-amino-7′-(2-cyclopropylvinyl)-1,3′,3′-trimethyl-3′,4′-dihydro-2′H-spiro[imidazole-4,1′-naphthalen]-5(1H)-one

(E)-2-Amino-7′-(2-cyclopropylvinyl)-1,3′,3′-trimethyl-3′,4′-dihydro-2′H-spiro[imidazole-4,1′-naphthalen]-5(1H)-one (76% yield) was prepared according to Example 2, Step I, substituting (E)-2-(2-cyclopropylvinyl)-4,4,5,5-tetramethyl-1,3,2-dioxaborolane in place of 3-methoxyphenylboronic acid and dioxane in place of toluene. ¹H NMR (400 MHz, CDCl₃) δ 7.19 (m, 1H), 6.99 (d, J=8 Hz, 1H), 6.79 (m, 1H), 6.35 (d, J=16 Hz, 1H), 5.63 (dd, J=9, 16 Hz, 1H) 3.22 (s, 3H), 2.73 (d, J=16 Hz, 1H), 2.51 (d, J=16 Hz, 1H), 2.26 (d, J=14 Hz, 1H), 1.78 (d, J=14 Hz, 1H), 1.51 (m, 1H), 1.13 (s, 3H), 0.97 (s, 3H), 0.79 (m, 2H), 0.48 (m, 2H); m/z (APCI-pos) M+1=324.1.

### Example 16

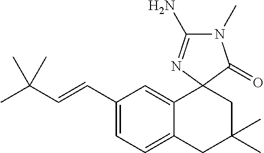

### (E)-2-amino-7′-(3,3-dimethylbut-1-enyl)-1,3′,3′-trimethyl-3′,4′-dihydro-2′H-spiro[imidazole-4,1′-naphthalen]-5(1H)-one

(E)-2-Amino-7′-(3,3-dimethylbut-1-enyl)-1,3′,3′-trimethyl-3′,4′-dihydro-2′H-spiro[imidazole-4,1′-naphthalen]-5(1H)-one (76% yield) was prepared according to Example 2, Step I, substituting (E)-3,3-dimethylbut-1-enylboronic acid in place of 3-methoxyphenylboronic acid and dioxane in place of toluene. ¹H NMR (400 MHz, CDCl₃) δ 7.28 (m, 1H), 7.01 (d, J=8 Hz, 1H), 6.82 (m, 1H), 6.19 (d, J=16 Hz, 1H), 6.13 (d, J=16 Hz, 1H), 3.23 (s, 3H), 2.75 (d, J=16 Hz, 1H), 2.51 (d, J=16 Hz, 1H), 2.27 (d, J=14 Hz, 1H), 1.79 (d, J=14 Hz, 1H), 1.13 (s, 3H), 1.09 (s, 9H), 0.96 (s, 3H); m/z (APCI-pos) M+1=340.2.

### Example 17

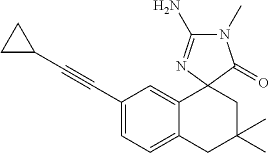

### 2-amino-7′-(cyclopropylethynyl)-1,3′,3′-trimethyl-3′,4′-dihydro-2′H-spiro[imidazole-4,1′-naphthalen]-5(1H)-one

Tri-tert-butylphosphine (0.0516 mL, 0.0178 mmol), bis(acetonitrile)palladium(II) chloride (0.00231 g, 0.00892 mmol), copper (I) iodide (0.00113 g, 0.00595 mmol), diisopropylamine (0.0500 mL, 0.357 mmol), and ethynylcyclopropane (about 0.2 mL, excess) were added to a solution of 2-amino-7′-bromo-1,3′,3′-trimethyl-3′,4′-dihydro-2′H-spiro[imidazole-4,1′-naphthalen]-5(1H)-one (0.100 g, 0.297 mmol) in degassed acetonitrile (1 mL). The reaction was sealed and stirred at ambient temperature overnight. It was then heated to 40° C. for 1 day, and it was loaded directly onto a Biotage SP1 system and purified by silica gel chromatography to yield 2-amino-7′-(cyclopropylethynyl)-1,3′,3′-trimethyl-3′,4′-dihydro-2′H-spiro[imidazole-4,1′-naphthalen]-5(1H)-one (0.062 g, 0.193 mmol, 64.9% yield). ¹H NMR (400 MHz, CDCl₃) δ 7.2 (m, 1H), 7.0 (m, 2H), 3.2 (s, 3H), 2.74 (d, J=17 Hz, 1H), 2.52 (d, J=17 Hz, 1H), 2.25 (d, J=14 Hz, 1H), 1.78 (d, J=14 Hz, 1H), 1.4 (m, 1H), 1.13 (s, 3H), 0.96 (s, 3H), 0.8 (m, 4H); m/z (APCI-pos) M+1=322.2.

### Example 18

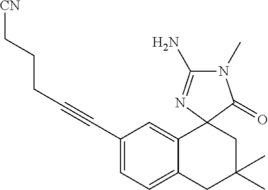

### 6-(2-amino-1,3′,3′-trimethyl-5-oxo-1,3′,4′,5-tetrahydro-2′H-spiro[imidazole-4,1′-naphthalene]-7′-yl)hex-5-ynenitrile

6-(2-Amino-1,3′,3′-trimethyl-5-oxo-1,3′,4′,5-tetrahydro-2′H-spiro[imidazole-4,1′-naphthalene]-7′-yl)hex-5-ynenitrile (15% yield) was prepared according to Example 17 substituting 5-hexynenitrile for ethynylcyclopropane and bis(tri-tert-butylphosphine)palladium(0) for bis(acetonitrile)palladium(II) chloride and tri-tert-butylphosphine. ¹H NMR (400 MHz, CDCl₃) δ 7.3 (m, 1H), 7.1 (m, 1H), 6.9 (m, 1H), 3.3 (s, 1H), 2.8 (m, 1H), 2.6-1.8 (m, 9H), 1.1 (s, 3H), 1.0 (s, 3H); m/z (APCI-pos) M+1=349.2.

### Example 19

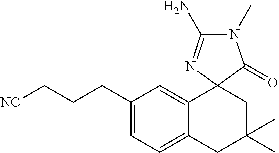

### 4-(2-amino-1,3′,3′-trimethyl-5-oxo-1,3′,4′,5-tetrahydro-2′H-spiro[imidazole-4,1′-naphthalene]-7′-yl)butanenitrile

Bis(tri-tert-butylphosphine)palladium(0) (0.0076 g, 0.015 mmol) was added to a solution of 2-amino-7′-bromo-1,3′,3′-trimethyl-3′,4′-dihydro-2′H-spiro[imidazole-4,1′-naphthalen]-5(1H)-one (0.050 g, 0.15 mmol) and (3-cyanopropyl)zinc (II) bromide (0.65 mL, 0.33 mmol) in THF (1 mL) degassed with argon, and the reaction was sealed and heated to 70° C. for 16 hours. It was loaded directly onto a Biotage SP1 system and purified by silica gel chromatography. It was further purified on a Gilson preparatory HPLC (MeOH/water with 0.1% formic acid) to yield 4-(2-amino-1,3′,3′-trimethyl-5-oxo-1,3′,4′,5-tetrahydro-2′H-spiro[imidazole-4,1′-naphthalene]-7′-yl)butanenitrile (0.018 g, 0.055 mmol, 37% yield). m/z (APCI-pos) M+1=325.2.

### Example 20

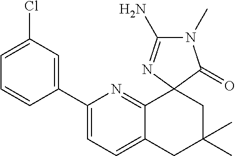

### 2-amino-2′-(3-chlorophenyl)-1,6′,6′-trimethyl-6′,7′-dihydro-5′H-spiro[imidazole-4,8′-quinolin]-5(1H)-one

Step A: A mixture of commercially available 4,4-dimethylcyclohex-2-enone (46 g, 370.97 mmol, 1.00 equiv) and Pd/C (4.6 g) in tetrahydrofuran (250 mL) was stirred for 15 hours at room temperature under a hydrogen atmosphere. The solid was filtered out. The filtrate was concentrated under vacuum. This resulted in 4,4-dimethylcyclohexanone (46 g, 98%) as a solid.

Step B: A solution of 4,4-dimethylcyclohexanone (46 g, 365.08 mmol, 1.00 equiv) in toluene (400 mL), pyrrolidine (77.8 g, 1.10 mol, 3.00 equiv) and 4-methylbenzenesulfonic acid (4.6 g, 26.74 mmol, 0.07 equiv) was placed into a 1000-mL 3-necked round-bottom flask. The resulting solution was heated to reflux for 5 hours in an oil bath. The resulting mixture was cooled and concentrated under vacuum. This resulted in crude 1-(4,4-dimethylcyclohex-1-enyl)pyrrolidine (65.3 g) as an oil.

Step C: A solution of 1-(4,4-dimethylcyclohex-1-enyl)pyrrolidine (65.3 g, 364.80 mmol, 1.00 equiv) in 1,4-dioxane (400 mL) and acrylonitrile (73.3 g, 1.38 mol, 3.78 equiv) was placed into a 1000-mL round-bottom flask purged and maintained with an inert atmosphere of nitrogen. The resulting solution was heated to reflux overnight. After cooling to room temperature, water (100 mL) was added in portions to the reaction mixture. The resulting solution was heated to reflux for an additional 1 hour. The resulting mixture was cooled and concentrated under vacuum. The residue was diluted with water (200 mL) and then extracted with ether (2×200 mL). The organic layers were combined, washed with HCl (0.1M, 1×200 mL) and brine (200 mL). The organic phase was dried over anhydrous sodium sulfate and concentrated under vacuum. The residue was applied onto a silica gel column and eluted with ethyl acetate/petroleum ether (1:40). This resulted in 3-(5,5-dimethyl-2-oxocyclohexyl)propanenitrile (46.5 g, 71%) as an oil.

Step D: Sulfuric acid (260 mL) was placed into a 1000-mL 3-necked round-bottom flask, then 3-(5,5-dimethyl-2-oxocyclohexyl)propanenitrile (46.5 g, 259.78 mmol, 1.00 equiv) was added dropwise with stirring at 0° C. The resulting solution was stirred overnight at room temperature, then quenched by the addition of ice water (300 mL). The resulting solution was extracted with dichloromethane (2×200 mL). The aqueous layer was adjusted to a pH of about 9 to 10 with NH₄OH. The resulting solution was extracted with dichloromethane (2×300 mL). The organic layers were combined, washed with brine (1×300 mL), dried over anhydrous sodium sulfate and concentrated under vacuum. The residue was applied onto a silica gel column and eluted with dichloromethane/methanol (40:1). This resulted in 6,6-dimethyl-5,6,7,8-tetrahydroquinolin-2(1H)-one (24 g, 52%) as a solid.

Step E: A solution of 6,6-dimethyl-5,6,7,8-tetrahydroquinolin-2(1H)-one (24 g, 135.59 mmol, 1.00 equiv) in CH₃CN (300 mL) and phosphoryl tribromide (116.5 g, 405.92 mmol, 3.00 equiv) was placed into a 1000-mL round-bottom flask. The resulting solution was heated to reflux overnight. The reaction mixture was then cooled and quenched by the addition of ice water (200 mL). The pH value of the solution was adjusted to about 8 to 9 with sodium carbonate solution (1 M). The resulting solution was extracted with ethyl acetate (3×200 mL). The organic layers were combined, washed with brine (1×200 mL), dried over anhydrous sodium sulfate and concentrated under vacuum. The residue was applied onto a silica gel column and eluted with ethyl acetate/petroleum ether (1:100). This resulted in 2-bromo-6,6-dimethyl-5,6,7,8-tetrahydroquinoline (12 g, 37%) as an oil.

Step F: A solution of 2-bromo-6,6-dimethyl-5,6,7,8-tetrahydroquinoline (12 g, 49.98 mmol, 1.00 equiv) in acetic acid (45 mL) and H₂O₂ (9 mL) was placed into a 500-mL round-bottom flask. The resulting solution was stirred for 6 hours at 70° C. A second portion of H₂O₂ (9 mL) was added to the reaction mixture and stirred overnight at 70° C. The resulting mixture was cooled and concentrated under vacuum. The residue was dissolved in dichloromethane (100 mL), and then sodium carbonate (28 g) was added and stirred for 1 hour at room temperature. The solid was filtered out. The filtrate was concentrated under vacuum. The residue was dissolved in (CH₃CO)₂O (120 mL) and stirred overnight at 90° C. The resulting mixture was cooled and concentrated under vacuum. The residue was applied onto a silica gel column and eluted with ethyl acetate/petroleum ether (1:50). This resulted in 2-bromo-6,6-dimethyl-5,6,7,8-tetrahydroquinolin-8-yl acetate (3.2 g, 21%) as an oil.

Step G: 2-Bromo-6,6-dimethyl-5,6,7,8-tetrahydroquinolin-8-yl acetate (3.2 g, 10.73 mmol, 1.00 equiv) and HCl (10%, 30 mL) were placed into a 250-mL round-bottom flask. The resulting solution was heated to reflux for 1 hour. The reaction mixture was cooled to room temperature, and then adjusted to a pH of about 7 to 8 with addition of sodium hydroxide solution (1 mol/L). The resulting solution was extracted with dichloromethane (3×50 mL). The organic layers were combined, washed with brine (1×50 mL), dried over anhydrous sodium sulfate and concentrated under vacuum. This resulted in 2-bromo-6,6-dimethyl-5,6,7,8-tetrahydroquinolin-8-ol (2.4 g, 87%) as an oil.

Step H: A solution of 2-bromo-6,6-dimethyl-5,6,7,8-tetrahydroquinolin-8-ol (2.4 g, 9.37 mmol, 1.00 equiv) in dichloromethane (40 mL) and MnO₂ (6.9 g, 8.50 equiv) were placed into a 250-mL round-bottom flask. The resulting solution was stirred overnight at room temperature. The solid was filtered out. The filtrate was concentrated under vacuum. The residue was applied onto a silica gel column and eluted with ethyl acetate/petroleum ether (1:20). This resulted in 2-bromo-6,6-dimethyl-6,7-dihydroquinolin-8(5H)-one (0.924 g, 39%) as a solid. ¹H NMR (400 MHz, CDCl₃) δ 7.59-7.61 (d, 2H), 7.51-7.53 (d, 1H), 2.88 (s, 2H). m/z (ESI-pos) M+1=254.

Step I: 2-Amino-2′-bromo-1,6′,6′-trimethyl-6′,7′-dihydro-5′H-spiro[imidazole-4,8′-quinolin]-5(1H)-one was prepared from 2-bromo-6,6-dimethyl-6,7-dihydroquinolin-8(5H)-one according to Example 2, Steps E-G. m/z (APCI-pos) M+1=337.1.

Step J: 2-Amino-2′-bromo-1,6′,6′-trimethyl-6′,7′-dihydro-5′H-spiro[imidazole-4,8′-quinolin]-5(1H)-one (0.040 g, 0.119 mmol), 3-chlorophenylboronic acid (0.0223 g, 0.142 mmol) and Pd(PPh₃)₄ (0.00685 g, 0.00593 mmol) in dioxane (1 mL) and 2M Na₂CO₃ (0.237 mL, 0.474 mmol) were added to a sealable vial. The vial was degassed with N₂, sealed, and stirred at 80° C. for 16 hours. The reaction mixture was cooled to room temperature and diluted with CH₂Cl₂. Na₂SO₄ was added, the filtrate was decanted off the Na₂SO₄, rinsed with CH₂Cl₂, decanted again, and the combined filtrate was concentrated. The crude product was purified on silica gel (10:1 CH₂Cl₂/7N N14₃ in MeOH) to provide 2-amino-2′-(3-chlorophenyl)-1,6′,6′-trimethyl-6′,7′-dihydro-5′H-spiro[imidazole-4,8′-quinolin]-5(1H)-one (0.026 g, 0.0705 mmol, 59.4% yield) as a foam. m/z (APCI-pos) M+1=369.2.

### Example 21

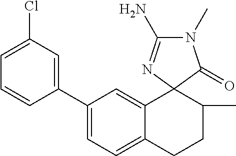

### 2-amino-7′-(3-chlorophenyl)-1,2′-dimethyl-3′,4′-dihydro-2′H-spiro[imidazole-4,1′-naphthalen]-5(1H)-one

Step A: Butyllithium (5.60 mL, 14.0 mmol) was added to a solution of diisopropylamine (2.07 mL, 14.7 mmol) in THF (30 mL, 13.3 mmol) at −78° C. under N₂. This was stirred for 30 minutes and then at 0° C. for 10 minutes. 7-Bromo-3,4-dihydronaphthalen-1(2H)-one (3.0 g, 13.3 mmol) was then added and stirred for 1 hour at −78° C. Iodomethane (1.08 mL, 17.3 mmol) was slowly added, and the reaction mixture was allowed to come to room temperature overnight. TLC (“thin layer chromatography”) showed both mono and di addition. The reaction was then partitioned between EtOAc and water. The aqueous layer was washed with EtOAc twice, and the combined organics were washed with brine and then dried with Na₂SO₄. The mixture was then concentrated down and purified on a column using EtOAc:hexane to give the 7-bromo-2-methyl-3,4-dihydronaphthalen-1(2H)-one (0.56 g, 2.36 mmol, 17%) as an oil.

Step B: A mixture of 7-bromo-2-methyl-3,4-dihydronaphthalen-1(2H)-one (0.563 g, 2.35 mmol), KCN (0.307 g, 4.71 mmol), NaHSO₃ (0.245 g, 2.35 mmol) and ammonium carbonate (1.81 g, 18.8 mmol) in ethanol (4 mL, 2.35 mmol) in a bomb was heated to 130° C. overnight. The mixture was poured onto ice. Concentrated HCl was added to this to bring the pH down to about 3. The mixture was then stirred for an hour, and then the solid was filtered off to give T-bromo-2′-methyl-3′,4′-dihydro-2′H-spiro[imidazolidine-4,1′-naphthalene]-2,5-dione (0.71 g, 2.29 mmol, 97%).

Step C: Iodomethane (0.143 mL, 2.29 mmol) was added to a solution of 7′-bromo-2′-methyl-3′,4′-dihydro-2′H-spiro[imidazolidine-4,1′-naphthalene]-2,5-dione (0.708 g, 2.29 mmol) and K₂CO₃ (0.475 g, 3.44 mmol) in DMF (10 mL, 2.29 mmol). This was stirred at room temperature overnight. The mixture was taken up in EtOAc and water. The organics were washed with water five times and then dried with brine and Na₂SO₄. This was then concentrated down and purified on a column using EtOAc:hexanes to give 7′-bromo-1,2′-dimethyl-3′,4′-dihydro-2′H-spiro[imidazolidine-4,1′-naphthalene]-2,5-dione (0.58 g, 1.79 mmol, 78%) as a solid.

Step D: Lawesson's Reagent (0.436 g, 1.08 mmol) was added to a solution of 7′-bromo-1,2′-dimethyl-3′,4′-dihydro-2′H-spiro[imidazolidine-4,1′-naphthalene]-2,5-dione (0.580 g, 1.79 mmol) in toluene (25 mL, 1.79 mmol). This was heated to reflux overnight. The mixture was partitioned between DCM and water. The aqueous layer was washed twice with DCM, and the combined organics were washed with brine and dried with Na₂SO₄. This was concentrated down and purified on a column using EtOAc:hexanes to give the 7′-bromo-1,2′-dimethyl-2-thioxo-3′,4′-dihydro-2′H-spiro[imidazolidine-4,1′-naphthalen]-5-one (0.246 g, 0.725 mmol, 40%) as a solid.

Step E: 2-Hydroperoxy-2-methylpropane (3.11 mL, 21.8 mmol) was added to a solution of 7′-bromo-1,2′-dimethyl-2-thioxo-3′,4′-dihydro-2′H-spiro[imidazolidine-4,1′-naphthalen]-5-one (0.246 g, 0.725 mmol) in MeOH (8 mL, 197 mmol; d. 0.791), and then 14.1 M NH₄OH (3.09 mL, 43.5 mmol) was added. This was stirred at 50° C. for 3 hours and then at room temperature for 4 hours. The mixture was then partitioned between DCM and water. The aqueous layer was washed with DCM twice. The combined organics were washed with brine and dried with Na₂SO₄. This was then concentrated down and purified on a column using DCM:MeOH:NH₄OH (89:10:1). This gave 2-amino-7′-bromo-1,2′-dimethyl-3′,4′-dihydro-2′H-spiro[imidazole-4,1′-naphthalen]-5(1H)-one (0.136 g, 0.422 mmol, 58%) as a solid. The product was a 60:40 mixture of diastereomers.

Step F: A mixture of 2-amino-7′-bromo-1,2′-dimethyl-3′,4′-dihydro-2′H-spiro[imidazole-4,1′-naphthalen]-5(1H)-one (0.030 g, 0.0931 mmol), 3-chlorophenylboronic acid (0.0160 g, 0.102 mmol), Pd(PPh₃)₄ (0.0108 g, 0.00931 mmol) and Na₂CO₃ (0.0931 mL, 0.186 mmol) in dioxane (1 mL, 0.0931 mmol) was stirred at 90° C. overnight. The mixture was then filtered through Celite®. The filtrate was then concentrated down to be purified on reverse phase prep HPLC. This gave 2-amino-7′-(3-chlorophenyl)-1,2′-dimethyl-3′,4′-dihydro-2′H-spiro[imidazole-4,1′-naphthalen]-5(1H)-one (0.013 g, 0.0376 mmol, 40%) as a solid. MS (APCI-pos)=354 (M+1).

### Example 22

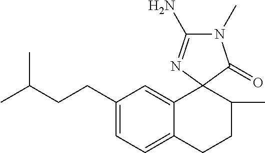

### 2-amino-7′-isopentyl-1,2′-dimethyl-3′,4′-dihydro-2′H-spiro[imidazole-4,1′-naphthalen]-5(1H-one

A mixture of bis(tri-t-butylphosphine)palladium (0) (0.00634 g, 0.0124 mmol) and 2-amino-7′-bromo-1,2′-dimethyl-3′,4′-dihydro-2′H-spiro[imidazole-4,1′-naphthalen]-5(1H)-one (0.040 g, 0.124 mmol) in isopentylzinc (II) bromide (0.621 mL, 0.310 mmol) was stirred at 90° C. overnight. The mixture was then filtered through Celite®. The filtrate was then concentrated down to be purified on reverse phase prep HPLC. This gave 2-amino-7′-isopentyl-1,2′-dimethyl-3′,4′-dihydro-2′H-spiro[imidazole-4,1′-naphthalen]-5(1H)-one (0.0152 g, 0.0485 mmol, 39%) as a solid. MS (APCI-pos)=314 (M+1).

### Example 23

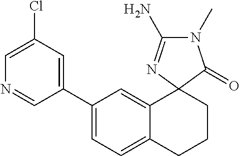

### 2-amino-7′-(5-chloropyridin-3-yl)-1-methyl-3′,4′-dihydro-2′H-spiro[imidazole-4,1′-naphthalen]-5(1H)-one

Step A: A 25 mL acid digestion Parr stainless steel bomb was charged with 7-bromo-3,4-dihydronaphthalen-1(2H)-one (1.50 g, 6.66 mmol), KCN (0.868 g, 13.3 mmol), ammonium carbonate (4.48 g, 46.6 mmol), and absolute EtOH (8 mL). The bomb was sealed, and the reaction mixture was heated in a 130° C. oil bath for 24 hours. The reaction mixture was then cooled and rinsed into a flask with water, causing precipitation, and the mixture was slowly acidified to about pH 2 with 1M HCl (using caution as HCN was generated). The mixture was sparged with nitrogen for 30 minutes. Then the solids were isolated by vacuum filtration through qualitative filter paper on a Buchner funnel, rinsed with water, air dried, and dried in vacuo to give 7′-bromo-3′,4′-dihydro-2′H-spiro[imidazolidine-4,1′-naphthalene]-2,5-dione (1.82 g, 92% yield) as a powder.

Step B: K₂CO₃ (0.852 g, 6.17 mmol) and MeI (0.385 mL, 6.17 mmol) were added to a solution of 7′-bromo-3′,4′-dihydro-2′H-spiro[imidazolidine-4,1′-naphthalene]-2,5-dione (1.82 g, 6.17 mmol) in DMF (21 mL). The reaction mixture was stirred at room temperature for 22 hours, after which it was concentrated to ⅓ volume and water was added, causing precipitation. The solids were isolated by vacuum filtration through qualitative filter paper on a Buchner funnel, rinsed with water, air dried, and dried in vacuo to give 7′-bromo-1-methyl-3′,4′-dihydro-2′H-spiro[imidazolidine-4,1′-naphthalene]-2,5-dione (1.87 g, 98% yield) as a powder.

Step C: 7′-Bromo-1-methyl-3′,4′-dihydro-2′H-spiro[imidazolidine-4,1′-naphthalene]-2,5-dione (1.87 g, 6.05 mmol) and Lawesson's Reagent (1.59 g, 3.93 mmol) with toluene (17 mL) were combined in a 75 mL sealable reaction tube, and the mixture was heated in a 140° C. sand bath and stirred for 20 hours. The toluene was removed in vacuo, the resulting oil was dissolved in DCM, and saturated NaHCO₃ was added. The mixture was extracted with DCM (2×), and the combined extracts were dried (Na₂SO₄), filtered, and concentrated. The crude was purified on silica gel (0-5% ethyl acetate in DCM gradient) to give 7′-bromo-1-methyl-2-thioxo-3′,4′-dihydro-2′H-spiro[imidazolidine-4,1′-naphthalen]-5-one (0.784 g, 40% yield) as a foam.

Step D: t-Butyl hydroperoxide (70% aqueous, 5.01 mL, 36.2 mmol) and 30% NH₄OH (9.39 mL, 72.3 mmol) were added sequentially to a 75 mL sealable reaction tube containing a solution of 7′-bromo-1-methyl-2-thioxo-3′,4′-dihydro-2′H-spiro[imidazolidine-4,1′-naphthalen]-5-one (0.784 g, 2.41 mmol) in MeOH (10 mL) and THF (6 mL), and the reaction mixture was heated in a 40° C. sand bath and stirred for 5 hours. The reaction mixture was diluted with brine (4 mL), the organics were removed in vacuo, and the resulting mixture was treated with saturated NH₄Cl. The mixture was extracted with DCM (2×), and the combined extracts were dried (Na₂SO₄), filtered, and concentrated. The crude was purified on silica gel (2-20% MeOH in DCM gradient) to give 2-amino-7′-bromo-1-methyl-3′,4′-dihydro-2′H-spiro[imidazole-4,1′-naphthalen]-5(1H)-one (0.364 g, 49% yield) as a solid.

Step E: 2-Amino-7′-bromo-1-methyl-3′,4′-dihydro-2′H-spiro[imidazole-4,1′-naphthalen]-5(1H)-one (0.050 g, 0.162 mmol), 5-chloropyridin-3-ylboronic acid (0.0306 g, 0.195 mmol), and Pd(PPh₃)₄ (0.0187 g, 0.0162 mmol) were combined with dioxane (0.8 mL) and 2M Na₂CO₃ (0.324 mL, 0.649 mmol) (both degassed with nitrogen sparge for 45 minutes prior to use), and the reaction mixture was heated in a 90° C. reaction block and stirred for 18 hours. The reaction mixture was diluted with DCM, Na₂SO₄ was added, the mixture was stirred 10 minutes, and it was filtered through a cotton-plugged pipet topped with Na₂SO₄, rinsed with DCM, and the filtrate was concentrated. The crude was purified by preparative TLC (1 mm plate, 9:1 DCM:7N NH₃ in MeOH) to give 2-amino-7′-(5-chloropyridin-3-yl)-1-methyl-3′,4′-dihydro-2′H-spiro[imidazole-4,1′-naphthalen]-5(1H)-one (0.020 g, 36.2% yield) as a powder. ¹H NMR (400 MHz, CDCl₃) δ 8.62 (s, 1H), 8.51 (s, 1H), 7.75 (s, 1H), 7.38 (d, J=7.0 Hz, 1H), 7.29-7.24 (m, 1H), 7.11 (s, 1H), 3.20 (s, 3H), 2.98-2.85 (m, 2H), 2.38-2.26 (m, 1H), 2.26-2.16 (m, 1H), 2.06-1.84 (m, 2H); m/z (APCI+) M+1=341.

### Example 24

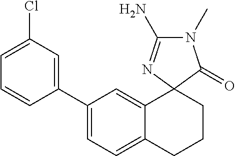

### 2-amino-7′-(3-chlorophenyl)-1-methyl-3′,4′-dihydro-2′H-spiro[imidazole-4,1′-naphthalen]-5(1H)-one

2-Amino-7′-(3-chlorophenyl)-1-methyl-3′,4′-dihydro-2′H-spiro[imidazole-4,1′-naphthalen]-5(1H)-one was prepared according to the procedures of Example 23, in which 3-chlorophenylboronic acid was used in place of 5-chloropyridin-3-ylboronic acid in Step E. ¹H NMR (400 MHz, CDCl₃) δ 7.45 (s, 1H), 7.38 (d, J=7.8 Hz, 1H), 7.36-7.31 (m, 2H), 7.31-7.29 (m, 1H), 7.22 (d, J=7.8 Hz, 1H), 7.10 (s, 1H), 3.19 (s, 3H), 2.95-2.85 (m, 2H), 2.35-2.26 (m, 1H), 2.2226-2.17 (m, 1H), 2.01-1.82 (m, 2H); m/z (APCI+) M+1=340.

### Example 25

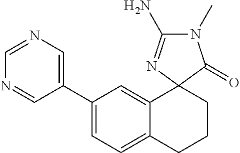

### 2-Amino-1-methyl-7′-(pyrimidin-5-yl)-3′,4′-dihydro-2′H-spiro[imidazole-4,1′-naphthalen]-5(1H)-one

2-Amino-1-methyl-7′-(pyrimidin-5-yl)-3′,4′-dihydro-2′H-spiro[imidazole-4,1′-naphthalen]-5(1H)-one was prepared according to the procedures of Example 23, in which pyrimidin-5-ylboronic acid was used in place of 5-chloropyridin-3-ylboronic acid in Step E. 1H NMR (400 MHz, CDCl₃) δ 9.14 (s, 1H), 8.88 (s, 2H), 7.41 (dd, J=7.8, 1.6 Hz, 1H), 7.27 (d, J=7.8 Hz, 1H), 7.17 (s, 1H), 3.19 (s, 3H), 3.00-2.80 (m, 2H), 2.36-2.24 (m, 1H), 2.21-2.13 (m, 1H), 1.97-1.82 (m, 2H); m/z (APCI+) M+1=308.

### Example 26

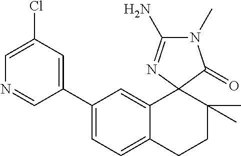

### 2-amino-7′-(5-chloropyridin-3-yl)-1,2′,2′-trimethyl-3′,4′-dihydro-2′H-spiro[imidazole-4,1′-naphthalen]-5(1H)-one

Step A: KOtBu (19.94 g, 177.7 mmol) was added to a solution of 7-bromo-3,4-dihydronaphthalen-1(2H)-one (10.0 g, 44.43 mmol) in THF (90 mL). The resulting suspension was heated to reflux and stirred for 6 hours (turns homogeneous with heating), then cooled to room temperature. Neat iodomethane (22.2 mL, 355 mmol) was added dropwise by addition funnel over 15 minutes, and the reaction mixture was heated in a 50° C. sand bath and stirred for 3 hours. The reaction mixture was then cooled to 0° C., water was added, and the mixture was extracted with ethyl acetate (2×). The combined extracts were dried (Na₂SO₄), filtered, and concentrated. The crude was purified on silica gel (1-10% ethyl acetate in hexanes gradient) to give 7-bromo-2,2-dimethyl-3,4-dihydronaphthalen-1(2H)-one (10.2 g, 91% yield) as an oil.

Step B: 2-Amino-7′-(5-chloropyridin-3-yl)-1,2′,2′-trimethyl-3′,4′-dihydro-2′H-spiro[imidazole-4,1′-naphthalen]-5(1H)-one was prepared according to the procedures of Example 23, in which 7-bromo-2,2-dimethyl-3,4-dihydronaphthalen-1(2H)-one was used in place of 7-bromo-3,4-dihydronaphthalen-1(2H)-one in Step A. 1H NMR (400 MHz, CDCl₃) δ 8.63 (s, 1H), 8.48 (s, 1H), 7.77 (s, 1H), 7.39 (d, J=7.0 Hz, 1H), 7.30 (d, J=8.6 Hz, 1H), 7.24 (s, 1H), 3.12 (s, 3H), 3.02-2.93 (m, 2H), 2.79-2.64 (m, 1H), 1.64-1.54 (m, 1H), 1.02 (s, 3H), 0.97 (s, 3H), 0.93-0.83 (m, 1H); m/z (APCI+) M+1=369.

### Example 27

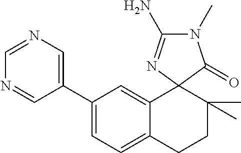

### 2-amino-1,2′,2′-trimethyl-7′-(pyrimidin-5-yl)-3′,4′-dihydro-TH-spiro[imidazole-4,1′-naphthalen]-5(1H)-one

2-Amino-1,2′,2′-trimethyl-7′-(pyrimidin-5-yl)-3′,4′-dihydro-2′H-spiro[imidazole-4,1′-naphthalen]-5(1H)-one was prepared according to the procedures of Example 26, in which pyrimidin-5-ylboronic acid was used in place of 5-chloropyridin-3-ylboronic. ¹H NMR (400 MHz, CDCl₃) δ 9.15 (s, 1H), 8.88 (s, 2H), 7.41 (dd, J=7.8, 2.3 Hz, 1H), 7.33 (d, J=7.8 Hz, 1H), 7.27-7.25 (m, 1H), 3.09 (s, 3H), 3.01-2.95 (m, 2H), 2.77-2.67 (m, 1H), 1.62-1.55 (m, 1H), 1.01 (s, 3H), 0.97 (s, 3H); m/z (APCI+) M+1=336.

### Example 28

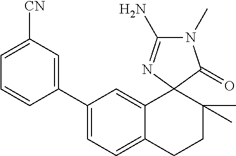

### 3-(2-amino-1,2′,2′-trimethyl-5-oxo-1,3′,4′,5-tetrahydro-2′H-spiro[imidazole-4,1′-naphthalene]-7′-yl)benzonitrile

3-(2-Amino-1,2′,2′-trimethyl-5-oxo-1,3′,4′,5-tetrahydro-2′H-spiro[imidazole-4,1′-naphthalene]-7′-yl)benzonitrile was prepared according to the procedures of Example 26, in which 3-cyanophenylboronic acid was used in place of 5-chloropyridin-3-ylboronic. ¹H NMR (400 MHz, CDCl₃) δ 7.80-7.77 (m, 1H), 7.74-7.70 (m, 1H), 7.59 (d, J=8.6 Hz, 1H), 7.49 (dd, J=7.4, 7.4 Hz, 1H), 7.39 (dd, J=7.8, 1.6 Hz, 1H), 7.28 (d, J=8.6 Hz, 1H), 7.26-7.23 (m, 1H), 3.11 (s, 3H), 3.00-2.92 (m, 2H), 2.76-2.66 (m, 1H), 1.62-1.54 (m, 1H), 1.00 (s, 3H), 0.97 (s, 3H); m/z (APCI+) M+1=359.

### Example 29

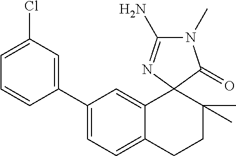

### 2-amino-7′-(3-chlorophenyl)-1,2′,2′-trimethyl-3′,4′-dihydro-2′H-spiro[imidazole-4,1′-naphthalen]-5(1H)-one

2-Amino-7′-(3-chlorophenyl)-1,2′,2′-trimethyl-3′,4′-dihydro-2′H-spiro[imidazole-4,1′-naphthalen]-5(1H)-one was prepared according to the procedures of Example 26, in which 3-chlorophenylboronic acid was used in place of 5-chloropyridin-3-ylboronic. m/z (APCI+) M+1=368.

### Example 30

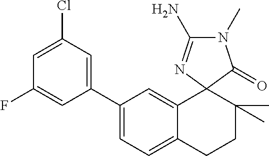

### 2-amino-7′-(3-chloro-5-fluorophenyl)-1,2′,2′-trimethyl-3′,4′-dihydro-2′H-spiro[imidazole-4,1′-naphthalen]-5(1H)-one

2-Amino-7′-(3-chloro-5-fluorophenyl)-1,2′,2′-trimethyl-3′,4′-dihydro-2′H-spiro[imidazole-4,1′-naphthalen]-5(1H)-one was prepared according to the procedures of Example 26, in which 3-chloro-5-fluorophenylboronic acid was used in place of 5-chloropyridin-3-ylboronic. m/z (APCI+) M+1=386.

### Example 31

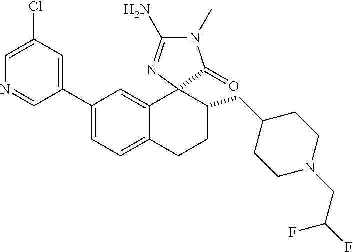

### (1′S,2′S)-2-amino-7′-(5-chloropyridin-3-yl)-2′-((1-(2,2-difluoroethyl)piperidin-4-yl)methyl)-1-methyl-3′,4′-dihydro-2′H-spiro[imidazole-4,1′-naphthalen]-5(1H)-one

Step A: Intermediate ethyl 7-bromo-1-oxo-1,2,3,4-tetrahydronaphthalene-2-carboxylate was prepared in a similar fashion to Chackal-Catoen, Sarah, et al. “Dicationic DNA-targeted antiprotozoal agents: Naphthalene replacement of benzimidazole.” Biorg. Med. Chem., 14(22) (2006): pp. 7434-7445. Diethyl carbonate (21.5 mL, 178 mmol) was added to a suspension of 60% (oil dispersion) sodium hydride (2.67 g, 66.6 mmol) in dry toluene (50 mL) under nitrogen. No exotherm was observed. This mixture was heated to 65° C., and a solution of 7-bromo-3,4-dihydronaphthalen-1(2H)-one (5.0 g, 22 mmol) in dry toluene (25 mL) was added dropwise over a period of 20 minutes. After the addition was completed, the mixture was heated to 80° C. The reaction became quite thick after 30 minutes, so more toluene (100 mL) was added to ensure stirring. The mixture was stirred for 3 hours at 80° C. After cooling in an ice bath, acetic acid (5 mL) was added dropwise. The mixture was carefully added to an Erlenmeyer flask containing ice under a blanket of N₂ and stirred for 30 minutes. The organic layer was separated, and the aqueous layer was extracted with ethyl acetate (50 mL). The combined organic layers were washed with ice-cold water (50 mL), brine (50 mL), dried (MgSO₄), filtered, and concentrated. The resulting solid was triturated with absolute ethanol (20 mL), and the solid was filtered (3.9 g). The mother liquor was concentrated, and the residue was triturated a second time with absolute EtOH (5 mL). The second crop was filtered, giving ethyl 7-bromo-1-oxo-1,2,3,4-tetrahydronaphthalene-2-carboxylate (5.2 g, 77% yield).

Step B: A dry round bottomed flask with stir bar was charged with DMF (20 mL) and sodium hydride (0.73 g, 18 mmol; 60% oil dispersion) and was stirred under N₂. Then ethyl 7-bromo-1-oxo-1,2,3,4-tetrahydronaphthalene-2-carboxylate (4.2 g, 14 mmol) dissolved in DMF (10 mL) was added. The mixture was stirred at room temperature for 15 minutes, and then tert-butyl 4-(bromomethyl)piperidine-1-carboxylate (5.1 g, 18 mmol) was added. The mixture was stirred at 80° C. for 15 hours. After cooling to room temperature, the mixture was carefully poured into ice cold water (50 mL). The product was extracted with EtOAc (2×30 mL). The combined organic phases were washed with brine (50 mL), dried (MgSO₄), filtered, and concentrated. The crude was purified by Biotage Flash 65 silica gel chromatography, eluting with 20%-30% EtOAc/hexanes to yield tert-butyl 4-((7-bromo-2-(ethoxycarbonyl)-1-oxo-1,2,3,4-tetrahydronaphthalen-2-yl)methyl)piperidine-1-carboxylate (2.1 g, 29%).

Step C: A round bottom flask with stir bar was charged with tert-butyl 4-((7-bromo-2-(ethoxycarbonyl)-1-oxo-1,2,3,4-tetrahydronaphthalen-2-yl)methyl)piperidine-1-carboxylate (2.1 g, 4.2 mmol), aqueous concentrated HCl (25 mL) and acetic acid (25 mL). The mixture was heated to 110° C. for 15 hours under N₂ with attached reflux condenser (water cooled). The mixture was cooled to room temperature. Toluene was used to azeotrope residual acids (3×20 mL). The residue was dissolved in aqueous 3N HCl (200 mL). The organic by-products were extracted with diethyl ether (50 mL). The aqueous phase was basified with NaOH pellets (pH 12-13). The desired product was extracted with 2:1 EtOAc/diethyl ether (2×100 mL). The combined organic phases were washed with brine (100 mL), dried (MgSO₄), filtered, and concentrated to yield 7-bromo-2-(piperidin-4-ylmethyl)-3,4-dihydronaphthalen-1(2H)-one (1.0 g, 70%).

Step D: A thick walled glass pressure tube was charged with 7-bromo-2-(piperidin-4-ylmethyl)-3,4-dihydronaphthalen-1(2H)-one (1.2 g, 3.7 mmol), DMF (10 mL), potassium carbonate (0.77 g, 5.6 mmol), and 1,1-difluoro-2-iodoethane (1.1 g, 5.6 mmol). The mixture was stirred at 70° C. for 15 hours. After cooling to room temperature, the reaction was diluted with EtOAc (20 mL) and water (20 mL). The phases were separated. The aqueous phase was re-extracted with EtOAc (10 mL). The combined organic phases were washed with water (2×20 mL), brine (20 mL), dried (MgSO₄), filtered, and concentrated to yield 7-bromo-2-((1-(2,2-difluoroethyl)piperidin-4-yl)methyl)-3,4-dihydronaphthalen-1(2H)-one (1.33 g, 78%).

Step E: A stainless steel bomb (19 mL capacity) with teflon insert was charged with EtOH (3 mL) and 7-bromo-2-((1-(2,2-difluoroethyl)piperidin-4-yl)methyl)-3,4-dihydronaphthalen-1(2H)-one (1.1 g, 2.8 mmol). Next, ammonium carbonate (1.4 g, 14 mmol), KCN (0.37 g, 5.7 mmol), and sodium hydrogensulfite (0.074 g, 0.71 mmol) were added. The reaction mixture was degassed with N₂. The reaction was heated to 150° C. for 15 hours with stirring. After cooling to room temperature, the reaction contents were transferred to an Erlenmeyer flask with EtOAc (20 mL) and water (20 mL). The pH was lowered to about 6 to 7 with aqueous concentrated HCl (in well ventilated hood), and then bubbled N₂ through mixture for 15 minutes to sparge HCN. The phases were separated. The aqueous phase was re-extracted with EtOAc (20 mL). The combined organic phases were washed with brine (50 mL), dried (MgSO₄), filtered, and concentrated. The crude was triturated with diethyl ether (5 mL), and the solid was filtered. The filtrate was concentrated and triturated with more diethyl ether (2 mL). A second crop was filtered, and combined with the first crop to yield 7′-bromo-2′-((1-(2,2-difluoroethyl)piperidin-4-yl)methyl)-3′,4′-dihydro-2′H-spiro[imidazolidine-4,1′-naphthalene]-2,5-dione (830 mg, 57%) as a 2:1 ratio of diastereomers.

Step F: A round bottomed flask with stir bar was charged with 7′-bromo-2′-((1-(2,2-difluoroethyl)piperidin-4-yl)methyl)-3′,4′-dihydro-2′H-spiro[imidazolidine-4,1′-naphthalene]-2,5-dione (0.82 g, 1.80 mmol), DMF (10 mL), potassium carbonate (0.27 g, 1.9 mmol), and lastly iodomethane (0.12 mL, 2.0 mmol). The reaction mixture was stirred at room temperature for 3 days. The reaction mixture was worked up by partitioning between EtOAc (20 mL) and water (20 mL). The phases were separated, and the aqueous phase was re-extracted with EtOAc (10 mL). The combined organic phases were washed with water (2×20 mL), brine (20 mL), dried (MgSO₄), filtered, and concentrated to yield 7′-bromo-2′-((1-(2,2-difluoroethyl)piperidin-4-yl)methyl)-1-methyl-3′,4′-dihydro-2′H-spiro[imidazolidine-4,1′-naphthalene]-2,5-dione (0.63 g, 63%).

Step G: A thick walled glass pressure tube was charged with 7′-bromo-2′-((1-(2,2-difluoroethyl)piperidin-4-yl)methyl)-1-methyl-3′,4′-dihydro-2′H-spiro[imidazolidine-4,1′-naphthalene]-2,5-dione (630 mg, 1.34 mmol), Lawesson's Reagent (325 mg, 0.804 mmol), and toluene (10 mL). The mixture was degassed with N₂. The reaction mixture was heated to 110° C. for 15 hours. After cooling to room temperature, the reaction was diluted with EtOAc (20 mL) and washed with saturated aqueous NaHCO₃ (20 mL). The aqueous phase was re-extracted with EtOAc (10 mL). The combined organic phases were washed again with saturated aqueous NaHCO₃ (20 mL), brine (20 mL), dried (MgSO₄), filtered, and concentrated. The crude was purified by Biotage Flash 40L silica gel chromatography, eluting with 20%-30% EtOAc/hexanes to yield 7′-bromo-2′-((1-(2,2-difluoroethyl)piperidin-4-yl)methyl)-1-methyl-2-thioxo-3′,4′-dihydro-2′H-spiro[imidazolidine-4,1′-naphthalen]-5-one (280 mg, 33%) as a 1:1 mixture of diastereomers.

Step H: A round bottomed flask with stir bar was charged with 7′-bromo-2′-((1-(2,2-difluoroethyl)piperidin-4-yl)methyl)-1-methyl-2-thioxo-3′,4′-dihydro-2′H-spiro[imidazolidine-4,1′-naphthalen]-5-one (308 mg, 0.633 mmol), MeOH (5 mL), 70% aqueous t-butyl hydroperoxide (1.3 mL, 9.5 mmol), and 30% aqueous NH₄OH (2.5 mL, 19 mmol). The reaction mixture was stirred for 15 hours at room temperature. Water (2 mL) was added and concentrated in vacuo. The mixture was partitioned between EtOAc (10 mL) and water (10 mL). The phases were separated. The aqueous phase was re-extracted with EtOAc (5 mL). The combined organic phases were washed with brine (20 mL), dried (MgSO₄), filtered, and concentrated. The crude was purified by Biotage Flash 40L silica gel chromatography, eluting with 5%-10% MeOH in DCM to yield 2-amino-7′-bromo-2′-((1-(2,2-difluoroethyl)piperidin-4-yl)methyl)-1-methyl-3′,4′-dihydro-2′H-spiro[imidazole-4,1′-naphthalen]-5(1H)-one (88 mg, 30%).

Step I: A 2 dram vial was charged with 2-amino-7′-bromo-2′-((1-(2,2-difluoroethyl)piperidin-4-yl)methyl)-1-methyl-3′,4′-dihydro-2′H-spiro[imidazole-4,1′-naphthalen]-5(1H)-one (30 mg, 0.064 mmol), dioxane (0.7 mL), 5-chloropyridin-3-ylboronic acid (12 mg, 0.077 mmol), Pd(PPh₃)₄ (7.4 mg, 0.0064 mmol), and aqueous 2N Na₂CO₃ (80 μL, 0.16 mmol). The mixture was sparged with N₂ for 1 minute and then heated to 90° C. for 15 hours. The reaction mixture was loaded directly on to a preparative TLC plate (0.5 mm thickness, Rf=0.45) and eluted with 10% MeOH (containing 7N NH₃) in DCM. The diastereomers were separated to yield (1′S,2′S)-2-amino-7′-(5-chloropyridin-3-yl)-2′-((1-(2,2-difluoroethyl)piperidin-4-yl)methyl)-1-methyl-3′,4′-dihydro-2′H-spiro[imidazole-4,1′-naphthalen]-5(1H)-one (9 mg, 26%). 1H NMR (400 MHz, CDCl₃) δ 8.89 (br s, 1H), 8.40 (s, 1H), 7.79 (s, 1H), 7.43 (d, J=6 Hz, 1H), 7.18-7.25 (m, 2H), 6.33 (br s, 2H), 5.85 (tt, J=4, 56 Hz, 1H), 3.38 (s, 3H), 2.89 (m, 4H), 2.71 (td, J=4, 15 Hz, 2H), 2.27 (m, 1H), 2.17 (m, 3H), 1.66 (m, 3H), 1.37 (m, 3H), 1.14 (m, 1H), 0.91 (m, 1H); m/z (APCI-pos) M+1=502.

### Example 32

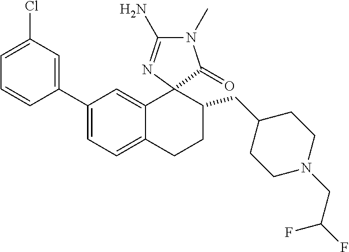

### (1′S,2′S)-2-amino-7′-(3-chlorophenyl)-2′-((1-(2,2-difluoroethyl)piperidin-4-yl)methyl)-1-methyl-3′,4′-dihydro-2′H-spiro[imidazole-4,1′-naphthalen]-5(1H)-one

(1′S,2′S)-2-Amino-7′-(3-chlorophenyl)-2′-((1-(2,2-difluoroethyl)piperidin-4-yl)methyl)-1-methyl-3′,4′-dihydro-2′H-spiro[imidazole-4,1′-naphthalen]-5(1H)-one (6 mg, 15%) was prepared from 2-amino-7′-bromo-2′-((1-(2,2-difluoroethyl)piperidin-4-yl)methyl)-1-methyl-3′,4′-dihydro-2′H-spiro[imidazole-4,1′-naphthalen]-5(1H)-one (30 mg, 0.064 mmol; Example 31, Step H) according to the procedure described for Example 31, Step I, substituting 3-chlorophenylboronic acid (12 mg, 0.077 mmol) for 5-chloropyridin-3-ylboronic acid. The diastereomers were separated by preparative TLC (0.5 mm thickness, Rf=0.62) eluting with 10% MeOH (containing 7N NH₃) in DCM. ¹H NMR (400 MHz, CDCl₃) δ 7.42 (s, 1H), 7.39 (m, 1H), 7.31 (m, 3H), 7.20 (d, J=8 Hz, 1H), 6.96 (d, J=2 Hz, 1H), 5.85 (tt, J=4, 56 Hz, 1H), 4.80 (br s, 2H), 3.24 (s, 3H), 2.89 (m, 4H), 2.70 (td, J=5, 15 Hz, 2H), 2.25 (m, 1H), 2.13 (m, 3H), 1.71 (m, 1H), 1.57 (m, 2H), 1.29 (m, 3H), 1.15 (m, 1H), 0.93 (m, 1H); m/z (APCI-pos) M+1=501.

### Example 33

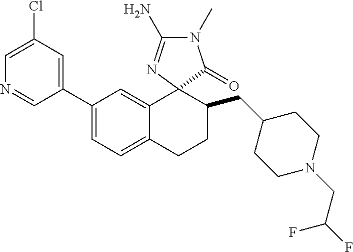

### (1′S,2′R)-2-amino-7′-(5-chloropyridin-3-yl)-2′-((1-(2,2-difluoroethyl)piperidin-4-yl)methyl)-1-methyl-3′,4′-dihydro-2′H-spiro[imidazole-4,1′-naphthalen]-5(1H)-one

(1′S,2′R)-2-Amino-7′-(5-chloropyridin-3-yl)-2′4(1-(2,2-difluoroethyl)piperidin-4-yl)methyl)-1-methyl-3′,4′-dihydro-2′H-spiro[imidazole-4,1′-naphthalen]-5(1H)-one (10 mg, 26%) was prepared from 2-amino-7′-bromo-2′4((1-(2,2-difluoroethyl)piperidin-4-yl)methyl)-1-methyl-3′,4′-dihydro-2′H-spiro[imidazole-4,1′-naphthalen]-5(1H)-one (30 mg, 0.064 mmol; Example 31, Step H) according to the procedure described for Example 31, Step I. The diastereomers were separated by preparative TLC (0.5 mm thickness, Rf=0.32) eluting with 10% MeOH (containing 7N NH₃) in DCM. ¹H NMR (400 MHz, CDCl₃) δ 8.80 (s, 1H), 8.31 (s, 1H), 7.78 (s, 1H), 7.39 (m, 2H), 7.29 (d, J=8 Hz, 1H), 6.63 (br s, 2H), 5.87 (tt, J=4, 56 Hz, 1H), 3.40 (s, 3H), 3.02 (m, 1H), 2.93 (m, 3H), 2.72 (td, J=4, 15 Hz, 2H), 2.33 (m, 1H), 2.17 (m, 3H), 1.98 (m, 1H), 1.67 (m, 2H), 1.41 (m, 2H), 1.26 (m, 2H), 1.11 (m, 1H)); m/z (APCI-pos) M+1=502.

### Example 34

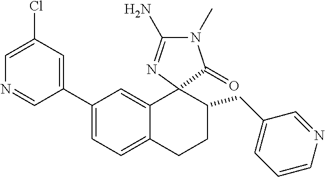

### (1′S,2′S)-2-amino-7′-(5-chloropyridin-3-yl)-1-methyl-2′-(pyridin-3-ylmethyl)-3′,4′-dihydro-2′H-spiro[imidazole-4,1′-naphthalen]-5(1H)-one

Step A: Intermediate (E)-7-bromo-2-(pyridin-3-ylmethylene)-3,4-dihydronaphthalen-1(2H)-one was prepared according to the procedure described in EP0073663. A dry round bottomed flask with stir bar was charged with 7-bromo-3,4-dihydronaphthalen-1(2H)-one (5.7 g, 25 mmol), nicotinaldehyde (2.7 g, 25 mmol), acetic acid (2.5 mL) and piperidine (3 mL). The mixture was heated to 100° C. for 6 hours. After cooling to room temperature, the mixture was concentrated in vacuo, using toluene to azeotrope residual volatiles (3×30 mL). The residue was suspended in aqueous 1N HCl (200 mL) and extracted with diethyl ether (50 mL). The organic phase was washed with aqueous 2N HCl (100 mL). The aqueous phases were basified with NaOH pellets to pH 12, and the solid was filtered. The solid was washed with water (3×10 mL) and then dried solid by acetonitrile azeotrope on the rotovap (3×50 mL) to provide (E)-7-bromo-2-(pyridin-3-ylmethylene)-3,4-dihydronaphthalen-1(2H)-one (7.3 g, 89%).

Step B: A round bottomed flask with stir bar was charged with (E)-7-bromo-2-(pyridin-3-ylmethylene)-3,4-dihydronaphthalen-1(2H)-one (5.0 g, 16 mmol), EtOH (100 mL), and platinum on carbon (6.2 g, 0.80 mmol; Degussa type). The mixture was vacuum purged with N₂ (3×). The mixture was stirred under an H₂ balloon while heating to 50° C. for 6 hours. Then, the reaction was left at room temperature for 3 days under an H₂ balloon, which was refilled after the first 18 hours at room temperature. The reaction was vacuum purged with N₂ (3×), filtered through Celite® and rinsed with DCM (3×30 mL). The filtrate was concentrated in vacuo to provide 7-bromo-2-(pyridin-3-ylmethyl)-3,4-dihydronaphthalen-1(2H)-one (4.2 g, 63%).

Step C: (1′S,2′S)-2-Amino-7′-(5-chloropyridin-3-yl)-1-methyl-2′-(pyridin-3-ylmethyl)-3′,4′-dihydro-2′H-spiro[imidazole-4,1′-naphthalen]-5(1H)-one was prepared using the same procedures as described in Example 31, Steps E-I using 7-bromo-2-(pyridin-3-ylmethyl)-3,4-dihydronaphthalen-1(2H)-one for (1′S,2′S)-2-amino-7′-(5-chloropyridin-3-yl)-2′-((1-(2,2-difluoroethyl)piperidin-4-yl)methyl)-1-methyl-3′,4′-dihydro-2′H-spiro[imidazole-4,1′-naphthalen]-5(1H)-one.

### Example 35

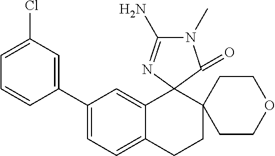

### 2-amino-7″-(3-chlorophenyl)-1-methyl-2′,3′,5′,6′-tetrahydro-3″,4″-dihydro-2″H-dispiro[midazol-4,1″-naphthalen-2″,4′-pyran]-5(1H)-one

Step A: 7-Bromo-3,4-dihydro-1(2H)-naphthalenone (4.0 g, 17.77 mmol) and 2-bromoethyl ether (2.90 mL, 23.1 mmol) were diluted with benzene (100 mL) followed by the addition of KOtBu (4.19 g, 37.3 mmol). The reaction was heated to reflux and stirred for 3 hours. The reaction was allowed to cool and diluted with ether and water. The layers were separated, and the organic layer was dried over MgSO₄, filtered and concentrated. The material was purified on silica gel eluting with 10-40% ethyl acetate/hexanes to yield 7-bromo-2′,3,3′,4,5′,6′-hexahydro-1H-spiro[naphthalene-2,4′-pyran]-1-one (1.6 g, 5.421 mmol, 30.5% yield).

Step B: 7-Bromo-2′,3,3′,4,5′,6′-hexahydro-1H-spiro[naphthalene-2,4′-pyran]-1-one (1.3 g, 4.40 mmol), KCN (0.574 g, 8.81 mmol), ammonium carbonate (2.96 g, 30.8 mmol) and NaHSO₃ (0.458 g, 4.40 mmol) were diluted in ethanol (4 mL). The reaction vessel was sealed, heated to 130° C. and stirred for 12 hours. The reaction was allowed to cool and poured onto ice water. The pH was adjusted to about 6, and the material was stirred for 30 minutes. The material was filtered and dried under vacuum. The material was triturated with 10% methanol/DCM to produce a solid (428 mg, 1.17 mmol, 26.6% yield).

Step C: The product of Step B (400 mg, 1.10 mmol) was diluted with DMF (5 mL) followed by the addition of K₂CO₃ (182 mg, 1.31 mmol) and MeI (68.3 μL, 1.10 mmol; d 2.275). After stirring for 6 hours, the reaction was diluted with ethyl acetate and water. The organic was washed with water and brine. The organics were dried over MgSO₄, filtered and concentrated. The material was purified on silica gel eluting with 10-70% ethyl acetate/hexanes to yield the product (334 mg, 0.881 mmol, 80.4% yield).

Step D: The product of Step C (334 mg, 0.881 mmol) was diluted with toluene (4 mL) followed by the addition of Lawesson's Reagent (267 mg, 0.661 mmol). The reaction was refluxed for 12 hours. The reaction was diluted with ethyl acetate and washed with saturated sodium bicarbonate, water and brine. The organics were dried over MgSO₄, filtered and concentrated. The material was purified using on silica gel eluting with 10-50% ethyl acetate/hexanes to yield the product (100 mg, 0.253 mmol, 28.7% yield).

Step E: The product of Step D (100 mg, 0.253 mmol) was diluted with methanol (2 mL) followed by the addition of tert-butyl hydroperoxide (543 μL, 3.79 mmol) and NH₄OH (1093 μl, 9.36 mmol). The reaction was heated to 40° C. and stirred for 2 hours, and the reaction was left to stir overnight at ambient temperature. The reaction was concentrated down and diluted with DCM and water. The layers were separated, and the organics were dried over MgSO₄, filtered and concentrated. The material was purified on silica gel eluting with 1-10% methanol/DCM (1% NH4OH) to yield the product (50 mg, 0.132 mmol, 52.3% yield).

Step F: The product of Step E (22 mg, 0.058 mmol) and 3-chlorophenylboronic acid (12 mg, 0.076 mmol) were diluted with dioxane (1 mL) followed by the addition of Pd(PPh₃)₄ (3.4 mg, 0.0029 mmol) and Na₂CO₃ (87 μL, 0.17 mmol). The reaction was sealed, heated to 85° C. and stirred for 12 hours. The reaction was loaded directly onto silica gel and eluted with 1-10% methanol/DCM (1% NH₄OH) to afford 2-amino-7″-(3-chlorophenyl)-1-methyl-5-oxo-2′,3′,5′,6′-tetrahydro-3′,4″-dihydro-2″H-dispiro[imidazol-4,1″-naphthalen-2″,4′-pyran] (8 mg, 0.020 mmol, 34% yield). MS (APCI-pos)=410.2 (M+1).

### Example 36

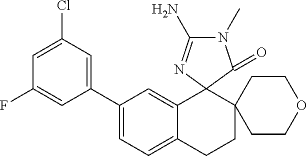

### 2-amino-7″-(3-chloro-5-fluorophenyl)-1-methyl-2′,3′,5′,6′-tetrahydro-3″,4″-dihydro-2″H-dispiro[imidazol-4,1″-naphthalen-2″,4′-pyran]-5(1H)-one

2-Amino-7″-(3-chloro-5-fluorophenyl)-1-methyl-2′,3′,5′,6′-tetrahydro-3″,4″-dihydro-2″H-dispiro[imidazol-4,1″-naphthalen-2″,4′-pyran]-5(1H)-one (8 mg, 32%) was prepared according to Example 35, substituting 3-chloro-5-fluorophenylboronic acid for 3-chlorophenylboronic acid. MS (APCI-pos)=428.2 (M+1).

### Example 37

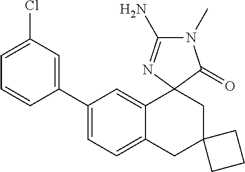

### 2-amino-7′-(3-chlorophenyl)-1-methyl-5-oxo-3′,4′-dihydro-2′H-dispiro[imidazol-4,1′-naphthalen-3′,1″-cyclobutyl]-5 (1H)-one

Step A: 1.0 M Titanium (IV) chloride in dichloromethane (88.5 mL, 88.5 mmol) was added to a solution of diethyl malonate (12.3 mL, 80.5 mmol), cyclobutanone (6 mL, 80.5 mmol) and pyridine (13.0 mL, 161 mmol; 0.978) in toluene (161 mL, 80.5 mmol). The reaction was stirred at room temperature under a nitrogen atmosphere. After 15 hours, the reaction was concentrated and diluted with ethyl acetate. The ethyl acetate suspension was treated with 1N hydrochloric acid, and the organic layer was separated. The organic layer was washed with saturated sodium bicarbonate and saturated sodium chloride. It was dried over anhydrous sodium sulfate, filtered, and concentrated. The crude oil was purified by silica gel chromatography (gradient: 1-30% EtOAc/hexanes) to provide diethyl 2-cyclobutylidenemalonate (11.5 g, 54.2 mmol, 67.3% yield) as an oil.

Step B: Diethyl 2-(1-(4-bromobenzyl)cyclobutyl)malonate (59% yield) was prepared according to Example 2, Step A, substituting diethyl 2-cyclobutylidenemalonate for diethyl 2-(propan-2-ylidene)malonate.

Step C: 2-(1-(4-Bromobenzyl)cyclobutyl)malonic acid (81% yield) was prepared according to Example 2, Step B, substituting diethyl 2-(1-(4-bromobenzyl)cyclobutyl)malonate for diethyl 2-(1-(4-bromophenyl)-2-methylpropan-2-yl)malonate.

Step D: 2-(1-(4-Bromobenzyl)cyclobutyl)acetic acid (100% yield) was prepared according to Example 2, Step C, substituting 2-(1-(4-bromobenzyl)cyclobutyl)malonic acid for 2-(1-(4-bromophenyl)-2-methylpropan-2-yl)malonic acid.

Step E: 6′-Bromo-1′H-spiro[cyclobutane-1,2′-naphthalen]-4′(3′H)-one (72% yield) was prepared according to Example 2, Step D substituting 2-(1-(4-bromobenzyl)cyclobutyl)acetic acid for 4-(4-bromophenyl)-3,3-dimethylbutanoic acid.

Step F: The product (45% yield) was prepared according to Example 2, Step E, substituting 6′-bromo-1′H-spiro[cyclobutane-1,2′-naphthalen]-4′(3′H)-one for 7-bromo-3,3-dimethyl-3,4-dihydronaphthalen-1(2H)-one and with heating to 150° C. for 4 days instead of 3 days.

Step G: Potassium carbonate (0.330 g, 2.39 mmol) and iodomethane (0.142 mL, 2.28 mmol) were added to a solution of the product of Step F (0.727 g, 2.17 mmol) in DMF (11 mL), and the reaction was allowed to stir at ambient temperature overnight. The reaction mixture was precipitated with water, and the product was collected via filtration (0.594 g, 1.70 mmol, 78.4% yield).

Step H: Lawesson's Reagent (0.413 g, 1.02 mmol) was added to a solution of the product of Step G (0.594 g, 1.70 mmol) in dichloroethane (8 mL), and the reaction was heated to 80° C. for 4 hours. It was cooled to ambient temperature and was loaded directly onto a Biotage SP1 system and purified by silica gel chromatography to produce a product (0.262 g, 0.717 mmol, 42.2% yield).

Step I: Ammonia (2.56 mL, 17.9 mmol) in MeOH and 2-hydroperoxy-2-methylpropane (0.513 mL, 3.59 mmol) were added to a solution of the product of Step H (0.262 g, 0.717 mmol) in dichloromethane (3 mL), and the reaction was allowed to stir for 2 days. At this point, it was recharged with both ammonia in MeOH (2.5 mL) and 2-hydroperoxy-2-methylpropane (0.513 mL, 3.59 mmol), and it was allowed to stir for another week. It was quenched by the addition of water and saturated sodium sulfite. It was extracted twice with dichloromethane, and the combined organics were dried over anhydrous sodium sulfate, filtered, and concentrated. It was purified by silica gel chromatography on a Biotage SP1 system to produce a product (0.178 g, 0.511 mmol, 71.3% yield).

Step J: 3-Chlorophenylboronic acid (0.026 g, 0.17 mmol) and 20% aqueous sodium carbonate (0.21 g, 0.40 mmol) were added to a solution of the product of Step I (0.045 g, 0.13 mmol) in dioxane (1 mL), and the reaction was degassed with argon. Next, tetrakis(triphenylphosphine)palladium(0) (0.0090 g, 0.0078 mmol) was added, and the reaction was sealed and heated to 100° C. overnight. It was cooled to ambient temperature and was loaded directly onto a Biotage SP1 system and purified by silica gel chromatography to yield 2-amino-7′-(3-chlorophenyl)-1-methyl-5-oxo-3′,4′-dihydro-2′H-dispiro[imidazol-4,1′-naphthalen-3′,1″-cyclobutyl]-5(1H)-one (0.041 g, 0.11 mmol, 84% yield). m/z (APCI-pos) M+1=380.2.

### Example 38

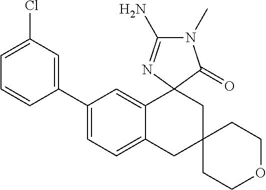

### 2-amino-7″-(3-chlorophenyl)-1-methyl-2′,3′,5′,6′-tetrahydro-3″,4″-dihydro-2″H-dispiro[imidazol-4,1″-naphthalen-3″,4′-pyran]-5(1H)-one

Step A: Pyridine (35.1 mL, 434 mmol) and titanium (IV) chloride (217 mL, 217 mmol) in toluene were added to a solution of dihydro-2H-pyran-4(3H)-one (10.0 mL, 108.6 mmol) and diethyl malonate (16.5 mL, 108.6 mmol) in toluene (200 mL), and the reaction was allowed to stir at ambient temperature for 2 days. The reaction mixture was diluted with toluene and washed with water and saturated sodium chloride. The reaction was dried over anhydrous sodium sulfate, filtered, and concentrated. The reaction was purified by silica gel chromatography on a Biotage SP1 system to yield diethyl 2-(2H-pyran-4(3H,5H,6H)-ylidene)malonate (7.7 g, 31.8 mmol, 29.3% yield).

Step B: Diethyl 2-(4-(4-bromobenzyl)tetrahydro-2H-pyran-4-yl)malonate was prepared according to Example 2, Step A, substituting diethyl 2-(2H-pyran-4(3H,5H,6H)-ylidene)malonate for diethyl 2-(propan-2-ylidene)malonate.

Step C: 2-(4-(4-Bromobenzyl)tetrahydro-2H-pyran-4-yl)malonic acid was prepared according to Example 2, Step B, substituting diethyl 2-(4-(4-bromobenzyl)tetrahydro-2H-pyran-4-yl)malonate for diethyl 2-(1-(4-bromophenyl)-2-methylpropan-2-yl)malonate.

Step D: 2-(4-(4-Bromobenzyl)tetrahydro-2H-pyran-4-yl)acetic acid was prepared according to Example 2, Step C, substituting 2-(4-(4-bromobenzyl)tetrahydro-2H-pyran-4-yl)malonic acid for 2-(1-(4-bromophenyl)-2-methylpropan-2-yl)malonic acid.

Step E: 6-Bromo-2′,3′,5′,6′-tetrahydro-1H-spiro[naphthalene-2,4′-pyran]-4(3H)-one was prepared according to Example 2, Step D, substituting 2-(4-(4-bromobenzyl)tetrahydro-2H-pyran-4-yl)acetic acid for 4-(4-bromophenyl)-3,3-dimethylbutanoic acid.

Step F: The product (44% yield) was prepared according to Example 2, Step E, substituting 6-bromo-2′,3′,5′,6′-tetrahydro-1H-spiro[naphthalene-2,4′-pyran]-4(3H)-one for 7-bromo-3,3-dimethyl-3,4-dihydronaphthalen-1(2H)-one.

Step G: Potassium carbonate (0.102 g, 0.735 mmol) and iodomethane (0.0438 mL, 0.702 mmol) was added to a solution of the product of Step F (0.244 g, 0.668 mmol) in dimethylformamide (3 mL), and the reaction was allowed to stir at ambient temperature for 16 hours. It was quenched by the addition of saturated ammonium chloride and was extracted twice with ethyl acetate. The combined organics were dried over anhydrous sodium sulfate, filtered, and concentrated. It was purified by silica gel chromatography on a Biotage SP1 system to produce a product (0.234 g, 0.617 mmol, 92.4% yield).

Step H: A suspension of the product of Step G (1.2 g, 3.16 mmol) in dichloroethane (15 mL) was heated to 80° C., then Lawesson's Reagent (0.768 g, 1.90 mmol) was added. The reaction was heated to 80° C. overnight. It was cooled to ambient temperature and was loaded directly on a Biotage SP1 system and purified by silica gel chromatography to produce a product (0.636 g, 1.61 mmol, 50.8% yield).

Step I: 7M Ammonia in methanol (8.04 mL, 56.3 mmol) and 2-hydroperoxy-2-methylpropane (1.15 mL, 8.04 mmol) was added to a suspension of the product of Step H (0.636 g, 1.61 mmol) in dichloromethane (7 mL), and the reaction was allowed to stir for 4 days. It was recharged with 7M ammonia in methanol (5 mL) and 2-hydroperoxy-2-methylpropane (0.5 mL), allowed to stir another 2 days, was recharged again with the same amounts of 7M ammonia in methanol and 2-hydroperoxy-2-methylpropane, and stirred two more days. It was quenched by the addition of 25% aqueous sodium sulfite, and it was stirred for 2 hours. It was diluted with water and extracted twice with dichloromethane. The extracts were dried over anhydrous sodium sulfate, filtered, and concentrated. It was purified on the by silica gel chromatography on a Biotage SP1 system to produce a product (0.385 g, 1.02 mmol, 63.3% yield).

Step J: Tetrakis(triphenylphosphine)palladium(0) (0.0076 g, 0.0066 mmol) was added to a solution of the product of Step I (0.050 g, 0.13 mmol), 3-chlorophenylboronic acid (0.027 g, 0.17 mmol), and 20% aqueous sodium carbonate (0.18 g, 0.34 mmol) in dioxane (1 mL), and the reaction was briefly degassed with argon. The reaction mixture was heated to 95° C. in a sealed vial overnight. The reaction was loaded directly onto a biotage SP1 system and purified by silica gel chromatography to yield 2-amino-7″-(3-chlorophenyl)-1-methyl-2′,3′,5′,6′-tetrahydro-3″,4″-dihydro-2″H-dispiro[imidazol-4,1″-naphthalen-3″,4′-pyran]-5(1H)-one (0.054 g, 0.13 mmol, 100% yield). ¹H NMR (400 MHz, CDCl₃) δ 7.5-7.2 (m, 6H), 7.0 (m, 1H), 3.8-3.6 (m, 4H), 3.2 (s, 3H), 2.98 (d, J=15 Hz, 1H), 2.87 (d, J=15 Hz, 1H), 2.23 (d, J=14 Hz, 1H), 2.03 (d, J=14 Hz, 1H), 1.6 (m, 3H), 1.5 (m, 1H); m/z (APCI-pos) M+1=410.2.

### Example 39

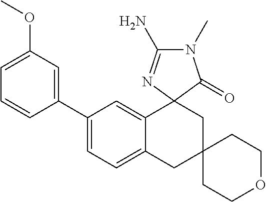

### 2-amino-7″-(3-methoxyphenyl)-1-methyl-2′,3′,5′,6′-tetrahydro-3″,4″-dihydro-2″H-dispiro[imidazol-4,1″-naphthalen-3″,4′-pyran]-5(1H)-one

2-Amino-7″-(3-methoxyphenyl)-1-methyl-2′,3′,5′,6′-tetrahydro-3″,4″-dihydro-2″ H-dispiro[imidazol-4,1″-naphthalen-3″,4′-pyran]-5(1H)-one (100% yield) was prepared according to Example 38, Step J, substituting 3-methoxyphenylboronic acid in place of 3-chlorophenylboronic acid. ¹H NMR (400 MHz, CDCl₃) δ 7.41 (d, J=8 Hz, 1H), 7.31 (dd, J=7, 8 Hz, 1H), 7.19 (d, J=7 Hz, 1H), 7.05 (m, 3H), 6.87 (dd, J=3, 8 Hz, 1H), 3.83 (s, 3H), 3.8-3.6 (m, 4H), 3.17 (s, 3H), 2.99 (d, J=16 Hz, 1H), 2.77 (d, J=16 Hz, 1H), 2.25 (d, J=14 Hz, 1H), 2.03 (d, J=14 Hz, 1H), 1.7-1.4 (m, 4H); m/z (APCI-pos) M+1=406.2.

### Example 40

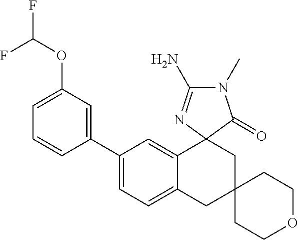

2-amino-7″-(3-(difluoromethoxy)phenyl)-1-methyl-2′,3′,5′,6′-tetrahydro-3″,4″-dihydro-2″H-dispiro[imidazol-4,1″-naphthalen-3″,4′-pyran]-5(1H)-one

2-Amino-7″-(3-(difluoromethoxy)phenyl)-1-methyl-2′,3′,5′,6′-tetrahydro-3″,4″-dihydro-2″H-dispiro[imidazol-4,1″-naphthalen-3″,4′-pyran]-5(1H)-one (100% yield) was prepared according to Example 38, Step J, substituting 3-(difluoromethoxy)phenylboronic acid in place of 3-chlorophenylboronic acid. ¹H NMR (400 MHz, CDCl₃) δ 7.4-7.2 (m, 5H), 7.05 (m, 2H), 6.53 (t, J=74 Hz, 1H), 3.8-3.6 (m, 4H), 3.18 (s, 3H), 2.99 (d, J=16 Hz, 1H), 2.79 (d, J=17 Hz, 1H), 2.24 (d, J=14 Hz, 1H), 2.04 (d, J=14 Hz, 1H), 1.7-1.4 (m, 4H); m/z (APCI-pos) M+1=442.2.

### Example 41

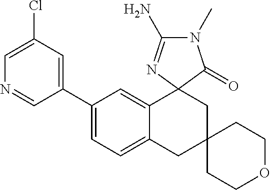

### 2-amino-7″-(5-chloropyridin-3-yl)-1-methyl-2′, 3′,5′,6′-tetrahydro-3″,4″-dihydro-2″H-dispiro[imidazol-4,1″-naphthalen-3″,4′-pyran]-5(1H)-one

2-Amino-7″-(5-chloropyridin-3-yl)-1-methyl-2′,3′,5′,6′-tetrahydro-3″,4″-dihydro-2″H-dispiro[imidazol-4,1″-naphthalen-3″,4′-pyran]-5(1H)-one (99% yield) was prepared according to Example 38, Step J, substituting 5-chloropyridin-3-ylboronic acid in place of 3-chlorophenylboronic acid. ¹H NMR (400 MHz, CDCl₃) δ 8.59 (d, J=2 Hz, 1H), 8.55 (d, J=2 Hz, 1H), 7.74 (m, 1H), 7.38 (d, J=8 Hz, 1H), 7.25 (m, 1H), 7.02 (m, 1H), 3.8-3.6 (m, 4H), 3.19 (s, 3H), 2.99 (d, J=16 Hz, 1H), 2.82 (d, J=16 Hz, 1H), 2.22 (d, J=14 Hz, 1H), 20.5 (d, J=14 Hz, 1H), 1.7-1.4 (m, 4H); m/z (APCI-pos) M+1=411.2.

### Example 42

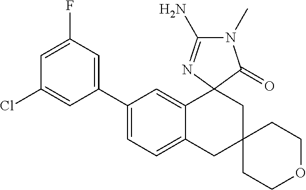

### 2-amino-7″-(3-fluoro-5-chlorophenyl)-1-methyl-2′,3′,5′,6′-tetrahydro-3″,4″-dihydro-2″H-dispiro[imidazol-4,1″-naphthalen-3″,4′-pyran]-5(1H)-one

2-Amino-7″-(3-fluoro-5-chlorophenyl)-1-methyl-2′,3′,5′,6′-tetrahydro-3″,4″-dihydro-2″H-dispiro[imidazol-4,1″-naphthalen-3″,4′-pyran]-5(1H)-one (100% yield) was prepared according to Example 38, Step J, substituting 3-fluoro-5-chlorophenylboronic acid in place of 3-chlorophenylboronic acid. ¹H NMR (400 MHz, CDCl₃) δ 7.37 (m, 1H), 7.22 (m, 2H), 7.1-6.9 (m, 3H), 3.8-3.6 (m, 4H), 3.19 (s, 3H), 2.99 (d, J=15 Hz, 1H), 2.80 (d, J=15 Hz, 1H), 2.22 (d, J=14 Hz, 1H), 2.03 (d, J=14 Hz, 1H), 1.7-1.4 (m, 4H); m/z (APCI-pos) M+1=428.2.

### Example 43

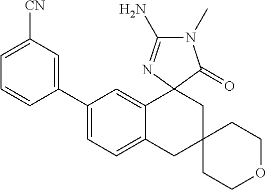

### 3-(2-amino-1-methyl-5-oxo-2′,3′,5′,6′-tetrahydro-1,3″,4″,5-tetrahydro-2″H-dispiro[imidazol-4,1″-naphthalen-3″,4′-pyran]-7″-yl)benzonitrile

3-(2-Amino-1-methyl-5-oxo-2′,3′,5′,6′-tetrahydro-1,3″,4″,5-tetrahydro-2″H-dispiro[imidazol-4,1″-naphthalen-3″,4′-pyran]-7″-yl)benzonitrile (21% yield) was prepared according to Example 38, Step J, substituting 3-cyanophenylboronic acid in place of 3-chlorophenylboronic acid. 1H NMR (400 MHz, CDCl₃) δ 7.8-7.4 (m, 5H), 7.3 (m, 1H), 7.0 (m, 1H), 3.8-3.6 (m, 4H), 3.34 (s, 3H), 3.03 (d, J=16 Hz, 1H), 2.84 (d, J=16 Hz, 1H), 2.25 (m, 2H), 1.7-1.5 (m, 4H); m/z (APCI-pos) M+1=401.2.

### Example 44

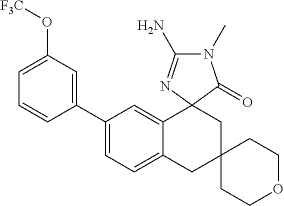

### 2-amino-7″ 43-(trifluoromethoxy)phenyl)-1-methyl-2′,3′,5′,6′-tetrahydro-3″,4″-dihydro-2″H-dispiro[imidazol-4,1″-naphthalen-3″,4′-pyran]-5(1H)-one

2-Amino-7″-(3-(trifluoromethoxy)phenyl)-1-methyl-2′,3′,5′,6′-tetrahydro-3″,4″-dihydro-2″H-dispiro[imidazol-4,1″-naphthalen-3″,4′-pyran]-5(1H)-one (21% yield) was prepared according to Example 38, Step J, substituting 3-(trifluoromethoxy)phenylboronic acid in place of 3-chlorophenylboronic acid. ¹H NMR (400 MHz, CDCl₃) δ 7.5-7.2 (m, 6H), 7.0 (m, 1H), 3.9-3.7 (m, 4H), 3.30 (s, 3H), 3.01 (d, J=16 Hz, 1H), 2.27 (d, J=14 Hz, 1H), 2.21 (d, J=14 Hz, 1H), 1.7-1.5 (m, 4H); m/z (APCI-pos) M+1=460.2.

### Example 45

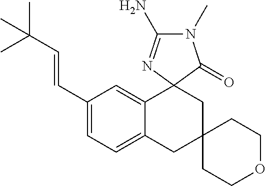

### (E)-2-amino-7″-(3,3-dimethylbut-1-enyl)-1-methyl-2′,3′,5′,6′-tetrahydro-3″,4″-dihydro-2″H-dispiro[imidazol-4,1″-naphthalen-3″,4′-pyran]-5(1H)-one

(E)-2-Amino-7″-(3,3-dimethylbut-1-enyl)-1-methyl-2′,3′,5′,6′-tetrahydro-3″,4″-dihydro-2″H-dispiro[imidazol-4,1″-naphthalen-3″,4′-pyran]-5(1H)-one (21% yield) was prepared according to Example 38, Step J, substituting (E)-3,3-dimethylbut-1-enylboronic acid in place of 3-chlorophenylboronic acid. ¹H NMR (400 MHz, CDCl₃) δ 7.35 (d, J=8 Hz, 1H), 7.10 (d, J=8 Hz, 1H), 6.72 (s, 1H), 6.21 (d, J=16 Hz, 1H), 6.15 (d, J=16 Hz, 1H), 3.8-3.6 (m, 4H), 3.31 (s, 3H), 2.94 (d, J=15 Hz, 1H), 2.73 (d, J=15 Hz, 1H), 2.2 (m, 2H), 1.7-1.4 (m, 4H), 1.10 (s, 9H); m/z (APCI-pos) M+1=382.2.

### Example 46

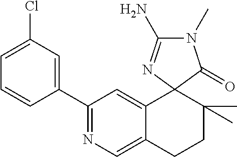

### 2-amino-3′-(3-chlorophenyl)-1,6′,6′-trimethyl-7′,8′-dihydro-6′H-spiro[imidazole-4,5′-isoquinolin]-5(1H)-one

Step A: Intermediate 3-chloro-7,8-dihydroisoquinolin-5(6H)-one oxime was prepared according to the method described in WO 2009/010488. Potassium t-butoxide 1M in THF (35.8 mL, 35.8 mmol) was added to a solution of 3-chloro-5,6,7,8-tetrahydroisoquinoline (3 g, 17.9 mmol) in THF (10 mL), and the resulting mixture was stirred at room temperature for 18 hours. The resulting mixture was cooled to 0° C. and treated dropwise with tert-butyl nitrite (7.57 mL, 57.3 mmol). Once the addition was complete, the ice bath was removed, and the mixture was stirred at room temperature for 4 hours. The mixture was then poured into brine (50 mL) and extracted with EtOAc (4×50 mL). The combined organic layers were dried (MgSO₄), filtered, and concentrated in vacuo. The residue obtained was triturated with CH₂Cl₂ to provide 3-chloro-7,8-dihydroisoquinolin-5(6H)-one oxime (3.2 g, 91% yield) as a solid. ¹HNMR (400 MHz, CDCl₃) δ 8.44 (s, 1H), 7.81 (s, 1H), 2.96 (t, J=6.26 Hz, 2H), 2.71 (t, J=6.26 Hz, 2H), 2.22-2.16 (m, 2H); LCMS (APCI+) m/z 197 (M+H)+.

Step B: A solution of 3-chloro-7,8-dihydroisoquinolin-5(6H)-one oxime (3 g, 15.3 mmol) in acetone (33.9 mL, 15.3 mmol) and concentrated HCl (27.7 mL, 305 mmol) was stirred at reflux for 6 hours. The mixture then was then cooled to room temperature and poured into an ice cold solution of 2M Na₂CO₃ (100 mL). The resulting suspension was extracted into EtOAc (3×40 mL). The combined organic layers were dried (MgSO₄), filtered and concentrated in vacuo. The residue obtained was purified by flash chromatography on silica gel eluting with 25% EtOAc/hexane to provide 3-chloro-7,8-dihydroisoquinolin-5(6H)-one (2.3 g, 83% yield) as a solid. ¹H NMR (400 MHz, CDCl₃) δ 8.45 (s, 1H), 7.80 (s, 1H), 2.96 (t, J=6.20 Hz, 2H), 2.72 (t, 6.20 Hz, 2H), 2.19 (m, 2H).

Step C: A resealable glass pressure tube was charged with a mixture of 3-chloro-7,8-dihydroisoquinolin-5(6H)-one (250 mg, 1.38 mmol), 3-chlorophenylboronic acid (204 mg, 1.31 mmol), PdCl₂(dppf)*dcm (56 mg, 0.069 mmol), 20% aqueous sodium carbonate (2.9 mL, 5.51 mmol), and 1,4-dioxane (5.5 mL, 1.38 mmol). N₂ was bubbled through the mixture for 5 minutes, and then the tube was sealed with a Teflon screw cap and stirred at 90° C. for 6 hours. The reaction mixture was cooled to room temperature and concentrated in vacuo. The resulting residue was purified by flash chromatography on silica gel (Biotage Flash 40S+) eluting with 10% EtOAc/hexane to provide 3-(3-chlorophenyl)-7,8-dihydroisoquinolin-5(6H)-one (310 mg, 87.4% yield) as a solid. ¹H NMR (400 MHz, CDCl₃) δ 8.74 (s, 1H), 8.21 (s, 1H), 8.07 (s, 1H), 7.92-7.89 (m, 1H), 7.40-7.39 (m, 2H), 3.02 (t, J=6.26 Hz, 2H), 2.75 (t, 6.26 Hz, 2H), 2.26-2.19 (m, 2H); LCMS (APCI+) m/z 258 (M+H)+.

Step D: Iodomethane (141 μL, 2.26 mmol) was added to a solution of 3-(3-chlorophenyl)-7,8-dihydroisoquinolin-5(6H)-one (265 mg, 1.03 mmol) in THF at 0° C. The mixture was then treated portionwise with NaH 60% in mineral oil (91 mg, 2.3 mmol). The resulting mixture was stirred at 0° C. for 3 hours, then quenched with ice water (5 mL) and extracted in to EtOAc (50 mL) Brine (30 mL) was added to the aqueous phase and extracted with 5% MeOH/EtOAc. The combined organic phases were dried (MgSO₄), filtered and concentrated in vacuo. The residue obtained was purified by flash chromatography on silica gel (Biotage Flash 40S+) eluting with 15% EtOAc/hexane to provide 3-(3-chlorophenyl)-6,6-dimethyl-7,8-dihydroisoquinolin-5(6H)-one (80 mg, 27% yield) as a solid. ¹H NMR (400 MHz, CDCl₃) δ 8.70 (s, 1H), 8.21 (s, 1H), 8.06 (m, 1H), 7.91-7.88 (m, 1H), 7.41-7.38 (m, 2H), 3.03 (t, J=6.26 Hz, 2H), 2.06 (t, J=6.26 Hz, 2H), 1.26 (s, 6H).

Step E: A metal bomb was charged with a mixture of 3-(3-chlorophenyl)-6,6-dimethyl-7,8-dihydroisoquinolin-5(6H)-one (160 mg, 0.56 mmol), ammonium carbonate (592 mg, 6.16 mmol), potassium cyanide (91 mg, 1.4 mmol), sodium bisulfite (11.7 mg, 0.112 mmol) and 200 proof ethanol (560 μL, 0.56 mmol). The bomb was sealed and stirred at 130° C. for 24 hours and allowed to cool to room temperature. The contents were then suspended in water (3×3 mL) and transferred to a 250 mL Erlenmeyer flask. The suspension was diluted with additional water (10 mL) and slowly acidified to a pH of about 2 to 3 with 2M HCl. During this time, the mixture was sparged with N₂ and allowed to stir at room temperature for 30 minutes. The solid formed was filtered, washed with water (3×10 mL) and dried to provide 3′-(3-chlorophenyl)-6′,6′-dimethyl-7′,8′-dihydro-6′H-spiro[imidazolidine-4,5′-isoquinoline]-2,5-dione (165 mg, 83% yield) as a solid. ¹H NMR (400 MHz, (CD₃)₂SO) δ 10.93 (br s, 1H), 8.56 (s, 1H), 8.52 (s, 1H), 8.04-8.03 (m, 1H), 7.94-7.92 (m, 1H), 7.59 (s, 1H), 7.55-7.48 (m, 2H), 2.93-2.84 (m, 2H), 2.57-2.52 (m, 1H), 1.61-1.55 (m, 1H), 0.97 (s, 3H), 0.86 (s, 3H); MS (APCI+) m/z 356 (M+H)+.

Step F: Solid potassium carbonate (62.2 mg, 0.45 mmol) was added to a solution of 3′-(3-chlorophenyl)-6′,6′-dimethyl-7′,8′-dihydro-6′H-spiro[imidazolidine-4,5′-isoquinoline]-2,5-dione (160 mg, 0.45 mmol) in N,N-dimethylformamide (1.8 mL, 0.45 mmol) at room temperature. The mixture was stirred for 5 minutes and treated dropwise with iodomethane (28 μL, 0.45 mmol). The resulting mixture was stirred at room temperature for 18 hours and poured into water (20 mL). After 30 minutes the solid formed was suction filtered, washed with additional water (3×20 mL) and evaporated from CH₃CN to provide 3′-(3-chlorophenyl)-1,6′,6′-trimethyl-7′,8′-dihydro-6′H-spiro[imidazolidine-4,5′-isoquinoline]-2,5-dione (158 mg, 88.4% yield) as a solid. ¹H NMR data (400 MHz, (CD₃)₂SO) 8.81 (s, 1H), 8.56 (s, 1H), 8.05 (s, 1H), 7.98-7.95 (m, 1H), 7.60 (s, 1H), 7.52-7.47 (m, 2H), 3.18-3.16 (m, 2H), 2.96-2.91 (m, 1H), 1.65-1.58 (m, 1H), 0.89-0.87 (m, 6H); LCMS (APCI+) m/z 370 (M+H)+.

Step G: A suspension of 3′-(3-chlorophenyl)-1,6′,6′-trimethyl-7′,8′-dihydro-6′H-spiro[imidazolidine-4,5′-isoquinoline]-2,5-dione (154 mg, 0.416 mmol) in toluene (6 mL) was stirred at reflux until a clear solution was obtained. Then Lawesson's Reagent (93 mg, 0.23 mmol) was added in one portion, and the resulting solution was stirred at 110° C. for 20 hours. The reaction mixture was concentrated in vacuo. The resulting residue was diluted with EtOAc (50 mL) and poured into water (20 mL). The layers were separated, and the organic later was dried (MgSO₄), filtered, and concentrated in vacuo. The residue obtained was purified by flash chromatography on silica gel (Biotage Flash 40S+) eluting with 20% EtOAc/hexane to provide 3′-(3-chlorophenyl)-1,6′,6′-trimethyl-2-thioxo-7′,8′-dihydro-6′H-spiro[imidazolidine-4,5′-isoquinolin]-5-one (97 mg, 60.4% yield) as a solid. (APCI+) m/z 386, 388 (M+H)+.

Step H: Ammonia 7M in methanol (1.1 mL, 7.39 mmol) and tert butylhydroperoxide 70% in water (511 μL, 3.69 mmol) were sequentially added to a stirred solution of 3′-(3-chlorophenyl)-1,6′,6′-trimethyl-2-thioxo-7′,8′-dihydro-6′H-spiro[imidazolidine-4,5′-isoquinolin]-5-one (95 mg, 0.25 mmol) in MeOH (1 mL). The mixture was stirred at room temperature for 48 hours. Water (20 mL) was then added to the mixture, and the solid formed was suction filtered and purified by prep TLC eluting with 10% MeOH/DCM to provide 2-amino-3′-(3-chlorophenyl)-1,6′,6′-trimethyl-7′,8′-dihydro-6′H-spiro[imidazole-4,5′-isoquinolin]-5(1H)-one (29 mg, 32% yield) as a solid. 1H NMR (400 MHz, (CD₃)₂SO) δ 8.47 (s, 1H), 7.91 (s, 1H), 7.81 (d, J=7.04 Hz, 1H), 7.50-7.43 (m, 2H), 7.30 (s, 1H), 2.92 (s, 3H), 2.89-2.79 (m, 2H), 2.56-2.50 (m, 1H), 1.56-1.49 (m, 1H), 0.86 (s, 3H), 0.76 (s, 3H); LCMS (APCI+) m/z 369 (M+H)+.

### Example 47

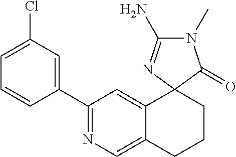

### 2-amino-3′-(3-chlorophenyl)-1-methyl-7′,8′-dihydro-6′H-spiro[imidazole-4,5′-isoquinolin]-5(1H)-one

Step A: A mixture of 3-chloro-7,8-dihydroisoquinolin-5(6H)-one (250 mg, 1.38 mmol), ammonium carbonate (1.5 g, 15 mmol), potassium cyanide (224 mg, 3.44 mmol), sodium bisulfate (28.6 mg, 0.275 mmol) and 200 proof ethanol (1.4 mL, 1.38 mmol) was processed as described in Example 46, Step E, to provide 3′-chloro-7′,8′-dihydro-6′H-spiro[imidazolidine-4,5′-isoquinoline]-2,5-dione (240 mg, 69.3% yield) as a solid. LCMS (APCI+) m/z 252 (M+H)+.

Step B: 3′-Chloro-1-methyl-7′,8′-dihydro-6′H-spiro[imidazolidine-4,5′-isoquinoline]-2,5-dione (380 mg, 97% yield) was prepared from 3′-chloro-7′,8′-dihydro-6′H-spiro[imidazolidine-4,5′-isoquinoline]-2,5-dione (370 mg, 1.470 mmol) and iodomethane (86.95 μL, 1.397 mmol) according to the general method described in Example 46, Step F. LCMS (APCI+) m/z 261 (M+H)+.

Step C: A mixture of 3′-chloro-1-methyl-7′,8′-dihydro-6′H-spiro[imidazolidine-4,5′-isoquinoline]-2,5-dione (50 mg, 0.19 mmol), 3-chlorophenylboronic acid (28 mg, 0.18 mmol), PdCl₂(dppf)*dcm (7.7 mg, 0.0094 mmol), 2M aqueous sodium carbonate (376 μL, 0.75 mmol), and 1,4-dioxane (753 μl, 0.19 mmol) was processed according to the general method described for the preparation of Example 46, Step C, to provide 3′-(3-chlorophenyl)-1-methyl-7′,8′-dihydro-6′H-spiro[imidazolidine-4,5′-isoquinoline]-2,5-dione (65 mg, 92% yield) as solid. LCMS (APCI+) m/z 342 (M+H)+.

Step D: A suspension of 3′-(3-chlorophenyl)-1-methyl-7′,8′-dihydro-6′H-spiro[imidazolidine-4,5′-isoquinoline]-2,5-dione (64 mg, 0.19 mmol) and Lawesson's Reagent (45 mg, 0.11 mmol) in toluene (1248 μL, 0.19 mmol) was processed as described for Example 46, Step G, to provide 3′-(3-chlorophenyl)-1-methyl-2-thioxo-7′,8′-dihydro-6′H-spiro[imidazolidine-4,5′-isoquinolin]-5-one as a solid. LCMS (APCI−) m/z 356 (M−H)−.

Step E: Crude 3′-(3-chlorophenyl)-1-methyl-2-thioxo-7′,8′-dihydro-6′H-spiro[imidazolidine-4,5′-isoquinolin]-5-one (30 mg, 0.0838 mmol) in methanol (1 mL) was treated with tert butylhydroperoxide 70% in water (58.0 μL, 0.419 mmol) and ammonia 7M in MeOH (359 μL, 2.51 mmol) as described for Example 46, Step H, to provide 2-amino-3′-(3-chlorophenyl)-1-methyl-7′,8′-dihydro-6′H-spiro[imidazole-4,5′-isoquinolin]-5(1H)-one (4 mg, 14.0% yield) as a solid. ¹H NMR (400 MHz, CDCl₃) δ 8.53 (s, 1H), 7.87 (s, 1H), 7.76-7.73 (m, 1H), 7.37-7.35 (m, 2H), 7.18 (s, 1H), 3.25 (s, 3H), 2.95-2.90 (m, 2H), 2.40-2.31 (m, 1H), 2.45-2.17 (m, 1H), 2.11-1.95 (m, 2H); LCMS (APCI+) m/z, 341 (M+H)+.

### Example 48

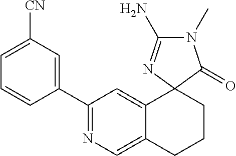

### 3-(2-amino-1-methyl-5-oxo-1,5,7′,8′-tetrahydro-6′H-spiro[imidazole-4,5′-isoquinoline]-3′-yl)benzonitrile

Step A: A mixture of 3-chloro-7,8-dihydroisoquinolin-5(6H)-one (750 mg, 4.13 mmol), ammonium carbonate (4365 mg, 45.4 mmol), potassium cyanide (672 mg, 10.3 mmol), sodium bisulfite (85.9 mg, 0.826 mmol) and ethanol (4130 μL, 4.13 mmol) were processed as described in Example 46, Step E, to provide 3′-chloro-7′,8′-dihydro-6′H-spiro[imidazolidine-4,5′-isoquinoline]-2,5-dione (900 mg, 87% yield) as a solid. MS (APCI−) m/z 250, 252 (M−H)−.

Step B: A mixture of 3′-chloro-7′,8′-dihydro-6′H-spiro[imidazolidine-4,5′-isoquinoline]-2,5-dione (895 mg, 3.556 mmol) and K₂CO₃ (491.5 mg, 3.556 mmol) in N,N-dimethylformamide (11854 μL, 3.556 mmol) was treated with iodomethane (221.4 μl, 3.556 mmol) as described in Example 46, Step F, to provide 3′-chloro-1-methyl-7′,8′-dihydro-6′H-spiro[imidazolidine-4,5′-isoquinoline]-2,5-dione (876 mg, 92.7% yield) as a solid. LCMS (APCI−) m/z 264 (M−H)−.

Step C: A suspension of 3′-chloro-1-methyl-7′,8′-dihydro-6′H-spiro[imidazolidine-4,5′-isoquinoline]-2,5-dione (595 mg, 2.24 mmol) in xylenes (22 mL) was stirred at 145° C. for 10 minutes and treated with solid Lawesson's Reagent (498 mg, 1.23 mmol). The mixture was stirred at 145° C. for 8 hours and allowed to stir at ambient temperature for 12 hours. The reaction mixture was diluted with EtOAc (150 mL) and poured into water (60 mL). The layers were separated, and the organic later was washed with additional water (3×50 mL), then dried (MgSO₄), filtered, and concentrated in vacuo to provide the crude 3′-chloro-1-methyl-2-thioxo-7′,8′-dihydro-6′H-spiro[imidazolidine-4,5′-isoquinolin]-5-one (1.36 g, 2.51 mmol, 112% yield) as a gum. MS (APCI−) 299.9, 282 (M−H)—.

Step D: The crude 3′-chloro-1-methyl-2-thioxo-7′,8′-dihydro-6′H-spiro[imidazolidine-4,5′-isoquinolin]-5-one (1.36 g, 2.51 mmol) was treated with tert butylhydroperoxide 70% in water (1.22 mL, 8.78 mmol) and ammonia 7M in methanol (6.45 ml, 45.2 mmol) as described in Example 46, Step E, to provide 2-amino-3′-chloro-1-methyl-7′,8′-dihydro-6′H-spiro[imidazole-4,5′-isoquinolin]-5(1H)-one (210 mg, 31.6% yield) as a solid. LCMS (APCI+) m/z 265 (M+H)+.

Step E: A mixture of 2-amino-3′-chloro-1-methyl-7′,8′-dihydro-6′H-spiro[imidazole-4,5′-isoquinolin]-5(1H)-one (50 mg, 0.19 mmol), 3-cyanophenylboronic acid (33 mg, 0.23 mmol), dichloro[1,1′-bis(diphenylphosphino)ferrocene]palladium (II) dichloromethane adduct (7.8 mg, 0.0094 mmol), 20% aqueous Na₂CO₃ (350 μL, 0.66 mmol), and 1,4-dioxane (1889 μL, 0.19 mmol) was processed as described in Example 46, Step C, to provide 3-(2-amino-1-methyl-5-oxo-1,5,7′,8′-tetrahydro-6′H-spiro[imidazole-4,5′-isoquinoline]-3′-yl)benzonitrile (2.5 mg, 4.0% yield) as a solid. LCMS (APCI+) m/z 332 (M+H)+.

### Example 49

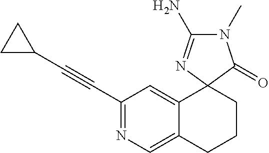

### 2-amino-3′-(cyclopropylethynyl)-1-methyl-7′,8′-dihydro-6′H-spiro[imidazole-4,5′-isoquinolin]-5(1H)-one

Step A: A 4 mL screw cap glass vial was charged with PdCl₂(MeCN)₂ (0.490 mg, 0.00189 mmol), dicyclohexyl(2′,4′,6′-triisopropylbiphenyl-2-yl)phosphine (X-Phos) (2.70 mg, 0.00567 mmol), Cs₂CO₃ (123 mg, 0.378 mmol), anhydrous acetonitrile (378 μl, 0.189 mmol), and 2-amino-3′-chloro-1-methyl-7′,8′-dihydro-6′H-spiro[imidazole-4,5′-isoquinolin]-5(1H)-one (50 mg, 0.189 mmol). The resulting suspension was sparged with nitrogen for 5 minutes and allowed to stir at room temperature for 30 minutes. The mixture was cooled to −78° C., and ethynylcyclopropane (160 μL, 1.89 mmol) was slowly injected through a rubber septum. The vial was capped, allowed to come to room temperature and then stirred at 75° C. for 18 hours. The reaction mixture was then cooled to ambient temperature, diluted with EtOAc (20 mL) and washed with water (2×5 mL). The organic layer was separated, dried (MgSO₄), filtered and concentrated in vacuo. The residue obtained was purified by silica gel prep TLC (0.5 mm pre-coated plate) eluting with 8% MeOH/CH₂Cl₂+NH₃ to provide 2-amino-3′-(cyclopropylethynyl)-1-methyl-7′,8′-dihydro-6′H-spiro[imidazole-4,5′-isoquinolin]-5(1H)-one (12 mg, 21.6% yield) as a solid. LCMS (APCI+) m/z 295 (M+H)+.

The following compounds in Table 1 were prepared according to the above procedures using appropriate intermediates.

TABLE 1

| Ex. # | Structure | Name | NMR/MS |
|---|---|---|---|
| 50 | 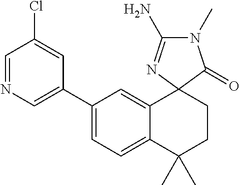 | 2-amino-7′-(5-chloropyridin- 3-yl)-1,4′,4′-trimethyl-3′,4′- dihydro-2′H-spiro[imidazole- 4,1′-naphthalen]-5(1H)-one | MS (APCI- pos) = 369.2 (M + 1) |
| 51 | 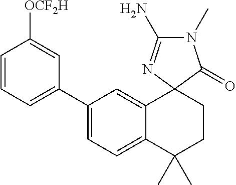 | 2-amino-7′-(3- (difluoromethoxy)phenyl)- 1,4′,4′-trimethyl-3′,4′- dihydro-2′H-spiro[imidazole- 4,1′-naphthalen]-5(1H)-one | MS (APCI- pos) = 400.2 (M + 1) |
| 52 | 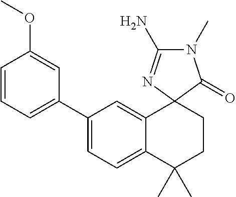 | 2-amino-7′-(3- methoxyphenyl)-1,4′,4′- trimethyl-3′,4′-dihydro-2′H- spiro[imidazole-4,1′- naphthalen]-5(1H)-one | MS (APCI- pos) = 364.2 (M + 1) |
| 53 | 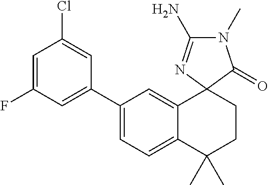 | 2-amino-7′-(3-chloro-5- fluorophenyl)-1,4′,4- trimethyl-3′,4′-dihydro-2′H- spiro[imidazole-4,1′- naphthalen]-5(1H)-one | MS (APCI- pos) = 386.2 (M + 1) |
| 54 | 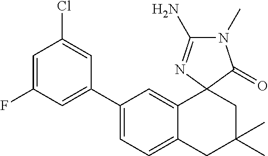 | 2-amino-2′-(3-chloro-5- fluorophenyl)-1,6′,6′- trimethyl-6′,7′-dihydro-5′H- spiro[imidazole-4,8′- quinolin]-5(1H)-one | MS (APCI- pos) = 387.2 (M + 1) |
| 55 | 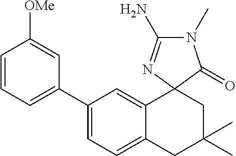 | 2-amino-2′-(3- methoxyphenyl)-1,6′,6′- trimethy1-6,7′-dihydro-5′H- spiro[imidazole-4,8′- quinolin]-5(1H)-one | MS (APCI- pos) = 365.2 (M + 1) |
| 56 | 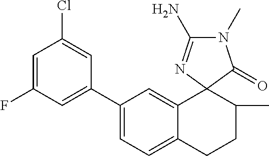 | 2-amino-7′-(3-chloro-5- fluorophenyl)-1,2′-dimethyl- 3′,4′-dihydro-2′H- spiro[imidazole-4,1′- naphthalen]-5(1H)-one | MS (APCI- pos) = 372 (M + 1) |
| 57 | 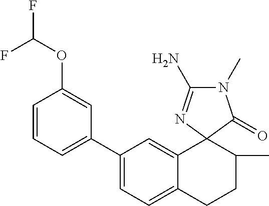 | 2-amino-7′-(3- (difluoromethoxy)phenyl)- 1,2′-dimethy1-3′,4-dihydro- 2′H-spiro[imidazole-4,1′- naphthalen]-5(1H)-one | MS (APCI- pos) = 386 (M + 1) |
| 58 | 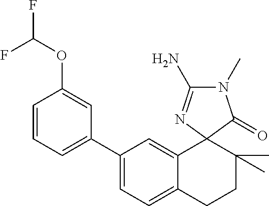 | 2-amino-7′-(3- (difluoromethoxy)phenyl)- 1,2′,2′-trimethyl-3′,4′- dihydro-2′H-spiro[imidazole- 4,1′-naphthalen]-5(1H)-one | m/z (APCI+) M + 1 = 400 |
| 59 | 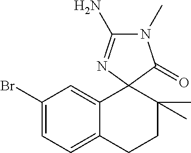 | 2-amino-7′-bromo-1,2′,2′- trimethyl-3′,4′-dihydro-2′H- spiro[imidazole-4,1′- naphthalen]-5(1H)-one | m/z (APCI+) M + 1 = 336/338 |
| 60 | 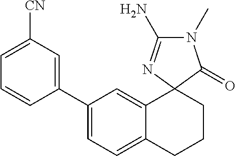 | 3-(2-amino-1-methyl-5-oxo- 1,3′,4′,5-tetrahydro-2′H- spiro[imidazole-4,1′- naphthalene]-7′- yl)benzonitrile | m/z (APCI+) M + 1 = 331 |
| 61 | 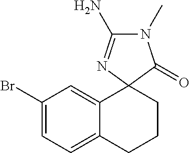 | 2-amino-7′-bromo-1-methyl- 3′,4′-dihydro-2′H- spiro[imidazole-4,1′- naphthalen]-5(1H)-one | m/z (APCI+) M + 1 = 308/310 |
| 62 | 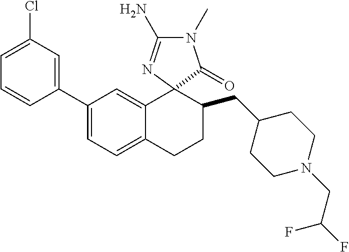 | (1′S,2′R)-2-amino-7′-(3- chlorophenyl)-2′-((1-(2,2- difluoroethyl)piperidin-4- yl)methyl)-1-methyl-3′,4′- dihydro-2′H-spiro[imidazole- 4,1′-naphthalen]-5(1H)-one | MS (APCI- pos) = 501 (M + 1) |
| 63 | 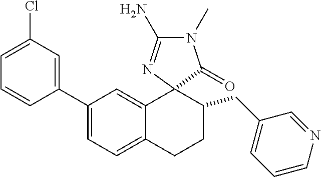 | (1′S,2′S)-2-amino-7′-(3- chlorophenyl)-1-methyl-2′- (pyridin-3-ylmethyl)-3′,4′- dihydro-2′H-spiro[imidazole- 4,1′-naphthalen]-5(1H)-one | MS (APCI- pos) = 431 (M + 1) |
| 64 | 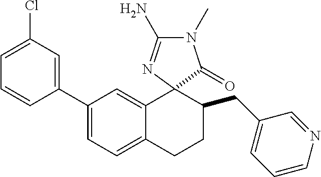 | (1′S,2′R)-2-amino-7′-(3- chlorophenyl)-1-methyl-2′- (pyridin-3-ylmethyl)-3′,4′- dihydro-2′H-spiro[imidazole- 4,1′-naphthalen]-5(1H)-one | MS (APCI- pos) = 431 (M + 1) |
| 65 | 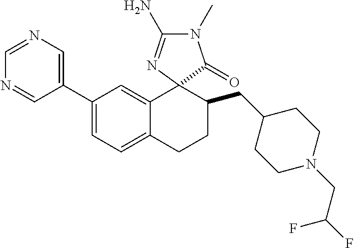 | (1′S,2′R)-2-amino-2′-((1-(2,2- difluoroethyl)piperidin-4- yl)methyl)-1-methyl-7′- (pyrimidin-5-yl)-3′,4′- dihydro-2′H-spiro[imidazole- 4,1′-naphthalen]-5(1H)-one | MS (APCI- pos) = 469 (M + 1) |
| 66 | 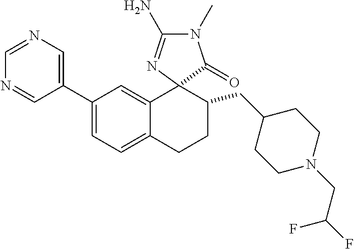 | (1′S,2′S)-2-amino-2′-((1-(2,2- difluoroethyl)piperidin-4- yl)methyl)-1-methyl-7′- (pyrimidin-5-yl)-3′,4′- dihydro-2′H-spiro[imidazole- 4,1′-naphthalen]-5(1H)-one | MS (APCI- pos) = 469 (M + 1) |
| 67 | 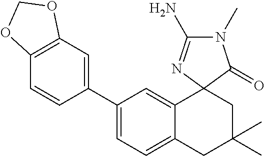 | 2-amino-7′- (benzo[d][1,3]dioxol-5-yl)- 1,3′,3′-trimethyl-3′,4′- dihydro-2′H-spiro[imidazole- 4,1′-naphthalen]-5(1H)-one | MS (APCI- pos) = 378.2 (M + 1) |
| 68 | 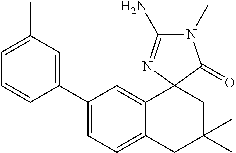 | 2-amino-1,3′,3′-trimethyl-7′- m-tolyl-3′,4′-dihydro-2′H- spiro[imidazole-4,1′- naphthalen]-5(1H)-one | MS (APCI- pos) = 348.2 (M + 1) |
| 69 | 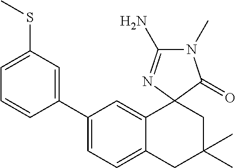 | 2-amino-1,3′,3′-trimethyl-7′- (3-(methylthio)phenyl)-3′,4′- dihydro-2′H-spiro[imidazole- 4,1′-naphthalen]-5(1H)-one | MS (APCI- pos) = 380.1 (M + 1) |
| 70 | 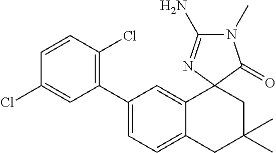 | 2-amino-7′-(2,5- dichiorophenyl)-1,3′,3′- trimethyl-3′,4′-dihydro-2′H- spiro[imidazole-4,1′- naphthalen]-5(1H)-one | MS (APCI- pos) = 402.1 (M + 1) |
| 71 | 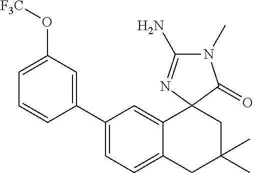 | 2-amino-1,3′,3′-trimethyl-7′- (3- (trifluoromethoxy)phenyl)- 3′,4′-dihydro-2′H- spiro[imidazole-4,1′- naphthalen]-5(1H)-one | MS (APCI- pos) = 418.2 (M + 1) |
| 72 | 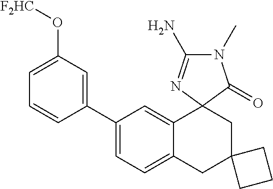 | 2-amino-7′-(3- (difluoromethoxy)phenyl)-1- methyl-5-oxo-3′,4′-dihydro- 2′H-dispiro[imidazol-4,1′- naphthalen-3′,1″-cyclobutyl]- 5(1H)-one | MS (APCI- pos) = 412.2 (M + 1) |
| 73 | 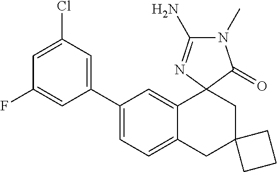 | 2-amino-7′-(3-chloro-5- fluorophenyl)-1-methyl-5- oxo-3′,4′-dihydro-2′H- dispiro[imidazol-4,1′- naphthalen-3′,1″-cyclobutyl]- 5(1H)-one | MS (APCI- pos) = 398.2 (M + 1) |
| 74 | 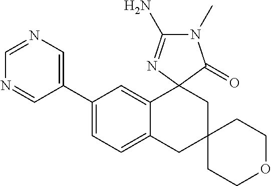 | 2-amino-7″-(pyrimidin-5-yl)- 1-methyl-2′,3′,5′,6′- tetrahydro-3″,4″-dihydro- 2″H-dispiro[imidazol-4,1″- naphthalen-3″,4′-pyran]- 5(1H)-one | MS (APCI- pos) = 378.3 (M + 1) |
| 75 | 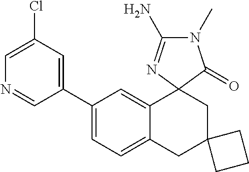 | 2-amino-7′-(5-chloropyridin- 3-yl)-1-methyl-5-oxo-3′,4′- dihydro-2′H- dispiro[imidazol-4,1′- naphthalen-3′,1″-cyclobutyl]- 5(1H)-one | MS (APCI- pos) = 381.3 (M + 1) |
| 76 | 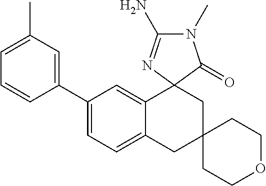 | 2-amino-7″-(3- methyiphenyl)-1-methyl- 2′,3′,5′,6′-tetrahydro-3″,4″- dihydro-2″H- dispiro[imidazol-4,1″- naphthalen-3″,4′-pyran]- 5(1H)-one | MS (APCI- pos) = 390.2 (M + 1) |
| 77 | 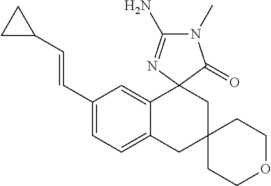 | (E)-2-amino-7″-(2- cyclopropylvinyl)-1-methyl- 2′,3′,5′,6′-tetrahydro-3″,4″- dihydro-2″H- dispiro[imidazol-4,1″- naphthalen-3″,4′-pyran]- 5(1H)-one | MS (APCI- pos) = 366.2 (M + 1) |

### Example 78

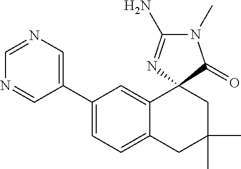

### (R)-2-amino-1,3′,3′-trimethyl-7′-(pyrimidin-5-yl)-3′,4′-dihydro-2′H-spiro[imidazole-4,1′-naphthalen]-5(1H)-one

Step A: SFC separation of racemic 2-amino-7′-bromo-1,3′,3′-trimethyl-3′,4′-dihydro-2′H-spiro[imidazole-4,1′-naphthalen]-5(1H)-one (133 g, 397 mmol) was performed on a Lux Cellulose-4 (3×25 cm) column eluting with 35% methanol (0.1% NH₄OH)/CO₂ at 100 bar at a flow rate of 200 mL/minute (injection volume 2 mL, 309 mg/mL methanol). The peaks isolated were analyzed on a Lux Cellulose-4 (0.46×5 cm, 3 μm) column eluting with 25% methanol (0.1% NH₄OH)/CO₂ at 120 bar (flow rate 5 mL/minute, 210 nm). From this separation, (R)-2-amino-7′-bromo=1,3′,3′-trimethyl-3′,4′-dihydro-2′H-spiro[imidazole-4,1′-naphthalen]-5(1H)-one (peak-1, 46.55 g, chemical purity >99%, ee >99%) was isolated.

Step B: (R)-2-Amino-7′-bromo-1,3′,3′-trimethyl-3′,4′-dihydro-2′H-spiro[imidazole-4,1′-naphthalen]-5(1H)-one (1.00 g, 2.97 mmol), pyrimidin-5-ylboronic acid (0.479 g, 3.87 mmol), and Pd(PPh₃)₄ (0.0859 g, 0.0744 mmol) were combined with dioxane (15 mL) and 2M Na₂CO₃ (3.72 mL, 7.44 mmol) (both degassed with nitrogen sparge for 30 min prior to use), and the reaction mixture was heated in a 100° C. reaction block and stirred for 17 hours. The reaction mixture was then concentrated, and the residue was combined with ethyl acetate and water. The mixture was extracted with ethyl acetate (2×), and the combined extracts were dried (Na₂SO₄), filtered, and concentrated. The crude was purified on silica gel (5-20% MeOH in dichloromethane gradient) to give (R)-2-amino-1,3′,3′-trimethyl-7′-(pyrimidin-5-yl)-3′,4′-dihydro-2′H-spiro[imidazole-4,1′-naphthalen]-5 (1H)-one (0.755 g, 2.25 mmol, 75.7% yield) as a powder. ¹H NMR (400 MHz, CDCl₃) δ 9.16 (s, 1H), 8.83 (s, 2H), 7.40 (dd, J=7.8, 2.0 Hz, 1H), 7.25 (d, J=7.8 Hz, 1H), 7.08 (d, J=1.6 Hz, 1H), 5.09 (br s, 2H), 3.18 (s, 3H), 2.85 (d, J=16.4 Hz, 1H), 2.62 (dd, J=16.0, 2.3 Hz, 1H), 2.29 (d, J=13.7 Hz, 1H), 1.80 (dd, J=13.7, 2.3 Hz, 1H), 1.17 (s, 3H), 1.01 (s, 3H); m/z (APCI-pos) M+1=336.

### Example 79

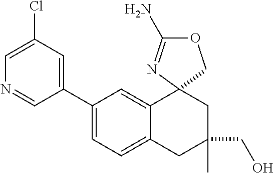

### ((1S*,3R*)-2′-amino-7-(5-chloropyridin-3-yl)-3-methyl-3,4-dihydro-2H,5′H-spiro[naphthalene-1,4′-oxazole]-3-yl)methanol

Step A: A round bottomed flask plus stir bar equipped with a Dean Stark trap was charged with 1-(4-bromophenyl)propan-2-one (100 g, 469 mmol), toluene (300 mL), and ethyl 2-cyanoacetate (53.1 g, 469 mmol). Next, ammonium acetate (17.4 g, 225 mmol) was added, followed by acetic acid (26 mL, 451 mmol). The reaction mixture was heated to reflux (bath temp=125° C.), and collected water in the Dean Stark trap (total of 32 mL of water collected) for 5 hours. After cooling to room temperature, the mixture was diluted with EtOAc (500 mL) and washed with water (200 mL). The aqueous layer was re-extracted with EtOAc (100 mL). The combined organic phases were washed again with water (200 mL), brine (200 mL), dried (MgSO₄), filtered, and concentrated to yield (E)-ethyl 4-(4-bromophenyl)-2-cyano-3-methylbut-2-enoate (160 g, 99%). No purification was performed.

Step B: Following a procedure similar to that described in Ogawa, Yutaka, et al. “Stereochemical studies on 3,4-benzobicyclo[4.1.0]hept-3-en-2-ol systems and solvolytic studies on its p-nitrobenzoates.” J. Org. Chem. 43 (1978): p. 849-855, 853, an aqueous solution (150 mL) of KCN (33.2 g, 510 mmol) was added with stirring and cooling in an ice bath to maintain internal temperature below 20° C. to a solution of (E)-ethyl 4-(4-bromophenyl)-2-cyano-3-methylbut-2-enoate (143 g, 464 mmol) in MeOH (300 mL). The ice bath was removed after complete addition of KCN solution, and the reaction was allowed to warm to room temperature and stirred for 1 hour. The mixture was carefully acidified with aqueous 3N HCl (250 mL), then N₂ was bubbled through the mixture to sparge excess HCN for 1 hour (hood sashes closed to minimize exposure to HCN). The product was extracted with diethyl ether (2×200 mL). The combined organic phases were washed with brine (200 mL), dried (MgSO₄), filtered, and concentrated to yield ethyl 4-(4-bromophenyl)-2,3-dicyano-3-methylbutanoate (148 g, 83%) that was present as a 1:1 mixture with methyl 4-(4-bromophenyl)-2,3-dicyano-3-methylbutanoate. The mixture was carried forward without purification at this step.

Step C: Following a procedure similar to that described in Ogawa, supra at 853, a round bottomed flask plus stir bar containing a 1:1 mixture of ethyl 4-(4-bromophenyl)-2,3-dicyano-3-methylbutanoate and methyl 4-(4-bromophenyl)-2,3-dicyano-3-methylbutanoate (148 g, 442 mmol) was charged with aqueous concentrated HCl (600 mL) and acetic acid (300 mL). The mixture was heated to reflux for 16 hours. After cooling to room temperature, the mixture was diluted with water (500 mL) and extracted with diethyl ether (2×150 mL). The combined organic phases were washed with brine (200 mL), dried (Na₂SO₄), filtered, and concentrated to obtain a 1:1 mixture of 2-(4-bromobenzyl)-2-methylsuccinic acid and 4-(4-bromophenyl)-3-cyano-3-methylbutanoic acid (165 g, 93%) that was carried forward to the next step without purification.

Step D: A round bottomed flask plus stir bar was charged with a 1:1 mixture of 2-(4-bromobenzyl)-2-methylsuccinic acid and 4-(4-bromophenyl)-3-cyano-3-methylbutanoic acid (123 g, 436 mmol; that had been azeotroped with 3×200 mL toluene on the rotovap to remove residual acetic acid), EtOH (500 mL), and then sodium hydroxide (87.2 g, 2180 mmol) dissolved in water (150 mL). The mixture was heated with stirring to reflux for 18 hours. The suspension was cooled in an ice bath to 5-10° C. internal temperature. The mixture was acidified with aqueous concentrated HCl (approximately 150 mL). The mixture was transferred to a separatory funnel with EtOAc (400 mL) and water (400 mL). The phases were separated. The aqueous was re-extracted with EtOAc (2×200 mL). The combined organic phases were washed with brine (300 mL), dried (MgSO₄), filtered, and concentrated. The residue (130 g) was azeotroped with toluene (2×200 mL) to remove residual solvents and water. The residue was triturated with toluene (200 mL) by heating and mixing with a spatula to obtain a suspension. The suspension was cooled in an ice bath, filtered, rinsing solids with toluene to yield 2-(4-bromobenzyl)-2-methylsuccinic acid (36.7 g, 27%).

Step E: A round bottomed flask plus stir bar was charged with 2-(4-bromobenzyl)-2-methylsuccinic acid (40.8 g, 135 mmol), and then carefully added neat H₂SO₄ (200 mL). The reaction mixture was stirred at room temperature for 16 hours. To drive the reaction to completion, the mixture was heated to 60° C. for 2 hours. After cooling to room temperature, the mixture was poured on to ice, and extracted with EtOAc (3×200 mL). The combined organic phases were washed with brine (300 mL), dried (MgSO₄), filtered, and concentrated to yield 6-bromo-2-methyl-4-oxo-1,2,3,4-tetrahydronaphthalene-2-carboxylic acid (28.6 g, 67%). The product was carried forward without purification.

Step F: A round bottomed flask plus stir bar was charged with toluene (300 mL) followed by 6-bromo-2-methyl-4-oxo-1,2,3,4-tetrahydronaphthalene-2-carboxylic acid (27.4 g, 96.8 mmol). The mixture was cooled in an ice bath. Under N₂, BH₃-THF complex (290 mL, 290 mmol) was added dropwise until foaming ceased (much gas evolution during first third of addition), then added the BH₃-THF in 10 mL portions until addition was finished. An internal temperature below 10° C. was maintained during the addition of BH₃-THF. The ice bath was removed. The reaction mixture was stirred for 2 hours at room temperature. 10% Aqueous citric acid solution (500 mL) was added to a second flask that was chilled in an ice bath with stirring. The reaction mixture was quenched by pouring into the citric acid solution in portions (much gas evolution, placed an N₂ line over the top of the mixture to continually flush out H₂ gas), maintaining the internal quench solution below 10° C. After complete addition of the reaction mixture to the quench solution, the solution was stirred for 2 hours at room temperature. The phases were separated, and the aqueous was re-extracted with EtOAc (2×200 mL). The combined organics were washed with brine (500 mL), dried (MgSO₄), filtered, and concentrated to yield 7-bromo-3-(hydroxymethyl)-3-methyl-1,2,3,4-tetrahydronaphthalen-1-ol (27 g, 72%), obtained as a 60:40 mixture of diastereomers. No purification was performed at this step.

Step G: A round bottomed flask plus stir bar was charged with 7-bromo-3-(hydroxymethyl)-3-methyl-1,2,3,4-tetrahydronaphthalen-1-ol (18 g, 66 mmol), CHCl₃ (500 mL), and then manganese (IV) oxide (58 g, 664 mmol). The reaction mixture was heated to 50° C. with stirring for 22 hours. The mixture was filtered through Celite®, rinsing with DCM. The filtrate was concentrated. The crude was purified by silica gel chromatography on a Biotage Flash 65 system, eluting with 30% EtOAc/hexanes, followed by 1:1 EtOAc/hexanes to yield 7-bromo-3-(hydroxymethyl)-3-methyl-3,4-dihydronaphthalen-1(2H)-one (13.0 g, 60%).

Step H: A stirred solution of 7-bromo-3-(hydroxymethyl)-3-methyl-3,4-dihydronaphthalen-1(2H)-one (19.1 g, 71.0 mmol) and tert-butylchlorodimethylsilane (10.7 g, 71.0 mmol) in DCM (200 mL) were cooled in an ice bath followed by portion-wise addition of imidazole (9.66 g, 142 mmol). The reaction was allowed to stir for 3 days at room temperature. The reaction was transferred to a separatory funnel and washed with saturated aqueous NH₄Cl (200 mL), brine (200 mL), dried (Na₂SO₄), filtered and concentrated to yield 7-bromo-3-((tert-butyldimethylsilyloxy)methyl)-3-methyl-3,4-dihydronaphthalen-1(2H)-one (26.0 g, 85%). The product was carried forward without purification.

Step I: A round bottomed flask plus stir bar was charged with sodium hydride (0.42 g, 10 mmol; 60% in oil) and anhydrous DMSO (“dimethylsulfoxide”) (20 mL). The mixture was heated to 75° C. for 30 minutes with stirring. The mixture was cooled in an ice bath under N₂, and methyltriphenylphosphonium bromide (3.7 g, 10 mmol) was added dropwise in warm DMSO (10 mL; warming of DMSO required to dissolve the Wittig reagent). The mixture was removed from the ice bath and stirred for 30 minutes at room temperature. Then, 7-bromo-3-((tert-butyldimethylsilyloxy)methyl)-3-methyl-3,4-dihydronaphthalen-1(2H)-one (2.0 g, 5.2 mmol) was added dropwise in DMSO (10 mL) at room temperature. The mixture continued stirring for 18 hours. The mixture was worked up by partitioning between EtOAc (50 mL) and water (50 mL). The phases were separated, and the aqueous was re-extracted with EtOAc (30 mL). The combined organic phases were washed with water (2×50 mL), brine (50 mL), dried (MgSO₄), filtered, and concentrated to obtain crude ((6-bromo-2-methyl-4-methylene-1,2,3,4-tetrahydronaphthalen-2-yl)methoxy)(tert-butyl)dimethylsilane (4.4 g). The crude material was purified by silica gel chromatography on a Biotage Flash 65 system, eluting with isocratic hexanes to a final yield of 1.36 g (67%).

Step J: A stirred solution of ((6-bromo-2-methyl-4-methylene-1,2,3,4-tetrahydronaphthalen-2-yl)methoxy)(tert-butyl)dimethylsilane (1.4 g, 3.67 mmol) in diethyl ether (20 mL) was cooled to 0° C. under N₂. In a separate flask, silver cyanate (2.20 g, 14.7 mmol) was suspended in CH₃CN (10 mL), and to this suspension, iodine (1.86 g, 7.34 mmol) in THF (10 mL) was added. The resulting mixture was shaken for 30 seconds. This suspension was then poured into the alkene-containing solution at 0° C. The reaction mixture was then removed from the ice bath and allowed to stir at room temperature for 1 hour. The reaction mixture was filtered through Celite®, rinsing with diethyl ether, and the filtrate was concentrated. The residue was dissolved in THF (20 mL) and aqueous NH₄OH (2 mL) was added. The resulting mixture was stirred at room temperature for 2 hours. The reaction mixture was partitioned between ethyl acetate (50 mL) and saturated Na₂S₂O₃ (30 mL). After shaking and then separating the phases, the aqueous layer was re-extracted with ethyl acetate (30 mL). The combined organic layers were washed with brine (30 mL), dried (MgSO₄), filtered, and concentrated to give a waxy solid that was consistent with a 3:1 ratio of diastereomers by ¹H NMR of 7-bromo-3-((tert-butyldimethylsilyloxy)methyl)-3-methyl-3,4-dihydro-2H,5′H-spiro[naphthalene-1,4′-oxazol]-2′-amine (1.55 g, 87%). The product was pure enough to take forward without purification.

Step K: A round bottomed flask plus stir bar was charged with 7-bromo-3-((tert-butyldimethylsilyloxy)methyl)-3-methyl-3,4-dihydro-2H,5′H-spiro[naphthalene-1,4′-oxazol]-2′-amine (1.45 g, 3.30 mmol), THF (5 mL), and tetrabutylammonium fluoride (3.6 mL, 3.6 mmol; 1N in THF). The reaction mixture was stirred at room temperature for 2 hours. The reaction mixture was concentrated in vacuo. The crude was purified by silica gel chromatography on a Biotage Flash 65 system, eluting with EtOAc, followed by a gradient of 5-10% MeOH/EtOAc to yield (2′-amino-7-bromo-3-methyl-3,4-dihydro-2H,5′H-spiro[naphthalene-1,4′-oxazole]-3-yl)methanol (863 mg, 79%).

Step L: A 2 dram vial was charged with (2′-amino-7-bromo-3-methyl-3,4-dihydro-2H,5′H-spiro[naphthalene-1,4′-oxazole]-3-yl)methanol (50 mg, 0.15 mmol), dioxane (1 mL), 5-chloropyridin-3-ylboronic acid (24 mg, 0.15 mmol), Pd(PPh₃)₄ (18 mg, 0.015 mmol), and 2N aqueous Na₂CO₃ (192 μL, 0.38 mmol). The reaction mixture was sparged with N₂ for 30 seconds, then heated to 90° C. for 16 hours. The mixture was loaded directly on to a preparative TLC plate (1 mm thickness, Rf=0.42) and eluted with 10% MeOH (containing 7N NH₃)/DCM. The diastereomers were separated. A yield of ((1S*,3R*)-2′-amino-7-(5-chloropyridin-3-yl)-3-methyl-3,4-dihydro-2H,5′H-spiro[naphthalene-1,4′-oxazole]-3-yl)methanol (16 mg, 28%) was obtained. m/z (APCI-pos) M+1=358.

### Example 80

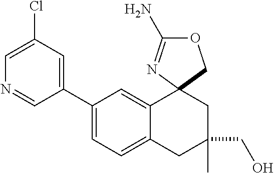

### ((1R*,3R*)-2′-amino-7-(5-chloropyridin-3-yl)-3-methyl-3,4-dihydro-2H,5′H-spiro[naphthalene-1,4′-oxazole]-3-yl)methanol

((1R*,3R*)-2′-Amino-7-(5-chloropyridin-3-yl)-3-methyl-3,4-dihydro-2H,5′H-spiro[naphthalene-1,4′-oxazole]-3-yl)methanol (6 mg, 10%) was synthesized by the procedure in Example 79, steps A-L, and separated from its diastereomer by preparative TLC (1 mm thickness, Rf=0.32) eluting with 10% MeOH (containing 7N NH₃)/DCM. m/z (APCI-pos) M+1=358.

### Example 81

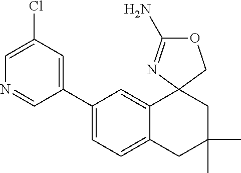

### 5-(2′-amino-3,3-dimethyl-3,4-dihydro-2H,5′H-spiro[naphthalene-1,4′-oxazo]-7-yl)nicotinonitrile

Step A: Sodium hydride (1.3 g, 55 mmol; 95%, dry) was added to dimethylsulfoxide (100 mL) in a round bottom flask. The reaction was heated to 75° C. and stirred for 1 hour. The reaction was cooled to 0° C., and methyltriphenylphosphonium bromide (20 g, 55 mmol) was added dropwise in warm dimethylsulfoxide (30 mL). After stirring for 15 minutes, 7-bromo-3,3-dimethyl-3,4-dihydronaphthalen-1(2H)-one (7 g, 28 mmol) was added dropwise in dimethylsulfoxide (20 mL). The reaction was allowed to warm to ambient temperature and stirred overnight. The reaction was diluted with ethyl acetate and washed twice with water. The organics were dried over MgSO₄, filtered and concentrated. The residue was purified on silica gel eluting with hexanes to yield 7-bromo-3,3-dimethyl-1-methylene-1,2,3,4-tetrahydronaphthalene (6.5 g, 26 mmol, 94% yield).

Step B: 7-Bromo-3,3-dimethyl-1-methylene-1,2,3,4-tetrahydronaphthalene (2.86 g, 11.4 mmol) was diluted with diethyl ether (25 mL) followed by the addition of silver cyanate (5.12 g, 34.2 mmol). The reaction was placed under nitrogen and cooled to 0° C. I₂ (2.89 g, 11.4 mmol) was added, and the reaction was stirred for 1 hour at 0° C. The reaction was filtered through glass microfibre filter (“GF/F”) paper and concentrated. The residue was taken up in acetone (20 mL) and NH₄OH (5 mL). After stirring for 12 hours, the reaction was diluted with ethyl acetate and water. The layers were separated, and the organics were dried over MgSO₄, filtered and concentrated. The residue was purified on C-18 silica gel, eluting with 5-95% ACN/water (0.1% TFA) to afford 7-bromo-3,3-dimethyl-3,4-dihydro-2H,5′H-spiro[naphthalene-1,4′-oxazol]-2′-amine (1.0 g, 3.23 mmol, 28.4% yield).

Step C: 7-Bromo-3,3-dimethyl-3,4-dihydro-2H,5′H-spiro[naphthalene-1,4′-oxazol]-2′-amine (25 mg, 0.081 mmol) and 5-cyanopyridin-3-ylboronic acid (18 mg, 0.12 mmol) were diluted with dioxane (1 mL) followed by the addition of Pd(PPh₃)₄ (4.7 mg, 0.0040 mmol) and Na₂CO₃ (141 μL, 0.28 mmol). The reaction was sealed, heated to 85° C. and stirred for 12 hours. The reaction was loaded onto silica gel and eluted with 1-10% methanol/DCM (1% NH₄OH) to yield 5-(2′-amino-3,3-dimethyl-3,4-dihydro-2H,5′H-spiro[naphthalene-1,4′-oxazole]-7-yl)nicotinonitrile (4 mg, 0.012 mmol, 15% yield). ¹H NMR (400 MHz, CDCl₃) δ 9.00 (d, 1H), 8.82 (d, 1H), 8.10 (t, 1H), 7.45 (d, 1H), 7.37 (dd, 1H), 7.20 (d, 1H), 4.4 (dd, 1H), 2.71 (d, 1H), 2.59 (d, 1H), 2.09 (d, 1H), 1.85 (d, 1H), 1.12 (s, 3H), 0.99 (s, 3H); m/z (APCI-pos) M+1=333.1.

### Example 82

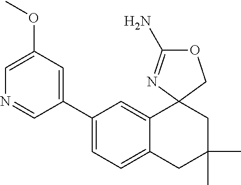

### 7-(5-methoxypyridin-3-yl)-3,3-dimethyl-3,4-dihydro-2H,5′H-spiro[naphthalene-1,4′-oxazol]-2′-amine

7-(5-Methoxypyridin-3-yl)-3,3-dimethyl-3,4-dihydro-2H,5′H-spiro[naphthalene-1,4′-oxazol]-2′-amine (10 mg, 0.030 mmol, 37% yield) was prepared according to Example 81, substituting 5-methoxypyridin-3-ylboronic acid for 5-cyanopyridin-3-ylboronic acid. ¹H NMR (400 MHz, CDCl₃) δ 8.44 (d, 1H), 8.27 (d, 1H), 7.47 (d, 1H), 7.35 (m, 2H), 7.12 (d, 1H), 4.37 (s, 2H), 3.90 (s, 3H), 2.70 (d, 1H), 2.59 (d, 1H), 2.09 (d, 1H), 1.85 (d, 1H), 1.12 (s, 3H), 0.99 (s, 3H); m/z (APCI-pos) M+1=338.1.

### Example 83

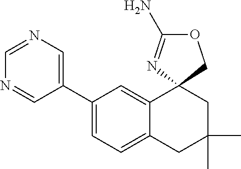

### (R)-3,3-dimethyl-7-(pyrimidin-5-yl)-3,4-dihydro-2H,5′H-spiro[naphthalene-1,4′-oxazol]-2′-amine

(R)-3,3-Dimethyl-7-(pyrimidin-5-yl)-3,4-dihydro-2H,5′H-spiro[naphthalene-1,4′-oxazol]-2′-amine (600 mg, 1.95 mmol, 40% yield) was prepared according to Example 81, substituting pyrimidin-5-ylboronic acid for 5-cyanopyridin-3-ylboronic acid. Compound was purified on a SFC system using a chiral column (Chiralpak AD-H, 2×15 cm). Eluting with 35% methanol (20 nM NH₃)/CO₂, 100 bar, 60 mL/minute, monitoring at 220 nM. ¹H NMR (400 MHz, CDCl₃) δ 9.18 (s, 1H), 8.87 (s, 2H), 7.46 (d, 1H), 7.37 (dd, 1H), 7.18 (d, 1H), 4.39 (dd, 2H), 4.36 (br s, 2H), 2.70 (d, 1H), 2.59 (d, 1H), 2.09 (d, 1H), 1.85 (d, 1H), 1.12 (s, 3H), 0.99 (s, 3H); m/z (APCI-pos) M+1=309.1.

### Example 84

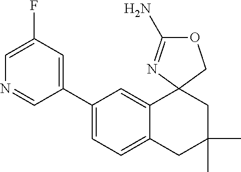

### 7-(5-fluoropyridin-3-yl)-3,3-dimethyl-3,4-dihydro-2H,5′H-spiro[naphthalene-1,4′-oxazol]-2′-amine

7-(5-Fluoropyridin-3-yl)-3,3-dimethyl-3,4-dihydro-2H,5′H-spiro[naphthalene-1,4′-oxazol]-2′-amine (10 mg, 0.031 mmol, 38% yield) was prepared according to Example 81, substituting 5-fluoropyridin-3-ylboronic acid for 5-cyanopyridin-3-ylboronic acid. m/z (APCI-pos) M+1=326.1.

### Example 85

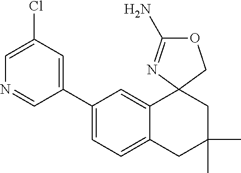

### 7-(5-chloropyridin-3-yl)-3,3-dimethyl-3,4-dihydro-2H,5′H-spiro[naphthalene-1,4′-oxazol]-2′-amine

7-(5-Chloropyridin-3-yl)-3,3-dimethyl-3,4-dihydro-2H,5′H-spiro[naphthalene-1,4′-oxazol]-2′-amine (8 mg, 0.023 mmol, 29% yield) was prepared according to Example 81, substituting 5-chloropyridin-3-ylboronic acid for 5-cyanopyridin-3-ylboronic acid. m/z (APCI-pos) M+1=342.1.

### Example 86

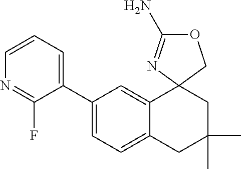

### 7-(2-fluoropyridin-3-yl)-3,3-dimethyl-3,4-dihydro-2H,5′H-spiro[naphthalene-1,4′-oxazol]-2′-amine

7-(2-Fluoropyridin-3-yl)-3,3-dimethyl-3,4-dihydro-2H,5′H-spiro[naphthalene-1,4′-oxazol]-2′-amine (25 mg, 0.077 mmol, 95% yield) was prepared according to Example 81, substituting 2-fluoropyridin-3-ylboronic acid for 5-cyanopyridin-3-ylboronic acid. m/z (APCI-pos) M+1=326.1.

### Example 87

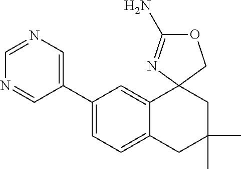

### 3,3-dimethyl-7-(pyrimidin-5-yl)-3,4-dihydro-2H,5′H-spiro[naphthalene-1,4′-oxazol]-2′-amine

3,3-Dimethyl-7-(pyrimidin-5-yl)-3,4-dihydro-2H,5′H-spiro[naphthalene-1,4′-oxazol]-2′-amine (18 mg, 0.058 mmol, 72% yield) was prepared according to Example 81, substituting pyrimidin-5-ylboronic acid for 5-cyanopyridin-3-ylboronic acid. m/z (APCI-pos) M+1=309.1.

### Example 88

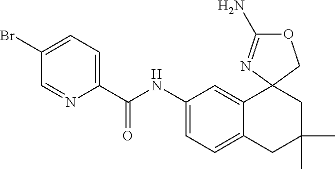

### N-(2′-amino-3,3-dimethyl-3,4-dihydro-2H,5′H-spiro[naphthalene-1,4′-oxazole]-7-yl)-5-bromopicolinamide

Step A: 7-Bromo-3,3-dimethyl-3,4-dihydro-2H,5′H-spiro[naphthalene-1,4′-oxazol]-2′-amine (900 mg, 2.91 mmol; from Example 81, Step B) was diluted with THF (5 mL), followed by the addition of Boc₂O (720 mg, 3.20 mmol) and TEA (446 μL, 3.20 mmol; d. 0.726). After stirring for 12 hours, the reaction was purified on silica gel eluting with 10-50% ethyl acetate/hexanes to yield tert-butyl 7-bromo-3,3-dimethyl-3,4-dihydro-2H,5′H-spiro[naphthalene-1,4′-oxazole]-2′-ylcarbamate (670 mg, 1.64 mmol, 56.2% yield).

Step B: tert-Butyl 7-bromo-3,3-dimethyl-3,4-dihydro-2H,5′H-spiro[naphthalene-1,4′-oxazole]-2′-ylcarbamate (670 mg, 1.64 mmol), Pd₂ dba₃ (74.9 mg, 0.0818 mmol), and biphenyl-2-yldicyclohexylphosphine (57.4 mg, 0.164 mmol) were diluted with LiHMDS (4092 μL, 4.09 mmol, in toluene). The reaction was purged with argon, sealed and heated to 80° C. overnight. The reaction was allowed to cool, transferred with minimal dioxanes and treated with 1N HCl (1 mL) for 15 minutes. The reaction was diluted with ethyl acetate and 10% aqueous sodium carbonate. The layers were separated, and the organics were dried over MgSO₄, filtered and concentrated. The material was purified on silica gel eluting with 10-50% ethyl acetate/hexanes to yield tert-butyl 7-amino-3,3-dimethyl-3,4-dihydro-2H,5′H-spiro[naphthalene-1,4′-oxazole]-2′-ylcarbamate (120 mg, 0.347 mmol, 21.2% yield).

Step C: tert-Butyl 7-amino-3,3-dimethyl-3,4-dihydro-2H,5′H-Spiro[naphthalene-1,4′-oxazole]-2′-ylcarbamate (12 mg, 0.035 mmol), 5-bromopicolinic acid (7.7 mg, 0.038 mmol), and 4-(4,6-dimethoxy[1.3.5]triazin-2-yl)-4-methylmorpholinium chloride hydrate (13 mg, 0.045 mmol) were diluted with methanol and stirred for 12 hours. The reaction was diluted with ethyl acetate and 10% aqueous sodium carbonate. The layers were separated, and the organic layer was dried over MgSO₄, filtered and concentrated. The residue was purified on silica gel eluting with 10-90% ethyl acetate/hexanes to yield tert-butyl 7-(5-bromopicolinamido)-3,3-dimethyl-3,4-dihydro-2H,5′H-spiro[naphthalene-1,4′-oxazole]-2′-ylcarbamate (10 mg, 0.019 mmol, 54% yield).

Step D: tert-Butyl 7-(5-bromopicolinamido)-3,3-dimethyl-3,4-dihydro-2H,5′H-spiro[naphthalene-1,4′-oxazole]-2′-ylcarbamate (10 mg, 0.019 mmol) was diluted DCM (500 μL) followed by the addition of TFA (500 μL). After stirring for 1 hour, the reaction was concentrated to afford N-(2′-amino-3,3-dimethyl-3,4-dihydro-2H,5′H-spiro[naphthalene-1,4′-oxazole]-7-yl)-5-bromopicolinamide (8 mg, 0.019 mmol, 99% yield). ¹H NMR (400 MHz, CD₃OD) δ 8.80 (d, 1H), 8.22 (dd, 1H), 8.13 (d, 1H), 8.05 (d, 1H), 7.62 (dd, 1H), 7.20 (d, 1H), 4.95 (d, 1H), 4.89 (d, 1H), 2.70 (d, 1H), 2.60 (d, 1H), 2.20 (d, 1H), 2.10 (d, 1H), 1.15 (s, 3H), 0.99 (s, 3H); m/z (APCI-pos) M+1=431.0.

### Example 89

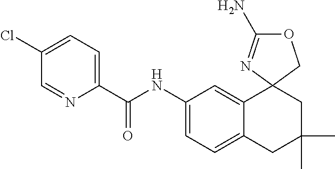

### N-(2′-amino-3,3-dimethyl-3,4-dihydro-2H,5′H-spiro[naphthalene-1,4′-oxazole]-7-yl)-5-chloropicolinamide

N-(2′-Amino-3,3-dimethyl-3,4-dihydro-2H,5′H-spiro[naphthalene-1,4′-oxazole]-7-yl)-5-chloropicolinamide was prepared according to Example 88, substituting 5-chloropicolinic acid for 5-bromopicolinic acid. ¹H NMR (400 MHz, CD₃OD) δ 8.70 (d, 1H), 8.20 (d, 1H), 8.13 (m, 2H), 7.62 (dd, 1H), 7.20 (d, 1H), 4.95 (d, 1H), 4.89 (d, 1H), 2.70 (d, 1H), 2.60 (d, 1H), 2.20 (d, 1H), 2.10 (d, 1H), 1.15 (s, 3H), 0.99 (s, 3H); m/z (APCI-pos) M+1=385.1.

### Example 90

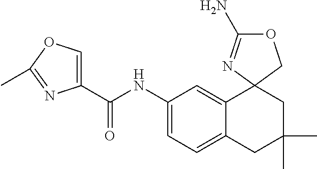

### N-(2′-amino-3-dimethyl-3,4-dihydro-2H,5′H-spiro[naphthalene-1,4′-oxazole]-7-yl)-2-methyloxazole-4-carboxamide

N-(2′-Amino-3,3-dimethyl-3,4-dihydro-2H,5′H-spiro[naphthalene-1,4′-oxazole]-7-yl)-2-methyloxazole-4-carboxamide was prepared according to Example 88, substituting 2-methyloxazole-4-carboxylic acid for 5-bromopicolinic acid. ¹H NMR (400 MHz, CD₃OD) δ 8.38 (s, 1H), 7.95 (d, 1H), 7.55 (dd, 1H), 7.18 (d, 1H), 4.95 (d, 1H), 4.89 (d, 1H), 2.70 (d, 1H), 2.60 (d, 1H), 2.52 (s, 3H), 2.20 (d, 1H), 2.10 (d, 1H), 1.15 (s, 3H), 0.99 (s, 3H); m/z (APCI-pos) M+1=355.1.

### Example 91

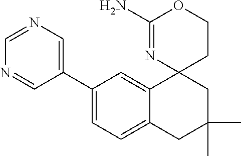

### 3,3-dimethyl-7-(pyrimidin-5-yl)-3,4,5′,6′-tetrahydro-2H-spiro[naphthalene-1,4′,[1,3]oxazin]2′-amine

Step A: Methyl 2-(1-amino-7-bromo-3,3-dimethyl-1,2,3,4-tetrahydronaphthalen-1-yl)acetate (0.52 g, 1.6 mmol) was dissolved in THF (7 mL) and cooled to 0° C. A solution in THF of LAH (“lithium aluminum hydride”) (1.5 mL, 1.5 mmol) was added slowly and stirred for 1 hour. After one hour, the reaction was quenched by the dropwise addition of water (60 μL), then a 15% NaOH solution (60 μL), then water (180 μL) and stirred for 1 hour. The mixture was filtered through Celite®, washed with EtOAc and concentrated to provide 2-(1-amino-7-bromo-3,3-dimethyl-1,2,3,4-tetrahydronaphthalen-1-yl)ethanol (0.42 g, 88%), which was used without further purification.

Step B: 2-(1-Amino-7-bromo-3,3-dimethyl-1,2,3,4-tetrahydronaphthalen-1-yl)ethanol (0.050 g, 0.168 mmol) was dissolved in EtOH (1 mL), and then a 5M CH₃CN solution of cyanic bromide (0.034 mL, 0.17 mmol) was added. After stirring overnight, the reaction was diluted with EtOH (10 mL) and heated to 75° C. After heating overnight, the reaction was concentrated and purified by silica gel column chromatography (4% MeOH/CH₂Cl₂+0.1% NH₄OH) to provide 7-bromo-3,3-dimethyl-3,4,5′,6′-tetrahydro-2H-spiro[naphthalene-1,4′-[1,3]oxazin]-2′-amine (19.5 mg, 36%).

Step C: 7-Bromo-3,3-dimethyl-3,4,5′,6′-tetrahydro-2H-spiro[naphthalene-1,4′-[1,3]oxazin]-2′-amine (0.0195 g, 0.0603 mmol) and pyrimidin-5-ylboronic acid (0.011 g, 0.090 mmol) were dissolved in dioxane (0.8 mL), and a saturated sodium carbonate (0.09 g, 0.170 mmol) solution was added. The reaction was degassed with Ar for 10 minutes. PdCl₂ (dppf)₂ (0.002 mg, 0.002 mmol) was added, and the vial was sealed under Ar and heated at 80° C. for 14 hours. The reaction was diluted with EtOAc, washed with water (2×), brine, dried (MgSO₄) and concentrated. The crude product was purified by column chromatography (5 to 10% MeOH/CH₂Cl₂ with 5% NH₄OH in MeOH) to provide 3,3-dimethyl-7-(pyrimidin-5-yl)-3,4,5′,6′-tetrahydro-2H-spiro[naphthalene-1,4′-[1,3]oxazin]-2′-amine (15 mg, 77%). MS: m/z (APCI-pos) M+1=323.

### Example 92

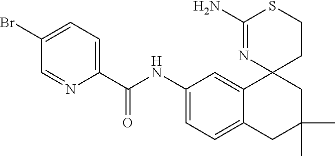

### N-(2′-amino-3,3-dimethyl-3,4,5′,6′-tetrahydro-2H-spiro[naphthalene-1,4′-[1,3]thiazine]-7-yl)-5-bromopicolinamide

Step A: A solution of vinylmagnesium bromide 1M in THF (39.5 mL, 39.5 mmol) was added dropwise to a solution of 7-bromo-3,3-dimethyl-3,4-dihydronaphthalen-1(2H)-one (5 g, 19.8 mmol) in THF at 0° C. under N₂. After 2 hours, the reaction mixture was poured into ice cold saturated NH₄Cl solution (150 mL) and extracted into EtOAc (2×100 mL). The combined organic layers were washed with brine, then dried (MgSO₄) and concentrated in vacuo to provide the crude 7-bromo-3,3-dimethyl-1-vinyl-1,2,3,4-tetrahydronaphthalen-1-ol as a liquid (5.7 g).

Step B: Thionyl chloride (2.88 mL, 39.5 mmol) was added to a solution of crude 7-bromo-3,3-dimethyl-1-vinyl-1,2,3,4-tetrahydronaphthalen-1-ol (5.55 g, 19.7 mmol) in CH₃CN (50 mL) at 0° C. The reaction mixture was stirred at 0° C. for 10 minutes and treated with thiourea (3.00 g, 39.5 mmol) in one portion. The mixture was then allowed to stir at ambient temperature for 10 minutes and then at 50° C. for 1 hour. The resulting mixture was allowed to stir at ambient temperature over the weekend. The solid formed was filtered and washed with additional CH₃CN (3×5 mL) and dried to provide (E)-2-(7-bromo-3,3-dimethyl-3,4-dihydronaphthalen-1(2H)-ylidene)ethyl carbamimidothioate hydrochloride (7.9 g, 90.5% yield) as a solid. LCMS (APCI+) m/z 400 (M+H)+.

Step C: A solution of (E)-2-(7-bromo-3,3-dimethyl-3,4-dihydronaphthalen-1(2H)-ylidene)ethyl carbamimidothioate hydrochloride (7.8 g, 21 mmol) in 2,2,2-trifluoroacetic acid (21 mL, 21 mmol) at 0° C. was treated dropwise with methanesulfonic acid (10 mL, 21 mmol). The resulting mixture was stirred at 0° C. for 30 minutes then at ambient temperature for one overnight. The mixture was then cooled to 0° C. and slowly poured into ice cold saturated Na₂CO₃ solution (200 mL). The resulting slurry was stirred at ambient temperature for 30 minutes. The solid formed was filtered, washed with copious amount of water, then triturated with hot MeOH and filtered. The filtrate collected was concentrated in vacuo and dried to provide 7-bromo-3,3-dimethyl-3,4,5′,6′-tetrahydro-2H-spiro[naphthalene-1,4′-[1,3]thiazin]-2′-amine (5.2 g, 74% yield) as a solid. LCMS (APCI+) m/z 340 (M+H)+.

Step D: di-tert-Butyl dicarbonate (1.92 g, 8.81 mmol) and triethylamine (2.83 mL, 20.3 mmol) were sequentially added to a solution of 7-bromo-3,3-dimethyl-3,4,5′,6′-tetrahydro-2H-spiro[naphthalene-1,4′-[1,3]thiazin]-2′-amine (2.3 g, 6.78 mmol) in dry THF (27.1 mL, 6.78 mmol). The resulting solution was stirred at ambient temperature for one overnight. The mixture was poured into water (50 mL) and extracted with EtOAc (3×70 mL). The organic layers were combined and washed with half saturated brine (3×30 mL), then dried (MgSO₄) and concentrated in vacuo. The residue obtained was purified by flash chromatography on silica gel (Ready Sep 120 g) eluting with 30% EtOAc/hexane to provide tert-butyl 7-bromo-3,3-dimethyl-3,4,5′,6′-tetrahydro-2H-spiro[naphthalene-1,4′-[1,3]thiazine]-2′-ylcarbamate (2.13 g, 71.5% yield) as a solid. LCMS (APCI+) m/z 439, 440 (M+H)+.

Step E: Two reactions were carried out each containing 250 mg of the bromide. A resealable glass pressure tube was charged with tert-butyl 7-bromo-3,3-dimethyl-3,4,5′,6′-tetrahydro-2H-spiro[naphthalene-1,4′-[1,3]thiazine]-2′-ylcarbamate (250 mg, 0.51 mmol), Pd₂ dba₃ (21 mg, 0.023 mmol), biphenyl-2-yldicyclohexylphosphine (16 mg, 0.046 mmol) and LiHMDS in 1M toluene (1.3 mL, 1.28 mmol). The reaction mixture was sparged with N₂ for 5 minutes, and then the tube was capped and stirred at 80° C. for 22 hours. The two reaction mixtures were combined in dioxane, cooled to 0° C. and treated with 1M HCl (5 mL). After 30 minutes, the reaction mixture was neutralized with saturated aqueous Na₂CO₃. The resulting suspension was extracted with EtOAc (3×20 mL). The organic layers were combined, washed with brine (20 mL), dried (MgSO₄) and concentrated in vacuo. The residue obtained was purified by flash chromatography on silica gel (Ready Sep 40g) eluting with 60% EtOAc/hexane to provide tert-butyl 7-amino-3,3-dimethyl-3,4,5′,6′-tetrahydro-2H-spiro[naphthalene-1,4′-[1,3]thiazine]-2′-ylcarbamate (195 mg, 50.7% yield) as a solid. LCMS (APCI+) m/z 376 (M+H)+.

Step F: A solution of tert-butyl 7-amino-3,3-dimethyl-3,4,5′,6′-tetrahydro-2H-spiro[naphthalene-1,4′-[1,3]thiazine]-2′-ylcarbamate (30 mg, 0.08 mmol) and 5-bromopicolinic acid (16 mg, 0.08 mmol) in methanol (1 mL) was treated with 4-(4,6-dimethoxy-1,3,5-triazin-2-yl)-4-methylmorpholin-4-ium chloride (33 mg, 0.12 mmol), and the resulting mixture was stirred at ambient temperature for 18 hours. The mixture was concentrated in vacuo, and the crude was purified by flash chromatography on silica gel (Ready Sep 12g) eluting with 30% EtOAc/hexane to provide tert-butyl 7-(5-bromopicolinamido)-3,3-dimethyl-3,4,5′,6′-tetrahydro-2H-spiro[naphthalene-1,4′-[1,3]thiazine]-2′-ylcarbamate (35 mg, 86% yield) as a solid. LCMS (APCI+) m/z 512 (M+H)+.

Step G: tert-Butyl 7-(5-bromopicolinamido)-3,3-dimethyl-3,4,5′,6′-tetrahydro-2H-spiro[naphthalene-1,4′-[1,3]thiazine]-2′-ylcarbamate (32 mg, 0.057 mmol) in DCM (0.3 mL) at 0° C. was treated with TFA (0.3 mL). The resulting solution was stirred at ambient temperature for 2 hours and concentrated in vacuo. The crude obtained was purified by C-18 reverse phase flash chromatography (Biotage Flash 12S+) eluting with a step gradient of 5%-30% CH₃CN/water+0.1% TFA to provide N-(2′-amino-3,3-dimethyl-3,4,5′,6′-tetrahydro-2H-spiro[naphthalene-1,4′-[1,3]thiazine]-7-yl)-5-bromopicolinamide (15 mg, 57% yield) as a solid. ¹H NMR (400 MHz, acetone-d₆) δ: 12.01 (br s, 1H), 11.29 (br s, 0.5H), 10.27 (br s, 1H), 8.75 (d, d, J1=0.783 Hz, J2=2.35 Hz, 1H), 8.27 (dd, J1=2.35 Hz, J2=8.61 Hz, 1H), 8.16 (d, J=7.825 Hz, 1H), 7.97 (dd, J1=1.96 Hz, J2=8.21 Hz, 1H), 7.94 (d, J=1.96 Hz, 1H), 7.19 (d, J=8.22 Hz, 1H), 3.49-3.43 (m, 1H), 3.28-3.22 (m, 1H), 2.70-2.55 (m, 2H), 2.52-2.45 (m, 1H), 2.32-2.26 (m, 1H), 2.11 (dd, J1=1.17 Hz, J2=14.08 Hz, 1H), 2.03-1.99 (m, 1H), 1.16 (s, 3H), 0.99 (s, 3H); LCMS (APCI+) m/z 459, 460 (M+H)+.

### Example 93

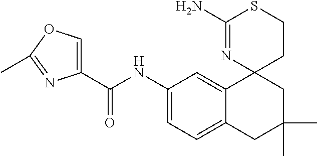

### N-(2′-amino-3,3-dimethyl-3,4,5′,6′-tetrahydro-2H-spiro[naphthalene-1,4′-[1,3]thiazine]-7-yl)-2-methyloxazole-4-carboxamide

Step A: A solution of tert-butyl 7-amino-3,3-dimethyl-3,4,5′,6′-tetrahydro-2H-spiro[naphthalene-1,4′-[1,3]thiazine]-2′-ylcarbamate (30 mg, 0.08 mmol) and 2-methyloxazole-4-carboxylic acid (10 mg, 0.080 mmol) were processed as described in Example 92, Step F, to provide tert-butyl 3,3-dimethyl-7-(2-methyloxazole-4-carboxamido)-3,4,5′,6′-tetrahydro-2H-spiro[naphthalene-1,4′-[1,3]thiazine]-2′-ylcarbamate (31 mg, 68% yield) as a solid. LCMS (APCI+) m/z 485 (M+H)+.

Step B: tert-Butyl 3,3-dimethyl-7-(2-methyloxazole-4-carboxamido)-3,4,5′,6′-tetrahydro-2H-spiro[naphthalene-1,4′-[1,3]thiazine]-2′-ylcarbamate (30 mg, 0.0619 mmol) was treated with TFA (1 mL) as described in Example 92, Step G, to provide the TFA salt of N-(2′-amino-3,3-dimethyl-3,4,5′,6′-tetrahydro-2H-spiro[naphthalene-1,4′-[1,3]thiazine]-7-yl)-2-methyloxazole-4-carboxamide (18.5 mg, 76.9% yield) as a solid. 1H NMR (400 MHz, acetone-d₆) δ 12.003 (br s, 1H), 9.31 (br s, 1H), 8.34 (s, 1H), 7.91 (dd, J1=2.44 Hz, J2=8.29 Hz, 1H), 7.88 (s, 1H), 7.16 (d, J=7.81 Hz, 1H), 3.46-3.41 (m, 1H), 3.26-3.21 (m, 1H), 2.68-2.57 (m, 2H), 2.48 (s, 3H), 2.47-2.44 (m, 1H), 2.29-2.25 (m, 1H), 2.10 (d, J=14.15 Hz, 1H), 2.0 (d, J=14.15 Hz, 1H), 1.16 (s, 3H), 0.98 (s, 3H); LCMS (APCI+) m/z 385 (M+H)+.

### Example 94

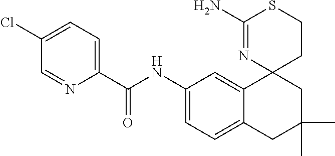

### N-(2′-amino-3,3-dimethyl-3,4,5′,6′-tetrahydro-2H-spiro[naphthalene-1,4′-[1,3]thiazine]-7-yl)-5-chloropicolinamide

Step A: tert-Butyl 7-amino-3,3-dimethyl-3,4,5′,6′-tetrahydro-2H-spiro[naphthalene-1,4′-[1,3]thiazine]-2′-ylcarbamate (30 mg, 0.08 mmol) and 5-chloropicolinic acid (13 mg, 0.08 mmol) were processed as described in Example 92, Step F, to provide tert-butyl 7-(5-chloropicolinamido)-3,3-dimethyl-3,4,5′,6′-tetrahydro-2H-spiro[naphthalene-1,4′-[1,3]thiazine]-2′-ylcarbamate (32 mg, 73% yield) as a solid. LCMS (APCI+) m/z 515, 517 (M+H)+.

Step B: tert-Butyl 7-(5-chloropicolinamido)-3,3-dimethyl-3,4,5′,6′-tetrahydro-2H-spiro[naphthalene-1,4′4′,3]thiazine]-2′-ylcarbamate (31 mg, 0.060 mmol) in DCM (0.3 mL) was treated with TFA (0.3 mL) as described in Example 92, Step G, to provide N-(2′-amino-3,3-dimethyl-3,4,5′,6′-tetrahydro-2H-spiro[naphthalene-1,4′-[1,3]thiazine]-7-yl)-5-chloropicolinamide (16 mg, 64% yield) as a solid. ¹H NMR (400 MHz, acetone-d₆): 11.93 (s, 1H), 10.26 (s, 1H), 8.66 (s, 1H), 8.23 (d, J=8.22 Hz, 1H), 8.13-8.11 (m, 1H), 7.98-7.94 (m, 2H), 7.20 (D, J=8.22 Hz, 1H), 3.50-3.44 (m, 2H), 3.29-3.23 (m, 2H), 2.52-2.45 (m, 1H), 2.33-2.27 (m, 1H), 2.13-2.0 (m, 2H), 1.16 (s, 3H0, 0.99 (s, 3H); LCMS (APCI+) m/z 415 (M+H)+.

### Example 95

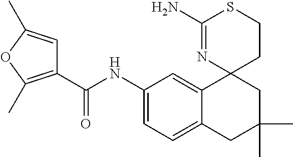

### N-(2′-amino-3,3-dimethyl-3,4,5′,6′-tetrahydro-2H-spiro[naphthalene-1,4′-[1,3]thiazine]-7-yl)-2,5-dimethylfuran-3-carboxamide

A solution of 2,5-dimethylfuran-3-carbonyl chloride in DCM (2 mL) was added dropwise to a solution of tert-butyl 7-amino-3,3-dimethyl-3,4,5′,6′-tetrahydro-2H-spiro[naphthalene-1,4′4′,3]thiazine]-2′-ylcarbamate (25 mg, 0.07 mmol) in DCM and triethylamine (37 μL, 0.26 mmol) at 0° C. under N₂. The ice bath was then removed, and the mixture was stirred at ambient temperature for 5 minutes. The reaction was quenched with half saturated Na₂CO₃ solution (2 mL), and the product was extracted into DCM (3×3 mL). The organic layers were combined, dried (MgSO₄) and concentrated in vacuo. The residue obtained was treated with neat TFA at ambient temperature. After 2 hours, TFA was removed in vacuo, and the crude was purified by C-18 reverse phase HPLC (Gilson Unipoint) eluting with a gradient of 5-95% CH₃CN/water+0.1% TFA to provide TFA salt of N-(2′-amino-3,3-dimethyl-3,4,5′,6′-tetrahydro-2H-spiro[naphthalene-1,4′-[1,3]thiazine]-7-yl)-2,5-dimethylfuran-3-carboxamide (5.6 mg, 16.4% yield) as a solid. ¹H MR (500 MHz, CD₂Cl₂) δ: 7.47 (d, J=7.75 Hz, 1H), 7.34 (s, 1H), 7.10-7.07 (m, 1H), 6.20 (s, 1H), 3.34-3.28 (m, 1H), 3.08-3.01 (m, 1H), 2.64-2.61 (m, 1H), 2.58 (s, 3H), 2.56-2.53 (m, 1H), 2.31 (s, 3H), 1.97-1.91 (m, 2H), 1.88-1.81 (m, 1H), 1.73-1.67 (m, 1H), 1.12 (s, 3H), 0.938 (s, 3H); LCMS (APCI+) m/z 398 (M+H⁺).

### Example 96

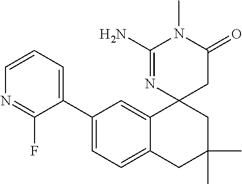

### 2′-amino-7-(2-fluoropyridin-3-yl)-1′,3,3-trimethyl-3,4-dihydro-1′H,2H-spiro[naphthalene-1,4′-pyrimidin]-6′(5′H)-one

Step A: Freshly distilled Ti(OEt)₄ (9.01 g, 39.5 mmol) was added in one portion to a solution of 7-bromo-3,3-dimethyl-3,4-dihydronaphthalen-1(2H)-one (5 g, 19.8 mmol) and 2-methylpropane-2-sulfinamide (3.11 g, 25.7 mmol) in THF (65.8 mL, 19.8 mmol). The resulting mixture was refluxed for 18 hours. The mixture was cooled to ambient temperature and poured into saturated NaHCO₃ (500 mL) solution. The resulting suspension was shaken with EtOAc (300 mL) and filtered through a pad of Celite®. The solid particles in the filter funnel were crushed with a spatula and washed well with EtOAc (˜200 mL). The filtrate was transferred to a separatory funnel, and the layers were separated. The aqueous layer was extracted once with EtOAc (100 mL). The organic layers were combined, washed with brine (100 mL), dried (MgSO₄), filtered and then concentrated in vacuo. The residue obtained was purified by flash chromatography on silica gel (Ready Sep 120 g) eluting with 15% EtOAc, followed by 20% EtOAc/hexane (500 mL) to provide (E)-N-(7-bromo-3,3-dimethyl-3,4-dihydronaphthalen-1(2H)-ylidene)-2-methylpropane-2-sulfinamide (4.2 g, 59.7% yield) as a solid. LCMS (APCI+) m/z 356, 359 (M+H)+.

Step B: A round bottom flask equipped with a N₂ inlet, rubber septum and an internal temperature probe was charged with a solution of diisopropylamine (3.44 mL, 24.5 mmol) in THF (25 mL). The solution was cooled to −78° C. and treated dropwise with n-butyllithium 2.5M in hexanes (9.78 mL, 24.5 mmol). Once the addition was complete, the cooling bath was replaced with an ice bath, and the mixture was allowed to stir at 0° C. for 40 minutes. Meanwhile, a separate round bottom flask equipped with a N₂ inlet, rubber septum and an internal temperature probe was charged with a solution of methyl acetate (2.04 mL, 25.6 mmol) in THF (20 mL). The mixture was cooled to −78° C. Then above prepared LDA solution in THF was slowly added to this solution via a cannula maintaining internal temperature below −74° C. The resulting mixture was stirred at −78° C. for 1 hour and treated dropwise with a solution of (E)-N-(7-bromo-3,3-dimethyl-3,4-dihydronaphthalen-1(2H)-ylidene)-2-methylpropane-2-sulfinamide (4.15 g, 11.6 mmol) in THF (70 mL). Once the addition was complete, the mixture was stirred at −78° C. for 3 hours. The mixture was then poured into a saturated NaHCO₃ solution (100 mL) and extracted into EtOAc (3×50 mL). The organic layers were combined, dried (MgSO₄) and concentrated in vacuo. The residue obtained was first crystallized from DCM, and the filtrate was purified by flash chromatography on silica gel (Ready Sep 220 g) eluting with a step gradient of 20%-50% EtOAc/hexane then with 30% EtOAc/hexane to provide methyl 2-(7-bromo-1-(1,1-dimethylethylsulfinamido)-3,3-dimethyl-1,2,3,4-tetrahydronaphthalen-1-yl)acetate as a solid (2.28 g, 45% yield). LCMS (APCI+) m/z 429.8, 431.8 (M+H)+.

Step C: A solution of methyl 2-(7-bromo-1-(1,1-dimethylethylsulfinamido)-3,3-dimethyl-1,2,3,4-tetrahydronaphthalen-1-yl)acetate (2.27 g, 5.27 mmol) in dioxane (10 mL) was treated with hydrogen chloride (6.6 mL, 26.4 mmol). After 2.5 hours, TFA was removed in vacuo, and the residue was evaporated from DCM. The residue obtained was dried under high vacuum to provide methyl 2-(1-amino-7-bromo-3,3-dimethyl-1,2,3,4-tetrahydronaphthalen-1-yl)acetate (1.7 g, 98.8% yield). LCMS (APCI+) m/z 325.8, 327.7 (M+H)+.

Step D: A suspension of methyl 2-(1-amino-7-bromo-3,3-dimethyl-1,2,3,4-tetrahydronaphthalen-1-yl)acetate (500 mg, 1.53 mmol), EDCI (“1-ethyl-3-(3-dimethylaminopropyl)carbodiimide”) (529 mg, 2.76 mmol) and methylcarbamothioylcarbamate (437 mg, 2.30 mmol) in N,N-dimethylformamide (7663 μL, 1.53 mmol) was treated with N-ethyl-N-isopropylpropan-2-amine (1303 μL, 7.66 mmol) and stirred at ambient temperature for 18 hours. The mixture was then poured into water (50 mL) and extracted with EtOAc (3×40 mL). The organic layers were combined, dried (MgSO₄) and concentrated in vacuo. The residue obtained was crystallized from MeOH to provide tert-butyl 7-bromo-1′,3,3-trimethyl-6′-oxo-3,4,5′,6′-tetrahydro-1′H,2H-spiro[naphthalene-1,4′-pyrimidine]-2′-ylcarbamate (480 mg, 69.5% yield) as a solid. LCMS (APCI+) m/z 450, 451 (M+H)+.

Step E: A resealable glass pressure tube was charged with tert-butyl 7-bromo-1′,3,3-trimethyl-6′-oxo-3,4,5′,6′-tetrahydro-1′H,2H-spiro[naphthalene-1,4′-pyrimidine]-2′-ylcarbamate (63 mg, 0.14 mmol), 2-fluoropyridin-3-ylboronic acid (24 mg, 0.17 mmol), dichloro[1,1′-bis(diphenylphosphino)ferrocene]palladium (II) dichloromethane adduct (5.8 mg, 0.0070 mmol), 20% aqueous Na₂CO₃ (259 μL, 0.49 mmol), and 1,4-dioxane (1399 μL, 0.14 mmol). The reaction was sparged with N₂ for 5 minutes, capped, and stirred at 90° C. for 18 hours and allowed to cool temperature. The mixture was diluted with EtOAc (40 mL) and washed with brine (2×10 mL). The organic layer was dried (MgSO₄) and concentrated in vacuo. The residue obtained was purified by flash chromatography (Ready Sep 40 g) eluting with 10% MeOH/DCM+NH₃ followed by C-18 reverse phase HPLC (Gilson Unipoint) eluting with a gradient of 5-95% CH₃CN/water containing 0.1% TFA to provide the TFA salt of 2′-amino-7-(2-fluoropyridin-3-yl)-1′,3,3-trimethyl-3,4-dihydro-1′H,2H-spiro[naphthalene-1,4′-pyrimidin]-6′(5′H)-one (35 mg, 68% yield) as a solid. LCMS (APCI+) m/z 367 (M+H)+ (single peak) for the desired product.

The following compounds in Table 2 were prepared according to the above procedures using appropriate intermediates.

TABLE 2

| Ex. # | Structure | Name | NMR/MS |
|---|---|---|---|
| 97 | 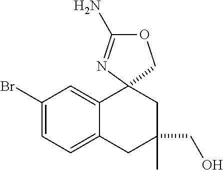 | ((1S*,3R*)-2′-amino-7- bromo-3-methyl-3,4-dihydro- 2H,5′H-spiro[naphthalene- 1,4′-oxazole]-3-yl)methanol | 325, 327 |
| 98 | 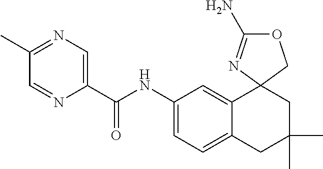 | N-(2′-amino-3,3-dimethyl- 3,4-dihydro-2H,5′H- spiro[naphthalene-1,4′- oxazole]-7-yl)-5- methylpyrazine-2- carboxamide | m/z (APCI- pos) M + 1 = 366.1 |
| 99 | 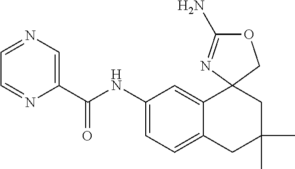 | N-(2′-amino-3,3-dimethyl- 3,4-dihydro-2H,5′H- spiro[naphthalene-1,4′- oxazole]-7-yl)pyrazine-2- carboxamide | m/z (APCI- pos) M + 1 = 352.1 |
| 100 | 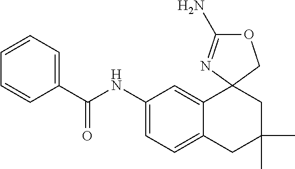 | N-(2′-amino-3,3-dimethyl- 3,4-dihydro-2H,5′H- spiro[naphthalene-1,4′- oxazole]-7-yl)benzamide | m/z (APCI- pos) M + 1 = 350.2 |
| 101 | 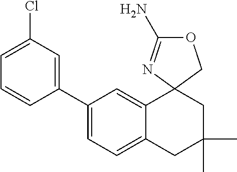 | 7-(3-chlorophenyl)-3,3- dimethyl-3,4-dihydro- 2H,5′H-spiro[naphthalene- 1,4′-oxazol]-2′-amine | m/z (APCI- pos) M + 1 = 341.1 |
| 102 | 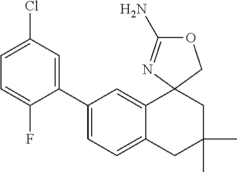 | 7-(5-chloro-2-fluorophenyl)- 3,3-dimethyl-3,4-dihydro- 2H,5′H-spiro[naphthalene- 1,4′-oxazol]-2′-amine | m/z (APCI- pos) M + 1 = 359.1 |
| 103 | 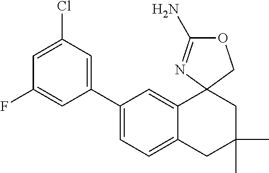 | 7-(3-chloro-5-fluorophenyl)- 3,3-dimethyl-3,4-dihydro- 2H,5′H-spiro[naphthalene- 1,4′-oxazol]-2′-amine | m/z (APCI- pos) M + 1 = 359.1 |
| 104 | 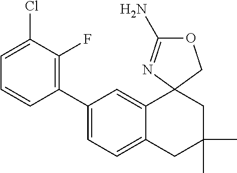 | 7-(3-chloro-2-fluorophenyl)- 3,3-dimethyl-3,4-dihydro- 2H,5′H-spiro[naphthalene- 1,4′-oxazol]-2′-amine | m/z (APCI- pos) M + 1 = 359.1 |
| 105 | 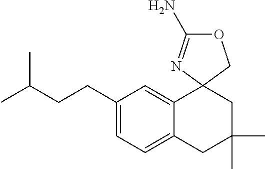 | 7-isopentyl-3,3-dimethyl-3-4- dihydro-2H,5′H- spiro[naphthalene-1,4′- oxazol]-2′-amine | m/z (APCI- pos) M + 1 = 301.2 |
| 106 | 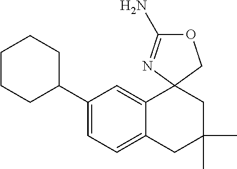 | 7-cyclohexyl-3,3-dimethyl- 3,4-dihydro-2H,5′H- spiro[naphthalene-1,4′- oxazol]-2′-amine | m/z (APCI- pos) M + 1 = 313.2 |
| 107 | 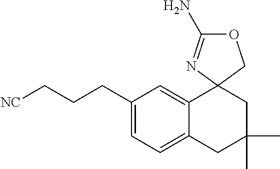 | 4-(2′-amino-3,3-dimethyl- 3,4-dihydro-2H,5′H- spiro[naphthalene-1,4′- oxazole]-7-yl)butanenitrile | m/z (APCI- pos) M + 1 = 298.2 |
| 108 | 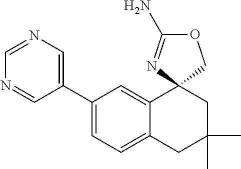 | (S)-3,3-dimethyl-7- (pyrimidin-5-yl)-3,4-dihydro- 2H,5′H-spiro[naphthalene- 1,4′-oxazol]-2′-amine | m/z (APCI- pos) M + 1 = 309.1 |
| 109 | 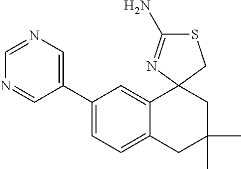 | 3,3-dimethyl-7-(pyrimidin-5- yl)-3,4-dihydro-2H,5′H- spiro[naphthalene-1,4′- thiazol]-2′-amine | m/z (APCI- pos) M + 1 = 325.1 |
| 110 | 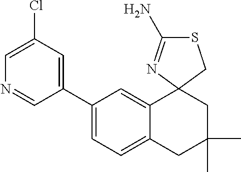 | 7-(5-chloropyridin-3-yl)-3,3- dimethyl-3,4-dihydro- 2H,5′H-spiro[naphthalene- 1,4′-thiazol]-2′-amine | m/z (APCI- pos) M + 1 = 358.1 |
| 111 | 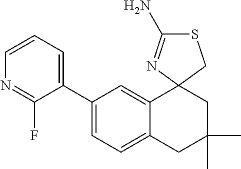 | 7-(2-fluoropyridin-3-yl)-3,3- dimethyl-3,4-dihydro- 2H,5′H-spiro[naphthalene- 1,4′-thiazol]-2′-amine | m/z (APCI- pos) M + 1 = 342.1 |
| 112 | 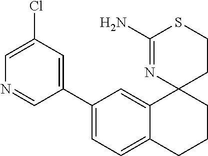 | 7-(5-chloropyridin-3-yl)- 3,4,5′,6′-tetrahydro-2H- spiro[naphthalene-1,4′- [1,3]thiazin]-2′-amine | m/z (APCI+) M + 1 = 344 |
| 113 | 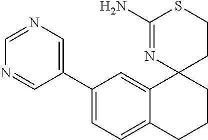 | 7-(pyrimidin-5-yl)-3,4,5′,6′- tetrahydro-2H- spiro[naphthalene-1,4′- [1,3]thiazin]-2′-amine | m/z (APCI+) M + 1 = 311 |
| 114 | 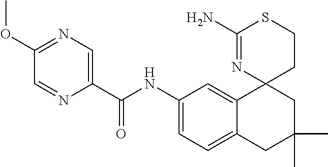 | N-(2′-amino-3,3-dimethyl- 3,4,5′,6′-tetrahydro-2H- spiro[naphthalene-1,4′- [1,3]thiazine]-7-yl)-5- methoxypyrazine-2- carboxamide | m/z (APCI+) M + 1 = 412 |
| 115 | 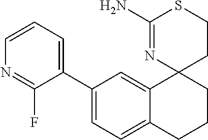 | 7-(2-fluoropyridin-3-yl)- 3,4,5′,6′-tetrahydro-2H- spiro[naphthalene-1,4′- [1,3]thiazin]-2′-amine | m/z (APCI+) M + 1 = 328 |
| 116 | 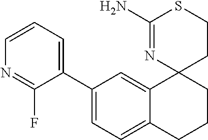 | 3,3-dimethyl-7-(pyrimidin-5- yl)-3,4,5′,6′-tetrahydro-2H- spiro[naphthalene-1,4′- [1,3]thiazin]-2′-amine | m/z (APCI+) M + 1 = 339 |
| 117 | 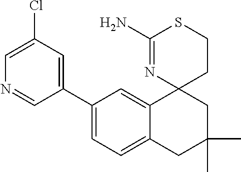 | 7-(5-chloropyridin-3-yl)-3,3- dimethyl-3,4,5′,6′-tetrahydro- 2H-spiro[naphthalene-1,4′- [1,3]thiazin]-2′-amine | m/z (APCI+) M + 1 = 372 |
| 118 | 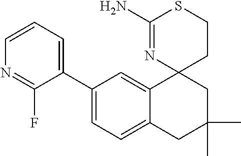 | 7-(2-fluoropyridin-3-yl)-3,3- dimethyl-3,4,5′,6′-tetrahydro- 2H-spiro[naphthalene-1,4′- [1,3]thiazin]-2′-amine | m/z (APCI+) M + 1 = 412 |
| 119 | 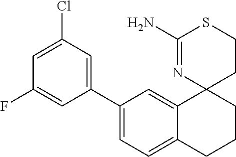 | 7-(3-chloro-5-fluorophenyl)- 3,4,5′,6′-tetrahydro-2H- spiro[naphthalene-1,4′- [1,3]thiazin]-2′-amine | m/z (APCI+) M + 1 = 361 |
| 120 | 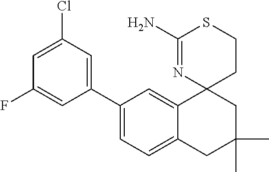 | 7-(3-chloro-5-fluorophenyl)- 3,3-dimethyl-3,4,5′,6′- tetrahydro-2H- spiro[naphthalene-1,4′- [1,3]thiazin]-2′-amine | m/z (APCI+) M + 1 = 389, 391 |
| 121 | 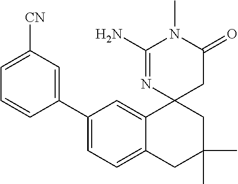 | 3-(2′-amino-1′,3,3-trimethyl- 6′-oxo-3,4,5′,6′-tetrahydro- 1′H,2H-spiro[naphthalene- 1,4′-pyrimidine]-7- yl)benzonitrile | m/z (APCI+) M + 1 = 373 |
| 122 | 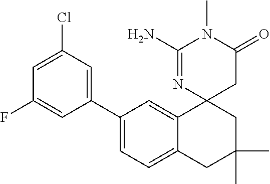 | 2′-amino-7-(3-chloro-5- fluorophenyl)-1′,3,3- trimethyl-3,4-dihydro- 1′H,2H-spiro[naphthalene- 1,4′-pyrimidin]-6′(5′H)-one | m/z (APCI+) M + 1 = 400,402 |
| 123 | 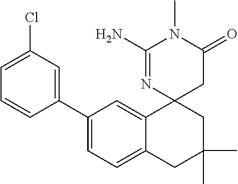 | 2′-amino-7-(3-chlorophenyl)- 1′,3,3-trimethyl-3,4-dihydro- 1′H,2H-spiro[naphthalene- 1,4′-pyrimidin]-6′(5′H)-one | m/z (APCI+) M + 1 = 382 |
| 124 | 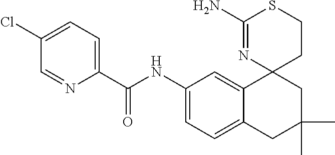 | N-(2′-amino-3,3-dimethyl- 3,4,5′,6′-tetrahydro-2H- spiro[naphthalene-1,4′- [1,3]thiazine]-7-yl)-5- chloropicolinamide | m/z (APCI+) M + 1 = 415 |
| 125 | 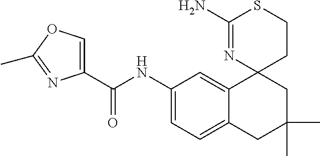 | N-(2′-amino-3,3-dimethyl- 3,4,5′,6′-tetrahydro-2H- spiro[naphthalene-1,4′- [1,3]thiazine]-7-yl)-2- methyloxazole-4- carboxamide | m/z (APCI+) M + 1 = 385 |
| 126 | 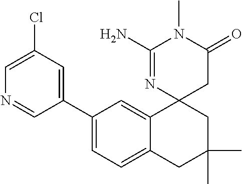 | 2′-amino-7-(5-chloropyridin- 3-yl)-1′,3,3-trimethyl-3,4- dihydro-1′H,2H- spiro[naphthalene-1,4′- pyrimidin]-6′(5′H)-one | m/z (APCI+) M + 1 = 383 |
| 127 | 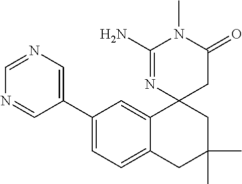 | 2′-amino-1′,3,3-trimethyl-7- (pyrimidin-5-yl)-3,4-dihydro- 1′H,2H-spiro[naphthalene- 1,4′-pyrimidin]-6′(5′H)-one | m/z (APCI+) M + 1 = 350 |
| 128 | 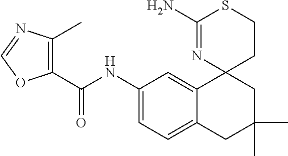 | N-(2′-amino-3,3-dimethyl- 3,4,5′,6′-tetrahydro-2H- spiro[naphthalene-1,4′- [1,3]thiazine]-7-yl)-4- methyloxazole-5- carboxamide | m/z (APCI+) M + 1 = 385 |
| 129 | 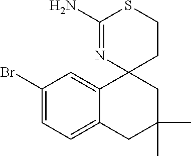 | 7-bromo-3,3-dimethyl- 3,4,5′,6′-tetrahydro-2H- spiro[naphthalene-1,4′- [1,3]thiazin]-2′-amine | m/z (APCI+) M + 1 = 340 |

### Example 130

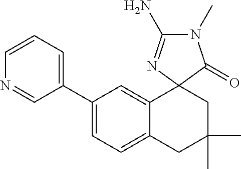

### 2-amino-1,3′,3′-trimethyl-7′-(pyridin-3-yl)-3′,4′-dihydro-2′H-spiro[imidazole-4,1′-naphthalen]-5(1H)-one

2-Amino-1,3′,3′-trimethyl-7′-(pyridin-3-yl)-3′,4′-dihydro-2′H-spiro[imidazole-4,1′-naphthalen]-5(1H)-one was prepared according to the procedures of Example 2, in which pyridin-3-ylboronic acid was used in place of 3-methoxyphenylboronic acid in Step I. ¹H-NMR (400 MHz, CDCl₃) δ 8.58-8.54 (m, 1H), 7.78-7.74 (m, 1H), 7.43-7.39 (m, 1H), 7.32 (dd, J=7.8, 4.7 Hz, 1H), 7.21 (d, J=8.2 Hz, 1H), 7.10 (s, 1H), 3.19 (s, 3H), 2.84 (d, J=16.0 Hz, 1H), 2.61 (dd, J=16.2, 2.2 Hz, 1H), 2.31 (d, J=14.0 Hz, 1H), 1.82 (dd, J=14.1, 2.0 Hz, 1H), 1.17 (s, 3H), 1.02 (s, 3H); m/z (APCI+) M+1=335.

### Example 131

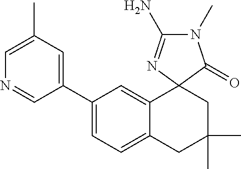

### 2-amino-1,3′,3′-trimethyl-7′-(5-methylpyridin-3-yl)-3′,4′-dihydro-2′H-spiro[imidazole-4,1′-naphthalen]-5 (1H)-one

2-Amino-1,3′,3′-trimethyl-7′-(5-methylpyridin-3-yl)-3′,4′-dihydro-2′H-spiro[imidazole-4,1′-naphthalen]-5(1H)-one was prepared according to the procedures of Example 2, in which 5-methylpyridin-3-ylboronic acid was used in place of 3-methoxyphenylboronic acid in Step I. 1H NMR (400 MHz, CDCl₃) δ 8.51 (d, J=2.3 Hz, 1H), 8.40-8.37 (m, 1H), 7.57-7.54 (m, 1H), 7.39 (dd, J=7.8, 1.6 Hz, 1H), 7.19 (d, J=7.8 Hz, 1H), 7.09 (d, J=1.6 Hz, 1H), 3.19 (s, 3H), 2.83 (d, J=16.0 Hz, 1H), 2.61 (dd, J=16.4, 2.3 Hz, 1H), 2.38 (s, 3H), 2.31 (d, J=14.1 Hz, 1H), 1.82 (dd, J=14.1, 2.3 Hz, 1H), 1.17 (s, 3H), 1.02 (s, 3H); m/z (APCI+) M+1=349.

### Example 132

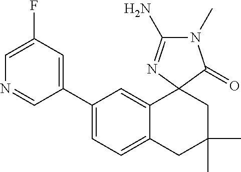

### 2-amino-7′-(5-fluoropyridin-3-yl)-1,3′,3′-trimethyl-3′,4′-dihydro-2′H-spiro[imidazole-4,1′-naphthalen]-5(1H)-one

2-Amino-7′-(5-fluoropyridin-3-yl)-1,3′,3′-trimethyl-3′,4′-dihydro-2′H-spiro[imidazole-4,1′-naphthalen]-5(1H)-one was prepared according to the procedures of Example 2, in which 5-fluoropyridin-3-ylboronic acid was used in place of 3-methoxyphenylboronic acid in Step I. ¹H NMR (400 MHz, CDCl₃) δ 8.56 (t, J=1.6 Hz, 1H), 8.42 (d, J=2.7 Hz, 1H), 7.48 (ddd, J=9.6, 2.6, 1.9 Hz, 1H), 7.40 (dd, J=8.0, 1.8 Hz, 1H), 7.23 (t, J=7.8 Hz, 1H), 7.09 (d, J=2.0 Hz, 1H), 3.20 (s, 3H), 2.84 (d, J=16.4 Hz, 1H), 2.62 (dd, J=16.0, 2.3 Hz, 1H), 2.30 (d, J=14.1 Hz, 1H), 1.81 (dd, J=13.7, 2.3 Hz, 1H), 1.18 (s, 3H), 1.03 (s, 3H); m/z (APCI+) M+1=353.

### Example 133

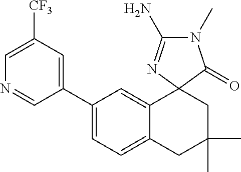

### 2-amino-1,3′,3′-trimethyl-7′-(5-(trifluoromethyl)pyridin-3-yl)-3′,4′-dihydro-2′H-spiro[imidazole-4,1′-naphthalen]-5(1H)-one

2-Amino-1,3′,3′-trimethyl-7′-(5-(trifluoromethyl)pyridin-3-yl)-3′,4′-dihydro-2′H-spiro[imidazole-4,1′-naphthalen]-5(1H)-one was prepared according to the procedures of Example 2, in which 5-(trifluoromethyl)pyridin-3-ylboronic acid was used in place of 3-methoxyphenylboronic acid in Step I. ¹H NMR (400 MHz, CDCl₃) δ 8.90 (d, J=2.0 Hz, 1H), 8.85-8.80 (m, 1H), 7.98-7.94 (m, 1H), 7.42 (dd, J=7.8, 2.0 Hz, 1H), 7.10 (d, J=2.0 Hz, 1H), 3.19 (s, 3H), 2.86 (d, J=16.0 Hz, 1H), 2.63 (dd, J=16.4, 2.3 Hz, 1H), 2.30 (d, J=14.1 Hz, 1H), 1.81 (dd, J=13.7, 2.3 Hz, 1H), 1.18 (s, 3H), 1.03 (s, 3H); m/z (APCI+) M+1=403.

### Example 134

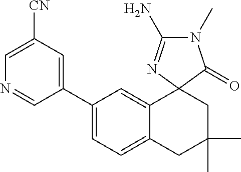

### 5-(2-amino-1,3′,3′-trimethyl-5-oxo-1,3′,4′,5-tetrahydro-2′H-spiro[imidazole-4,1′-naphthalene]-7′-yl)nicotinonitrile

5-(2-Amino-1,3′,3′-trimethyl-5-oxo-1,3′,4′,5-tetrahydro-2′H-spiro[imidazole-4,1′-naphthalen]-7′-yDnicotinonitrile was prepared according to the procedures of Example 2, in which 5-(4,4,5,5-tetramethyl-1,3,2-dioxaborolan-2-yl)nicotinonitrile was used in place of 3-methoxyphenylboronic acid in Step I. ¹H NMR (400 MHz, CDCl₃) δ 8.92 (d, J=2.3 Hz, 1H), 8.83 (d, J=2.0 Hz, 1H), 8.03 (t, J=2.2 Hz, 1H), 7.40 (dd, J=8.0, 1.8 Hz, 1H), 7.28-7.24 (m, 1H), 7.07 (d, J=2.0 Hz, 1H), 3.22 (s, 3H), 2.86 (d, J=16.0 Hz, 1H), 2.64 (dd, J=16.4, 2.3 Hz, 1H), 2.30 (d, J=14.1 Hz, 1H), 1.82 (dd, J=14.1, 2.3 Hz, 1H), 1.18 (s, 3H), 1.03 (s, 3H); m/z (APCI+) M+1=360.

### Example 135

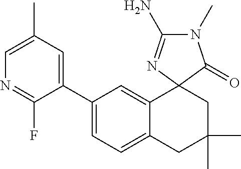

### 2-amino-7′-(2-fluoro-5-methylpyridin-3-yl)-1,3′,3′-trimethyl-3′,4′-dihydro-2′H-spiro[imidazole-4,1′-naphthalen]-5(1H)-one

2-Amino-7′-(2-fluoro-5-methylpyridin-3-yl)-1,3′,3′-trimethyl-3′,4′-dihydro-2′H-spiro[imidazole-4,1′-naphthalen]-5(1H)-one was prepared according to the procedures of Example 2, in which 2-fluoro-5-methyl-3-(4,4,5,5-tetramethyl-1,3,2-dioxaborolan-2-yl)pyridine was used in place of 3-methoxyphenylboronic acid in Step I. ¹H NMR (400 MHz, CDCl₃) δ 7.96-7.93 (m, 1H), 7.56 (dd, J=9.8, 2.3 Hz, 1H), 7.40 (dt, J=7.8, 1.6 Hz, 1H), 7.19 (d, J=7.8 Hz, 1H), 7.12-7.08 (m, 1H), 3.19 (s, 3H), 2.83 (d, J=16.0 Hz, 1H), 2.61 (dd, J=16.2, 2.2 Hz, 1H), 2.35 (s, 3H), 2.32 (d, J=13.7 Hz, 1H), 1.82 (dd, J=14.1, 2.3 Hz, 1H), 1.17 (s, 3H), 1.03 (s, 3H); m/z (APCI+) M+1=367.

### Example 136

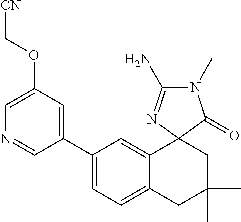

### 2-(5-(2-amino-1,3′,3′-trimethyl-5-oxo-1,3′,4′,5-tetrahydro-2′H-spiro[imidazole-4,1′-naphthalene]-7′-yl)pyridin-3-yloxy)acetonitrile

Step A: 2-Amino-7′-bromo-1,3′,3′-trimethyl-3′,4′-dihydro-2′H-spiro[imidazole-4,1′-naphthalen]-5(1H)-one (0.500 g, 1.49 mmol), 4,4,4′,4′,5,5,5′,5′-octamethyl-2,2′-bi(1,3,2-dioxaborolane) (0.755 g, 2.97 mmol), PdCl₂(dppf)-DCM adduct (0.0607 g, 0.0744 mmol) and KOAc (0.438 g, 4.46 mmol) in DMF (7.4 mL; degassed with nitrogen sparge for 30 minutes prior to use) were combined into a 48 mL sealable pressure tube. The headspace was purged with nitrogen, the tube was sealed, sonicated, and the reaction mixture was heated in a 110° C. sand bath and stirred for 4 hours. The reaction mixture was then concentrated and dried in vacuo. The resulting residue was sonicated with DCM, and the solids were removed by vacuum filtration through GF/F paper and rinsed with DCM. The filtrate was concentrated and dried in vacuo to give a foam. The crude was combined with hexanes (40 mL), heated to 40° C., and stirred for 15 minutes. The mixture was cooled to room temperature, and the solids were isolated by vacuum filtration through a 0.45 micron nylon filter membrane, rinsed with hexanes, and dried in vacuo to give 2-amino-1,3′,3′-trimethyl-7′-(4,4,5,5-tetramethyl-1,3,2-dioxaborolan-2-yl)-3′,4′-dihydro-2′H-spiro[imidazole-4,1′-naphthalen]-5(1H)-one (0.363 g, 63.7% yield) as a semi-pure powder consisting of 65% desired product, 10% of the corresponding boronic acid, and 10% of the corresponding des-bromo starting material, which was used without further purification.

Step B: 2-Amino-1,3′,3′-trimethyl-7′-(4,4,5,5-tetramethyl-1,3,2-dioxaborolan-2-yl)-3′,4′-dihydro-2′H-spiro[imidazole-4,1′-naphthalen]-5(1H)-one (0.040 g, 0.104 mmol), 2-(5-bromopyridin-3-yloxy)acetonitrile (0.0278 g, 0.130 mmol), and Pd(PPh₃)₄ (0.00965 g, 0.00835 mmol) were combined with 2M Na₂CO₃ (0.157 mL, 0.313 mmol) and dioxane (0.7 mL; both degassed 20 minutes prior to use), and the reaction mixture was heated in a 100° C. reaction block and stirred for 13 hours. The reaction mixture was concentrated under nitrogen stream, and the residue was combined with DCM/MeOH and loaded directly onto a preparative TLC plate (2 mm plate, 9:1 DCM:7N NH₃/MeOH). The isolated product was only about 70% pure, and was thus purified again by preparative TLC (1 mm plate, 4:1 DCM:MeOH) to give 2-(5-(2-amino-1,3′,3′-trimethyl-5-oxo-1,3′,4′,5-tetrahydro-2′H-spiro[imidazole-4,1′-naphthalene]-7′-yl)pyridin-3-yloxy)-acetonitrile (0.0036 g, 8.86% yield) as a powder. ¹H NMR (400 MHz, CDCl₃) δ 8.50 (d, J=1.6 Hz, 1H), 8.37-8.34 (m, 1H), 7.43 (dd, J=8.0, 1.8 Hz, 1H), 7.40 (dd, J=2.9, 1.8 Hz, 1H), 7.23 (d, J=7.8 Hz, 1H), 7.09 (d, J=1.6 Hz, 1H), 4.87 (s, 2H), 3.20 (s, 3H), 2.85 (d, J=16.4 Hz, 1H), 2.63 (dd, J=16.2, 2.2 Hz, 1H), 2.31 (d, J=13.7 Hz, 1H), 1.83 (dd, J=14.1, 2.3 Hz, 1H), 1.18 (s, 3H), 1.04 (s, 3H); m/z (APCI+) M+1=390.

### Example 137

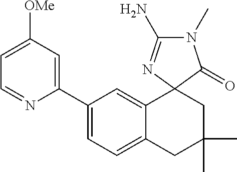

### 2-amino-7′-(4-methoxypyridin-2-yl)-1,3′,3′-trimethyl-3′,4′-dihydro-2′H-spiro[imidazole-4,1′-naphthalen]-5(1H)-one

2-Amino-7′-(4-methoxypyridin-2-yl)-1,3′,3′-trimethyl-3′,4′-dihydro-2′H-spiro[imidazole-4,1′-naphthalen]-5(1H)-one was prepared according to the procedures of Example 136, in which 2-bromo-4-methoxypyridine was used in place of 2-(5-bromopyridin-3-yloxy)acetonitrile in Step B. ¹H NMR (400 MHz, CDCl₃) δ 8.46 (d, J=5.9 Hz, 1H), 7.75 (dd, J=8.0, 1.8 Hz, 1H), 7.56 (s, 1H), 7.19 (d, J=7.8 Hz, 1H), 7.09 (d, J=2.3 Hz, 1H), 6.75 (dd, J=5.5, 2.3 Hz, 1H), 3.88 (s, 3H), 3.19 (s, 3H), 2.84 (d, J=16.4 Hz, 1H), 2.60 (dd, J=16.2, 2.2 Hz, 1H), 2.32 (d, J=13.7 Hz, 1H), 1.83 (dd, J=14.1, 2.3 Hz, 1H), 1.16 (s, 3H), 1.00 (s, 3H); m/z (APCI+) M+1=365.

### Example 138

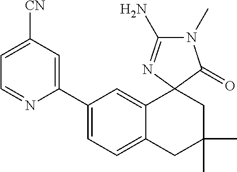

### 2-(2-amino-1,3′,3′-trimethyl-5-oxo-1,3′,4′,5-tetrahydro-2′H-spiro[imidazole-4,1′-naphthalene]-7′-yl)isonicotinonitrile

2-(2-Amino-1,3′,3′-trimethyl-5-oxo-1,3′,4′,5-tetrahydro-2′H-spiro[imidazole-4,1′-naphthalen]-7′-yDisonicotinonitrile was prepared according to the procedures of Example 136, in which 2-bromoisonicotinonitrile was used in place of 2-(5-bromopyridin-3-yloxy)acetonitrile in Step B. ¹H NMR (400 MHz, CDCl₃) δ 8.82-8.79 (m, 1H), 7.84-7.82 (m, 1H), 7.84-7.80 (m, 1H), 7.61 (d, J=1.6 Hz, 1H), 7.43-7.40 (m, 1H), 7.28-7.23 (m, 1H), 3.25 (s, 3H), 2.86 (d, J=16.4 Hz, 1H), 2.64 (dd, J=16.2, 2.2 Hz, 1H), 2.33 (d, J=14.1 Hz, 1H), 1.85 (dd, J=14.1, 2.3 Hz, 1H), 1.18 (s, 3H), 1.03 (s, 3H); m/z (APCI+) M+1=360.

### Example 139

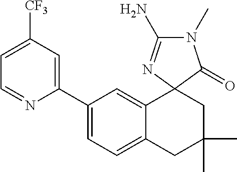

### 2-amino-1,3′,3′-trimethyl-7′-(4-(trifluoromethyl)pyridin-2-yl)-3′,4′-dihydro-2′H-spiro[imidazole-4,1′-naphthalen]-5(1H)-one

2-Amino-1,3′,3′-trimethyl-7′-(4-(trifluoromethyl)pyridin-2-yl)-3′,4′-dihydro-2′H-spiro[imidazole-4,1′-naphthalen]-5(1H)-one was prepared according to the procedures of Example 136, in which 2-bromo-4-(trifluoromethyl)pyridine was used in place of 2-(5-bromopyridin-3-yloxy)acetonitrile in Step B. ¹H NMR (400 MHz, CDCl₃) δ 8.81 (d, J=5.1 Hz, 1H), 7.81 (dd, J=8.2, 2.0 Hz, 1H), 7.80-7.78 (m, 1H), 7.67 (d, J=1.6 Hz, 1H), 7.42 (d, J=4.7 Hz, 1H), 7.24 (d, J=7.8 Hz, 1H), 3.23 (s, 3H), 2.86 (d, J=16.4 Hz, 1H), 2.64 (dd, J=16.4, 2.3 Hz, 1H), 2.33 (d, J=14.1 Hz, 1H), 1.85 (dd, J=13.9, 2.5 Hz, 1H), 1.18 (s, 3H), 1.02 (s, 3H); m/z (APCI+) M+1=403.

### Example 140

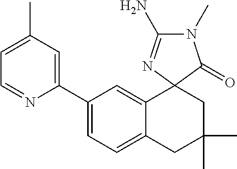

### 2-amino-1,3′,3′-trimethyl-7′-(4-methylpyridin-2-yl)-3′,4′-dihydro-2′H-spiro[imidazole-4,1′-naphthalen]-5(1H)-one

2-Amino-1,3′,3′-trimethyl-7′-(4-methylpyridin-2-yl)-3′,4′-dihydro-2′H-spiro[imidazole-4,1′-naphthalen]-5(1H)-one was prepared according to the procedures of Example 136, in which 2-bromo-4-methylpyridine was used in place of 2-(5-bromopyridin-3-yloxy)acetonitrile in Step B. ¹H NMR (400 MHz, CDCl₃) δ 8.48 (d, J=5.1 Hz, 1H), 7.78 (dd, J=8.0, 1.8 Hz, 1H), 7.57 (d, J=1.6 Hz, 1H), 7.40 (s, 1H), 7.19 (d, J=8.2 Hz, 1H), 7.03 (d, J=5.1 Hz, 1H), 3.20 (s, 3H), 2.84 (d, J=16.0 Hz, 1H), 2.60 (dd, J=16.0, 2.3 Hz, 1H), 2.39 (s, 3H), 2.35 (d, J=14.1 Hz, 1H), 1.83 (dd, J=13.9, 2.5 Hz, 1H), 1.17 (s, 3H), 0.99 (s, 3H); m/z (APCI+) M+1=349.

### Example 141

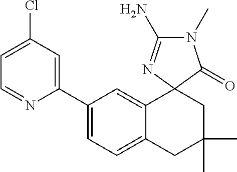

### 2-amino-7′-(4-chloropyridin-2-yl)-1,3′,3′-trimethyl-3′,4′-dihydro-2′H-spiro[imidazole-4,1′-naphthalen]-5(1H)-one

2-Amino-7′-(4-chloropyridin-2-yl)-1,3′,3′-trimethyl-3′,4′-dihydro-2′H-spiro[imidazole-4,1′-naphthalen]-5(1H)-one was prepared according to the procedures of Example 136, in which 2-bromo-4-chloropyridine was used in place of 2-(5-bromopyridin-3-yloxy)acetonitrile in Step B. ¹H NMR (400 MHz, CDCl₃) δ 8.53 (d, J=5.5 Hz, 1H), 7.78 (dd, J=8.0, 1.8 Hz, 1H), 7.61-7.58 (m, 2H), 7.23-7.21 (m, 1H), 7.21-7.19 (m, 1H), 3.22 (s, 3H), 2.85 (d, J=16.4 Hz, 1H), 2.62 (dd, J=16.2, 2.5 Hz, 1H), 2.33 (d, J=14.1 Hz, 1H), 1.83 (dd, J=13.9, 2.5 Hz, 1H), 1.17 (s, 3H), 1.00 (s, 3H); m/z (APCI+) M+1=369.

### Example 142

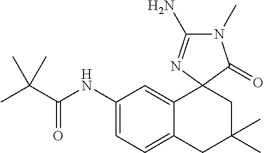

### N-(2-amino-1,3′,3′-trimethyl-5-oxo-1,3′,4′,5-tetrahydro-2′H-spiro[imidazole-4,1′-naphthalene]-7′-yl)pivalamide

Pivalamide (0.0156 g, 0.155 mmol), Cs₂CO₃ (0.0581 g, 0.178 mmol), Pd₂ dba₃ (0.00763 g, 0.00833 mmol), and Xantphos (0.00964 g, 0.0167 mmol) were combined with dioxane (0.6 mL; degassed with N₂ sparge 20 minutes prior to use), and the mixture was sonicated for 5 minutes. 2-Amino-7′-bromo-1,3′,3′-trimethyl-3′,4′-dihydro-2′H-spiro[imidazole-4,1′-naphthalen]-5(1H)-one (0.040 g, 0.119 mmol) was then added, and the reaction mixture was sonicated and heated in a 110° C. reaction block and stirred for 16 hours. The reaction mixture was then concentrated to ½ volume under a nitrogen stream, and the crude was loaded directly onto a preparative TLC plate for purification (2 mm plate, 10:1 DCM:7N NH₃/MeOH) to give N-(2-amino-1,3′,3′-trimethyl-5-oxo-1,3′,4′,5-tetrahydro-2′H-spiro[imidazole-4,1′-naphthalene]-7′-yl)pivalamide (0.0107 g, 25.2% yield) as a powder. ¹H NMR (400 MHz, CDCl₃) δ 7.59 (d, J=8.2 Hz, 1H), 7.44 (br s, 1H), 7.10 (s, 1H), 7.06 (d, J=8.2 Hz, 1H), 3.25 (s, 3H), 2.73 (d, J=16.0 Hz, 1H), 2.53 (d, J=16.4 Hz, 1H), 2.25 (d, J=13.7 Hz, 1H), 1.79 (dd, J=14.1, 2.0 Hz, 1H), 1.29 (s, 9H), 1.14 (s, 3H), 0.99 (s, 3H); m/z (APCI+) M+1=357.

### Example 143

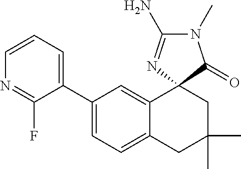

### (R)-2-amino-7′-(2-fluoropyridin-3-yl)-1,3′,3′-trimethyl-3′,4′-dihydro-2′H-spiro[imidazole-4,1′-naphthalen]-5(1H)-one

(R)-2-Amino-7′-(2-fluoropyridin-3-yl)-1,3′,3′-trimethyl-3′,4′-dihydro-2′H-spiro[imidazole-4,1′-naphthalen]-5(1H)-one was prepared according to the procedures of Example 78, in which 2-fluoropyridin-3-ylboronic acid was used in place of pyrimidin-5-ylboronic acid in Step B. ¹H NMR (400 MHz, CDCl₃) δ 8.16 (m, 1H), 7.78 (m, 1H), 7.42 (m, 1H), 7.2 (m, 2H), 7.10 (s, 1H), 3.20 (s, 1H), 2.83 (d, J=16 Hz, 1H), 2.61 (d, J=16 Hz, 1H), 2.31 (d, J=14 Hz, 1H), 1.83 (d, J=14 Hz, 1H), 1.17 (s, 3H), 1.03 (s, 3H); m/z (APCI-pos) M+1=353.

### Example 144

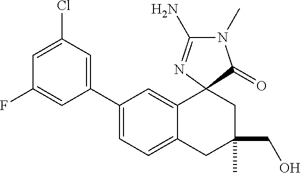

### (1′R,3′S)-2-amino-7′-(3-chloro-5-fluorophenyl)-3′-(hydroxymethyl)-1,3′-dimethyl-3′,4′-dihydro-2′H-spiro[imidazole-4,1′-naphthalen]-5(1H)-one

Step A: A 1 L round bottomed flask plus stir bar with attached Dean Stark trap was charged with 1-(4-bromophenyl)propan-2-one (100 g, 469 mmol), toluene (300 mL), and ethyl 2-cyanoacetate (53.1 g, 469 mmol). Next, ammonium acetate (17.4 g, 225 mmol) was added, followed by acetic acid (25.8 mL, 451 mmol). The mixture was heated to reflux (bath temp=125° C.), and collected water in the Dean Stark trap (total of 32 mL of water collected) for 5 hours. After cooling to room temperature, the mixture was diluted with EtOAc (500 mL) and washed with water (200 mL). The aqueous was re-extracted with EtOAc (100 mL). Combined organic phases were washed again with water (200 mL), brine (200 mL), dried (MgSO₄), filtered, and concentrated to yield (E)-ethyl 4-(4-bromophenyl)-2-cyano-3-methylbut-2-enoate (160 g, 105%). The product was carried forward without purification.

Step B: An aqueous solution (150 mL) of KCN (33.2 g, 510 mmol) was added to a solution of (E)-ethyl 4-(4-bromophenyl)-2-cyano-3-methylbut-2-enoate (143 g, 464 mmol) in MeOH (300 mL) with stirring and cooling in an ice bath to maintain internal temperature below 20° C. The ice bath was removed after addition of KCN solution, and the mixture was allowed to warm to room temperature and stir for 1 hour. Carefully acidified the mixture with aqueous 3N HCl (250 mL), then bubbled N₂ through the mixture to sparge excess HCN for 1 hour (hood sashes closed to minimize exposure to HCN). Extracted the product with diethyl ether (2×200 mL). Combined organic phases washed with brine (200 mL), dried (MgSO₄), filtered, and concentrated to yield (148 g, 83%) a 1:1 mixture of methyl 4-(4-bromophenyl)-2,3-dicyano-3-methylbutanoate and ethyl 4-(4-bromophenyl)-2,3-dicyano-3-methylbutanoate. The crude mixture was carried forward without purification.

Step C: A flask containing a 1:1 mixture of methyl 4-(4-bromophenyl)-2,3-dicyano-3-methylbutanoate and ethyl 4-(4-bromophenyl)-2,3-dicyano-3-methylbutanoate (148 g, 442 mmol) was charged with concentrated HCl (600 mL) and acetic acid (300 mL). The mixture was heated to reflux for 16 hours with stirring. After cooling to room temperature, the mixture was diluted with water (500 mL) and extracted with diethyl ether (2×150 mL). Combined organic phases were washed with brine (200 mL), dried (Na₂SO₄), filtered, and concentrated to yield a 1:1 mixture of 4-(4-bromophenyl)-3-cyano-3-methylbutanoic acid and 2-(4-bromobenzyl)-2-methylsuccinic acid (165 g, 96%). The mixture was carried forward without purification at this step.

Step D: A round bottomed flask plus stir bar was charged with a 1:1 mixture of 4-(4-bromophenyl)-3-cyano-3-methylbutanoic acid and 2-(4-bromobenzyl)-2-methylsuccinic acid (123 g, 436 mmol) (that had been azeotroped with toluene, 3×200 mL, to remove residual acetic acid present from last reaction), EtOH (500 mL), and aqueous sodium hydroxide (87.2 g, 2180 mmol) in water (150 mL). The mixture was heated to reflux for 18 hours. The suspension was cooled in an ice bath to 5-10° C. internal temperature. The mixture was acidified with concentrated HCl (approximately 150 mL). The mixture was transferred to a separatory funnel with EtOAc (400 mL) and water (400 mL). The phases were separated. Re-extracted aqueous with EtOAc (2×200 mL). Combined organic phases were washed with brine (300 mL), dried (MgSO₄), filtered, and concentrated. The residue (130 g) was azeotroped with toluene (2×200 mL) to remove residual solvents and water. The residue was triturated with toluene (200 mL) by heating and mixing with spatula to obtain a suspension. The suspension was cooled in an ice bath and filtered, rinsing solids with toluene to yield 2-(4-bromobenzyl)-2-methylsuccinic acid (36.7 g, 27%). The product was greater than 95% pure by ¹H NMR.

Step E: A round bottomed flask plus stir bar was charged with 2-(4-bromobenzyl)-2-methylsuccinic acid (40.8 g, 135 mmol; some of this 2-(4-bromobenzyl)-2-methylsuccinic acid had been previously synthesized on smaller scale by the same method as described above) and carefully added neat H₂SO₄ (200 mL). The mixture was heated to 60° C. for 2 hours with stirring. After cooling to room temperature, the reaction mixture was poured on to ice and extracted with EtOAc (3×200 mL). Combined organic phases were washed with brine (300 mL), dried (MgSO₄), filtered, and concentrated to yield 6-bromo-2-methyl-4-oxo-1,2,3,4-tetrahydronaphthalene-2-carboxylic acid (28.6 g, 67%). The product was carried forward without purification at this stage.

Step F: A round bottomed flask plus stir bar was charged with toluene (300 mL), followed by 6-bromo-2-methyl-4-oxo-1,2,3,4-tetrahydronaphthalene-2-carboxylic acid (27.4 g, 96.8 mmol). The mixture was cooled in an ice bath. A BH₃-THF complex (1M in THF, 290 mL, 290 mmol) was added dropwise to the stirring mixture under N₂ until foaming ceased (much gas evolution during first third of addition), then added the BH₃-THF in 10 mL portions until addition was finished. The internal temperature was maintained below 10° C. during the addition of BH₃-THF. The mixture was removed from the ice bath. The mixture was stirred for 2 hours at room temperature. 10% Aqueous citric acid solution (500 mL) was added to a second flask that was chilled in an ice bath with stirring. The reaction mixture was quenched by pouring into the citric acid solution in portions (much gas evolution, placed a N₂ line over the top of the mixture to continually flush out H₂ gas), maintaining the internal quench solution below 10° C. After complete addition of the reaction mixture to the quench solution, the solution was stirred for 2 hours at room temperature. The phases were separated, and the aqueous was re-extracted with EtOAc (2×200 mL). Combined organics were washed with brine (500 mL), dried (MgSO₄), filtered, and concentrated to yield 7-bromo-3-(hydroxymethyl)-3-methyl-1,2,3,4-tetrahydronaphthalen-1-ol (27 g, 72%). The product appeared to be a 60:40 mixture of diastereomers by ¹H NMR.

Step G: A round bottomed flask plus stir bar was charged with 7-bromo-3-(hydroxymethyl)-3-methyl-1,2,3,4-tetrahydronaphthalen-1-ol (18 g, 66 mmol), CHCl₃ (500 mL), and lastly manganese(IV) oxide (58 g, 664 mmol). The mixture was heated to 50° C. with stirring for 22 hours. The mixture was filtered through Celite®, rinsing with DCM. The filtrate was concentrated. Combined crude from this reaction with crude product (8.1 g) from smaller scale reactions, and purified by Biotage Flash 65 silica gel chromatography, eluting with 30% EtOAc/hexanes, followed by 1:1 EtOAc/hexanes to obtain 7-bromo-3-(hydroxymethyl)-3-methyl-3,4-dihydronaphthalen-1(2H)-one (19.1 g, 60% yield).

Step H: A solution of 7-bromo-3-(hydroxymethyl)-3-methyl-3,4-dihydronaphthalen-1(2H)-one (19.1 g, 71.0 mmol) and tert-butylchlorodimethylsilane (10.7 g, 71.0 mmol) in DCM (200 mL) were cooled in an ice bath and treated with portionwise addition of imidazole (9.66 g, 142 mmol) while stirring. The reaction was allowed to stir for 3 days at room temperature. The reaction was transferred to a separatory funnel and washed with saturated aqueous NH₄Cl (200 mL), brine (200 mL), dried (Na₂SO₄), filtered and concentrated to yield 7-bromo-3-((tert-butyldimethylsilyloxy)methyl)-3-methyl-3,4-dihydronaphthalen-1(2H)-one (26.0 g, 85%). The product was carried forward without purification at this step.

Step I: A stainless steel bomb (50 mL capacity) with teflon insert was charged with EtOH (13 mL) and 7-bromo-3-((tert-butyldimethylsilyloxy)methyl)-3-methyl-3,4-dihydronaphthalen-1(2H)-one (5 g, 13 mmol). Next, ammonium carbonate (6.3 g, 65 mmol), KCN (1.7 g, 26 mmol), and sodium hydrogensulfite (0.34 g, 3.3 mmol) were added. The reaction was heated to 150° C. for 15 hours with stirring. After cooling to room temperature, the reaction contents were transferred to an Erlenmeyer flask with EtOAc (30 mL) and water (20 mL). The pH was carefully neutralized (8-9) with saturated aqueous NH₄Cl. The phases were separated, and the aqueous phase was re-extracted with EtOAc (2×20 mL). Combined organic phases were washed with brine (20 mL), dried (MgSO₄), filtered, and concentrated to yield 7′-bromo-3′-((tert-butyldimethylsilyloxy)methyl)-3′-methyl-3′,4′-dihydro-2′H-spiro[imidazolidine-4,1′-naphthalene]-2,5-dione (5.4 g, 58%). The product was carried forward without purification at this step.

Step J: A round bottomed flask plus stir bar was charged with potassium carbonate (1.65 g, 11.9 mmol) and DMF (25 mL). T-Bromo-3′-((tert-butyldimethylsilyloxy)methyl)-3′-methyl-3′,4′-dihydro-2′H-spiro[imidazolidine-4,1′-naphthalene]-2,5-dione (5.4 g, 12 mmol) was added. Lastly, iodomethane (0.67 mL, 11 mmol) was added. The mixture was stirred at room temperature for 18 hours. The reaction mixture was worked up by partitioning between EtOAc (50 mL) and water (50 mL). The phases were separated, and the aqueous phase was re-extracted with EtOAc (50 mL). Combined organic phases were washed with water (50 mL), brine (50 mL), dried (MgSO₄), filtered, and concentrated to yield 7′-bromo-3′-((tert-butyldimethylsilyloxy)methyl)-1,3′-dimethyl-3′,4′-dihydro-2′H-spiro[imidazolidine-4,1′-naphthalene]-2,5-dione (5.8 g, 89%) as a 1:1 diastereomeric mixture. The product was carried forward without purification.

Step K: A thick walled glass pressure tube was charged with T-bromo-3′-((tert-butyldimethylsilyloxy)methyl)-1,3′-dimethyl-3′,4′-dihydro-2′H-spiro[imidazolidine-4,1′-naphthalene]-2,5-dione (5.6 g, 12 mmol), Lawesson's Reagent (2.9 g, 7.2 mmol), and toluene (50 mL). The mixture was degassed with N₂ and heated to 100° C. for 15 hours with stirring. After cooling to room temperature, the mixture was partitioned between EtOAc (50 mL) and saturated aqueous NaHCO₃ (50 mL). The phases were separated, and the aqueous phase was re-extracted with EtOAc (20 mL). Combined organic phases were washed with brine (50 mL), dried (MgSO₄), filtered, and concentrated. The product was partially purified by Biotage Flash 40 silica gel chromatography, eluting with 5% EtOAc/hexanes, followed by 10% EtOAc/hexanes to yield 7′-bromo-3′-((tert-butyldimethylsilyloxy)methyl)-1,3′-dimethyl-2-thioxo-3′,4′-dihydro-2′H-spiro[imidazolidine-4,1′-naphthalen]-5-one (3.5 g, 48%).

Step L: A round bottomed flask plus stir bar was charged with 7′-bromo-3′-((tert-butyldimethylsilyloxy)methyl)-1,3′-dimethyl-2-thioxo-3′,4′-dihydro-2′H-spiro[imidazolidine-4,1′-naphthalen]-5-one (3.5 g, 7.2 mmol), MeOH (60 mL), t-butyl hydroperoxide 70% aqueous (15 mL, 109 mmol), and 30% aqueous NH₄OH (28 mL, 217 mmol). The mixture was stirred for 15 hours at room temperature. The mixture was diluted with water (5 mL) and concentrated in vacuo. The mixture was partitioned between EtOAc (50 mL) and water (50 mL). The phases were separated, and the aqueous phase was re-extracted with EtOAc (20 mL). Combined organic phases were washed with brine (50 mL), dried (MgSO₄), filtered, and concentrated to yield a diastereomeric mixture of (1′R,3′R)-2-amino-7′-bromo-3′-((tert-butyldimethylsilyloxy)methyl)-1,3′-dimethyl-3′,4′-dihydro-2′H-spiro[imidazole-4,1′-naphthalen]-5(1H)-one (2.8 g, 58%). The product was carried forward without separation of diastereomers at this step.

Step M: A round bottomed flask plus stir bar was charged with (1′R,3′R)-2-amino-7′-bromo-3′-((tert-butyldimethylsilyloxy)methyl)-1,3′-dimethyl-3′,4′-dihydro-2′H-spiro[imidazole-4,1′-naphthalen]-5(1H)-one (1.0 g, 2.1 mmol), THF (5 mL) and TBAF (2.57 mL, 2.57 mmol, 1M in THF). The mixture was stirred at room temperature for 15 hours. The mixture was concentrated in vacuo. [Note: avoid aqueous workup as product is water soluble.] The crude was loaded directly on to Biotage Flash 65 silica gel chromatography column, eluting with a gradient of 5%-25% MeOH/DCM to yield (1′R,3′S)-2-amino-7′-bromo-3′-(hydroxymethyl)-1,3′-dimethyl-3′,4′-dihydro-2′H-spiro[imidazole-4,1′-naphthalen]-5(1H)-one (554 mg, 70%).

Step N: A vial plus stir bar was charged with (1′R,3′S)-2-amino-7′-bromo-3′-(hydroxymethyl)-1,3′-dimethyl-3′,4′-dihydro-2′H-spiro[imidazole-4,1′-naphthalen]-5(1H)-one (100 mg, 0.284 mmol), dioxane (1 mL), 3-chloro-5-fluorophenylboronic acid (40 mg, 0.23 mmol), Pd(PPh₃)₄ (33 mg, 0.028 mmol), and 2N aqueous Na₂CO₃ (360 μL, 0.71 mmol). The mixture was sparged with N₂ for 30 seconds and then heated to 90° C. for 15 hours with stirring. The reaction mixture was loaded directly on to a preparative TLC plate (2 mm thickness, R₁=0.31) and eluted with 15% MeOH/DCM. Repurified by preparative TLC (0.5 mm thickness) eluting with 10% MeOH (containing 7N NH₃)/DCM to yield (1′R,3′S)-2-amino-7′-(3-chloro-5-fluorophenyl)-3′-(hydroxymethyl)-1,3′-dimethyl-3′,4′-dihydro-2′H-spiro[imidazole-4,1′-naphthalen]-5(1H)-one (35 mg, 30%). Greater than 98% diastereomeric purity by ¹H NMR. ¹H NMR (400 MHz, CDCl₃) δ 7.36 (dd, J=2, 8 Hz, 1H), 7.23 (m, 1H), 7.18 (d, J=8 Hz, 1H), 7.02-7.07 (m, 3H), 3.97 (br s, 3H), 3.58 (d, J=11 Hz, 1H), 3.40 (d, J=11 Hz, 1H), 3.17 (s, 3H), 2.83 (d, J=16 Hz, 1H), 2.57 (d, J=16 Hz, 1H), 2.31 (d, J=14 Hz, 1H), 1.76 (dd, J=1, 14 Hz, 1H), 1.03 (s, 3H); m/z (APCI-pos) M+1=402.

### Example 145

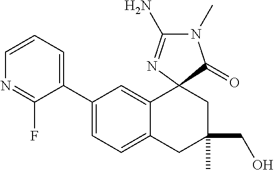

### (1′R,3′S)-2-amino-7′-(2-fluoropyridin-3-yl)-3′-(hydroxymethyl)-1,3′-dimethyl-3′,4′-dihydro-2′H-spiro[imidazole-4,1′-naphthalen]-5(1H)-one

A thick walled, glass pressure tube plus stir bar was charged with (1′R,3′S)-2-amino-7′-bromo-3′-(hydroxymethyl)-1,3′-dimethyl-3′,4′-dihydro-2′H-spiro[imidazole-4,1′-naphthalen]-5(1H)-one (554 mg, 1.57 mmol; Example 144, Step M), dioxane (10 mL), 2-fluoropyridin-3-ylboronic acid (244 mg, 1.73 mmol), Pd(PPh₃)₄ (91 mg, 0.079 mmol), and 2N aqueous Na₂CO₃ (2 mL, 4 mmol). The mixture was sparged with N₂ for 3 minutes and then heated to 90° C. for 15 hours with stirring. After cooling to room temperature, the mixture was partitioned between EtOAc (30 mL) and water (30 mL). The phases were separated, and the aqueous phase was re-extracted with EtOAc (10 mL). The combined organic phases were washed with brine (20 mL), dried (MgSO₄), filtered, and concentrated to obtain crude material (489 mg, 42%), which was a 60:40 mixture of desired product ((1′R,3′S)-2-amino-7′-(2-fluoropyridin-3-yl)-3′-(hydroxymethyl)-1,3′-dimethyl-3′,4′-dihydro-2′H-spiro[imidazole-4,1′-naphthalen]-5(1H)-one) and unreacted starting material ((1′R,3′S)-2-amino-7′-bromo-3′-(hydroxymethyl)-1,3′-dimethyl-3′,4′-dihydro-2′H-spiro[imidazole-4,1′-naphthalen]-5(1H)-one). As there was no separation between desired Suzuki product and unreacted starting material by silica gel chromatography, the mixture was resubjected to a second Suzuki reaction as follows. A thick walled, glass pressure tube plus stir bar was charged with the crude mixture of (1′R,3′S)-2-amino-7′-(2-fluoropyridin-3-yl)-3′-(hydroxymethyl)-1,3′-dimethyl-3′,4′-dihydro-2′H-spiro[imidazole-4,1′-naphthalen]-5(1H)-one and (1′R,3′S)-2-amino-7′-bromo-3′-(hydroxymethyl)-1,3′-dimethyl-3′,4′-dihydro-2′H-spiro[imidazole-4,1′-naphthalen]-5(1H)-one (489 mg, 1.39 mmol), dioxane (10 mL), pyrimidin-5-ylboronic acid (103 mg, 0.833 mmol), Pd(PPh₃)₄ (160 mg, 0.139 mmol), and 2N aqueous Na₂CO₃ (1.7 mL, 3.5 mmol). The mixture was sparged with N₂ for 3 minutes and then heated to 90° C. for 15 hours with stirring. After cooling to room temperature, the reaction mixture was loaded directly on to a preparative TLC plate (2 mm thickness) eluting with 15% MeOH (containing 7N NH₃)/DCM. The product containing bands was repurified by Biotage Flash 40 silica gel chromatography eluting with a gradient of 10%-30% MeOH/DCM. The two Suzuki products (1′R,3′S)-2-amino-7′-(2-fluoropyridin-3-yl)-3′-(hydroxymethyl)-1,3′-dimethyl-3′,4′-dihydro-2′H-spiro[imidazole-4,1′-naphthalen]-5(1H)-one and (1′R,3′S)-2-amino-3′-(hydroxymethyl)-1,3′-dimethyl-7′-(pyrimidin-5-yl)-3′,4′-dihydro-2′H-spiro[imidazole-4,1′-naphthalen]-5(1H)-one (Example 146) were separated. Yield of (1′R,3′S)-2-amino-7′-(2-fluoropyridin-3-yl)-3′-(hydroxymethyl)-1,3′-dimethyl-3′,4′-dihydro-2′H-spiro[imidazole-4,1′-naphthalen]-5(1H)-one (123 mg, 23%). ¹H NMR (400 MHz, CDCl₃) δ 8.16 (m, 1H), 7.78 (m, 1H), 7.40 (m, 1H), 7.23 (m, 2H), 7.11 (m, 1H), 3.61 (d, J=11 Hz, 1H), 3.45 (br s, 2H), 3.44 (d, J=11 Hz, 1H), 3.17 (s, 3H), 2.85 (d, J=16 Hz, 1H), 2.62 (d, J=16 Hz, 1H), 2.36 (d, J=14 Hz, 1H), 1.82 (m, 1H), 1.07 (s, 3H); m/z (APCI-pos) M+1=369.

### Example 146

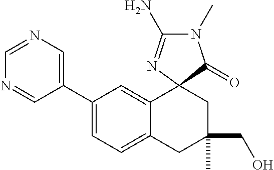

### (1′R,3′S)-2-amino-3′-(hydroxymethyl)-1,3′-dimethyl-7′-(pyrimidin-5-yl)-3′,4′-dihydro-2′H-spiro[imidazole-4,1′-naphthalen]-5(1H)-one

(1′R,3′S)-2-Amino-3′-(hydroxymethyl)-1,3′-dimethyl-7′-(pyrimidin-5-yl)-3′,4′-dihydro-2′H-spiro[imidazole-4,1′-naphthalen]-5(1H)-one (18 mg, 3%) was prepared and isolated as described in Example 145. ¹H NMR (400 MHz, CDCl₃+few drops of MeOD) δ 9.04 (s, 1H), 8.78 (s, 2H), 7.35 (dd, J=2, 8 Hz, 1H), 7.21 (d, J=8 Hz, 1H), 6.98 (m, 1H), 3.41 (d, J=11 Hz, 1H), 3.34 (d, J=11 Hz, 1H), 3.07 (s, 3H), 2.83 (d, J=16 Hz, 1H), 2.52 (d, J=16 Hz, 1H), 2.19 (d, J=14 Hz, 1H), 1.70 (m, 1H), 0.94 (s, 3H); m/z (APCI-pos) M+1=352.

### Example 147

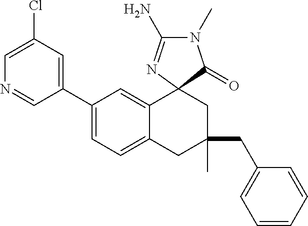

### (1′R,3′S)-2-amino-3′-benzyl-7′-(5-chloropyridin-3-yl)-1,3′-dimethyl-3′,4′-dihydro-2′H-spiro[imidazole-4,1′-naphthalen]-5(1H)-one

Step A: A round bottomed flask plus stir bar was charged with 1-(4-bromophenyl)propan-2-one (25 g, 117 mmol), diethyl malonate (17.8 mL, 117 mmol) and toluene (200 mL). Next, pyridine (38 mL, 469 mmol) was added, followed by TiCl₄ (44.5 g, 235 mmol). The reaction was quite exothermic, so the flask was submerged in an ice bath. The reaction stirred at room temperature for 1 day. The reaction mixture was poured on to ice, and extracted with EtOAc (2×100 mL). The combined organic phases were washed with brine (200 mL), dried (Na₂SO₄), filtered, and concentrated. The crude was purified by Biotage Flash 65 silica gel chromatography, eluting with 3% EtOAc/hexanes to obtain diethyl 2-(1-(4-bromophenyl)propan-2-ylidene)malonate (14.1 g, 18%).

Step B: 1-Bromo-4-(bromomethyl)benzene (20 g, 79 mmol) in diethyl ether (50 mL) was added at a rate to maintain a gentle reflux to a stirred suspension of magnesium turnings (1.9 g, 79 mmol) in diethyl ether (10 mL). After the addition was complete, it was refluxed for 30 minutes with stirring. It was then cooled to 0° C., and copper(I) chloride (0.12 g, 1.2 mmol) was added. It was allowed to stir for 10 minutes, after which diethyl 2-(1-(4-bromophenyl)propan-2-ylidene)malonate (14 g, 39 mmol) in diethyl ether (60 ml) was added dropwise over 30 minutes. The stirred mixture was allowed to warm to room temperature and stirred overnight. It was carefully poured onto 1M H₂SO₄ (50 mL), and the product was extracted with diethyl ether (3×50 mL). The combined organic phases were washed with brine (100 mL), dried (MgSO₄), filtered, and concentrated. The crude was purified by Biotage Flash 65 silica gel chromatography, eluting with 2% EtOAc/hexanes, then 5% EtOAc/hexanes to obtain diethyl 2-(1,3-bis(4-bromophenyl)-2-methylpropan-2-yl)malonate (8.2 g, 38%).

Step C: Potassium hydroxide (4.0 g, 72 mmol) was added to a stirred solution of diethyl 2-(1,3-bis(4-bromophenyl)-2-methylpropan-2-yl)malonate (8.2 g, 16 mmol) in 2:1 EtOH/water (50 mL), and the reaction was heated to 60° C. for 15 hours. After cooling to room temperature, it was diluted with water (100 mL) and washed with DCM (2×50 mL). The combined organics were re-extracted with 2N aqueous NaOH (50 mL). The combined aqueous phases were acidified with concentrated HCl, and extracted with DCM (3×50 mL). These combined organics were dried (Na₂SO₄), filtered, and concentrated to obtain 2-(1,3-bis(4-bromophenyl)-2-methylpropan-2-yl)malonic acid (6.8 g, 84%). The product was carried forward without purification at this step.

Step D: Neat 2-(1,3-bis(4-bromophenyl)-2-methylpropan-2-yl)malonic acid (6.8 g, 14 mmol) was heated to 185-195° C. for 1 hour, during which substantial bubbling was observed, due to the loss of carbon dioxide. It was cooled to room temperature to yield 3-(4-bromobenzyl)-4-(4-bromophenyl)-3-methylbutanoic acid (6.2 g, 101%). The product was carried forward without purification.

Step E: A stirred suspension of 3-(4-bromobenzyl)-4-(4-bromophenyl)-3-methylbutanoic acid (6.2 g, 15 mmol) in polyphosphoric acid (60 g) was heated to 120° C. for 2 hours. The mixture was cooled to room temperature and poured onto ice. The mixture was extracted with DCM (3×50 mL). The combined organics were dried (Na₂SO₄), filtered, and concentrated. The crude was purified crude by Biotage Flash 65 silica gel chromatography, eluting with a gradient of 2%-10% EtOAc/hexanes to obtain 7-bromo-3-(4-bromobenzyl)-3-methyl-3,4-dihydronaphthalen-1(2H)-one (1.6 g, 22%).

Step F: A thick walled glass pressure tube plus stir bar was charged with 7-bromo-3-(4-bromobenzyl)-3-methyl-3,4-dihydronaphthalen-1(2H)-one (1.6 g, 3.9 mmol), dioxane (40 mL), 5-chloropyridin-3-ylboronic acid (0.62 g, 3.9 mmol), Pd(PPh₃)₄ (0.453 g, 0.392 mmol), and 2N aqueous Na₂CO₃ (4.9 mL, 9.8 mmol). The mixture was sparged with N₂ for 3 minutes and then heated to 90° C. for 4 hours. After cooling to room temperature, the mixture was partitioned between EtOAc (100 mL) and water (100 mL). The phases were separated. The aqueous phase was re-extracted with EtOAc (50 mL). The combined organic phases were washed with brine (100 mL), dried (MgSO₄), filtered, and concentrated. The products were separated by Biotage Flash 40L silica gel chromatography, eluting with a gradient of 10%-40% EtOAc/hexanes, then neat EtOAc to isolate 3-(4-bromobenzyl)-7-(5-chloropyridin-3-yl)-3-methyl-3,4-dihydronaphthalen-1(2H)-one (720 mg, 33%).

Step G: 3′-(4-Bromobenzyl)-7′-(5-chloropyridin-3-yl)-1,3′-dimethyl-2-thioxo-3′,4′-dihydro-2′H-spiro[imidazolidine-4,1′-naphthalen]-5-one (309 mg, 0.57 mmol) was prepared according to the procedures described for Example 144, Steps I-K, replacing 7-bromo-3-((tert-butyldimethylsilyloxy)methyl)-3-methyl-3,4-dihydronaphthalen-1(2H)-one with 3-(4-bromobenzyl)-7-(5-chloropyridin-3-yl)-3-methyl-3,4-dihydronaphthalen-1(2H)-one (700 mg, 1.59 mmol).

Step H: A round bottomed flask plus stir bar was charged with 3′-(4-bromobenzyl)-7′-(5-chloropyridin-3-yl)-1,3′-dimethyl-2-thioxo-3′,4′-dihydro-2′H-spiro[imidazolidine-4,1′-naphthalen]-5-one (309 mg, 0.57 mmol), MeOH (12 mL), t-butyl hydroperoxide 70% aqueous (3.2 mL, 23 mmol), and 30% aqueous NH₄OH (6 mL, 46 mmol). The mixture was stirred for 15 hours at room temperature. The mixture was diluted with water (5 mL) and concentrated in vacuo. The mixture was partitioned between EtOAc (50 mL) and water (50 mL). The phases were separated. The aqueous phase was re-extracted with EtOAc (20 mL). The combined organic phases were washed with brine (50 mL), dried (MgSO₄), filtered, and concentrated. The crude was partially purified by Biotage Flash 40L silica gel chromatography, eluting with 2% MeOH (containing 7N NH₃)/DCM then 5% MeOH (containing 7N NH₃)/DCM to obtain a mixture of diastereomers (172 mg). The diastereomers were re-purified by preparative TLC (2 mm thickness, R_{f}=0.18 for desired diastereomer) eluting with 15% MeOH/EtOAc. (1′R,3′S)-2-Amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one was isolated (27 mg, 8% yield; Example 149), which was 90% diastereomerically pure by ¹H NMR. The diastereomer (1′R,3′R)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one (57 mg, R_{f}=0.41; Example 150) was also isolated.

Step I: A round bottomed flask plus stir bar was charged with (1′R,3′S)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one (23 mg, 0.044 mmol) and THF (0.2 mL). The stirring mixture was chilled to −78° C. (dry ice/acetone) under N₂. tert-Butyl lithium (100 μL, 0.18 mmol, 1.7M in pentane) was added. The mixture was stirred for 15 minutes. The reaction mixture was quenched with saturated aqueous NH₄Cl. The phases were separated, and the aqueous phase was extracted with EtOAc (2×5 mL). The combined organics were washed with brine (5 mL), dried (MgSO₄), filtered, and concentrated. The products were partially purified by preparative TLC (0.5 mm thickness, R_{f}=0.39) eluting with 7.5% MeOH (containing 7N NH₃) in DCM. ¹H NMR indicated a 63/37 mixture (7 mg) of desired product, (1′R,3′S)-2-amino-3′-benzyl-7′-(5-chloropyridin-3-yl)-1,3′-dimethyl-3′,4′-dihydro-2′H-spiro[imidazole-4,1′-naphthalen]-5(1H)-one, and the starting material, (1′R,3′S)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one, which were not separable by silica gel chromatography. The mixture was subjected to a Suzuki reaction as follow to remove the starting material. The 63/37 mixture (7 mg, 0.014 mmol) was transferred to a 2 dram vial and diluted with dioxane (0.1 mL), pyrimidin-5-ylboronic acid (1.2 mg, 0.0096 mmol), Pd(PPh₃)₄ (1.6 mg, 0.0014 mmol), and 2N aqueous Na₂CO₃ (17 μL, 0.034 mmol). The mixture was sparged with N₂ for 30 seconds and then heated to 90° C. for 15 hours. The reaction mixture was loaded directly on to a preparative TLC plate (0.5 mm thickness, R=0.50) and eluted with 7.5% MeOH (containing 7N NH₃)/DCM. The product (1′R,3′S)-2-amino-3′-benzyl-7′-(5-chloropyridin-3-yl)-1,3′-dimethyl-3′,4′-dihydro-2′H-spiro[imidazole-4,1′-naphthalen]-5(1H)-one and (1′R,3′S)-2-amino-7′-(5-chloropyridin-3-yl)-1,3′-dimethyl-3′-(4-(pyrimidin-5-yl)benzyl)-3′,4′-dihydro-2′H-spiro[imidazole-4,1′-naphthalen]-5(1H)-one (Example 148) were separated. Yield of (1′R,3′S)-2-amino-3′-benzyl-7′-(5-chloropyridin-3-yl)-1,3′-dimethyl-3′,4′-dihydro-2′H-spiro[imidazole-4,1′-naphthalen]-5(1H)-one (4 mg, 41%). ¹H NMR (400 MHz, CDCl₃) δ 8.59 (d, J=2 Hz, 1H), 8.51 (d, J=2 Hz, 1H), 7.73 (t, J=2 Hz, 1H), 7.44 (d, J=9 Hz, 1H), 7.37 (dd, J=2, 8 Hz, 1H), 7.31 (d, J=7 Hz, 1H), 7.19 (m, 3H), 7.04 (m, 2H), 3.20 (s, 3H), 2.96 (d, J=16 Hz, 1H), 2.79 (d, J=13 Hz, 1H), 2.71 (d, J=13 Hz, 1H), 2.55-2.65 (m, 2H), 2.37 (d, J=14 Hz, 1H), 1.00 (s, 3H); m/z (APCI-pos) M+1=445, 446.

### Example 148

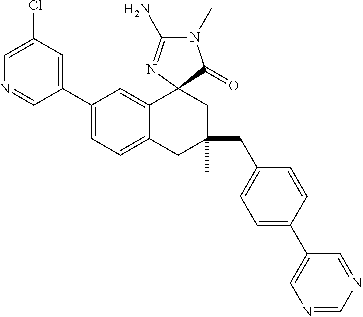

### (1′R,3′S)-2-amino-7′-(5-chloropyridin-3-yl)-1,3′-dimethyl-3′-(4-(pyrimidin-5-yl)benzyl)-3′,4′-dihydro-2′H-spiro[imidazole-4,1′-naphthalen]-5(1H)-one

(1′R,3′S)-2-Amino-7′-(5-chloropyridin-3-yl)-1,3′-dimethyl-3′-(4-(pyrimidin-5-yl)benzyl)-3′,4′-dihydro-2′H-spiro[imidazole-4,1′-naphthalen]-5(1H)-one (0.5 mg, 6%) was prepared in Example 147, Step I. It was separated by preparative TLC as described in that procedure. m/z (APCI-pos) M+1=523, 524.

### Example 149

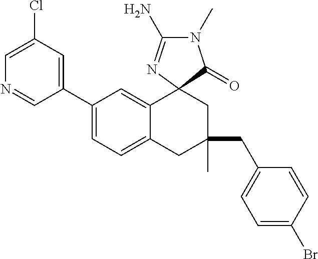

### (1′R,3′S)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one

(1′R,3′S)-2-Amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one (27 mg, 8%) was prepared in Example 147, Step H. 1H NMR (400 MHz, CDCl₃) δ 8.56 (s, 1H), 8.49 (s, 1H), 7.72 (s, 1H), 7.43 (d, J=8 Hz, 2H), 7.36 (d, J=8 Hz, 1H), 7.19 (d, J=8 Hz, 1H), 7.03 (m, 3H), 5.10 (br s, 2H), 3.16 (s, 3H), 2.92 (d, J=16 Hz, 1H), 2.73 (d, J=13 Hz, 1H), 2.65 (d, J=13 Hz, 1H), 2.55 (d, J=16 Hz, 1H), 2.30 (d, J=14 Hz, 1H), 1.77 (d, J=14 Hz, 1H), 0.96 (s, 3H); m/z (APCI-pos) M+1=523, 525.

### Example 150

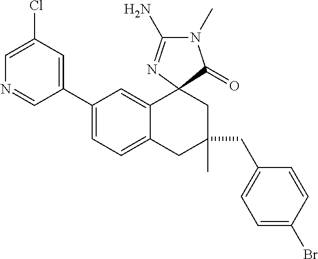

### (1′R,3′R)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one

(1′R,3′R)-2-Amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one (40 mg, 10%) was prepared in Example 147, Step H, and separated from its diastereomer by preparative TLC as described in that procedure. The title compound was repurified by preparative TLC (0.5 mm thickness, R_{f}=0.53) eluting with 5% MeOH (containing 7N NH₃)/DCM. ¹H NMR (400 MHz, CDCl₃) δ 8.62 (d, J=2 Hz, 1H), 8.51 (d, J=2 Hz, 1H), 7.77 (t, J=2 Hz, 1H), 7.42 (m, 1H), 7.36 (d, J=8 Hz, 2H), 7.22 (d, J=8 Hz, 1H), 7.07 (m, 1H), 6.88 (d, J=8 Hz, 2H), 4.35 (br s, 2H), 3.15 (s, 3H), 2.77 (d, J=13 Hz, 1H), 2.65 (m, 2H), 2.49 (d, J=13 Hz, 1H), 2.29 (d, J=14 Hz, 1H), 1.91 (d, J=14 Hz, 1H), 1.01 (s, 3H); m/z (APCI-pos) M+1=523, 525.

### Example 151

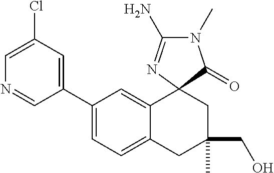

### (1′R,3′S)-2-amino-7′-(5-chloropyridin-3-yl)-3′-(hydroxymethyl)-1,3′-dimethyl-3′,4′-dihydro-2′H-spiro[imidazole-4,1′-naphthalen]-5 (1H)-one

(1′R,3′S)-2-Amino-7′-(5-chloropyridin-3-yl)-3′-(hydroxymethyl)-1,3′-dimethyl-3′,4′-dihydro-2′H-spiro[imidazole-4,1′-naphthalen]-5(1H)-one (23 mg, 31%) was prepared from (1′R,3′S)-2-amino-7′-bromo-3′-(hydroxymethyl)-1,3′-dimethyl-3′,4′-dihydro-2′H-spiro[imidazole-4,1′-naphthalen]-5(1H)-one (65 mg, 0.18 mmol; Example 144, Step M) according to the procedure for Example 144, Step N, replacing 3-chloro-5-fluorophenylboronic acid with 5-chloropyridin-3-ylboronic acid. ¹H NMR (400 MHz, CDCl₃) δ 8.60 (d, J=2 Hz, 1H), 8.52 (d, J=2 Hz, 1H), 7.75 (t, J=2 Hz, 1H), 7.38 (dd, J=2, 8 Hz, 1H), 7.23 (d, J=8 Hz, 1H), 7.07 (d, J=2 Hz, 1H), 3.61 (d, J=11 Hz, 1H), 3.44 (d, J=11 Hz, 1H), 3.25 (br s, 3H), 3.18 (s, 3H), 2.86 (d, J=16 Hz, 1H), 2.63 (d, J=16 Hz, 1H), 2.34 (d, J=14 Hz, 1H), 1.80 (dd, J=2, 14 Hz, 1H), 1.06 (s, 3H); m/z (APCI-pos) M+1=385.

### Example 152

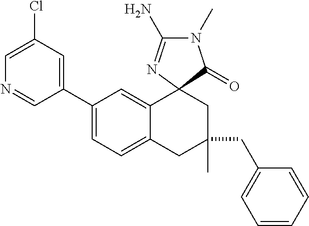

### (1′R,3′R)-2-amino-3′-benzyl-7′-(5-chloropyridin-3-yl)-1,3′-dimethyl-3′,4′-dihydro-2′H-Spiro[imidazole-4,1′-naphthalen]-5(1H)-one

Step A: Intermediate (1′R,3′R)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one (29 mg, 0.055 mmol) was prepared from 3′-(4-bromobenzyl)-7′-(5-chloropyridin-3-yl)-1,3′-dimethyl-2-thioxo-3′,4′-dihydro-2′H-spiro[imidazolidine-4,1′-naphthalen]-5-one (309 mg, 0.57 mmol; Example 147, Step G) according to Example 147, Step H, and separated from its diastereomer (1′R,3′S)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one by preparative TLC as described in that procedure.

Step B: (1′R,3′R)-2-Amino-3′-benzyl-7′-(5-chloropyridin-3-yl)-1,3′-dimethyl-3′,4′-dihydro-2′H-spiro[imidazole-4,1′-naphthalen]-5(1H)-one (4 mg, 43%) was prepared according to the procedure for Example 147, Step I, replacing (1′R,3′S)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one with (1′R,3′R)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one (29 mg, 0.055 mmol). ¹H NMR (400 MHz, CDCl₃) δ 8.64 (d, J=2 Hz, 1H), 8.53 (d, J=2 Hz, 1H), 7.79 (t, J=2 Hz, 1H), 7.44 (dd, J=2, 8 Hz, 1H), 7.17-7.27 (m, 4H), 7.11 (d, J=2 Hz, 1H), 7.00 (d, J=7 Hz, 2H), 3.64 (br s, 2H), 3.19 (s, 3H), 2.80 (d, J=13 Hz, 1H), 2.6-2.7 (m, 2H), 2.52 (d, J=13 Hz, 1H), 2.35 (d, J=14 Hz, 1H), 1.99 (dd, J=2, 14, Hz, 1H), 1.04 (s, 3H); m/z (APCI-pos) M+1=445, 446.

### Example 153

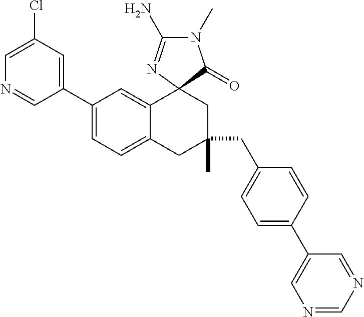

### (1′R,3′R)-2-amino-7′-(5-chloropyridin-3-yl)-1,3′-dimethyl-3′-(4-(pyrimidin-5-yl)benzyl)-3′,4′-dihydro-2′H-spiro[imidazole-4,1′-naphthalen]-5(1H)-one

(1′R,3′R)-2-Amino-7′-(5-chloropyridin-3-yl)-1,3′-dimethyl-3′-(4-(pyrimidin-5-yl)benzyl)-3′,4′-dihydro-2′H-spiro[imidazole-4,1′-naphthalen]-5(1H)-one (1 mg, 14%) was prepared according to the procedure described for Example 148, replacing (1′R,3′S)-2-amino-3′-(4-bromobenzyl)-7′-(5-chloropyridin-3-yl)-1,3′-dimethyl-3′,4′-dihydro-2′H-Spiro[imidazole-4,1′-naphthalen]-5(1H)-one with the diastereomer (1′R,3′R)-2-amino-3′-(4-bromobenzyl)-7′-(5-chloropyridin-3-yl)-1,3′-dimethyl-3′,4′-dihydro-2′H-spiro[imidazole-4,1′-naphthalen]-5(1H)-one (29 mg, 0.055 mmol; Example 150). ¹H NMR (400 MHz, CDCl₃) δ 9.19 (s, 1H), 8.95 (s, 2H), 8.66 (d, J=2 Hz, 1H), 8.53 (d, J=2 Hz, 1H), 7.80 (t, J=2 Hz, 1H), 7.48 (m, 3H), 7.29 (d, J=8 Hz, 1H), 7.17 (d, J=8 Hz, 2H), 7.11 (m, 1H), 3.23 (s, 3H), 2.90 (d, J=13 Hz, 1H), 2.74 (m, 2H), 2.62 (d, J=13 Hz, 1H), 2.37 (d, J=14 Hz, 1H), 2.01 (d, J=14 Hz, 1H), 1.09 (s, 3H); m/z (APCI-pos) M+1=523, 524.

### Example 154

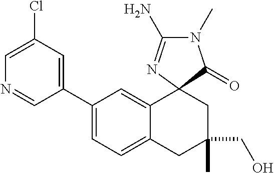

### (1′R,3′R)-2-amino-7′-(5-chloropyridin-3-yl)-3′-(hydroxymethyl)-1,3′-dimethyl-3′,4′-dihydro-2′H-spiro[imidazole-4,1′-naphthalen]-5(1H)-one

Step A: A round bottomed flask plus stir bar was charged with 7′-bromo-3′-((tert-butyldimethylsilyloxy)methyl)-1,3′-dimethyl-2-thioxo-3′,4′-dihydro-2′H-spiro[imidazolidine-4,1′-naphthalen]-5-one (1.4 g, 2.90 mmol; Example 144, Step K), MeOH (20 mL), t-butyl hydroperoxide 70% aqueous (6.0 mL, 43 mmol), and 30% aqueous NH₄OH (11 mL, 87 mmol). The mixture was stirred for 15 hours at room temperature. The mixture was diluted with water (5 mL) and concentrated in vacuo. The mixture was partitioned between EtOAc (50 mL) and water (50 mL). The phases were separated. The aqueous phase was re-extracted with EtOAc (20 mL). The combined organic phases were washed with brine (50 mL), dried (MgSO₄), filtered, and concentrated. The crude was partially purified by Biotage Flash 40L silica gel chromatography, eluting with neat EtOAc, 2% MeOH/EtOAc, then 5% MeOH/EtOAc to yield (1′R,3′R)-2-amino-7′-bromo-3′-((tert-butyldimethylsilyloxy)methyl)-1,3′-dimethyl-3′,4′-dihydro-2′H-spiro[imidazole-4,1′-naphthalen]-5(1H)-one (230 mg, 11%).

Step B: (1′R,3′R)-2-Amino-7′-(5-chloropyridin-3-yl)-3′-(hydroxymethyl)-1,3′-dimethyl-3′,4′-dihydro-2′H-spiro[imidazole-4,1′-naphthalen]-5(1H)-one (22 mg, 49%) was prepared from (1′R,3′R)-2-amino-7′-bromo-3′-((tert-butyldimethylsilyloxy)methyl)-1,3′-dimethyl-3′,4′-dihydro-2′H-spiro[imidazole-4,1′-naphthalen]-5(1H)-one according to the procedures described in Example 144, Steps M-N, replacing 3-chloro-5-fluorophenylboronic acid (Step M) with 5-chloropyridin-3-ylboronic acid. 1H NMR (400 MHz, CDCl₃) δ 8.57 (d, J=2 Hz, 1H), 8.50 (d, J=2 Hz, 1H), 7.74 (t, J=2 Hz, 1H), 7.36 (dd, J=2, 8 Hz, 1H), 7.20 (d, J=8 Hz, 1H), 6.89 (d, J=2 Hz, 1H), 4.49 (br s, 3H), 3.54 (d, J=12 Hz, 1H), 3.31 (d, J=12 Hz, 1H), 3.18 (s, 3H), 2.82 (d, J=17 Hz, 1H), 2.67 (d, J=17 Hz, 1H), 2.10 (d, J=14 Hz, 1H), 1.86 (dd, J=2, 14 Hz, 1H), 1.10 (s, 3H); m/z (APCI-pos) M+1=385.

### Example 155

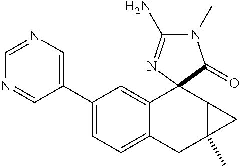

### (2R,7α′S)-2′-amino-1′,7α-dimethyl-4-(pyrimidin-5-yl)-1,1a,7,7α-tetrahydrospiro[cyclopropa[b]naphthalene-2,4′-imidazol]-5′(1′H)-one

Step A: A thick walled glass pressure vessel plus stir bar was charged with 7-bromo-3-(hydroxymethyl)-3-methyl-3,4-dihydronaphthalen-1(2H)-one (2.7 g, 10 mmol; Example 144, Step G), toluene (30 mL), triphenylphosphine (5.3 g, 20 mmol), I₂ (3.8 g, 15 mmol), and imidazole (1.4 g, 20 mmol). The mixture was heated to 90° C. for 1 hour. Then, Cs₂CO₃ (6.5 g, 20 mmol) was added, and the mixture was heated to 90° C. for more 2 hours. After cooling to room temperature, the reaction mixture was portioned between EtOAc (30 mL) and water (30 mL). The phases were separated, and the aqueous phase was re-extracted with EtOAc (20 mL). The combined organic phases were washed with brine (30 mL), dried (MgSO₄), filtered, and concentrated. The crude was purified by silica gel chromatography on a Biotage Flash 65 system, eluting with 10% EtOAc/hexanes, followed by 20% EtOAc/hexanes to yield 4-bromo-7α-methyl-7,7α-dihydro-1H-cyclopropa[b]naphthalen-2(1aH)-one (2.0 g, 80%).

Step B: Intermediate (2′R,7α′S)-4-bromo-1′,7α-dimethyl-1,1a,7,7α-tetrahydrospiro[cyclopropa[b]naphthalene-2,4′-imidazolidine]-2′,5′-dione was prepared according to the procedures described in Example 144, Steps I-J, replacing 7-bromo-3-((tert-butyldimethylsilyloxy)methyl)-3-methyl-3,4-dihydronaphthalen-1(2H)-one with 4-bromo-7α-methyl-7,7α-dihydro-1H-cyclopropa[b]naphthalen-2(1aH)-one (Step I). (2′R,7α′S)-4-Bromo-1′,7α-dimethyl-1,1a,7,7α-tetrahydrospiro[cyclopropa[b]naphthalene-2,4′-imidazolidine]-2′,5′-dione was separated from its diastereomer (2′R,7α′R)-4-bromo-1′,7α-dimethyl-1,1a,7,7α-tetrahydrospiro[cyclopropa[b]naphthalene-2,4′-imidazolidine]-2′,5′-dione by Biotage Flash 40L silica gel chromatography, eluting with a gradient of 10%-30% EtOAc/hexanes to yield (2′R,7α′S)-4-bromo-1′,7α-dimethyl-1,1a,7,7α-tetrahydrospiro[cyclopropa[b]naphthalene-2,4′-imidazolidine]-2′,5′-dione (400 mg, 21%). Stereochemistry arbitrarily assigned.

Step C: A thick walled, glass pressure vessel plus stir bar was charged with (2′R,7α′S)-4-bromo-1′,7α-dimethyl-1,1a,7,7α-tetrahydrospiro[cyclopropa[b]naphthalene-2,4′-imidazolidine]-2′,5′-dione (400 mg, 1.19 mmol), Lawesson's Reagent (290 mg, 0.716 mmol), and toluene (3 mL). The mixture was degassed with N₂ for several minutes and then heated to 100° C. for 18 hours with stirring. After cooling to room temperature, the mixture was partitioned between EtOAc (10 mL) and saturated aqueous NaHCO₃ (10 mL). The phases were separated, and the aqueous phase was re-extracted with EtOAc (10 mL). The combined organic phases were washed with brine (10 mL), dried (MgSO₄), filtered, and concentrated. Purified crude by Biotage Flash 40 silica gel chromatography, eluting with 5% EtOAc/hexanes, then 10% EtOAc/hexanes to yield (2′R,7α′S)-4-bromo-1′,7α-dimethyl-2′-thioxo-1,1a,7,7α-tetrahydrospiro[cyclopropa[b]naphthalene-2,4′-imidazolidin]-5′-one (130 mg, 12%).

Step D: A round bottomed flask plus stir bar was charged with (2′R,7α′S)-4-bromo-1′,7α-dimethyl-2′-thioxo-1,1a,7,7α-tetrahydrospiro[cyclopropa[b]naphthalene-2,4′-imidazolidin]-5′-one (130 mg, 0.370 mmol), MeOH (1.5 mL), t-butyl hydroperoxide 70% aqueous (0.36 mL, 2.6 mmol), and 30% aqueous NH₄OH (0.72 mL, 5.6 mmol). The mixture was heated to 50° C. for 2 hours with stirring. After cooling to room temperature, the mixture was diluted with water (2 mL) and concentrated in vacuo. The mixture was diluted with EtOAc (15 mL), and the phases were separated. The aqueous phase was re-extracted with EtOAc (2×5 mL). The combined organic phases were washed with brine (15 mL), dried (MgSO₄), filtered, and concentrated. The crude was partially purified by preparative TLC (1 mm thickness; R_{f}=0.48), eluting with 10% MeOH/DCM to yield (2′R,7α′S)-2′-amino-4-bromo-1′,7α-dimethyl-1,1a,7,7α-tetrahydrospiro[cyclopropa[b]naphthalene-2,4′-imidazol]-5′(1′H)-one (6 mg, 2%).

Step E: A vial charged with (2′R,7α′S)-2′-amino-4-bromo-1′,7α-dimethyl-1,1a,7,7α-tetrahydrospiro[cyclopropa[b]naphthalene-2,4′-imidazol]-5′(1′H)-one (6 mg, 0.018 mmol), dioxane (0.2 mL), pyrimidin-5-ylboronic acid (3.3 mg, 0.027 mmol), Pd(PPh₃)₄ (2.1 mg, 0.0018 mmol), and 2N aqueous Na₂CO₃ (27 μL, 0.054 mmol). The mixture was sparged with N₂ for 1 minute and then heated to 90° C. for 2 hours with stirring. After cooling to room temperature, the reaction mixture was loaded directly on to a preparative TLC plate (0.5 mm thickness, R_{f}=0.39), eluting with 10% MeOH (containing 7N NH₃)/DCM to yield (2′R,7α′S)-2′-amino-1′,7α-dimethyl-4-(pyrimidin-5-yl)-1,1a,7,7α-tetrahydrospiro[cyclopropa[b]naphthalene-2,4′-imidazol]-5′(1′H)-one (1 mg, 16%). m/z (APCI-pos) M+1=334.

### Example 156

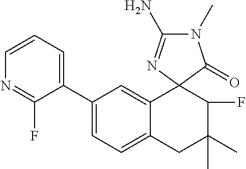

### 2-amino-2′-fluoro-7′-(2-fluoropyridin-3-yl)-1,3′,3′-trimethyl-3′,4′-dihydro-2′H-spiro[imidazole-4,1′-naphthalen]-5(1H)-one

Step A: A round bottomed flask plus stir bar was charged with 7-bromo-3,3-dimethyl-3,4-dihydronaphthalen-1(2H)-one (2.9 g, 11 mmol; Example 2, Step D), toluene (10 mL), racemic 1-phenylethanamine (2.1 g, 17 mmol), and lastly 3 drops of TFA. The reaction mixture was refluxed and stirred with azeotropic removal of water using a Dean-Stark apparatus (5 mL capacity) for 18 hours. More racemic 1-phenylethanamine (0.5 equivalents) was added, as well as 2 more drops of TFA. The mixture was heated for another day at reflux with the attached Dean Stark trap. After cooling to room temperature, the reaction mixture was eluted down a plug of silica gel (150 mL) with 20% EtOAc/hexanes to obtain (E)-N-(7-bromo-3,3-dimethyl-3,4-dihydronaphthalen-1(2H)-ylidene)-1-phenylethanamine (3.8 g, 82%).

Step B: A thick walled glass pressure tube plus stir bar was charged with (E)-N-(7-bromo-3,3-dimethyl-3,4-dihydronaphthalen-1(2H)-ylidene)-1-phenylethanamine (3.8 g, 10.7 mmol), anhydrous MeOH (50 mL), and Selectfluor (4.16 g, 11.7 mmol). The reaction mixture was heated to 60° C. for 15 hours with stirring. The mixture was cooled to room temperature, and concentrated aqueous HCl (5 mL) was added. Solids were observed. The mixture was stirred for 15 minutes at room temperature. The mixture was concentrated. The mixture was diluted with DCM (50 mL) and aqueous 1N HCl (50 mL). The phases were separated. The organic phase was washed again with aqueous 1N HCl (50 mL) and then with saturated aqueous NaHCO₃ (50 mL). The organic phase was dried (Na₂SO₄), filtered, and concentrated to obtain crude material (3 g). The crude was partially separated by Biotage Flash 40L, eluting with 3% EtOAc/hexanes then 5% EtOAc/hexanes to yield 7-bromo-2,2-difluoro-3,3-dimethyl-3,4-dihydronaphthalen-1(2H)-one (1.8 g, 37%, contaminated with a by-product that was removed at a subsequent step in the synthesis) and 7-bromo-2-fluoro-3,3-dimethyl-3,4-dihydronaphthalen-1(2H)-one (800 mg, 26%).

Step C: Intermediate T-bromo-2′-fluoro-1,3′,3′-trimethyl-3′,4′-dihydro-2′H-spiro[imidazolidine-4,1′-naphthalene]-2,5-dione (900 mg, 91%) was prepared according to the procedure described for Example 144, Steps I-J, replacing 7-bromo-3-((tert-butyldimethylsilyloxy)methyl)-3-methyl-3,4-dihydronaphthalen-1(2H)-one with 7-bromo-2-fluoro-3,3-dimethyl-3,4-dihydronaphthalen-1(2H)-one.

Step D: 2-Amino-2′-fluoro-7′-(2-fluoropyridin-3-yl)-1,3′,3′-trimethyl-3′,4′-dihydro-2′H-spiro[imidazole-4,1′-naphthalen]-5(1H)-one (77 mg, 62%) was prepared according to the procedure described for Example 155, Steps C-E, replacing (2′R,7α′S)-4-bromo-1′,7α-dimethyl-1,1a,7,7α-tetrahydrospiro[cyclopropa[b]naphthalene-2,4′-imidazolidine]-2′,5′-dione with 7′-bromo-2′-fluoro-1,3′,3′-trimethyl-3′,4′-dihydro-2′H-spiro[imidazolidine-4,1′-naphthalene]-2,5-dione (Step C) and replacing pyrimidin-5-ylboronic acid with 2-fluoropyridin-3-ylboronic acid (Step E). ¹H NMR (400 MHz, CDCl₃+MeOD) δ 8.15 (m, 1H), 7.83 (m, 1H), 7.42 (m, 1H), 7.31 (m, 1H), 7.22 (d, J=8 Hz, 1H), 7.03 (br s, 1H), 4.89 (d, J=47 Hz, 1H), 3.19 (s, 3H), 2.93 (d, J=17 Hz, 1H), 2.79 (dd, J=8, 17 Hz, 1H), 1.26 (s, 3H), 1.10 (s, 3H); m/z (APCI-pos) M+1=371.

### Example 157

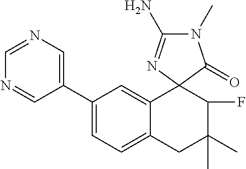

### 2-amino-2′-fluoro-1,3′,3′-trimethyl-7′-(pyrimidin-5-yl)-3′,4′-dihydro-2′H-spiro[imidazole-4,1′-naphthalen]-5(1H)-one

2-Amino-2′-fluoro-1,3′,3′-trimethyl-7′-(pyrimidin-5-yl)-3′,4′-dihydro-2′H-spiro[imidazole-4,1′-naphthalen]-5(1H)-one (84 mg, 68%) was prepared according to the procedure described for Example 156, replacing 2-fluoropyridin-3-ylboronic acid (Step D) with pyrimidin-5-ylboronic acid. ¹H NMR (400 MHz, CDCl₃+MeOD) δ 9.15 (s, 1H), 8.88 (s, 2H), 7.49 (m, 1H), 7.30 (d, J=8 Hz, 1H), 7.04 (s, 1H), 4.88 (d, J=47 Hz, 1H), 3.22 (s, 3H), 2.95 (d, J=17 Hz, 1H), 2.82 (dd, J=8, 17 Hz, 1H), 1.27 (s, 3H), 1.10 (s, 3H); m/z (APCI-pos) M+1=354.

### Example 158

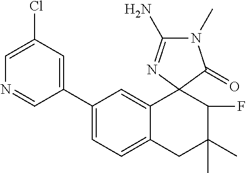

### 2-amino-7′-(5-chloropyridin-3-yl)-2′-fluoro-1,3′,3′-trimethyl-3′,4′-dihydro-2′H-spiro[imidazole-4,1′-naphthalen]-5(1H)-one

2-Amino-7′-(5-chloropyridin-3-yl)-2′-fluoro-1,3′,3′-trimethyl-3′,4′-dihydro-2′H-spiro[imidazole-4,1′-naphthalen]-5(1H)-one (61 mg, 43%) was prepared according to the procedure described for Example 156, replacing 2-fluoropyridin-3-ylboronic acid (Step D) with 5-chloropyridin-3-ylboronic acid. ¹H NMR (400 MHz, CDCl₃+MeOD) δ 8.56 (m, 1H), 8.50 (m, 1H), 7.83 (m, 1H), 7.44 (m, 1H), 7.25 (d, J=8 Hz, 1H), 7.00 (s, 1H), 4.88 (d, J=47 Hz, 1H), 3.22 (s, 3H), 2.94 (d, J=17 Hz, 1H), 2.80 (dd, J=8, 17 Hz, 1H), 1.26 (s, 3H), 1.10 (s, 3H); m/z (APCI-pos) M+1=387.

### Example 159

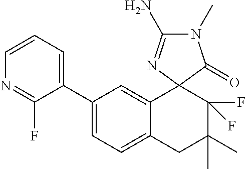

### 2-amino-2′,2′-difluoro-7′-(2-fluoropyridin-3-yl)-1,3′,3′-trimethyl-3′,4′-dihydro-TH-spiro[imidazole-4,1′-naphthalen]-5(1H)-one

Step A: Intermediate T-bromo-2′,2′-difluoro-1,3′,3′-trimethyl-3′,4′-dihydro-2′H-spiro[imidazolidine-4,1′-naphthalene]-2,5-dione was prepared according to the procedure described for Example 144, Steps I-J, replacing 7-bromo-3-((tert-butyldimethylsilyloxy)methyl)-3-methyl-3,4-dihydronaphthalen-1(2H)-one with 7-bromo-2,2-difluoro-3,3-dimethyl-3,4-dihydronaphthalen-1(2H)-one (Example 156, Step B). The crude was purified by Biotage Flash 40 silica gel chromatography, eluting with 10% EtOAc/hexanes followed by 20% EtOAc/hexanes to yield T-bromo-2′,2′-difluoro-1,3′,3′-trimethyl-3′,4′-dihydro-2′H-spiro[imidazolidine-4,1′-naphthalene]-2,5-dione (560 mg, 44%).

Step B: 2-Amino-2′,2′-difluoro-7′-(2-fluoropyridin-3-yl)-1,3′,3′-trimethyl-3′,4′-dihydro-2′H-spiro[imidazole-4,1′-naphthalen]-5(1H)-one (53 mg, 43%) was prepared according to the procedure described for Example 155, Steps C-E, replacing (2′R,7α′S)-4-bromo-1′,7α-dimethyl-1,1a,7,7α-tetrahydrospiro[cyclopropa[b]naphthalene-2,4′-imidazolidine]-2′,5′-dione with 7′-bromo-2′,2′-difluoro-1,3′,3′-trimethyl-3′,4′-dihydro-2′H-spiro[imidazolidine-4,1′-naphthalene]-2,5-dione (Step C) and replacing pyrimidin-5-ylboronic acid with 2-fluoropyridin-3-ylboronic acid (Step E). m/z (APCI-pos) M+1=389.

### Example 160

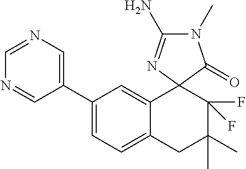

### 2-amino-2′,2′-difluoro-1,3′,3′-trimethyl-7′-(pyrimidin-5-yl)-3′,4′-dihydro-2′H-spiro[imidazole-4,1′-naphthalen]-5(1H)-one

2-Amino-2′,2′-difluoro-1,3′,3′-trimethyl-7′-(pyrimidin-5-yl)-3′,4′-dihydro-2′H-spiro[imidazole-4,1′-naphthalen]-5(1H)-one (73 mg, 61%) was prepared according to the procedure for Example 159, replacing 2-fluoropyridin-3-ylboronic acid with pyrimidin-5-ylboronic acid (Step B). m/z (APCI-pos) M+1=372.

### Example 161

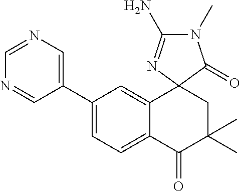

### 2-amino-1,3′,3′-trimethyl-7′-(pyrimidin-5-yl)-2′H-spiro[imidazole-4,1′-naphthalene]-4′,5(1H,3′H)-dione

Step A: A solution of 2-amino-7′-bromo-1,3′,3′-trimethyl-3′,4′-dihydro-2′H-spiro[imidazole-4,1′-naphthalen]-5(1H)-one (170 mg, 0.506 mmol) in DCM (2.5 mL) was prepared, CrO₃ (10.1 mg, 0.101 mmol) was added, and t-butyl hydroperoxide (70% aqueous, 1.3 mL, 0.506 mmol, 1 equivalent) was added every 4 hours until the reaction was complete by LCMS. The mixture was diluted with ethyl acetate, and the organic layer was washed thoroughly with water (3×), brine, dried, and concentrated to afford a crude material that was purified by semi-preparative C18 HPLC eluting with 5-95% ACN/H₂O+0.1% TFA to afford 2-amino-7′-bromo-1,3′,3′-trimethyl-2′H-spiro[imidazole-4,1′-naphthalene]-4′,5(1H,3′H)-dione (150 mg, 0.428 mmol, 85%).

Step B: A solution of 2-amino-7′-bromo-1,3′,3′-trimethyl-2′H-spiro[imidazole-4,1′-naphthalene]-4′,5(1H,3′H)-dione 2,2,2-trifluoroacetate (26 mg, 0.0560 mmol), pyrimidin-5-ylboronic acid (9.72 mg, 0.0784 mmol), Pd(PPh₃)₄ (6.47 mg, 0.00560 mmol), Na₂CO₃ (112 μL, 0.224 mmol; 2M aqueous) in dioxane (280 μL, 0.0560 mmol) was degassed with nitrogen for 5 minutes, sealed in a vial and stirred at 80° C. for 1 day. The reaction mixture was filtered, and the filtrate was purified by C18 semi-prep HPLC eluting with 5-95% ACN/H₂O+0.1% TFA. The product containing fractions was concentrated in vacuo to afford 2-amino-1,3′,3′-trimethyl-7′-(pyrimidin-5-yl)-2′H-spiro[imidazole-4,1′-naphthalene]-4′,5(1H,3′H)-dione 2,2,2-trifluoroacetate (22 mg, 0.0475 mmol, 85%). m/z 350.1 (100%), 351.1 (20%).

### Example 162

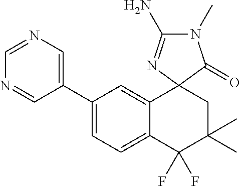

### 2-amino-4′,4′-difluoro-1,3′,3′-trimethyl-7′-(pyrimidin-5-yl)-3′,4′-dihydro-2′H-spiro[imidazole-4,1′-naphthalen]-5(1H)-one

Step A: Deoxofluor (20.3 μL, 0.110 mmol) was added to a solution in a plastic tube of 2-amino-7′-bromo-1,3′,3′-trimethyl-2′H-spiro[imidazole-4,1′-naphthalene]-4′,5(1H,3′H)-dione 2,2,2-trifluoroacetate (17 mg, 0.0366 mmol) in DCE (183 μL, 0.0366 mmol) at 0° C., and the resulting mixture was stirred at 0° C. for 15 minutes while warming to room temperature. The reaction mixture was concentrated, and the residue was purified by C18 semi-prep HPLC eluting with 5-95% ACN/H₂O+0.1% TFA. The product containing fractions was concentrated in vacuo to afford 2-amino-7′-bromo-4′,4′-difluoro-1,3′,3′-trimethyl-3′,4′-dihydro-2′H-spiro[imidazole-4,1′-naphthalen]-5(1H)-one 2,2,2-trifluoroacetate (3 mg, 6.2 μmol, 17%).

Step B: A solution of 2-amino-7′-bromo-4′,4′-difluoro-1,3′,3′-trimethyl-3′,4′-dihydro-2′H-spiro[imidazole-4,1′-naphthalen]-5(1H)-one 2,2,2-trifluoroacetate (3 mg, 0.00617 mmol), pyrimidin-5-ylboronic acid (1.07 mg, 0.00864 mmol), Pd(PPh₃)₄ (0.713 mg, 0.000617 mmol), Na₂CO₃ (12.3 μL, 0.0247 mmol; 2M aqueous) in dioxane (30.8 μL, 0.00617 mmol) was degassed with nitrogen for 1 minute, sealed in a vial and stirred at 80° C. for 1 day. The reaction mixture was filtered and purified by C18 semi-prep HPLC eluting with 5-95% ACN/H₂O+0.1% TFA. The product containing fractions were concentrated in vacuo to afford 2-amino-4′,4′-difluoro-1,3′,3′-trimethyl-7′-(pyrimidin-5-yl)-3′,4′-dihydro-2′H-spiro[imidazole-4,1′-naphthalen]-5(1H)-one 2,2,2-trifluoroacetate (2.6 mg, 0.00536 mmol, 87%). m/z 372.1 (40%), 352.1 (100%).

### Example 163

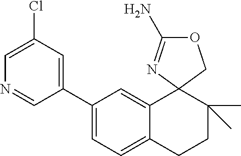

### 7-(5-chloropyridin-3-yl)-2,2-dimethyl-3,4-dihydro-2H,5′H-spiro[naphthalene-1,4′-oxazol]-2′-amine

Step A: Potassium tert-butoxide (39.88 g, 355.4 mmol) was added to a solution of 7-bromo-3,4-dihydronaphthalen-1(2H)-one (20.0 g, 88.8 mmol) in tetrahydrofuran (180 mL). The resulting brown suspension was heated to reflux for 6 hours and then cooled to room temperature. Iodomethane (44.36 mL, 710 mmol) was added dropwise by addition funnel over 15 minutes, and the reaction mixture was heated to 50° C. for 3 hours and then stirred at room temperature overnight. The reaction mixture was then cooled to 0° C., water was added, and the mixture was extracted with ethyl acetate. The combined extracts were dried anhydrous sodium sulfate, filtered, and concentrated. Purification by flash column chromatography provided 7-bromo-2,2-dimethyl-3,4-dihydronaphthalen-1(2H)-one (21.4 g, 95%). ¹H NMR (400 MHz, (CD₃)₂SO) δ 7.92 (d, J=2.0 Hz, 1H), 7.73 (dd, J=2.4, 8.4 Hz, 1H), 7.33 (d, J=8.0 Hz, 1H), 2.93 (t, J=6.4 Hz, 2H), 1.93 (t, J=6.4 Hz, 2H), 1.11 (s, 6H).

Step B: CH₃PPh₃Br (14.1 g, 39.5 mmol) and lithium bis(trimethylsilyl)amide (31.6 mL, 31.6 mmol, 1.0M) was added to a solution of 7-bromo-2,2-dimethyl-3,4-dihydronaphthalen-1(2H)-one (4.0 g, 16 mmol) in toluene (200 mL). The resulting mixture was heated to 80° C. After 1 hour, the reaction was quenched with NaHCO₃ and diluted with dichloromethane. The organic extract was washed with saturated aqueous sodium chloride solution. The collected organic was dried over anhydrous sodium sulfate, filtered, and concentrated. Purification by flash column chromatography afforded 7-bromo-2,2-dimethyl-1-methylene-1,2,3,4-tetrahydronaphthalene (3.7 g, 93%).

Step C: A solution of iodine (4.15 g, 16.3 mmol) in ethyl aceate (176 mL) was added to an ice-cooled solution of 7-bromo-2,2-dimethyl-1-methylene-1,2,3,4-tetrahydronaphthalene (3.7 g, 14.9 mmol) and silver cyanate (2.67 g, 17.8 mmol) in acetonitrile (82 mL)/ethyl acetate (74 mL). After 5 minutes, the reaction mixture was warmed to room temperature for 2 hours. The solids were filtered and rinsed with ethyl acetate. The filtrate was concentrated, and the resulting residue was dissolved in tetrahydrofuran (195 mL). Ammonium hydroxide (39 mL) was added, and the resulting reaction mixture was maintained at room temperature overnight. Saturated aqueous sodium bicarbonate was added, and the resulting mixture was extracted with dichloromethane. The collected extracts was washed with saturated aqueous sodium chloride solution, dried over anhydrous sodium sulfate, filtered, and concentrated. The resulting solid was triturated with petroleum ether to give 7-bromo-2,2-dimethyl-3,4-dihydro-2H,5′H-spiro[naphthalene-1,4′-oxazol]-2′-amine (3.2 g, 70%). ¹H NMR (400 MHz, CD₃OD) δ 7.46 (d, J=2.4 Hz, 1H), 7.27 (dd, J=2.0, 8.0 Hz, 1H), 7.00 (d, J=8.4 Hz, 1H), 4.55 (d, J=8.8 Hz, 1H), 3.86 (t, J=8.8 Hz, 1H), 2.85-2.68 (m, 2H), 1.76-1.64 (m, 2H), 0.92 (s, 3H), 0.88 (s, 3H).

Step D: A solution of 7-bromo-2,2-dimethyl-3,4-dihydro-2H,5′H-spiro[naphthalene-1,4′-oxazol]-2′-amine (170 mg, 0.55 mmol), 5-chloropyridin-3-ylboronic acid (156 mg, 0.99 mmol), Pd(PPh₃)₂Cl₂ (39 mg, 0.055 mmol) and sodium carbonate (117 mg, 1.1 mmol) in dioxane (4 mL) and water (1.6 mL) was stirred at 100° C. After 2 hours, water was added, and the resulting mixture was extracted with ethyl acetate. The collected organic extracts were concentrated. Purification by preparative high performance liquid chromatography provided 7-(5-chloropyridin-3-yl)-2,2-dimethyl-3,4-dihydro-2H,5′H-spiro[naphthalene-1,4′-oxazol]-2′-amine (60.6 mg, 32.2%). ¹H NMR (400 MHz, CD₃OD) δ 8.78 (d, J=1.6 Hz, 1H), 8.57 (d, J=2.0 Hz, 1H), 8.21 (t, J=2.0 Hz, 1H), 7.80 (d, J=1.6 Hz, 1H), 7.65 (dd, J=2.0, 8.0 Hz, 1H), 7.37 (d, J=8.0 Hz, 1H), 5.12 (d, J=9.2 Hz, 1H), 4.55 (d, J=9.6 Hz, 1H), 3.33-2.88 (m, 2H), 1.98-1.90 (m, 1H), 1.83-1.77 (m, 1H), 1.12 (s, 3H), 1.04 (s, 3H); LCMS (ESI): [MH]+=341.8.

### Example 164

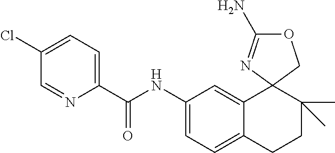

### N-(2′-amino-2,2-dimethyl-3,4-dihydro-2H,5′H-spiro[naphthalene-1,4′-oxazole]-7-yl)-5-chloropicolinamide

A solution of 7-bromo-2,2-dimethyl-3,4-dihydro-2H,5′H-spiro[naphthalene-1,4′-oxazol]-2′-amine (0.20 g, 0.65 mmol), 5-chloropicolinamide (152 mg, 0.97 mmol), copper (I) iodide (123 mg, 0.65 mmol), potassium carbonate (268 mg, 1.94 mmol) and N,N′-dimethylethylenediamine (57 mg, 0.65 mmol) in dioxane (5 mL) was stirred at 100° C. under N₂ overnight. The mixture was diluted with methanol and filtered. The fitrate was concentrated and purified by preparative high performance liquid chromatography to afford N-(2′-amino-2,2-dimethyl-3,4-dihydro-2H,5′H-spiro[naphthalene-1,4′-oxazole]-7-yl)-5-chloropicolinamide (35.4 mg, 14%). ¹H NMR (400 MHz, CD₃OD) δ 9.78 (s, 1H), 8.53 (d, J=2.0 Hz, 1H), 8.23 (d, J=8.4 Hz, 1H), 7.86-7.81 (m, 2H), 7.47 (d, J=1.6 Hz, 1H), 7.10 (d, J=8.0 Hz, 1H), 4.50-4.43 (m, 1H), 4.00 (d, J=8.8 Hz, 1H), 2.90-2.71 (m, 2H), 1.75-1.66 (m, 2H), 0.95 (s, 6H); LCMS (ESI): {MH}+=385.0.

### Example 165

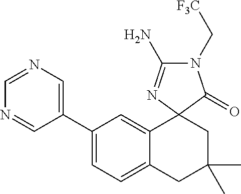

### 2-amino-3′,3′-dimethyl-7′-(pyrimidin-5-yl)-1-(2,2,2-trifluoroethyl)-3′,4′-dihydro-2′H-spiro[imidazole-4,1′-naphthalen]-5(1H)-one

Step A: Cesium carbonate (855 mg, 2.62 mmol) and trifluoroethanol triflate (0.189 mL, 1.31 mmol) were added to a solution of 7′-bromo-3′,3′-dimethyl-3′,4′-dihydro-2′H-spiro[imidazolidine-4,1′-naphthalene]-2,5-dione (424 mg, 1.31 mmol) in DMF (12.2 mL), and the reaction was allowed to stir overnight. The reaction mixture was diluted with ethyl acetate and washed with saturated sodium chloride. The organic layer was dried over anhydrous sodium sulfate, filtered, and concentrated. The crude mixture was purified by silica gel chromatography eluting with a linear gradient of 0-50% ethyl acetate/heptane to yield 7′-bromo-3′,3′-dimethyl-1-(2,2,2-trifluoroethyl)-3′,4′-dihydro-2′H-spiro[imidazolidine-4,1′-naphthalene]-2,5-dione (525 mg, 1.30 mmol, 98.8% yield).

Step B: Lawesson's Reagent (305 mg, 0.755 mmol) was added to a solution of 7′-bromo-3′,3′-dimethyl-1-(2,2,2-trifluoroethyl)-3′,4′-dihydro-2′H-spiro[imidazolidine-4,1′-naphthalene]-2,5-dione (510 mg, 1.26 mmol) in 1,4-dioxane (12.8 mL), and the reaction was heated to reflux overnight. The reaction mixture was cooled to room temperature, concentrated, and purified by silica gel chromatography eluting with a linear gradient of 0-40% ethyl acetate/heptane to yield 7′-bromo-3′,3′-dimethyl-2-thioxo-1-(2,2,2-trifluoroethyl)-3′,4′-dihydro-2′H-spiro[imidazolidine-4,1′-naphthalen]-5-one (135 mg, 0.320 mmol, 26% yield).

Step C: 30% Aqueous ammonium hydroxide (2.50 mL, 19.2 mmol) and t-butyl hydroperoxide 70% aqueous (0.878 mL, 6.41 mmol) were added to a solution of 7′-bromo-3′,3′-dimethyl-2-thioxo-1-(2,2,2-trifluoroethyl)-3′,4′-dihydro-2′H-spiro[imidazolidine-4,1′-naphthalen]-5-one (135 mg, 0.320 mmol) in methanol (6.5 mL), and the reaction was stirred at 40° C. overnight. The reaction mixture was diluted with dichloromethane and washed with saturated sodium chloride. The organic layer was dried over anhydrous sodium sulfate, filtered, and concentrated. The crude material was purified by silica gel chromatography eluting with a linear gradient of 0-6% dichloromethane/methanol+1% NH₄OH to yield 2-amino-7′-bromo-3′,3′-dimethyl-1-(2,2,2-trifluoroethyl)-3′,4′-dihydro-2′H-spiro[imidazole-4,1′-naphthalen]-5(1H)-one (89.0 mg, 0.220 mmol, 69% yield).

Step D: A vial was charged with 2-amino-7′-bromo-3′,3′-dimethyl-1-(2,2,2-trifluoroethyl)-3′,4′-dihydro-2′H-spiro[imidazole-4,1′-naphthalen]-5(1H)-one (25.5 mg, 0.063 mmol), 1,4-dioxane (0.49 mL), water (0.23 mL), pyrimidin-5-ylboronic acid (10.2 mg, 0.082 mmol), Pd(PPh₃)₄ (3.6 mg, 0.0032 mmol) and sodium carbonate (20 mg, 0.189 mmol). The mixture was sparged with N₂ for 1 minute and then heated to 80° C. for 1 hour with stirring. After cooling to room temperature, the reaction mixture was diluted with dichloromethane and washed with sodium chloride. The organic layer was dried over anhydrous sodium sulfate, filtered, and concentrated. The crude material was partially purified by silica gel chromatography eluting with a linear gradient of 0-6% dichloromethane/methanol+1% NH₄OH. The desired product was further purified by semi-preparative C18 HPLC eluting with 5-95% acetonitrile/water+0.1% NH₄OH to afford 2-amino-3′,3′-dimethyl-7′-(pyrimidin-5-yl)-1-(2,2,2-trifluoroethyl)-3′,4′-dihydro-2′H-spiro[imidazole-4,1′-naphthalen]-5(1H)-one (12 mg, 0.030 mmol, 47%). ¹H NMR (400 MHz, (CD₃)₂SO) δ 9.16 (s, 1H), 8.93 (s, 1H), 7.63 (d, J=8 Hz, 1H), 7.29 (d, J=8 Hz, 1H), 7.03 (s, 1H), 6.63 (s, 2H), 4.47 (q, J=8 Hz, 2H), 2.70 (d, J=16 Hz, 1H), 2.62 (d, J=16 Hz, 1H), 2.03 (d, J=12 Hz, 1H), 1.61 (d, J=12 Hz, 1H), 1.10 (s, 3H), 0.99 (s, 3H); m/z (APCI-pos) M+1=404.2.

### Example 166

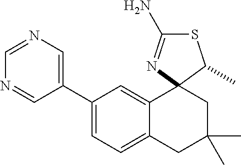

### 3,3,5′-trimethyl-7-(pyrimidin-5-yl)-3,4-dihydro-2H,5′H-spiro[naphthalene-1,4′-thiazol]-2′-amine

Step A: A round bottom flask was charged with 7-bromo-3,3-dimethyl-3,4-dihydronaphthalen-1(2H)-one (2.66 g, 10.5 mmol), 1,4-dioxane (32.8 mL), water (15.1 mL), pyrimidin-5-ylboronic acid (1.43 g, 11.6 mmol), Pd(PPh₃)₄ (607 mg, 0.525 mmol) and sodium carbonate (3.34 g, 31.5 mmol). The mixture was sparged with N₂ for 1 minute and then heated to 100° C. for 3 hours with stirring. After cooling to room temperature, the reaction mixture was diluted with dichloromethane and washed with saturated aqueous sodium chloride. The organic layer was dried over anhydrous sodium sulfate, filtered, and concentrated. The crude material was purified by silica gel chromatography eluting with a linear gradient of 0-70% heptane/ethyl acetate to yield 3,3-dimethyl-7-(pyrimidin-5-yl)-3,4-dihydronaphthalen-1(2H)-one (1.80 g, 7.13 mmol, 68% yield).

Step B: Ethyltriphenylphosphonium bromide (1.92 g, 5.18 mmol) and potassium bis(trimethylsilyl)amide (862 mg, 4.32 mmol) were added to a solution of 3,3-dimethyl-7-(pyrimidin-5-yl)-3,4-dihydronaphthalen-1(2H)-one (436 mg, 1.73 mmol) in toluene (22 mL), and the reaction was heated to 80° C. for 1.5 hours. After cooling to room temperature, the reaction mixture was diluted with saturated aqueous sodium bicarbonate and dichloromethane, followed by washing the organic layer with saturated aqueous sodium chloride. The organic layer was dried over anhydrous sodium sulfate, filtered, and concentrated. The crude material was purified by silica gel chromatography eluting with a linear gradient of 0-40% heptane/ethyl acetate to yield 5-(8-ethylidene-6,6-dimethyl-5,6,7,8-tetrahydronaphthalen-2-yl)pyrimidine (350 mg, 1.32 mmol, 77% yield) as a 10:1 mixture of olefin isomers (unassigned).

Step C: Silver thiocyanate (69.3 mg, 0.418 mmol) was added to a stirred solution of 5-(8-ethylidene-6,6-dimethyl-5,6,7,8-tetrahydronaphthalen-2-yl)pyrimidine (92.0 mg, 0.348 mmol) in ethyl acetate (0.40 mL) and acetonitrile (0.92 mL) cooled to 0° C. under N₂. In a separate flask, iodine (97.2 mg, 0.383 mmol) was dissolved in ethyl acetate (2.6 mL). This solution was added via syringe to the alkene-containing solution at 0° C. over 5 minutes. The reaction mixture was then removed from the ice bath and allowed to stir at room temperature for 1.5 hours. The reaction mixture was filtered through Celite®, rinsing with ethyl acetate, and the filtrate was concentrated. The residue was dissolved in acetone (4 mL) and aqueous NH₄OH (4 mL) was added. The resulting mixture was stirred at 50° C. for 1 hour. The reaction mixture was partitioned between dichloromethane and saturated sodium bicarbonate. After shaking and then separating the phases, the organic layer was washed with saturated aqueous sodium chloride, dried over anhydrous sodium sulfate, filtered, and concentrated. The crude material was partially purified by silica gel chromatography eluting with a linear gradient of 0-6% dichloromethane/methanol+1% NH₄OH. The desired product was further purified by semi-preparative C18 HPLC eluting with 5-95% acetonitrile/water+0.1% NH₄OH to afford 3,3,5′-trimethyl-7-(pyrimidin-5-yl)-3,4-dihydro-2H,5′H-spiro[naphthalene-1,4′-thiazol]-2′-amine (20.1 mg, 0.059 mmol, 17%) as a single diastereomer. The relative stereochemistry was assigned by nOe experiments (methyl to aromatic ring). 1H NMR (400 MHz, (CD₃)₂SO) δ 9.17 (s, 1H), 9.01 (s, 1H), 7.92 (s, 1H), 7.59 (d, J=8 Hz, 1H), 7.27 (d, J=8 Hz, 1H), 6.37 (s, 2H), 3.99 (q, J=8 Hz, 1H), 2.65 (d, J=16 Hz, 1H), 2.46 (d, J=16 Hz, 1H), 1.95 (d, J=16 Hz, 1H), 1.49 (d, J=12 Hz, 1H), 1.16 (s, 3H), 0.81 (s, 3H), 0.79 (s, 3H); m/z (APCI-pos) M+1=339.1.

### Example 167

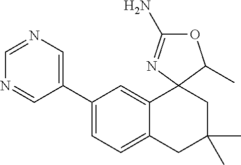

### 3,3,5′-trimethyl-7-(pyrimidin-5-yl)-3,4-dihydro-2H,5′H-spiro[naphthalene-1,4′-oxazol]-2′-amine

Silver cyanate (68.0 mg, 0.454 mmol) was added to a stirred solution of 5-(8-ethylidene-6,6-dimethyl-5,6,7,8-tetrahydronaphthalen-2-yl)pyrimidine (100.0 mg, 0.378 mmol) in ethyl acetate (0.50 mL) and acetonitrile (1.0 mL) cooled to 0° C. under N₂. In a separate flask, iodine (106 mg, 0.416 mmol) was dissolved in ethyl acetate (2.8 mL). This solution was added via syringe to the alkene-containing solution at 0° C. over 5 minutes. The reaction mixture was then removed from the ice bath and allowed to stir at room temperature for 30 minutes. The reaction mixture was filtered through Celite®, rinsing with ethyl acetate, and the filtrate was concentrated. The residue was dissolved in acetone (9 mL) and aqueous NH₄OH (1.5 mL) was added. The resulting mixture was stirred at room temperature overnight. The reaction mixture was partitioned between dichloromethane and saturated aqueous sodium bicarbonate. After shaking and then separating the phases, the organic layer was washed with saturated aqueous sodium chloride, dried over anhydrous sodium sulfate, filtered, and concentrated. The crude material was purified by silica gel chromatography eluting with a linear gradient of 0-6% dichloromethane/methanol+1% NH₄OH to yield 3,3,5′-trimethyl-7-(pyrimidin-5-yl)-3,4-dihydro-2H,5′H-spiro[naphthalene-1,4′-oxazol]-2′-amine (45 mg, 0.140 mmol, 37%) as a 10:1 mixture of diastereomers. ¹H NMR (major diastereomer, 400 MHz, (CD₃)₂SO) δ 9.17 (s, 1H), 9.01 (s, 1H), 7.56 (s, 1H), 7.54 (d, J=8 Hz, 1H), 7.19 (d, J=8 Hz, 1H), 5.83 (s, 2H), 4.76 (q, J=8 Hz, 1H), 2.54 (m, 2H), 1.88 (d, J=12 Hz, 1H), 1.62 (d, J=12 Hz, 1H), 1.24 (d, J=4 Hz, 3H), 1.04 (s, 3H), 0.97 (s, 3H); (APCI-pos) M+1=323.1.

The following compounds in Table 3 were prepared according to the above procedures using appropriate intermediates.

TABLE 3

| Ex. # | Structure | Name | NMR/MS |
|---|---|---|---|
| 168 | 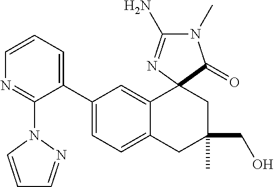 | (1′R,3′S)-7′-(2-(1H-pyrazol- 1-yl)pyridin-3-yl)-2-amino- 3′-(hydroxymethyl)-1,3′- dimethyl-3′,4′-dihydro-2′H- spiro[imidazole-4,1′- naphthalen]-5(1H)-one | m/z (APCI- pos) = 417 (M + 1) |
| 169 | 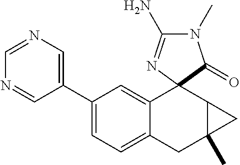 | (2R,7a′R)-2′-amino-1′,7a- dimethyl-4-(pyrimidin-5-yl)- 1,1a,7,7a-tetrahydrospiro [cyclopropa[b]naphthalene- 2,4′-imidazol]-5′(1′H)-one | m/z (APCI- pos) = 334 (M + 1) |
| 170 | 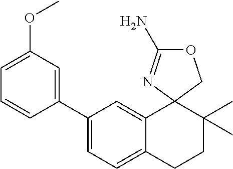 | 7-(3-methoxyphenyl)-2,2- dimethyl-3,4-dihydro- 2H,5′H-spiro[naphthalene- 1,4′-oxazol]-2′-amine | 336.9 |
| 171 | 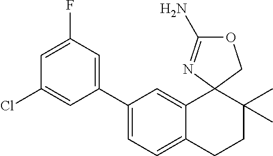 | 7-(3-chloro-5-fluorophenyl)- 2,2-dimethyl-3,4-dihydro- 2H,5′H-spiro[naphthalene- 1,4′-oxazol]-2′-amine | 358.9 |
| 172 | 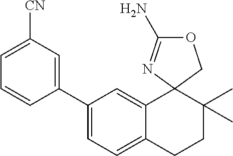 | 3-(2′-amino-2,2-dimethyl- 3,4-dihydro-2H,5′H- spiro[naphthalene-1,4′- oxazole]-7-yl)benzonitrile | 332.1 |
| 173 | 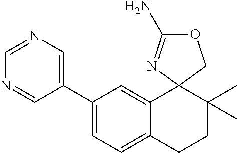 | 2,2-dimethyl-7-(pyrimidin-5- yl)-3,4-dihydro-2H,5′H- spiro[naphthalene-1,4′- oxazol]-2′-amine | 308.9 |

It will be understood that the enumerated embodiments are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

1. A compound selected from Formula a: 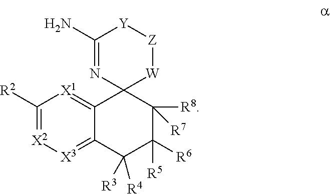
and stereoisomers, diastereomers, enantiomers, tautomers and pharmaceutically acceptable salts thereof, wherein:
W is a bond or CR¹⁰R¹¹;
Y is O, S or NR¹;
Z is CR¹²R¹³ or C(═O), provided when Z is C(═O) then Y is NR¹;
X¹, X² and X³ are independently selected from CR⁹ and N, wherein only one of X¹, X² or
X³ may be N;
R¹ is selected from hydrogen, alkyl, aralkyl, heteroaryl or heteroaralkyl;
R² is selected from hydroxy, halogen, amino, cyano, nitro, alkyl, alkoxy, acyl, acyloxy, alkoxycarbonyl, sulfonyl, sulfinyl, sulfanyl, aryloxy, a carbocycle or a heterocycle wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl, optionally substituted carbocycle or heterocycle;
R³ and R⁴ are independently selected from hydrogen, halogen and alkyl, or
R³ and R⁴ together form an oxo group;
R⁵ and R⁶ are independently hydrogen, hydroxy, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, an optionally substituted carbocycle and an optionally substituted heterocycle, or
R⁵ and R⁶ together form a 3 to 6 member carbocycle or heterocycle optionally substituted with hydroxy, halogen, amino, cyano, nitro, alkyl, alkoxy, acyl and haloalkyl;
R⁷ and R⁸ are independently hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, a carbocycle and an optionally substituted heterocycle, or
R⁷ and R⁸ together form a 3 to 6 member carbocycle or heterocycle optionally substituted with hydroxy, halogen, amino, cyano, nitro, alkyl, alkoxy, acyl and haloalkyl;
R⁹ is independently is hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl and haloalkyl,
or R⁵ and R⁷ together form a 3 to 4 member carbocycle or heterocycle optionally substituted with hydroxy, halogen, amino, cyano, nitro, alkyl, alkoxy, acyl and haloalkyl;
each R⁹ is independently selected from hydrogen, halogen or methyl;
R¹⁰ and R¹¹ are independently selected from hydrogen and alkyl, or
R¹⁰ and R¹¹ together with the atom to which they are attached form a 3 to 6 membered carbocycle or heterocycle; and
R¹² and R¹³ are independently selected from hydrogen, alkyl and a carbocycle.

2. A compound of claim 1, wherein:
W is a bond or CR¹⁰R¹¹;
Y is O, S or NR¹;
Z is CR¹²R¹³ or C(═O), provided when Z is C(═O) then Y is NR¹;
X¹, X² and X³ are independently selected from CR⁹ and N, wherein only one of X¹, X² or
X³ may be N;
R¹ is selected from hydrogen, benzyl or C₁-C₃ alkyl optionally substituted with R^{a};
R² is halogen, CN, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated heterocyclyl optionally substituted with R^{d}, a 3 to 6 membered saturated or unsaturated carbocyclyl optionally substituted with R^{d}, a 9 to 10 membered bicyclic heteroaryl optionally substituted with R^{c}, a 9 to 10 membered bicyclic heterocyclyl optionally substituted with R^{c}, phenylamino, phenoxy optionally substituted with R^{e}, or —NHC(═O)R^{o};
R³ and R⁴ are independently selected from hydrogen, halogen and C₁-C₆ alkyl, or
R³ and R⁴ together form an oxo group;
R⁵ and R⁶ are independently selected from hydrogen, a 3 to 6 membered saturated or unsaturated carbocyclyl, or C₁-C₆ alkyl optionally substituted with R^{f}, or
R⁵ and R⁶ together with the atom to which they are attached form a 3 to 6 membered carbocyclyl or heterocyclyl;
R⁷ and R⁸ are independently selected from hydrogen, halogen or C₁-C₆ alkyl optionally substituted with R^{f}, or
R⁷ and R⁸ together with the atom to which they are attached form a 3 to 6 membered carbocyclyl or heterocyclyl, or
R⁵ and R⁷ together with the atoms to which they are attached form a 3 to 4 membered carbocyclyl or heterocyclyl, wherein only one of the pairs of R⁵ and R⁶, R⁷ and R⁸ or R⁵ and R⁷ may together form a ring;
each R⁹ is independently selected from hydrogen, halogen or methyl;
R¹⁰ and R¹¹ are independently selected from hydrogen and C₁-C₃ alkyl, or
R¹⁰ and R¹¹ together with the atom to which they are attached form a 3 to 6 membered carbocycle or heterocycle;
R¹² and R¹³ are independently selected from hydrogen, C₁-C₆ alkyl and C₃-C₆ carbocyclyl;
each R^{a} is independently selected from OH, OCH₃, halogen, a 5 to 6 membered heteroaryl, and a 3-6 membered heterocyclyl optionally substituted with C₁-C₃ alkyl optionally substituted with oxo;
each R^{b} is independently selected from halogen, CN, OH, OCH₃, cyclopropyl and phenyl optionally substituted with halogen, OH or OCH₃;
each R^{c} is independently selected from halogen, CN, a 3 to 6 membered carbocyclyl, a 5 to 6 membered heteroaryl, a 3 to 6 membered heterocyclyl, phenyl, OR^{g}, SR^{h}, C₁-C₈ alkyl optionally substituted with R^{k}, C₁-C₈ alkynyl optionally substituted with R^{k};
each R^{d} is independently selected from halogen, oxo, C₁-C₆ alkyl, and C₁-C₆ alkoxycarbonyl;
each R^{e} is independently selected from halogen and benzyl;
each R^{f} is independently selected from halogen, oxo, OH, NR^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with halogen, C₁-C₆ alkyl optionally substituted with halogen, —O(C₁-C₆ alkyl) optionally substituted with halogen, phenyl or a 5 to 6 membered heteroaryl;
each R^{g} is independently selected from hydrogen and C₁-C₆ alkyl optionally substituted with halogen or phenyl;
each R^{h} is C₁-C₆ alkyl;
each Rⁱ and R^{j} are independently selected from hydrogen and C₁-C₆ alkyl;
each R^{k} is independently selected from halogen, OH, OCH₃, phenyl and a 3 to 6 membered carbocyclyl;
each R^{m} and Rⁿ are independently selected from hydrogen and C₁-C₆ alkyl; and
R^{o} is C₁-C₆ alkyl, C₃-C₆ cycloalkyl, a 4 to 6 membered heterocyclyl, phenyl or a 5 to 6 membered heteroaryl, wherein the alkyl, cycloalkyl, phenyl and heteroaryl are optionally substituted with halogen, C₁-C₃ alkyl, and C₁-C₃ alkoxyl.

3. A compound of claim 1, wherein:
W is a bond or CR¹⁰R¹¹;
Y is O, S or NR¹;
Z is CR¹²R¹³ or C(═O), provided when Z is C(═O) then Y is NR¹;
X¹ and X² are selected from CR⁹ and N, and X³ is CR⁹, wherein only one of X¹ or X² may be N;
R¹ is C₁-C₃ alkyl optionally substituted with R^{a};
R² is halogen, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated carbocyclyl optionally substituted with R^{d}, a 9 to 10 membered bicyclic heterocyclyl, or —NHC(═O)R^{o};
R³ and R⁴ are independently selected from hydrogen and C₁-C₆ alkyl, or
R³ and R⁴ together form an oxo group;
R⁵ and R⁶ are independently selected from hydrogen or C₁-C₆ alkyl optionally substituted with R^{f}, or
R⁵ and R⁶ together with the atom to which they are attached form a 3 to 6 membered carbocyclyl or heterocyclyl;
R⁷ and R⁸ are independently selected from hydrogen, halogen or C₁-C₆ alkyl optionally substituted with R^{f}, or
R⁷ and R⁸ together with the atom to which they are attached form a 3 to 6 membered heterocyclyl, or
R⁵ and R⁷ together with the atoms to which they are attached form a 3 to 4 membered carbocyclyl or heterocyclyl, wherein only one of the pairs of R⁵ and R⁶, R⁷ and R⁸ or R⁵ and R⁷ may together form a ring;
each R⁹ is hydrogen;
R¹⁹ and R¹¹ are hydrogen;
R¹² and R¹³ are independently selected from hydrogen and C₁-C₆ alkyl;
each R^{a} is halogen;
each R^{b} is independently selected from CN and cyclopropyl;
each R^{c} is independently selected from halogen, CN, OR^{g}, SR^{h}, C₁-C₈ alkyl and a 5 to 6 membered heteroaryl;
each R^{f} is independently selected from halogen, OH, phenyl, a 5 to 6 membered heteroaryl and a 4 to 6 membered heterocyclyl, wherein the phenyl, heteroaryl and heterocyclyl are optionally substituted with halogen, C₁-C₆ alkyl optionally substituted with halogen, or a 5 to 6 membered heteroaryl;
R^{g} is C₁-C₆ alkyl optionally substituted with halogen;
R^{h} is C₁-C₆ alkyl; and
R^{o} is phenyl or a 5 to 6 membered heteroaryl, wherein the phenyl and heteroaryl are optionally substituted with halogen, C₁-C₃ alkyl, and C₁-C₃ alkoxyl.

4. A compound of claim 1, having the structure of Formula I:

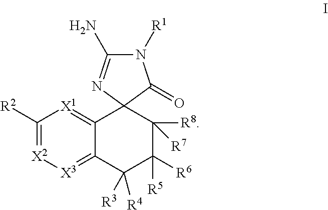

5. A compound of claim 1, having the structure of Formula II:

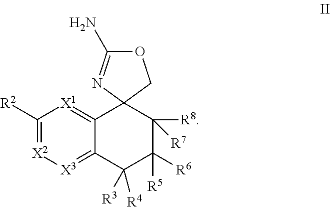

6. A compound of claim 1, having the structure of Formula III:

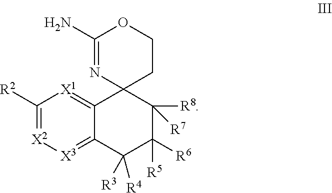

7. A compound of claim 1, having the structure of Formula IV:

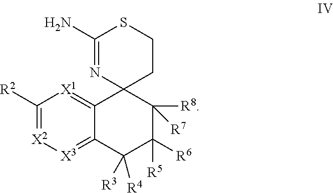

8. A compound of claim 1, having the structure of Formula V:

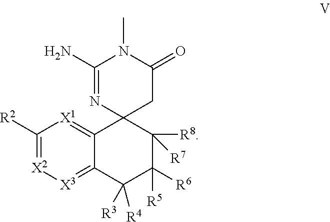

9. A compound of claim 1, having the structure of Formula VI:

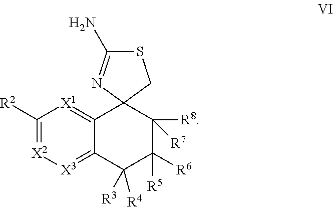

10. A compound of claim 1, wherein X¹, X² and X³ are CR⁹.

11. A compound of claim 1, wherein X¹ is N and X² and X³ are CR⁹.

12. A compound of claim 1, wherein X² is N and X¹ and X³ are CR⁹.

13. A compound of claim 1, wherein each R⁹ is hydrogen.

14. A compound of claim 1, wherein R² is Br, 4-(butanenitrile), isopentyl, cyclopropylvinyl, 3,3-dimethylbut-1-enyl, cyclopropylethynyl, 6-(hex-5-ynenitrile), 3-chlorophenyl, 3-methoxyphenyl, 3-chloro-5-fluorophenyl, 3-(difluoromethoxy)phenyl, 3-cyanophenyl (3-benzonitrile), 3-fluoro-5-methoxyphenyl, 3-chloro-2-fluorophenyl, 3-(trifluoromethoxy)phenyl, 3-methylphenyl, 3-(methylthio)phenyl, 2,5-dichlorophenyl, 5-chloro-2-fluorophenyl, pyridin-3-yl, 5-chloropyridin-3-yl, 5-methoxypyridin-3-yl, 2-fluoropyridin-3-yl, 5-nicotinonitrile (5-cyanopyridin-3-yl), 5-fluoropyridin-3-yl, 5-methylpyridin-3-yl, 5-trifluoromethylpyridin-3-yl, 2-fluoro-5-methylpyridin-3-yl, 2-(5-pyridin-3-yloxy)acetonitrile (5-cyanomethoxypyridin-3-yl), 2-(1H-pyrazol-1-yl)pyridin-3-yl, 4-methoxypyridin-2-yl, 2-isonicotinonitrile (4-cyanopyridin-2-yl), 4-trifluoromethylpyridin-2-yl, 4-methylpyridin-2-yl, 4-chloropyridin-2-yl, pyrimidin-5-yl, cyclohexyl, benzo[d][1,3]dioxol-5-yl, N-5-bromopicolinamide, N-5-chloropicolinamide, N-2-methyloxazole-4-carboxamide, N-2,5-dimethylfuran-3-carboxamide, N-5-methylpyrazine-2-carboxamide, N-pyrazine-2-carboxamide, N-benzamide, N-5-methoxypyrazine-2-carboxamide, N-4-methyloxazole-5-carboxamide and N-pivalamide.

15. A compound of claim 1, wherein R³ and R⁴ are independently selected from hydrogen, F and methyl.

16. A compound of claim 1, wherein R⁵ and R⁶ are independently selected from hydrogen, methyl, CH₂OH, benzyl, 4-bromobenzyl and 4-(pyrimidin-5-yl)benzyl.

17. A compound of claim 1, wherein R⁵ and R⁶ together with the atom to which they are attached form cyclobutyl or tetrahydropyran-4-yl.

18. A compound of claim 1, wherein R⁷ and R⁸ are independently selected from hydrogen, F, methyl, 1-(2,2-difluoroethyl)piperidin-4-yl)methyl, and pyridin-3-ylmethyl.

19. A compound of claim 1, wherein R⁷ and R⁸ together with the atom to which they are attached form tetrahydropyran-4-yl.

20. A compound of claim 1, wherein R¹ is selected from benzyl, methyl, ethyl, —CH₂CH₂OH, —CH₂CH₂CH₂OH, —CH₂CH₂OCH₃, —CH₂CH₂CH₂OCH₃, —CH₂CF₃, pyridin-2-ylmethyl, pyridin-4-ylmethyl and (1-acetylpiperidin-4-yl)methyl.

21. A compound of Formula I as defined in claim 1 and named in any one of Examples 1 to 77 herein, or a stereoisomer, diastereomer, enantiomer, tautomer or pharmaceutically acceptable salt thereof.

22. A compound of Formula α as defined in claim 1 and named in any one of Examples 1 to 129 herein, or a stereoisomer, diastereomer, enantiomer, tautomer or pharmaceutically acceptable salt thereof.

23. A compound of Formula α as defined in claim 1 and named in any one of Examples 78 to 129 herein, or a stereoisomer, diastereomer, enantiomer, tautomer or pharmaceutically acceptable salt thereof.

24. A compound of Formula α as defined in claim 1 and named in any one of Examples 130 to 173 herein, or a stereoisomer, diastereomer, enantiomer, tautomer or pharmaceutically acceptable salt thereof.

25. A compound of Formula α as defined in claim 1 and named in any one of Examples 1 to 173 herein, or a stereoisomer, diastereomer, enantiomer, tautomer or pharmaceutically acceptable salt thereof.

26. A method of inhibiting cleavage of APP by β-secretase in a mammal comprising administering to said mammal an effective amount of a compound of claim 1.

27. A method for treating a disease or condition mediated by the cleavage of APP by β-secretase in a mammal, comprising administering to said mammal an effective amount of a compound of claim 1.

28. The method of claim 27, wherein the disease is Alzheimer's disease.

29. A pharmaceutical compositions comprising a compound of claim 1 and a pharmaceutically acceptable carrier, diluent or excipient.

30-33. (canceled)

34. A process for preparing a compound of Formula I, comprising:
(a) reacting a compound of Formula A: 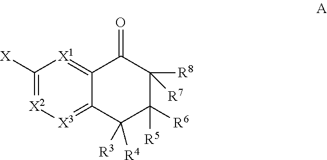
with cyanopotassium and ammonium carbonate to provide a compound of Formula B: 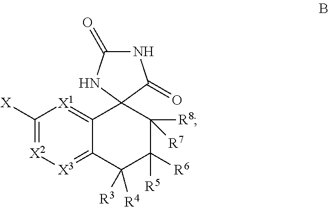
(b) reacting a compound of Formula B with I—R¹ to provide a compound of Formula C: 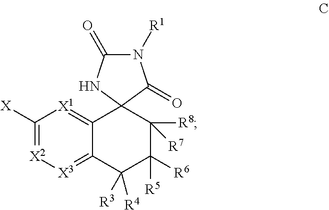
(c) reacting a compound of Formula C with Lawesson's Reagent to provide a compound of Formula D: 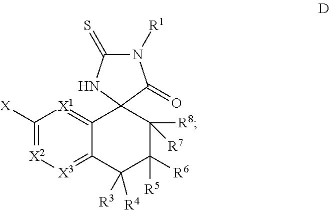
(d) reacting a compound of Formula D with ammonium hydroxide or ammonia in methanol to provide a compound of Formula E (which is a subset of Formula I, wherein R² is halogen): 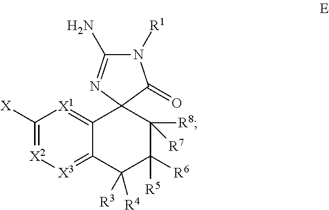
and
(e) optionally performing a Suzuki coupling before or after any of Steps (a) through (d) to convert X to R² (where desired Formula I compound has R² as not halogen) to prepare a compound of Formula I: 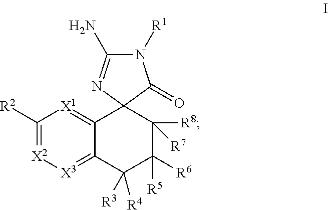
wherein:
X is halogen;
X¹, X² and X³ are independently selected from CR⁹ and N, wherein only one of X¹, X² or X³ may be N;
R¹ is selected from hydrogen, alkyl, aralkyl, heteroaryl or heteroaralkyl;
R² is selected from hydroxy, halogen, amino, cyano, nitro, alkyl, alkoxy, acyl, acyloxy, alkoxycarbonyl, sulfonyl, sulfinyl, sulfanyl, aryloxy, a carbocycle or a heterocycle wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl and optionally substituted carbocycle;
R³ and R⁴ are independently selected from hydrogen, halogen, and alkyl;
R⁵ and R⁶ are independently hydrogen, hydroxy, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, an optionally substituted carbocycle and an optionally substituted heterocycle, or
R⁵ and R⁶ together form a 3 to 6 member carbocycle or heterocycle optionally substituted with hydroxy, halogen, amino, cyano, nitro, alkyl, alkoxy, acyl and haloalkyl;
R⁷ and R⁸ are independently hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl, haloalkyl, a carbocycle and an optionally substituted heterocycle, or
R⁷ and R⁸ together form a 3 to 6 member carbocycle or heterocycle optionally substituted with hydroxy, halogen, amino, cyano, nitro, alkyl, alkoxy, acyl and haloalkyl;
R⁹ is independently is hydrogen, halogen, amino, cyano, nitro, alkyl, alkoxy, acyl, acyloxy, alkoxycarbonyl, sulfonyl, sulfinyl, sulfanyl, a carbocycle or a heterocycle wherein said alkyl, alkoxy, acyl, acyloxy, alkoxycarbonyl, sulfonyl, sulfinyl, sulfanyl, carbocycle and heterocycle are optionally substituted with hydroxy, halogen, amino, cyano, nitro, alkyl, alkoxy, acyl and haloalkyl.

35. The process of claim 34, wherein:
X¹, X² and X³ are independently selected from CR⁹ and N, wherein only one of X¹, X² or X³ may be N;
R¹ is selected from hydrogen, benzyl or C₁-C₃ alkyl optionally substituted with R^{a};
R² is halogen, CN, C₁-C₈ alkyl optionally substituted with R^{b}, C₁-C₈ alkenyl optionally substituted with R^{b}, C₁-C₈ alkynyl optionally substituted with R^{b}, phenyl optionally substituted with R^{c}, a 5 to 6 membered heteroaryl optionally substituted with R^{c}, a 3 to 6 membered saturated or unsaturated heterocyclyl optionally substituted with R^{d}, a 3 to 6 membered saturated or unsaturated carbocyclyl optionally substituted with R^{d}, a 9 to 10 membered bicyclic heteroaryl optionally substituted with R^{c}, a 9 to 10 membered bicyclic heterocyclyl optionally substituted with R^{c}, phenylamino, or phenoxy optionally substituted with R^{e};
R³ and R⁴ are independently selected from hydrogen, halogen and C₁-C₆ alkyl;
R⁵ and R⁶ are independently selected from hydrogen, a 3 to 6 membered saturated or unsaturated carbocyclyl, or C₁-C₆ alkyl optionally substituted with R^{f}, or
R⁵ and R⁶ together with the atom to which they are attached form a 3 to 6 membered carbocyclyl or heterocyclyl;
R⁷ and R⁸ are independently selected from hydrogen, halogen or C₁-C₆ alkyl optionally substituted with R^{f}, or
R⁷ and R⁸ together with the atom to which they are attached form a 3 to 6 membered carbocyclyl or heterocyclyl, wherein only one of the pairs of R⁵ and R⁶ or R⁷ and R⁸ may together form a ring;
each R⁹ is independently selected from hydrogen, halogen or methyl;
each R^{a} is independently selected from OH, OCH₃, halogen, a 5 to 6 membered heteroaryl, and a 3-6 membered heterocyclyl optionally substituted with C₁-C₃ alkyl optionally substituted with oxo;
each R^{b} is independently selected from halogen, CN, OH, OCH₃, cyclopropyl and phenyl optionally substituted with halogen, OH or OCH₃;
each R^{c} is independently selected from halogen, CN, a 3 to 6 membered carbocyclyl, OR^{g}, SR^{h}, NRⁱR^{j}, C₁-C₈ alkyl optionally substituted with R^{k}, C₁-C₈ alkynyl optionally substituted with R^{k};
each R^{d} is independently selected from halogen, oxo, C₁-C₆ alkyl, and C₁-C₆ alkoxycarbonyl;
each R^{e} is independently selected from halogen and benzyl;
each R^{f} is independently selected from halogen, oxo, NR^{m}Rⁿ, —O(C₁-C₆ alkyl) optionally substituted with halogen, phenyl, a 3 to 6 membered carbocyclyl, a 5 to 6 membered heteroaryl, and a 4 to 6 membered heterocyclyl, wherein the phenyl, carbocyclyl, heteroaryl and heterocyclyl are optionally substituted with C₁-C₆ alkyl optionally substituted with halogen and —O(C₁-C₆ alkyl) optionally substituted with halogen;
each R^{g} is independently selected from hydrogen and C₁-C₆ alkyl optionally substituted with halogen or phenyl;
each R^{h} is C₁-C₆ alkyl;
each Rⁱ and R^{j} are independently selected from hydrogen and C₁-C₆ alkyl;
each R^{k} is independently selected from halogen, OH, OCH₃, phenyl and a 3 to 6 membered carbocyclyl; and
each R^{m} and Rⁿ are independently selected from hydrogen and C₁-C₆ alkyl.

36. The process of any one of claim 34 or 35, wherein:
X¹ and X² are selected from CR⁹ and N, and X³ is CR⁹;
R¹ is C₁-C₃ alkyl;
R² is halogen, C₁-C₈ alkyl optionally substituted with R^{b}, C₁-C₈ alkenyl optionally substituted with R^{b}, C₁-C₈ alkynyl optionally substituted with R^{h}, phenyl optionally substituted with R^{c}, a 5 to 6 membered heteroaryl optionally substituted with R^{c}, a 9 to 10 membered bicyclic heterocyclyl;
R³ and R⁴ are independently selected from hydrogen and C₁-C₆ alkyl;
R⁵ and R⁶ are independently selected from hydrogen or C₁-C₆ alkyl, or
R⁵ and R⁶ together with the atom to which they are attached form a 3 to 6 membered carbocyclyl or heterocyclyl;
R⁷ and R⁸ are independently selected from hydrogen or C₁-C₆ alkyl optionally substituted with R^{f}, or
R⁷ and R⁸ together with the atom to which they are attached form a 3 to 6 membered heterocyclyl;
each R^{g} is hydrogen;
each R^{h} is independently selected from CN and cyclopropyl;
each R^{e} is independently selected from halogen, CN, OR, SR^{h}, and C₁-C₈ alkyl;
each R^{f} is independently selected from a 5 to 6 membered heteroaryl and a 4 to 6 membered heterocyclyl optionally substituted with C₁-C₆ alkyl optionally substituted with halogen;
R^{g} is C₁-C₆ alkyl optionally substituted with halogen; and
R^{h} is C₁-C₆ alkyl.
